(19) United States
(12) Patent Application Publication
(10) Pub. No.: US 2024/0301362 A1
(43) Pub. Date: Sep. 12, 2024

(54) ADENOVIRAL SEROTYPE 35 HELPER VECTORS

(71) Applicant: Ensoma, Inc., Boston, MA (US)

(72) Inventor: Soumitra Roy, Townsend, DE (US)

(21) Appl. No.: 18/268,393

(22) PCT Filed: Dec. 22, 2021
(86) PCT No.: PCT/US21/64954

§ 371 Date: Jun. 20, 2023

Related U.S. Application Data
(60) Provisional application No. 63/215,360, filed on Jun. 25, 2021.
(60) Provisional application No. 63/129,346, filed on Dec. 22, 2020.

(51) Int. Cl. C12N 7/00; C12N 15/86

(52) U.S. Cl. CPC C12N 7/00; C12N 15/86; C12N 2710/10021; C12N 2710/10343; C12N 2710/10344; C12N 2710/10352; C12N 2800/40

(57) ABSTRACT

The present disclosure provides, among other things, Ad35 helper genomes and vectors useful in gene therapy, e.g., for production of helper-dependent Ad35 donor vectors. Helper genomes of the present disclosure include a conditionally defective packaging sequence.

### PRIORITY APPLICATIONS

The present application claims the benefit of U.S. Provisional Patent Application No. 63/129,346, filed Dec. 22, 2020 and U.S. Provisional Patent Application No. 63/215,360 filed Jun. 25, 2021, the contents of which are hereby incorporated by reference herein in their entirety.

### BACKGROUND

Many medical conditions are caused by genetic mutation and/or are treatable, at least in part, by gene therapy. Some conditions are particularly treatable by modification of target cells such as hematopoietic stem cells (HSCs). Compositions and methods for gene therapy are therefore needed.

### SUMMARY

Gene therapy can treat many conditions that have a genetic component, including without limitation hemoglobinopathies, immune deficiencies, and cancers. In various gene therapies, hematopoietic stem cells (HSCs) are an important target. However, current methods and compositions for gene therapy, and particularly for modifying HSCs, are limited. For instance, some vectors for gene therapy such as lentiviral vectors have a relatively limited payload capacity. Others, such as adenoviral serotype 5 (Ad5) vectors, are characterized by substantial payload capacity but are sufficiently prevalent such that the majority of humans have antibodies directed against proteins of such vectors, some of which antibodies may be neutralizing. Ad35 is one of the rarest of the 57 known human adenoviral serotypes, with a seroprevalence of <7% and no cross-reactivity with Ad5. Ad35 is less immunogenic than Ad5, which is, in part, due to attenuation of T-cell activation by the Ad35 fiber knob. Further, after intravenous (iv) injection, there is only minimal transduction (e.g., only detectable by PCR) of certain tissues such as the liver in human CD46 transgenic (hCD46tg) mice and non-human primates. First-generation Ad35 vectors have been used clinically for vaccination purposes. The present disclosure provides Ad35 helper genomes and vectors useful in gene therapy, e.g., for production of helper-dependent Ad35 donor vectors.

Ad35 helper-dependent vectors are a type of vector that can be particularly useful for viral gene therapy, e.g., where the vector includes a donor genome that encodes a therapeutic payload for delivery to a recipient. Donor genomes of Ad35 helper-dependent vectors are engineered to remove viral coding sequences that are required for viral propagation and/or contribute to viral propagation, such that the helper-dependent vectors are deficient for propagation in recipients (e.g., human recipients receiving gene therapy including the helper-dependent vector). Because Ad35 helper-dependent donor genomes do not encode proteins used in viral production, they are dependent on other sources of viral proteins (e.g., expression from an Ad35 “helper” genome). For example, for packaging into vector, helper-dependent Ad35 genomes can be delivered to a cell that includes a nucleic acid sequence that provides Ad35 viral proteins in trans. Viral proteins can be provided by an Ad35 helper genome engineered to reduce or eliminate packaging of the helper genome into helper-dependent donor vectors. Packaging of Ad35 helper genome into Ad35 donor vectors risks propagation in the recipient. Ad35 helper vectors must be conditionally competent (i.e., conditionally deficient or conditionally defective) for propagation. One means of achieving conditional propagation deficiency is by engineering of a conditionally defective packaging sequence in the helper genome (e.g., a packaging sequence that can mediate packaging of the helper genome, or mediate packaging of the helper genome more efficiently, in a first state or condition as compared to a second state or condition). The present disclosure includes, among other things, Ad35 helper genomes that include two recombinase sites positioned such that the two recombinase sites flank a packaging sequence, where the two recombinase sites are sites for the same recombinase. Positions of such recombinase sites to produce a conditionally defective packaging sequence in an Ad35 helper vector cannot be predicted from existing knowledge relating to other vectors. To the contrary, relevant sequences of Ad35 are very different from, e.g., corresponding sequences of Ad5 (compare, e.g., the 5′ 600 to 620 nucleotides of Ad35 and Ad5). Moreover, packaging sequences are serotype-specific. The Ad35 packaging sequence includes sequences that correspond to at least Ad5 packaging signal sequences AI, AII, AIII, AIV, and AV, but are unique to Ad35. Accordingly, production of an Ad35 helper vector requires several unpredictable determinations, including (1) identification of the Ad35 packaging sequence to be flanked by recombinase sites (e.g., loxP sites) by inserting or positioning recombinase sites in the subject genome, which is not straightforward where sequence similarity is limited; (2) identification of recombinase site insertions or positions that do not negate propagation of the helper vector (under conditions where the flanked packaging sequence is not excised), which cannot be predicted; and/or (3) identification of spacing between the recombinase sites that permits efficient deletion of the packaging sequence while reducing helper virus packaging during production of HDAd35 donor vectors (e.g., in a cre recombinase-expressing cell line such as the 116 cell line). Thus, the present disclosure includes placement of recombinase sites (e.g., loxP recombinase sites) flanking Ad35 packaging sequences to produce conditionally defective packaging sequences in Ad35 helper genomes. In various embodiments, presence of the conditionally defective packaging sequence in an Ad35 helper genome renders the Ad35 helper genome conditionally defective for propagation, in that excision of the flanked Ad35 packaging sequence by recombination of the recombinase sites renders the Ad35 helper genome defective for packaging.

The present disclosure further includes the recognition that, in various embodiments, packaging sequence inversion can reduce the likelihood of mutations that bypass or disrupt conditionality of propagation and/or packaging. One problem that has characterized various donor vector production systems is that, when a helper genome is present in the same cell or system as a donor genome that includes a wild type or reference packaging sequence, all or a portion of a conditionally defective packaging sequence, or a genome fragment including the same, can be exchanged by homologous recombination with the donor genome for a corresponding fragment of the donor genome that includes the wild type or reference packaging sequence (which can be referred to herein as packaging sequence recombination). When packaging sequence recombination causes a modification of the helper genome that removes at least one of the recombinase sites flanking a packaging sequence of a conditionally defective packaging sequence, the event can be referred to as recombinase site-excising homologous recombination. When recombinase site-excising homologous recombination occurs, conditionality is lost. As a result, helper genomes can be packaged into vectors in the same manner as donor genomes (even in the presence of recombinases that would otherwise render the helper genome defective for packaging), and the production of donor vectors can be contaminated by production of vectors that include helper genomes.

Packaging sequence inversion as provided herein can reduce and/or eliminate recombinase site-excising homologous recombination at least in part by reducing overall homology between helper and donor genomes for any single strand orientation (particularly in packaging sequences and genome fragments including packaging sequences), thereby reducing the potential for packaging sequence recombination. While the present disclosure includes discussion of Ad35 vectors in particular, those of skill in the art will appreciate that packaging sequence inversion will be beneficial for helper genomes of diverse adenoviral serotypes and diverse types of viral vectors.

In at least one aspect, the present disclosure provides a recombinant adenoviral helper genome including: a 5′ Ad35 inverted terminal repeat (ITR); a 3′ Ad35 ITR; and recombinase direct repeats flanking an Ad35 packaging sequence, where the recombinase direct repeats include a first recombinase direct repeat between nucleotide positions corresponding to 136 and 249 of GenBank Accession No. AY128640 and a second recombinase direct repeat between nucleotide positions corresponding to 377 and 504 or 3175 and 3225 of GenBank Accession No. AY128640. In certain embodiments, the first recombinase direct repeat is not at position 206 and/or the second recombinase direct repeat is not at position 481. In certain embodiments, the first recombinase direct repeat is not at position 154 and/or the second recombinase direct repeat is not at position 481. In various embodiments, a recombinant adenoviral helper genome can include a stuffer sequence.

In at least one aspect, the present disclosure provides a recombinant adenoviral helper genome including: a 5′ Ad35 inverted terminal repeat (ITR); a 3′ Ad35 ITR; and recombinase direct repeats flanking an Ad35 packaging sequence, where the recombinase direct repeats include a first recombinase direct repeat between nucleotide positions corresponding to 151-155 (e.g., 151, 152, 153, 154, and/or 155) and 171 (e.g., 151-171, 152-171, 153-171, 154-171, 155-171, 151, 152, 153, 154, 155, or 151-153 to 171), 161 and 181, 185 and 205, or 214 and 234 of GenBank Accession No. AY128640 and a second recombinase direct repeat between nucleotide positions corresponding to 377 and 504 or 3175 and 3225 of GenBank Accession No. AY128640. In certain embodiments, the first recombinase direct repeat is not at position 154 and/or the second recombinase direct repeat is not at position 481. In various embodiments, a recombinant adenoviral helper genome can include a stuffer sequence.

In at least one aspect, the present disclosure provides a recombinant adenoviral helper genome including: a 5′ Ad35 inverted terminal repeat (ITR); a 3′ Ad35 ITR; and recombinase direct repeats flanking an Ad35 packaging sequence, where the recombinase direct repeats include a first recombinase direct repeat between nucleotide positions corresponding to 151-155 (e.g., 151, 152, 153, 154, and/or 155) and 171, 161 and 181, 185 and 205, or 214 and 234 of GenBank Accession No. AY128640 and a second recombinase direct repeat between nucleotide positions corresponding to 377 and 504 or 3175 and 3225 of GenBank Accession No. AY128640. In certain embodiments, the first recombinase direct repeat is not at position 154 and/or the second recombinase direct repeat is not at position 481. In various embodiments, a recombinant adenoviral helper genome can include a stuffer sequence.

In various embodiments, the recombinase direct repeats include a first recombinase direct repeat between nucleotide positions corresponding to 151-155 (e.g., 151, 152, 153, 154, and/or 155) and 171, 161 and 181, 185 and 205, or 214 and 234 of GenBank Accession No. AY128640 and a second recombinase direct repeat between nucleotide positions corresponding to 392 and 412 or 469 and 489 of GenBank Accession No. AY128640.

In various embodiments, the recombinase direct repeats include a first recombinase direct repeat between nucleotide positions corresponding to 151-155 (e.g., 151, 152, 153, 154, and/or 155) and 171, 161 and 181, 185 and 205, or 214 and 234 of GenBank Accession No. AY128640 and a second recombinase direct repeat between nucleotide positions corresponding to 3190 and 3210 of GenBank Accession No. AY128640. In various embodiments, the recombinase direct repeats include a first recombinase direct repeat between nucleotide positions corresponding to 151-155 (e.g., 151, 152, 153, 154, and/or 155) and 171 of GenBank Accession No. AY128640 and a second recombinase direct repeat between nucleotide positions corresponding to 3190 and 3210 of GenBank Accession No. AY128640. In various embodiments, the recombinase direct repeats include a first recombinase direct repeat between nucleotide positions corresponding to 161 and 181 of GenBank Accession No. AY128640 and a second recombinase direct repeat between nucleotide positions corresponding to 392 and 412 of GenBank Accession No. AY128640. In various embodiments, the recombinase direct repeats include a first recombinase direct repeat between nucleotide positions corresponding to 185 and 205 of GenBank Accession No. AY128640 and a second recombinase direct repeat between nucleotide positions corresponding to 469 and 489 of GenBank Accession No. AY128640. In various embodiments, the recombinase direct repeats include a first recombinase direct repeat between nucleotide positions corresponding to 214 and 234 of GenBank Accession No. AY128640 and a second recombinase direct repeat between nucleotide positions corresponding to 392 and 412 of GenBank Accession No. AY128640. In various embodiments, the recombinase direct repeats include a first recombinase direct repeat at a nucleotide position corresponding to 161, 171, 195, or 224 of GenBank Accession No. AY128640 and a second recombinase direct repeat at a nucleotide position corresponding to 402, 479, or 3200 of GenBank Accession No. AY128640. In various embodiments, the recombinase direct repeats include a first recombinase direct repeat at a nucleotide position corresponding to 161 of GenBank Accession No. AY128640 and a second recombinase direct repeat at a nucleotide position corresponding to 3200 of GenBank Accession No. AY128640. In various embodiments, the recombinase direct repeats include a first recombinase direct repeat at a nucleotide position corresponding to 171 of GenBank Accession No. AY128640 and a second recombinase direct repeat at a nucleotide position corresponding to 402 of GenBank Accession No. AY128640. In various embodiments, the recombinase direct repeats include a first recombinase direct repeat at a nucleotide position corresponding to 195 of GenBank Accession No. AY128640 and a second recombinase direct repeat at a nucleotide position corresponding to 479 of GenBank Accession No. AY128640. In various embodiments, the recombinase direct repeats include a first recombinase direct repeat at a nucleotide position corresponding to 224 of GenBank Accession No. AY128640 and a second recombinase direct repeat at a nucleotide position corresponding to 402 of GenBank Accession No. AY128640.

In various embodiments, the recombinase direct repeats that flank the Ad35 packaging sequence are FRT, loxP, rox, vox, AttB, or AttP sites. In various embodiments, the recombinase direct repeats that flank the Ad35 packaging sequence are loxP sites.

The present disclosure further provides a recombinant adenoviral helper vector including the Ad35 helper genome of the present disclosure. The present disclosure further provides a recombinant adenoviral vector production system including: (i) the Ad35 helper genome or the helper vector of the present disclosure, and (ii) an HDAd35 donor genome, the HDAd35 donor genome including: a 5′ Ad35 inverted terminal repeat (ITR); a 3′ Ad35 ITR; an Ad35 packaging sequence; and a nucleic acid sequence encoding at least one heterologous expression product. The present disclosure further provides a method of producing a recombinant helper-dependent adenoviral donor vector, the method including isolating the recombinant helper-dependent Ad35 donor vector from a culture of cells, where the cells include: a recombinant Ad35 helper genome or a recombinant adenoviral helper vector of the present disclosure; and a recombinant helper-dependent Ad35 donor genome including: a 5′ Ad35 inverted terminal repeat (ITR); a 3′ Ad35 ITR; an Ad35 packaging sequence; and a nucleic acid sequence encoding at least one heterologous expression product.

In various embodiments, a helper genome includes a nucleic acid sequence that encodes an Ad35 fiber knob. In various embodiments, the Ad35 fiber knob includes a mutation that increases affinity with CD46. In various embodiments, the Ad35 fiber knob includes one or more mutations: selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or including each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

In various embodiments, the helper genome is present in a cell that includes a nucleic acid encoding a recombinase for recombination of the direct repeats. In various embodiments, the recombinase is a Flp, Cre, Dre, Vika, or PhiC31 recombinase. In various embodiments, the cell is a HEK293 cell, optionally where the cell is a HEK293 cell that encodes or expresses Cre recombinase, optionally where the HEK293 cell that encodes or expresses Cre recombinase is a 116 cell.

In various embodiments, the Ad35 helper genome includes an inverted packaging sequence.

In at least one aspect, the present disclosure provides a recombinant adenoviral helper genome including: a 5′ Ad35 inverted terminal repeat (ITR); a 3′ Ad35 ITR; and recombinase direct repeats flanking an Ad35 packaging sequence, where the recombinase direct repeats include a first recombinase direct repeat between nucleotide positions corresponding to 136 and 249 of GenBank Accession No. AY128640 and a second recombinase direct repeat between nucleotide positions corresponding to 377 and 504 or 3175 and 3225 of GenBank Accession No. AY128640, where the Ad35 helper genome includes an inverted packaging sequence. In various embodiments, the recombinase direct repeats include a first recombinase direct repeat between nucleotide positions corresponding to 151 and 171, 161 and 181, 185 and 205, or 214 and 234 of GenBank Accession No. AY128640 and a second recombinase direct repeat between nucleotide positions corresponding to 392 and 412 or 469 and 489 of GenBank Accession No. AY128640. In various embodiments, the recombinase direct repeats include a first recombinase direct repeat between nucleotide positions corresponding to 151 and 171, 161 and 181, 185 and 205, or 214 and 234 of GenBank Accession No. AY128640 and a second recombinase direct repeat between nucleotide positions corresponding to 3190 and 3210 of GenBank Accession No. AY128640. In various embodiments, the recombinase direct repeats include a first recombinase direct repeat between nucleotide positions corresponding to 151 and 171 of GenBank Accession No. AY128640 and a second recombinase direct repeat between nucleotide positions corresponding to 3190 and 3210 of GenBank Accession No. AY128640. In various embodiments, a recombinant adenoviral helper genome can include a stuffer sequence.

In various embodiments, the recombinase direct repeats that flank the Ad35 packaging sequence are FRT, loxP, rox, vox, AttB, or AttP sites. In various embodiments, the recombinase direct repeats that flank the Ad35 packaging sequence are loxP sites. The present disclosure further provides a recombinant adenoviral helper vector including the Ad35 helper genome of the present disclosure. The present disclosure further provides a recombinant adenoviral vector production system including: (i) an Ad35 helper genome or helper vector of the present disclosure, and (ii) an HDAd35 donor genome, the HDAd35 donor genome including: a 5′ Ad35 inverted terminal repeat (ITR); a 3′ Ad35 ITR; an Ad35 packaging sequence; and a nucleic acid sequence encoding at least one heterologous expression product. The present disclosure further provides a method of producing a recombinant helper-dependent adenoviral donor vector, the method including isolating the recombinant helper-dependent Ad35 donor vector from a culture of cells, where the cells include: a recombinant Ad35 helper genome or a recombinant adenoviral helper vector of the present disclosure; and a recombinant helper-dependent Ad35 donor genome including: a 5′ Ad35 inverted terminal repeat (ITR); a 3′ Ad35 ITR; an Ad35 packaging sequence; and a nucleic acid sequence encoding at least one heterologous expression product. In various embodiments, the helper genome includes a nucleic acid sequence that encodes an Ad35 fiber knob. In various embodiments, the Ad35 fiber knob includes a mutation that increases affinity with CD46.

In various embodiments, the Ad35 fiber knob includes one or more mutations: selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or including each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

In various embodiments, the helper genome is present in a cell that includes a nucleic acid encoding a recombinase for recombination of the direct repeats. In various embodiments, the recombinase is a Flp, Cre, Dre, Vika, or PhiC31 recombinase. In various embodiments, the cell is a HEK293 cell, optionally where the cell is a HEK293 cell that encodes or expresses Cre recombinase, optionally where the HEK293 cell that encodes or expresses Cre recombinase is a 116 cell.

In various embodiments, the inverted packaging sequence includes the Ad35 packaging sequence and one or both of the first recombinase direct repeat and the second recombinase direct repeat. In various embodiments, the inverted packaging sequence includes, or includes a first end point at, a nucleotide position between nucleotide positions corresponding to 119 and 169 of AY128640 or 134 and 154 of AY128640. In various embodiments, the inverted packaging sequence includes, or includes a first end point at, a nucleotide position corresponding to position 144 of AY128640. In various embodiments, the inverted packaging sequence includes, or includes a second end point at, a nucleotide position between nucleotide positions corresponding to 3175 and 3225 of AY128640 or 3190 and 3210 of AY128640. In various embodiments, the inverted packaging sequence includes, or includes a second end point at, a nucleotide position corresponding to position 3200 of AY128640. In various embodiments, the inverted packaging sequence includes, or includes a second end point at, a nucleotide position between nucleotide positions corresponding to 455 and 505 of AY128640 or 470 and 490 of AY128640. In various embodiments, the inverted packaging sequence includes, or includes a second end point at, a nucleotide position corresponding to position 480 of AY128640.

In at least one aspect, the present disclosure provides a recombinant recombinase site-flanked adenoviral serotype 35 (Ad35) packaging sequence, where recombinase direct repeats flank an Ad35 packaging sequence, where the Ad35 packaging sequence corresponds to a fragment of GenBank Accession No. AY128640 having a first end point between nucleotide positions corresponding to 136 and 249 of GenBank Accession No. AY128640 and a second end point between nucleotide positions corresponding to 377 and 504 or 3175 and 3225 of GenBank Accession No. AY128640. In various embodiments, the first end point is between nucleotide positions corresponding to 151-155 (e.g., 151, 152, 153, 154, and/or 155) and 171, 161 and 181, 185 and 205, or 214 and 234 of GenBank Accession No. AY128640 and the second end point is between nucleotide positions corresponding to 392 and 412 or 469 and 489 of GenBank Accession No. AY128640. In various embodiments, the first end point is between nucleotide positions corresponding to 151-155 (e.g., 151, 152, 153, 154, and/or 155) and 171, 161 and 181, 185 and 205, or 214 and 234 of GenBank Accession No. AY128640 and the second end point is between nucleotide positions corresponding to 3190 and 3210 of GenBank Accession No. AY128640. In various embodiments, the first end point is between nucleotide positions corresponding to 151-155 (e.g., 151, 152, 153, 154, and/or 155) and 171 of GenBank Accession No. AY128640 and the second end point is between nucleotide positions corresponding to 3190 and 3210 of GenBank Accession No. AY128640. In various embodiments, the first end point is between nucleotide positions corresponding to 161 and 181 of GenBank Accession No. AY128640 and the second end point is between nucleotide positions corresponding to 392 and 412 of GenBank Accession No. AY128640. In various embodiments, the first end point is between nucleotide positions corresponding to 185 and 205 of GenBank Accession No. AY128640 and the second end point is between nucleotide positions corresponding to 469 and 489 of GenBank Accession No. AY128640. In various embodiments, the first end point is between nucleotide positions corresponding to 214 and 234 of GenBank Accession No. AY128640 and the second end point is between nucleotide positions corresponding to 392 and 412 of GenBank Accession No. AY128640. In various embodiments, the first end point is at a nucleotide position corresponding to 161, 162, 171, 172, 195, 196, 224, or 225 of GenBank Accession No. AY128640 and the second end point is at a nucleotide position corresponding to 402, 479, or 3200 of GenBank Accession No. AY128640. In various embodiments, the first end point is at a nucleotide position corresponding to 161 or 162 of GenBank Accession No. AY128640 and the second end point is at a nucleotide position corresponding to 3200 of GenBank Accession No. AY128640. The recombinant packaging sequence of claim 44, where the first end point is at a nucleotide position corresponding to 171 or 172 of GenBank Accession No. AY128640 and the second end point is at a nucleotide position corresponding to 402 of GenBank Accession No. AY128640. In various embodiments, the first end point is at a nucleotide position corresponding to 195 or 196 of GenBank Accession No. AY128640 and the second end point is at a nucleotide position corresponding to 479 of GenBank Accession No. AY128640. In various embodiments, the first end point is at a nucleotide position corresponding to 224 or 225 of GenBank Accession No. AY128640 and the second end point is at a nucleotide position corresponding to 402 of GenBank Accession No. AY128640. In various embodiments, the packaging sequence is present in an adenoviral genome and is inverted, optionally where the packaging sequence is inverted as compared to a 5′ ITR of the adenoviral genome.

In at least one aspect, the present disclosure provides a recombinant adenoviral helper genome including: a 5′ Ad35 inverted terminal repeat (ITR); a 3′ Ad35 ITR; and an inverted sequence including an Ad35 packaging sequence, where the inverted sequence includes, or includes a first end point at, a nucleotide position between nucleotide positions corresponding to 119 and 169 of AY128640 or 134 and 154 of AY128640, optionally where the inverted sequence includes, or includes a first end point at, a nucleotide position corresponding to position 144 of AY128640, and where (i) the inverted sequence includes, or includes a second end point at, a nucleotide position between nucleotide positions corresponding to 3175 and 3225 of AY128640 or 3190 and 3210 of AY128640, optionally where the inverted sequence includes, or includes a second end point at, a nucleotide position corresponding to position 3200 of AY128640, or (ii) the inverted sequence includes, or includes a second end point at, a nucleotide position between nucleotide positions corresponding to 455 and 505 of AY128640 or 470 and 490 of AY128640, optionally where the inverted sequence includes, or includes a second end point at, a nucleotide position corresponding to position 480 of AY128640. In various embodiments, recombinase direct repeats flank the Ad35 packaging sequence. In various embodiments, a recombinant adenoviral helper genome can include a stuffer sequence.

In various embodiments, the recombinant adenoviral vector production system can include a stuffer sequence. In any of the various embodiments provided herein, a helper vector can include a deletion of nucleotides corresponding to nucleotide positions 480-3199, 481-3199, or 482-3199 of AY128640, optionally wherein the deletion can be or can be referred to as an E1 deletion.

### Definitions

A, An, The: As used herein, “a”, “an”, and “the” refer to one or to more than one (i.e., to at least one) of the grammatical object of the article. By way of example, “an element” discloses embodiments of exactly one element and embodiments including more than one element.

About: As used herein, term “about”, when used in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referenced value.

Administration: As used herein, the term “administration” typically refers to administration of a composition to a subject or system to achieve delivery of an agent that is, or is included in, the composition.

Affinity: As used herein, “affinity” refers to the strength of the sum total of non-covalent interactions between a particular binding agent (e.g., a viral vector), and/or a binding moiety thereof, with a binding target (e.g., a cell). Unless indicated otherwise, as used herein, “binding affinity” refers to a 1:1 interaction between a binding agent and a binding target thereof (e.g., a viral vector with a target cell of the viral vector). Those of skill in the art appreciate that a change in affinity can be described by comparison to a reference (e.g., increased or decreased relative to a reference), or can be described numerically. Affinity can be measured and/or expressed in a number of ways known in the art, including, but not limited to, equilibrium dissociation constant (K_{D}) and/or equilibrium association constant (K_{A}). K_{D} is the quotient of k_{off}/kₒₙ, whereas K_{A} is the quotient of kₒₙ/k_{off}, where kₒₙ refers to the association rate constant of, e.g., viral vector with target cell, and k_{off} refers to the dissociation of, e.g., viral vector from target cell. The kₒₙ and k_{off} can be determined by techniques known to those of skill in the art.

Agent: As used herein, the term “agent” may refer to any chemical entity, including without limitation any of one or more of an atom, molecule, compound, amino acid, polypeptide, nucleotide, nucleic acid, protein, protein complex, liquid, solution, saccharide, polysaccharide, lipid, or combination or complex thereof.

Between or From: As used herein, the term “between” refers to content that falls between indicated upper and lower, or first and second, boundaries, inclusive of the boundaries. Thus, for the avoidance of doubt, the term “between” includes values that are exactly the provided upper or lower, or first or second, bound, as well as all values within the provided range. Similarly, the term “from”, when used in the context of a range of values, indicates that the range includes content that falls between indicated upper and lower, or first and second, boundaries, inclusive of the boundaries.

Binding: As used herein, the term “binding” refers to a non-covalent association between or among two or more agents. “Direct” binding involves physical contact between agents: indirect binding involves physical interaction by way of physical contact with one or more intermediate agents. Binding between two or more agents can occur and/or be assessed in any of a variety of contexts, including where interacting agents are studied in isolation or in the context of more complex systems (e.g., while covalently or otherwise associated with a carrier agents and/or in a biological system or cell).

Cancer: As used herein, the term “cancer” refers to a condition, disorder, or disease in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer can include one or more tumors. In some embodiments, a cancer can be or include cells that are precancerous (e.g., benign), malignant, pre-metastatic, metastatic, and/or non-metastatic. In some embodiments, a cancer can be or include a solid tumor. In some embodiments, a cancer can be or include a hematologic tumor.

Control expression or activity: As used herein, a first element (e.g., a protein, such as a transcription factor, or a nucleic acid sequence, such as promoter) “controls” or “drives” expression or activity of a second element (e.g., a protein or a nucleic acid encoding an agent such as a protein) if the expression or activity of the second element is wholly or partially dependent upon status (e.g., presence, absence, conformation, chemical modification, interaction, or other activity) of the first under at least one set of conditions. Control of expression or activity can be substantial control or activity, e.g., in that a change in status of the first element can, under at least one set of conditions, result in a change in expression or activity of the second element of at least 10% (e.g., at least 20%, 30%, 40%, 50%, 60%, 70%, 80%, 90%, 100%, 2-fold, 3-fold, 4-fold, 5-fold, 10-fold, 20-fold, 30-fold, 40-fold, 50-fold, 100-fold) as compared to a reference control.

Corresponding to: As used herein, the term “corresponding to” may be used to designate the position and/or identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of skill in the art appreciate that residues in a provided polypeptide or polynucleotide sequence are often designated (e.g., numbered or labeled) according to the scheme of a related reference sequence (even if, e.g., such designation does not reflect literal numbering of the provided sequence). By way of illustration, if a reference sequence includes a particular amino acid motif at positions 100-110, and a second related sequence includes the same motif at positions 110-120, the motif positions of the second related sequence can be said to “correspond to” positions 100-110 of the reference sequence. Accordingly, a provided amino acid or nucleic acid sequence can have, for example, added, removed, inserted, or deleted positions or units that differ from a reference sequence but do not limit the designation of other positions or units as corresponding to the reference. In nucleic acid sequences, for example, exemplary additions or insertions can include restriction enzyme site nucleotides or recombinase site nucleotides. Those of skill in the art appreciate that corresponding positions can be readily identified, e.g., by alignment of sequences, and that such alignment is commonly accomplished by any of a variety of known tools, strategies, and/or algorithms, including without limitation software programs such as, for example, BLAST, CS-BLAST, CUDASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE. Two sequences can be identified as corresponding if they are identical or if they share substantial identity, e.g., at least 80%, 85%, 90%, 91%, 92%, 93%, 94%, 95%, 96%, 97%, 98%, or 99% identity. In various embodiments, a nucleic acid sequence can correspond to a sequence that is identical or substantially identical (e.g., at least 80%, 85%, 90%, 91%, 92%, 93%, 94%, 95%, 96%, 97%, 98%, or 99% identical) to the complement of the nucleic acid sequence.

Downstream and Upstream: As used herein, the term “downstream” means that a first DNA region is closer, relative to a second DNA region, to the C-terminus of a nucleic acid that includes the first DNA region and the second DNA region. As used herein, the term “upstream” means a first DNA region is closer, relative to a second DNA region, to the N-terminus of a nucleic acid that includes the first DNA region and the second DNA region.

Effective amount: An “effective amount” is the amount of a formulation necessary to result in a desired physiological change in a subject. Effective amounts are often administered for research purposes.

Engineered: As used herein, the terms “engineered” and “recombinant” are used interchangeably herein to refer to compositions having been manipulated by the hand of man. For example, a polynucleotide is considered to be “engineered” when two or more sequences, that are not linked together in that order in nature, are manipulated by the hand of man to be directly linked to one another in the engineered polynucleotide. Those of skill in the art will appreciate that an “engineered” nucleic acid or amino acid sequence can be a recombinant nucleic acid or amino acid sequence, and can be referred to as “genetically engineered.” In some embodiments, an engineered polynucleotide includes a coding sequence and/or a regulatory sequence that is found in nature operably linked with a first sequence but is not found in nature operably linked with a second sequence, which is in the engineered polynucleotide operably linked in with the second sequence by the hand of man. In some embodiments, a cell or organism is considered to be “engineered” or “genetically engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution, deletion, or mating). As is common practice and is understood by those of skill in the art, progeny or copies, perfect or imperfect, of an engineered polynucleotide or cell are typically still referred to as “engineered” even though the direct manipulation was of a prior entity.

Expression: As used herein, “expression” refers individually and/or cumulatively to one or more biological process that result in production from a nucleic acid sequence of an encoded agent, such as a protein. Expression specifically includes either or both of transcription and translation.

Flank: As used herein, a first element (e.g., a nucleic acid sequence or amino acid sequence) present in a contiguous sequence with a second element and a third element is “flanked” by the second element and third element if it is positioned in the contiguous sequence between the second element and the third element. Accordingly, in such arrangement, the second element and third element can be referred to as “flanking” the first element. Flanking elements can be immediately adjacent to a flanked element or separated from the flanked element by one or more relevant units. In various examples in which the contiguous sequence is a nucleic acid or amino acid sequence, and the relevant units are bases or amino acid residues, respectively, the number of units in the contiguous sequence that are between a flanked element and, independently, first and/or second flanking elements can be, e.g., 50 units or less, e.g., no more than 50, 45, 40, 35, 30, 25, 20, 15, 10, 5, 4, 3, 2, 1, or 0 units.

Fragment: As used herein, “fragment” refers a structure that includes and/or consists of a discrete portion of a reference agent (sometimes referred to as the “parent” agent). In some embodiments, a fragment lacks one or more moieties found in the reference agent. In some embodiments, a fragment includes or consists of one or more moieties found in the reference agent. In some embodiments, the reference agent is a polymer such as a polynucleotide or polypeptide. In some embodiments, a fragment of a polymer includes or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) of the reference polymer. In some embodiments, a fragment is a sequence having a number of units having a lower bound selected from 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300 monomeric units and an upper bound selected from 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units. In some embodiments, a fragment of a polymer includes or consists of at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the reference polymer. A fragment of a reference polymer is not necessarily identical to a corresponding portion of the reference polymer. For example, a fragment of a reference polymer can be a polymer having a sequence of residues having at least 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more identity to the reference polymer. A fragment may, or may not, be generated by physical fragmentation of a reference agent. In some instances, a fragment is generated by physical fragmentation of a reference agent. In some instances, a fragment is not generated by physical fragmentation of a reference agent and can be instead, for example, produced by de novo synthesis or other means.

Gene, Transgene: As used herein, the term “gene” refers to a DNA sequence that is or includes coding sequence (i.e., a DNA sequence that encodes an expression product, such as an RNA product and/or a polypeptide product), optionally together with some or all of regulatory sequences that control expression of the coding sequence. In some embodiments, a gene includes non-coding sequence such as, without limitation, introns. In some embodiments, a gene may include both coding (e.g., exonic) and non-coding (e.g., intronic) sequences. In some embodiments, a gene includes a regulatory sequence that is a promoter. In some embodiments, a gene includes one or both of a (i) DNA nucleotides extending a predetermined number of nucleotides upstream of the coding sequence in a reference context, such as a source genome, and (ii) DNA nucleotides extending a predetermined number of nucleotides downstream of the coding sequence in a reference context, such as a source genome. In various embodiments, the predetermined number of nucleotides can be 500 bp, 1 kb, 2 kb, 3 kb, 4 kb, 5 kb, 10 kb, 20 kb, 30 kb, 40 kb, 50 kb, 75 kb, or 100 kb. As used herein, a “transgene” refers to a gene that is not endogenous or native to a reference context in which the gene is present or into which the gene may be placed by engineering.

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Host cell, target cell: As used herein, “host cell” refers to a cell into which exogenous DNA (recombinant or otherwise), such as a transgene, has been introduced. Those of skill in the art appreciate that a “host cell” can be the cell into which the exogenous DNA was initially introduced and/or progeny or copies, perfect or imperfect, thereof. In some embodiments, a host cell includes one or more viral genes or transgenes. In some embodiments, an intended or potential host cell can be referred to as a target cell.

In various embodiments, a host cell or target cell is identified by the presence, absence, or expression level of various surface markers.

A statement that a cell or population of cells is “positive” for or expressing a particular marker refers to the detectable presence on or in the cell of the particular marker. When referring to a surface marker, the term can refer to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

A statement that a cell or population of cells is “negative” for a particular marker or lacks expression of a marker refers to the absence of substantial detectable presence on or in the cell of a particular marker. When referring to a surface marker, the term can refer to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

Identity: As used herein, the term “identity” refers to the overall relatedness between polymeric molecules, e.g., between nucleic acid molecules (e.g., DNA molecules and/or RNA molecules) and/or between polypeptide molecules. Methods for the calculation of a percent identity as between two provided sequences are known in the art. The term “% sequence identity” refers to a relationship between two or more sequences, as determined by comparing the sequences. In the art, “identity” also means the degree of sequence relatedness between protein and nucleic acid sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. For instance, calculation of the percent identity of two nucleic acid or polypeptide sequences, for example, can be performed by aligning the two sequences (or the complement of one or both sequences) for optimal comparison purposes (e.g., gaps can be introduced in one or both of a first and a second sequences for optimal alignment and non-identical sequences can be disregarded for comparison purposes). The nucleotides or amino acids at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, optionally accounting for the number of gaps, and the length of each gap, which may need to be introduced for optimal alignment of the two sequences. The comparison of sequences and determination of percent identity between two sequences can be accomplished using a computational algorithm, such as BLAST (basic local alignment search tool). Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. “Default values” will mean any set of values or parameters, which originally load with the software when first initialized.

“Improve,” “increase,” “inhibit,” or “reduce”: As used herein, the terms “improve”, “increase”, “inhibit”, and “reduce”, and grammatical equivalents thereof, indicate qualitative or quantitative difference from a reference.

Isolated: As used herein, “isolated” refers to a substance and/or entity that has been (1) separated from at least some of the components with which it was associated when initially produced (whether in nature and/or in an experimental setting), and/or (2) designed, produced, prepared, and/or manufactured by the hand of man. Isolated substances and/or entities may be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated. In some embodiments, isolated agents are 80%, 85%, 90%, 91%, 92%, 93%, 94%, 95%, 96%, 97%, 98%, 99%, or more than 99% pure. As used herein, a substance is “pure” if it is substantially free of other components. In some embodiments, as will be understood by those skilled in the art, a substance may still be considered “isolated” or even “pure”, after having been combined with certain other components such as, for example, one or more carriers or excipients (e.g., buffer, solvent, water, etc.); in such embodiments, percent isolation or purity of the substance is calculated without including such carriers or excipients. To give but one example, in some embodiments, a biological polymer such as a polypeptide or polynucleotide that occurs in nature is considered to be “isolated” when, a) by virtue of its origin or source of derivation is not associated with some or all of the components that accompany it in its native state in nature; b) it is substantially free of other polypeptides or nucleic acids of the same species from the species that produces it in nature; c) is expressed by or is otherwise in association with components from a cell or other expression system that is not of the species that produces it in nature. Thus, for instance, in some embodiments, a polypeptide that is chemically synthesized or is synthesized in a cellular system different from that which produces it in nature is considered to be an “isolated” polypeptide. Alternatively or additionally, in some embodiments, a polypeptide that has been subjected to one or more purification techniques may be considered to be an “isolated” polypeptide to the extent that it has been separated from other components a) with which it is associated in nature; and/or b) with which it was associated when initially produced.

Reference: As used herein, “reference” refers to a standard or control relative to which a comparison is performed. For example, in some embodiments, an agent, sample, sequence, subject, animal, or individual, or population thereof, or a measure or characteristic representative thereof, is compared with a reference, an agent, sample, sequence, subject, animal, or individual, or population thereof, or a measure or characteristic representative thereof. In some embodiments, a reference is a measured value. In some embodiments, a reference is an established standard or expected value. In some embodiments, a reference is a historical reference. A reference can be quantitative of qualitative. Typically, as would be understood by those of skill in the art, a reference and the value to which it is compared represent comparable conditions. Those of skill in the art will appreciate when sufficient similarities are present to justify reliance on and/or comparison. In some embodiments, an appropriate reference may be an agent, sample, sequence, subject, animal, or individual, or population thereof, under conditions those of skill in the art will recognize as comparable, e.g., for the purpose of assessing one or more particular variables (e.g., presence or absence of an agent or condition), or a measure or characteristic representative thereof. Without wishing to be bound by any particular embodiment(s), in various embodiments a reference sequence can be a sequence associated with a sequence accession number provided herein, certain of which sequences associated with sequence accession numbers are provided in FIG. 17.

Subject: As used herein, the term “subject” refers to an organism, typically a mammal (e.g., a human, rat, or mouse). In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject is not suffering from a disease, disorder or condition. In some embodiments, a subject does not display any symptom or characteristic of a disease, disorder, or condition. In some embodiments, a subject has one or more features characteristic of susceptibility to or risk of a disease, disorder, or condition. In some embodiments, a subject is a subject that has been tested for a disease, disorder, or condition, and/or to whom therapy has been administered. In some instances, a subject to which an agent is administered can be interchangeably referred to as a “recipient.” In some instances, a human subject can be interchangeably referred to as a “patient” or “individual.”

Treatment: As used herein, the term “treatment” (also “treat” or “treating”) refers to administration of a therapy that partially or completely alleviates, ameliorates, relieves, inhibits, delays onset of, reduces severity of, and/or reduces incidence of one or more symptoms, features, and/or causes of a particular disease, disorder, or condition, or is administered for the purpose of achieving any such result. In some embodiments, such treatment can be of a subject who does not exhibit signs of the relevant disease, disorder, or condition and/or of a subject who exhibits only early signs of the disease, disorder, or condition. Alternatively or additionally, such treatment can be of a subject who exhibits one or more established signs of the relevant disease, disorder and/or condition. In some embodiments, treatment can be of a subject who has been diagnosed as suffering from the relevant disease, disorder, and/or condition. In some embodiments, treatment can be of a subject known to have one or more susceptibility factors that are statistically correlated with increased risk of development of the relevant disease, disorder, or condition. A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. A “therapeutic treatment” includes a treatment administered to a subject who displays symptoms or signs of a condition and is administered to the subject for the purpose of reducing the severity or progression of the condition.

### BRIEF DESCRIPTION OF THE DRAWINGS

FIG. 1 is a schematic showing alignment of the ‘left end’ sequences of wild type sequences of Ad3 (GenBank accession number NC_011203) and Ad35 (GenBank accession number AY128640) (the ‘left end’ being defined by the conventional representation of adenoviral maps, where the major late promoter transcribes the ‘top strand’). Alignment was used to identify putative packaging signals of Ad35 (boxed). Packaging signals A1, A2, A5, and A6 were identified in accordance with terminology set forth in Ostapchuk and Hearing, J Virol. 2001 75:45-51. The table shown in FIG. 1 provides four exemplary positions for placement of a 5′ loxP site, four exemplary positions for placement of a 3′ loxP site, and four exemplary pairings of a position for placement of a 5′ loxP site and a position for placement of a 3′ loxP site. LoxP sites were inserted in the Ad35 genome to the left of the packaging signal A1 at one of four positions indicated by black arrowheads (i.e., after nucleotide numbers 161, 171, 195, or 224) in combination with a loxP sequence inserted to the right of the packaging signal A6, e.g., at positions indicated by open arrowheads (shown after nucleotide numbers 402 or 479). The exemplified combinations are further described in Example 1. Because adenoviral sequences of the Examples were deleted between base pairs 480, 481, or 482 to 3199 to derive E1-deleted replication-incompetent vectors, one loxP insertion was placed at position 3200, but is not as distant from the other insertion sites as the numbering would suggest.

FIG. 2A is a schematic showing the ‘left end’ sequence of an Ad35 helper genome which corresponds to that shown in FIG. 1 (see also GenBank accession number AY128640) and includes (i) six nucleotides added to produce an FseI restriction site between Ad35 positions 143 and 144, (ii) loxP sites added after positions 224 and 402, and (iii) I-SceI and FseI sites added after position 480. Certain added sequences are shown within boxes.

FIG. 2B is a schematic showing the ‘left end’ sequence of an Ad35 helper genome which corresponds to that shown in FIG. 1 (see also GenBank accession number AY128640) and includes (i) six nucleotides added to produce an FseI restriction site between Ad35 positions 143 and 144, (ii) loxP sites added after positions 171 and 402, and (iii) I-SceI and FseI sites added after position 480. Certain added sequences are shown within boxes.

FIG. 2C is a schematic showing the ‘left end’ sequence of an Ad35 helper genome which corresponds to that shown in FIG. 1 (see also GenBank accession number AY128640) and includes (i) six nucleotides added to produce an FseI restriction site between Ad35 positions 143 and 144, (ii) loxP sites added after positions 195 and 479, and (iii) I-SceI and FseI sites added after position 480. Certain added sequences are shown within boxes.

FIG. 2D is a schematic showing the ‘left end’ sequence of an Ad35 helper genome which corresponds to that shown in FIG. 1 (see also GenBank accession number AY128640) and includes (i) six nucleotides added to produce an FseI restriction site between Ad35 positions corresponding to 143 and 144, (ii) loxP sites added after positions corresponding to 161 and 3200, and (iii) I-SceI and FseI sites added after the position corresponding to 480. Certain added sequences are shown within boxes. The position of the loxP site at position 3200 is so numbered in that the represented construct includes a deletion of nucleotide positions 481-3199 or 482-3199 corresponding to GenBank accession number AY128640 (accordingly, this loxP site could alternatively be described as added together with the I-SceI and FseI sites after the position corresponding to 480).

FIG. 2E is a schematic showing the ‘left end’ sequence of an Ad35 helper genome which corresponds to that shown in FIG. 1 (see also GenBank accession number AY128640) and includes sequences added after positions corresponding to 206 and 484 to introduce SwaI restriction sites and loxP sites. Certain added sequences are shown within boxes. pEN024 is a plasmid encoding a helper vector genome that includes the construct of this figure. As noted elsewhere herein, and as applicable throughout, where an inserted sequence (such as a loxP site, to provide one non-limiting example) includes terminal nucleotide positions identical in sequence with reference nucleotides that could be construed as displaced by the insertion, the site of the insertion can be represented, e.g., as occurring after any of such terminal nucleotide positions, or after the last nucleotide that does not correspond to the inserted sequence of interest. Thus, for example, the defining loxP insertion positions of pEN024 could alternative be identified, e.g., as after positions corresponding to 206 and 481.

FIG. 3 is an image of a gel showing digestion of Ad35 helper genomes and plasmids including Ad35 helper genomes, together with a table describing the gel. Lanes 1, 3, 6, and 8 of the gel show BsrGI digestion of helper virus genomes produced using pEN025, pEN026, pEN027, and pEN028, respectively, while lanes 2, 4, 7, and 9 of the gel show digestion of the respective starting plasmids with BsrGI and SwaI. Lane 5 includes a 1 Kb Plus ladder. The accompanying table included in the figure shows that pEN025, pEN026, pEN027, and pEN028 each include a conditional packaging sequence according to the present disclosure, in particular one of the 4 constructs described as Constructs 1-4 in Example 1 (i.e., pEN025 corresponds to Construct 1 and FIG. 2A, pEN026 corresponds to Construct 2 and FIG. 2B, pEN027 corresponds to Construct 3 and FIG. 2C, and pEN028 corresponds to Construct 4 and FIG. 2D), respectively. Expected band sizes were obtained in all lanes.

FIG. 4 is an image of a gel showing digestion of Ad35 helper genomes, together with a table describing the gel. The accompanying table included in the figure shows the plasmid and cell type used in producing the sample shown in each lane, as well as the expected band size. ApaI digestion produces a 2014 bp fragment from packaging-competent Ad35 genomes (flanked packaging sequence not excised), and a smaller fragment from Ad35 genomes from which a flanked packaging sequence has been excised. Lane 1 includes a 1 Kb Plus ladder. All band sizes were consistent with expectations.

FIG. 5 is a plasmid map depicting the structural organization of plasmid 5427, a plasmid that encodes a helper-dependent genome that includes terminal sequences derived from Ad35. The encoded helper-dependent genome includes a cassette for expression of beta-galactosidase. Digestion of plasmid 5427 with the restriction enzyme PmeI releases the helper-dependent genome from the plasmid backbone. At least because the 5′ and 3′ ends of plasmid 5427 include sequences derived from Ad35, the encoded helper-dependent genome can be packaged into vector particles produced using Ad35 helper genomes of the present disclosure.

FIG. 6A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN025) and a plasmid including a helper-dependent genome that includes terminal sequences derived from Ad35 (plasmid 5427).

FIG. 6B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN026) and a plasmid including a helper-dependent genome that includes terminal sequences derived from Ad35 (plasmid 5427).

FIG. 6C is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN027) and a plasmid including a helper-dependent genome that includes terminal sequences derived from Ad35 (plasmid 5427).

FIG. 6D is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN028) and a plasmid including a helper-dependent genome that includes terminal sequences derived from Ad35 (plasmid 5427).

FIG. 6E is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN024) and a plasmid including a helper-dependent genome that includes terminal sequences derived from Ad35 (plasmid 5427).

FIG. 7A is an image of a gel, together with a table describing the gel. The gel shows digestion of adenoviral genomes obtained from 116 cells transfected with a plasmid including an Ad35 helper genome according to the present disclosure (pEN025 or pEN026) and plasmid 5427, then purified by two successive rounds of cesium chloride gradient purification. As indicated in the table included in the figure, the purified adenoviral genomes were digested with SacII (lanes 4 and 6), while parental plasmids were also digested for comparison (lanes 2, 3, and 5). In lane 2, plasmid 5427 was digested with PmeI (releases the helper-dependent genome from the plasmid 5427 backbone) and SacII. In lanes 3 and 5, helper plasmids pEN025 and pEN026 were digested with SwaI (releases helper genome from the plasmid backbone of pEN025 and pEN026) and SacII.

FIG. 7B is an image of a gel, together with a table describing the gel. The gel shows digestion of adenoviral genomes obtained from 116 cells transfected with a plasmid including an Ad35 helper genome according to the present disclosure (pEN027 or pEN028) and plasmid 5427, then purified by two successive rounds of cesium chloride gradient purification. As indicated in the table included in the figure, the purified adenoviral genomes were digested with SacII (lanes 4 and 6), while parental plasmids were also digested for comparison (lanes 2, 3, and 5). In lane 2, plasmid 5427 was digested with PmeI (releases the helper-dependent genome from the plasmid 5427 backbone) and SacII. In lanes 3 and 5, helper plasmids pEN027 and pEN028 were digested with SwaI (releases helper genome from the plasmid backbone of pEN027 and pEN028) and SacII.

FIG. 7C is an image of a gel, together with a table describing the gel. The gel shows digestion of adenoviral genomes obtained from 116 cells transfected with a plasmid including an Ad35 helper genome according to the present disclosure (pEN024) and plasmid 5427, then purified by two successive rounds of cesium chloride gradient purification. As indicated in the table included in the figure, the purified adenoviral genomes were digested with SacII (lane 4), while parental plasmids were also digested for comparison (lanes 2 and 3). In lane 3, plasmid 5427 was digested with PmeI (releases the helper-dependent genome from the plasmid 5427 backbone) and SacII. In lane 2, helper plasmids pEN024 was digested with PmeI (releases helper genome from the plasmid backbone of pEN024) and SacII.

FIG. 8A is a schematic showing homologous recombination between an Ad35 helper genome (Helper Ad) and a helper-dependent Ad35 genome (HDAd) that results in elimination of one of the recombinase sites that flank a packaging sequence.

FIG. 8B is a schematic showing an Ad35 helper genome (Helper Ad) that includes a packaging sequence inversion. Packaging sequence inversion reduces and/or eliminate recombinase site-excising homologous recombination, and thereby prevents production of a constitutively packageable helper genome.

FIG. 9A is a schematic showing the ‘left end’ sequence of an Ad35 helper genome that includes a packaging sequence inversion. The sequence shown in FIG. 9A corresponds to the sequence of FIG. 2A and includes an inversion of nucleotides positioned between FseI sites of FIG. 2A.

FIG. 9B is a schematic showing the ‘left end’ sequence of an Ad35 helper genome that includes a packaging sequence inversion. The sequence shown in FIG. 9A corresponds to the sequence of FIG. 2B and includes an inversion of nucleotides positioned between FseI sites of FIG. 2B.

FIG. 9C is a schematic showing the ‘left end’ sequence of an Ad35 helper genome that includes a packaging sequence inversion. The sequence shown in FIG. 9C corresponds to the sequence of FIG. 2C and includes an inversion of nucleotides positioned between FseI sites of FIG. 2C.

FIG. 9D is a schematic showing the ‘left end’ sequence of an Ad35 helper genome that includes a packaging sequence inversion. The sequence shown in FIG. 9D corresponds to the sequence of FIG. 2D and includes an inversion of nucleotides positioned between FseI sites of FIG. 2D.

FIG. 10 is an image of a gel showing digestion of Ad35 helper genomes and plasmids including Ad35 helper genomes, together with a table describing the gel. Lanes 2 and 4 of the gel show XmnI digestion of helper virus genomes produced using pEN0056 and pEN0057, respectively, while lanes 1 and 3 of the gel show digestion of the respective starting plasmid with XmnI and SwaI. Lane 5 includes a 1 Kb Plus ladder. The accompanying table included in the figure shows that pEN0056 and pEN0057 each include an inverted conditional packaging sequence according to the present disclosure, in particular one of the constructs described as Constructs 7 and 8 in Example 5, respectively (i.e., pEN0056 corresponds to a plasmid including Construct 7 and pEN0057 corresponds to a plasmid including Construct 8). Expected band sizes were obtained in all lanes.

FIG. 11 is an image of a gel showing digestion of Ad35 helper genomes, together with a table describing the gel. The accompanying table included in the figure shows the plasmid and cell type used in producing the sample shown in each lane, as well as the expected band size. ApaI digestion produces a 2013 bp fragment from packaging-competent Ad35 genomes (inverted flanked packaging sequence not excised), and a smaller fragment from Ad35 genomes from which an inverted flanked packaging sequence has been excised. Lane 1 includes a 1 Kb Plus ladder. All band sizes were consistent with expectations.

FIG. 12 is a plasmid map depicting the structural organization of plasmid 5475, a plasmid that encodes a helper-dependent genome that includes terminal sequences derived from Ad35. The encoded helper-dependent genome includes a cassette for expression of beta-galactosidase. Digestion of plasmid 5475 with the restriction enzyme PmeI releases the helper-dependent genome from the plasmid backbone. At least because the 5′ and 3′ ends of plasmid 5475 include sequences derived from Ad35, the encoded helper-dependent genome can be packaged into vector particles produced using Ad35 helper genomes of the present disclosure.

FIG. 13A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0056) and a plasmid including a helper-dependent genome that includes terminal sequences derived from Ad35 (plasmid 5475).

FIG. 13B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including a helper-dependent genome that includes terminal sequences derived from Ad35 (plasmid 5475).

FIG. 14 is an image of a gel, together with a table describing the gel. The gel shows digestion of adenoviral genomes obtained from 116 cells transfected with a plasmid including an Ad35 helper genome according to the present disclosure (pEN0056 or pEN0057) and plasmid 5475, then purified by two successive rounds of cesium chloride gradient purification. As indicated in the table included in the figure, the purified adenoviral genomes were digested with SacII (lanes 4 and 5), while parental plasmids were also digest for comparison (lanes 2 and 3). In lane 3, plasmid 5475 was digested with PmeI (releases the helper-dependent genome from the plasmid 5475 backbone) and SacII. In lane 2, helper plasmid pEN0057 was digested with PmeI (releases helper genome from the plasmid backbone of pEN0057) and SacII. Digestion of helper plasmid pEN0056 is predicted to display a comparable restriction pattern to that of pEN0057.

FIG. 15A is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including an exemplary helper-dependent genome that includes terminal sequences derived from Ad35 (plasmid 1).

FIG. 15B is a pair of images showing cesium chloride gradient purification of helper-dependent adenovirus produced using an Ad35 helper genome according to the present disclosure. Images represent two successive rounds of purification. The HDAd preparation subjected to the cesium chloride gradient purification was produced by transfecting 116 cells with a plasmid including an Ad35 helper genome according to the present disclosure (pEN0057) and a plasmid including an exemplary helper-dependent genome that includes terminal sequences derived from Ad35 (plasmid 2).

FIG. 16 is an image of a gel, together with a table describing the gel. The gel shows digestion of adenoviral genomes obtained from 116 cells transfected with a plasmid including an Ad35 helper genome according to the present disclosure (pEN0057), and plasmid 1 or plasmid 2, then purified by two successive rounds of cesium chloride gradient purification. As indicated in the table included in the figure, the purified adenoviral genomes were digested with EcoRV (lanes 3, 5, and 6), while parental plasmids were also digest for comparison (lanes 2, 4, and 7). In lanes 4 and 7, plasmid 1 or plasmid 2 was digested with PmeI (releases the helper-dependent genome from the plasmid 5475 backbone) and EcoRV. In lane 2, helper plasmid pEN0057 was digested with SwaI (releases helper genome from the plasmid backbone of pEN0057) and EcoRV.

FIG. 17 is a listing of nucleic acid sequences and amino acid sequences corresponding to publicly available sequence accession numbers, certain of which sequences and/or sequence accession numbers are included and/or utilized, in whole and/or in part, in the present disclosure, and/or certain of which sequences and/or sequence accession numbers are included herein as references.

### DETAILED DESCRIPTION

The present disclosure includes adenoviral serotype 35 (Ad35) vectors and Ad35 genomes useful in gene therapy. Adenoviruses are large, icosahedral-shaped, non-enveloped viruses. While some viral vectors are characterized by relatively high immunogenicity in human populations and/or by relatively low payload capacity, Ad35 vectors are characterized by relatively low immunogenicity in human populations and relatively high payload capacity. However, engineering of Ad35 vectors and genomes for use in gene therapy is not straightforward. The present disclosure includes, among other things, engineering of Ad35 helper vectors useful in producing therapeutic Ad35 donor vectors and/or in methods of gene therapy.

Those of skill in the art will appreciate that, throughout the present disclosure, references to particular nucleotide positions and/or positions corresponding thereto disclose both the specific position identified and similar positions, e.g., positions within 10, 9, 8, 7, 6, 5, 4, 3, 2, or 1 nucleotides of an indicated position. Moreover, in various embodiments in which a heterologous sequence is inserted into or positioned within a sequence corresponding to a reference adenoviral genome, the specific point of insertion can be equivalently referred to by multiple positions if the inserted sequence includes nucleotides adjacent to reference sequence that are the same as would be found in the reference sequence. In various such embodiments, the insertion can be identified as an insertion after any nucleotide position that is contiguous with reference sequence nucleotides and identical in sequence to a corresponding nucleotide of the reference sequence.

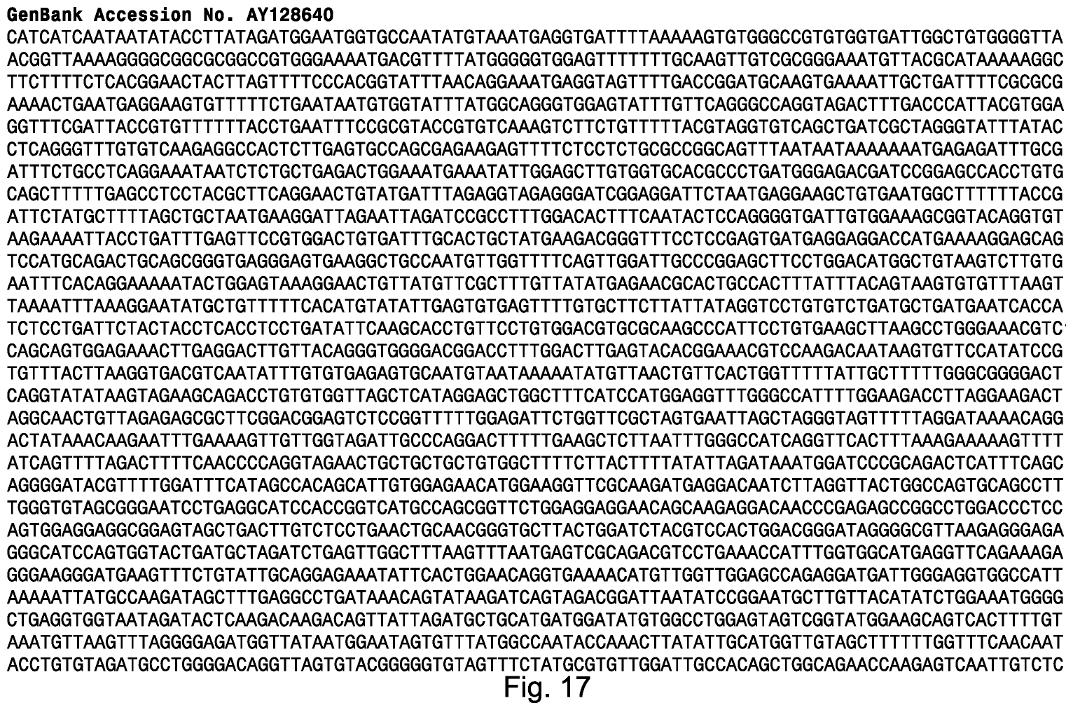

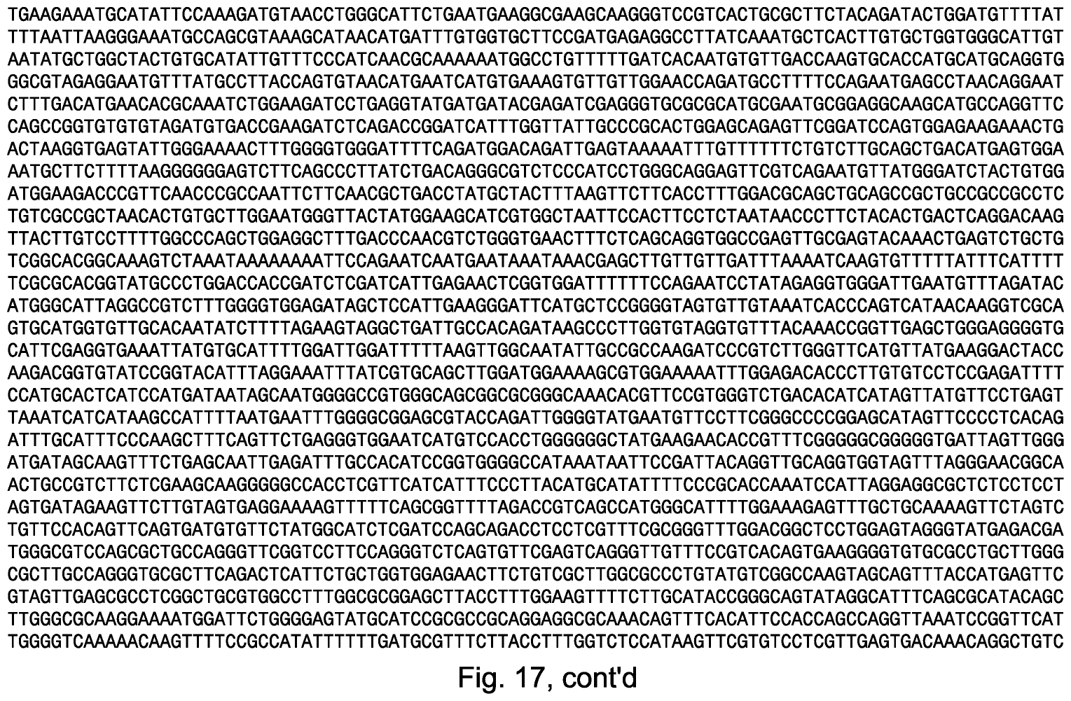

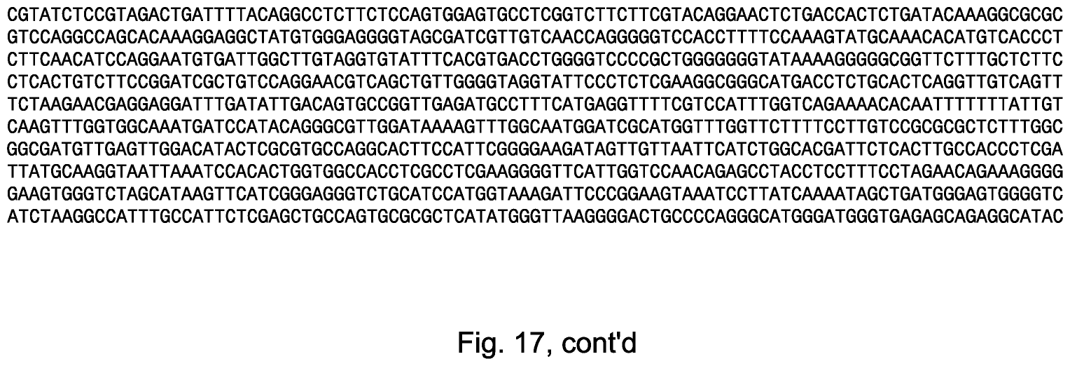

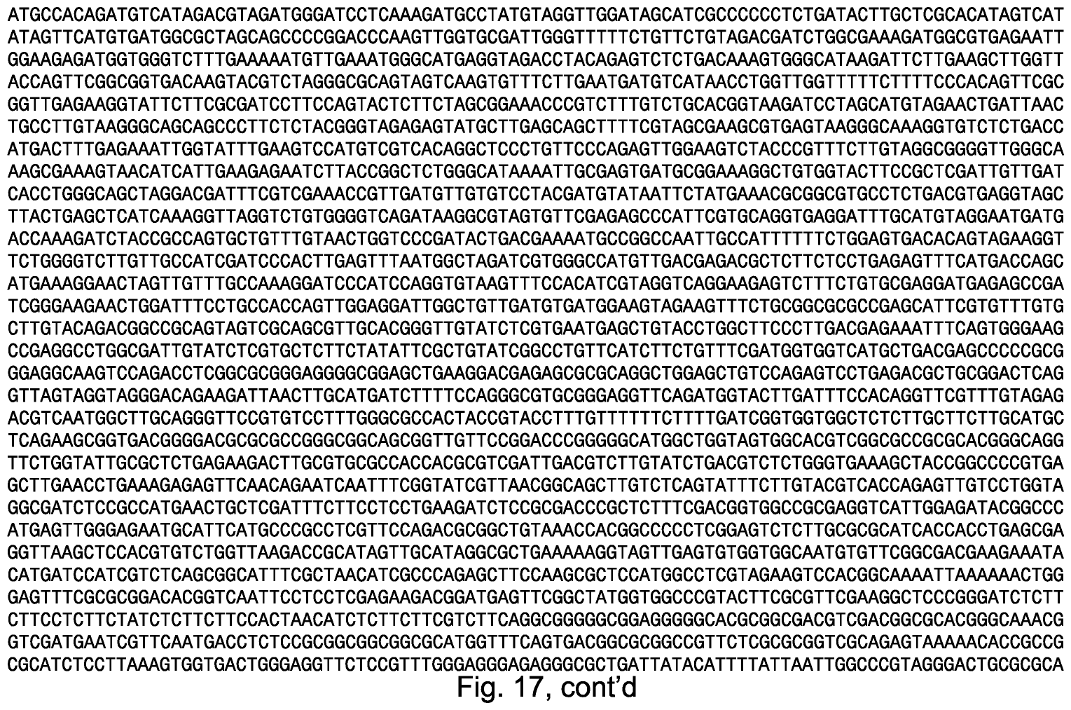

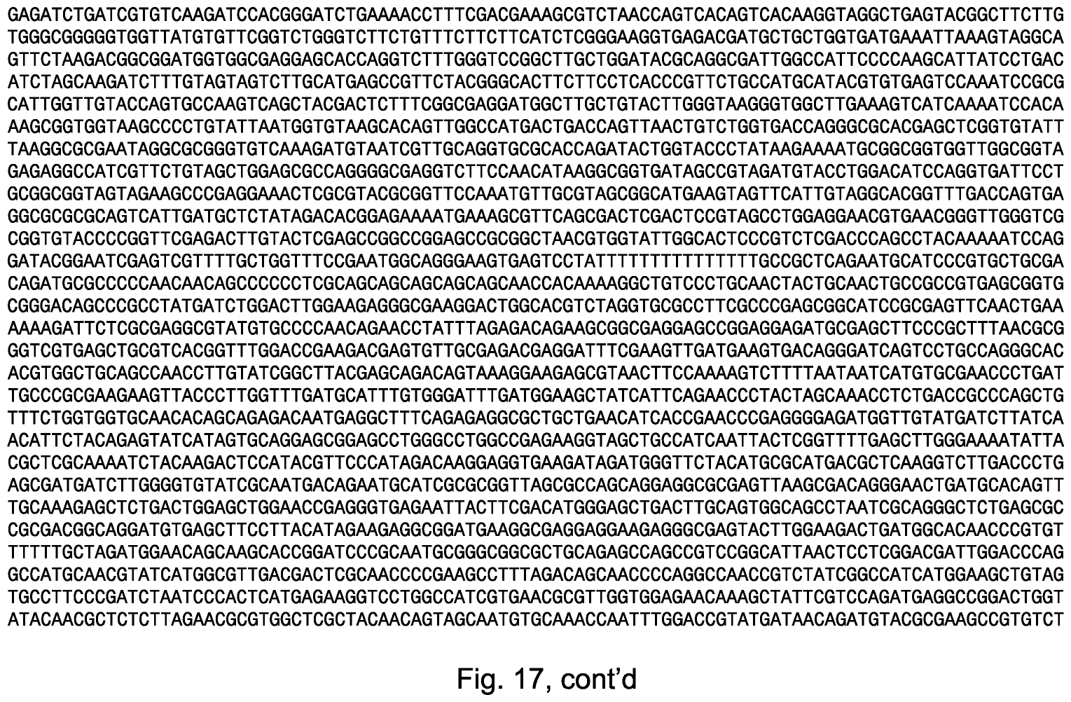

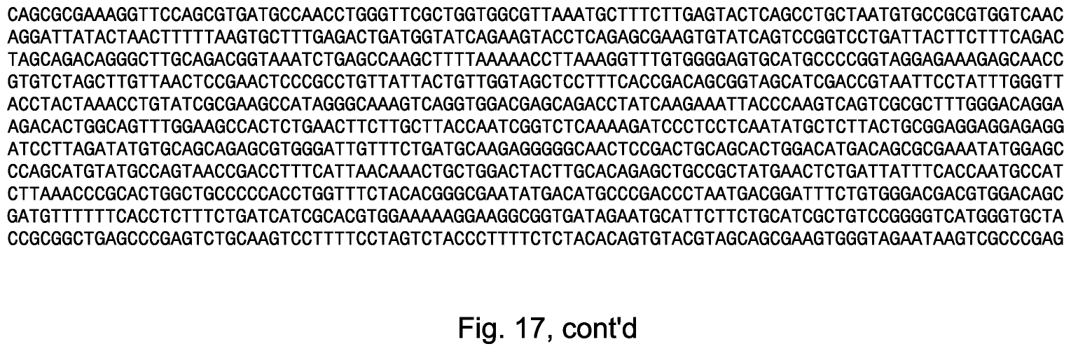

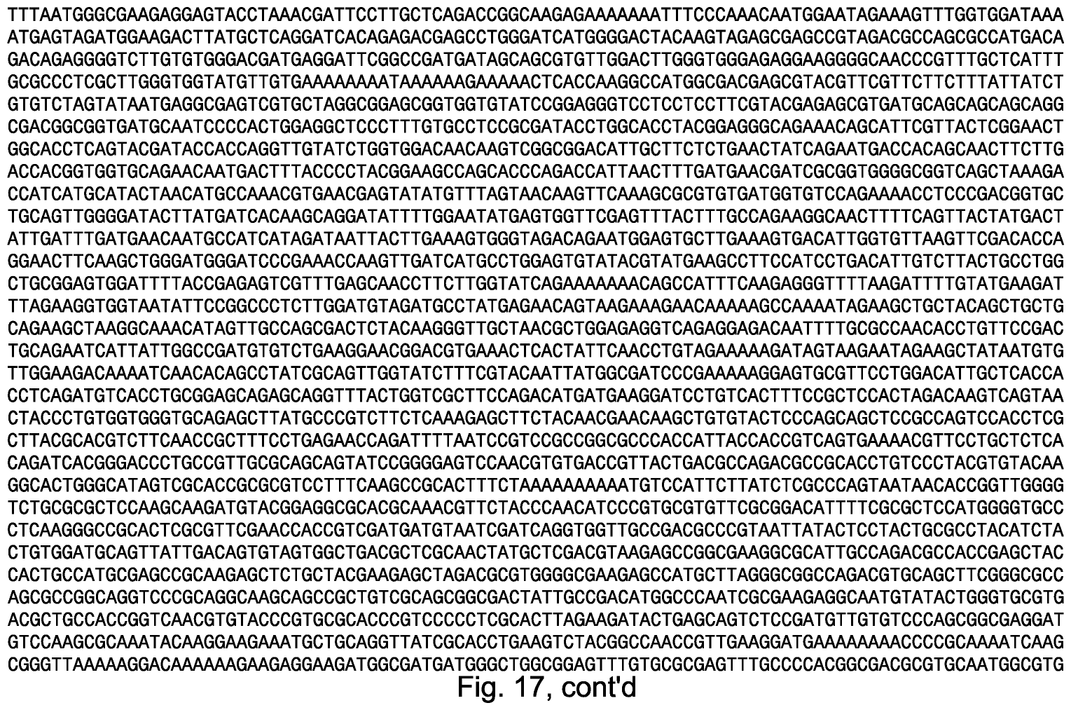

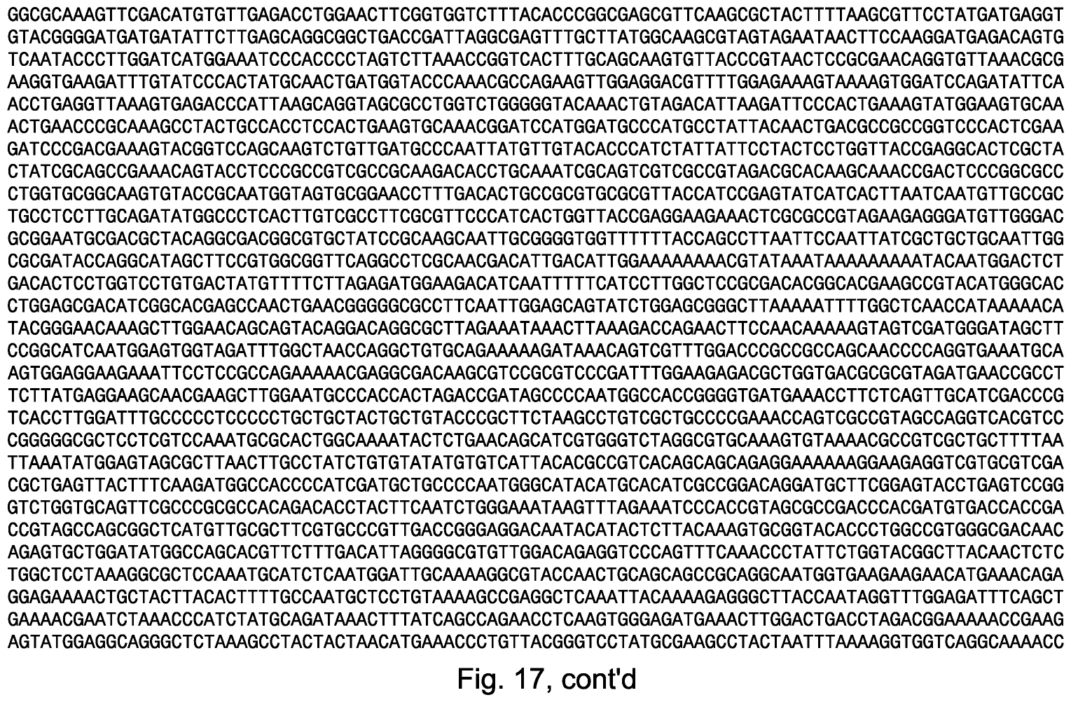

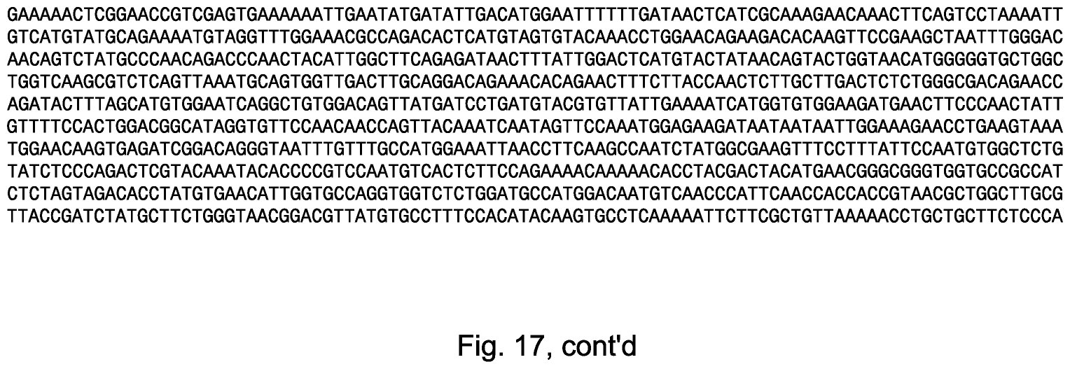

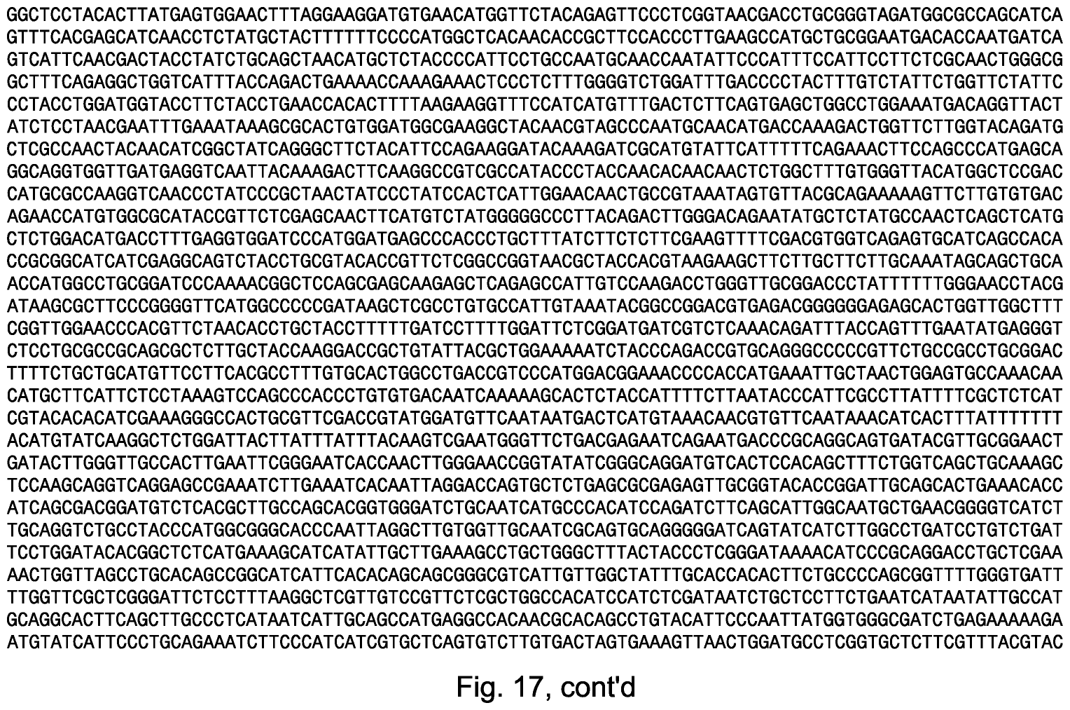

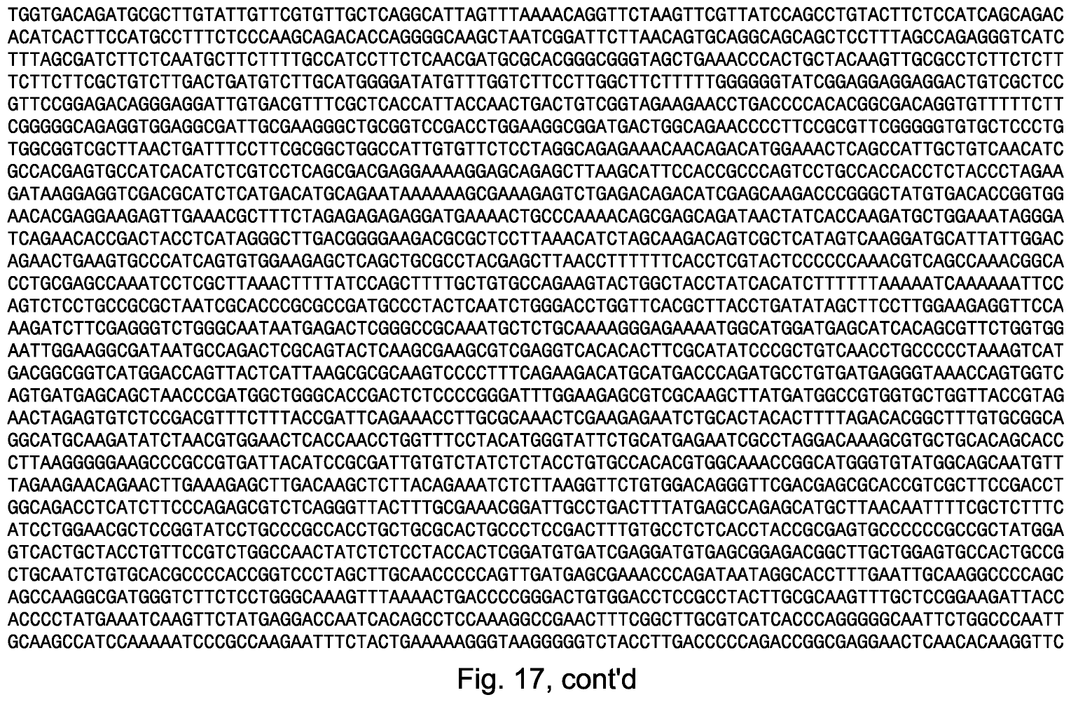

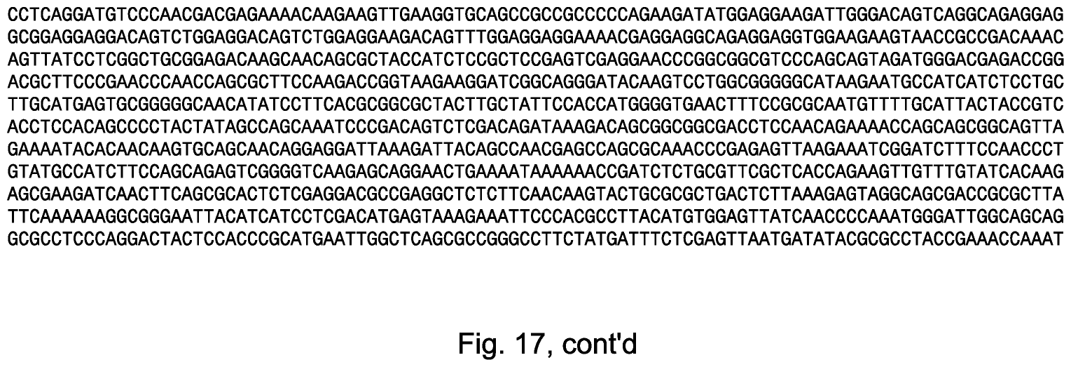

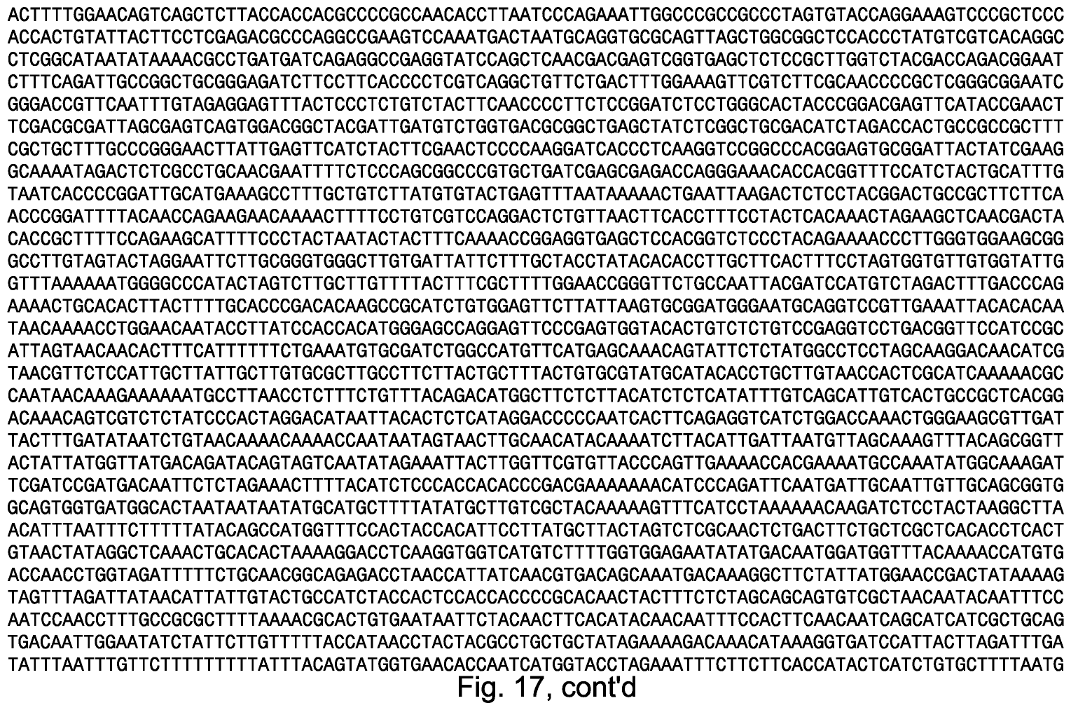

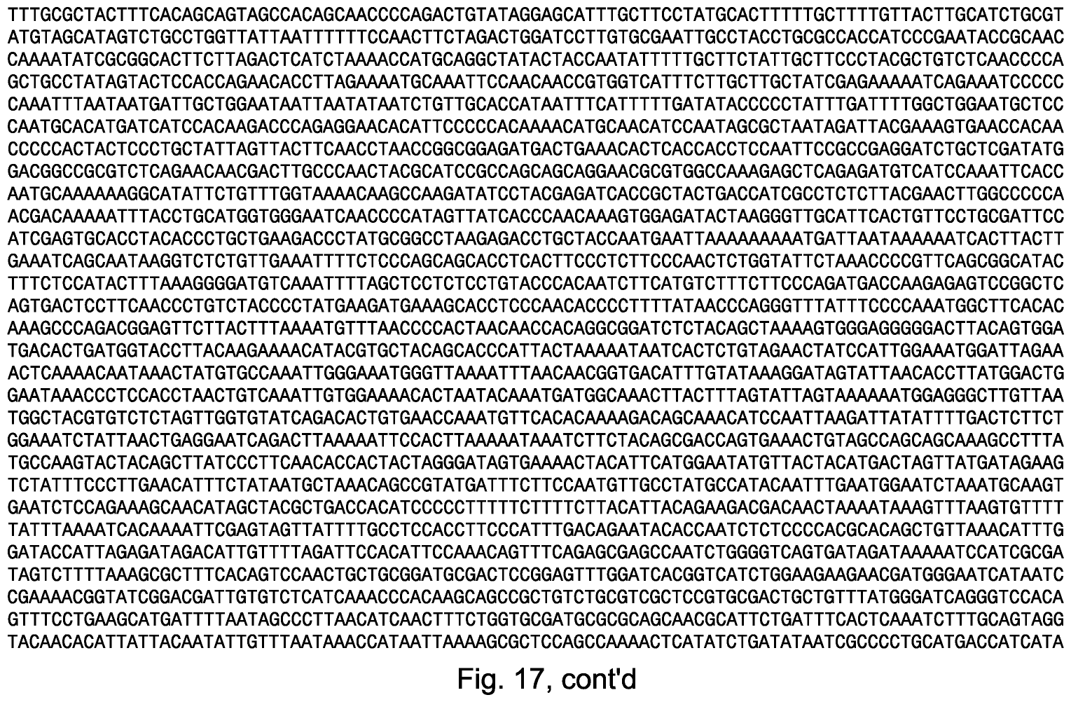

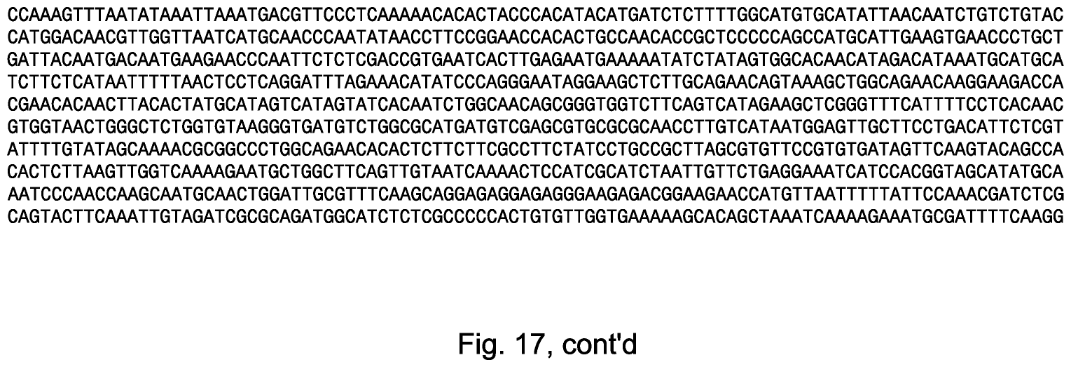

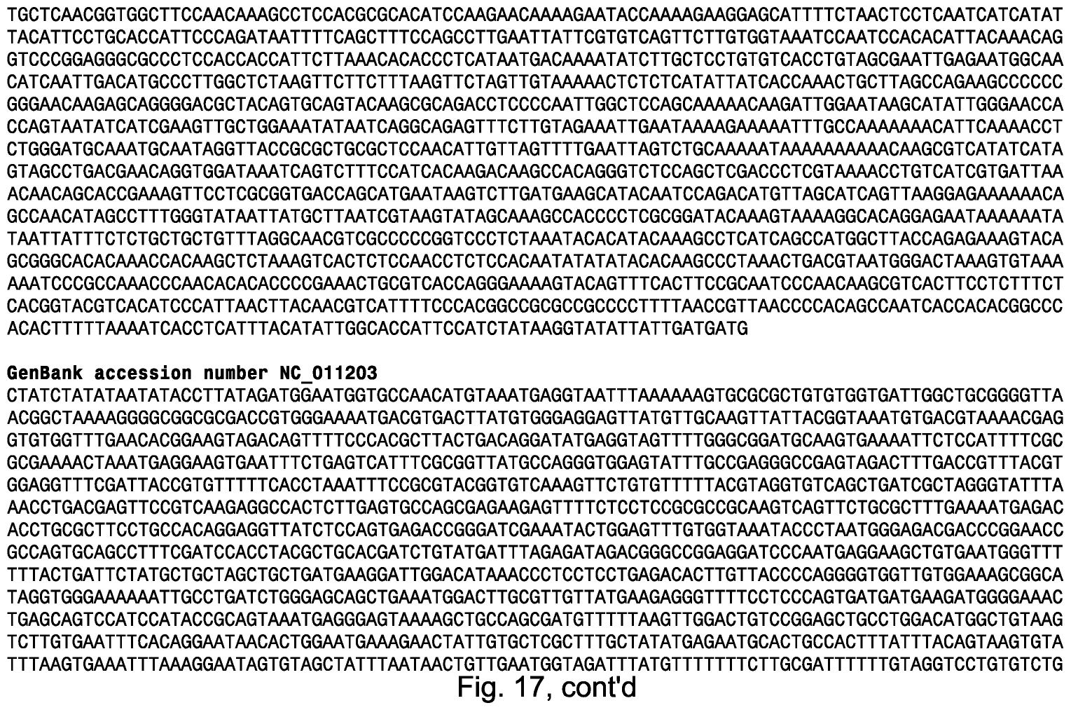

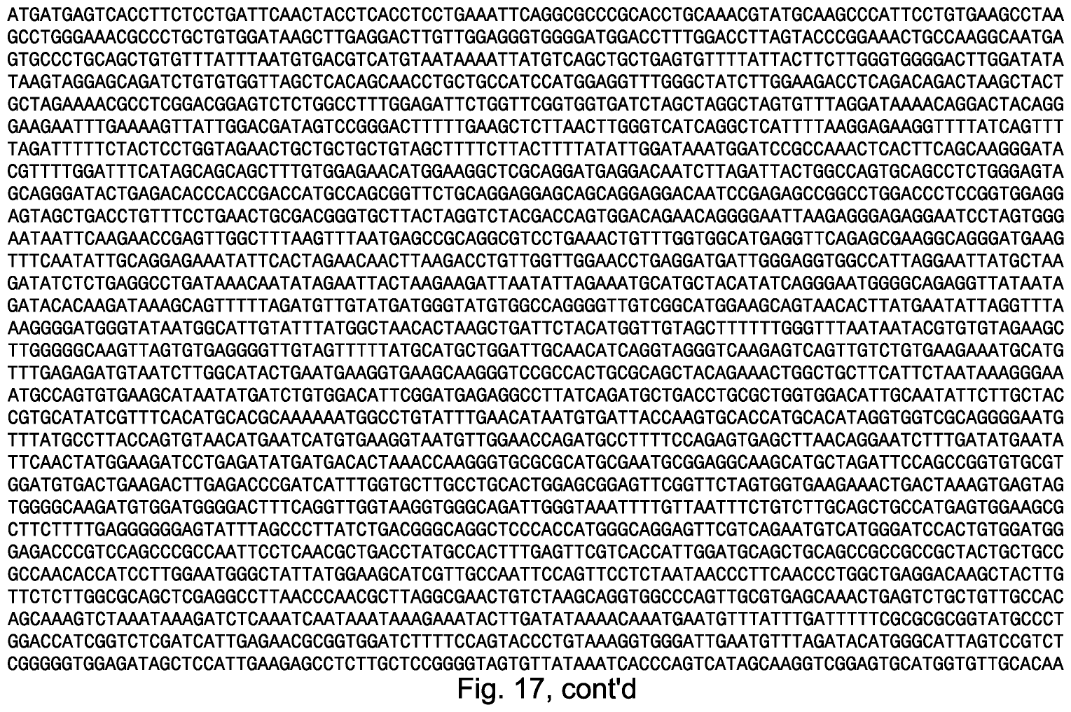

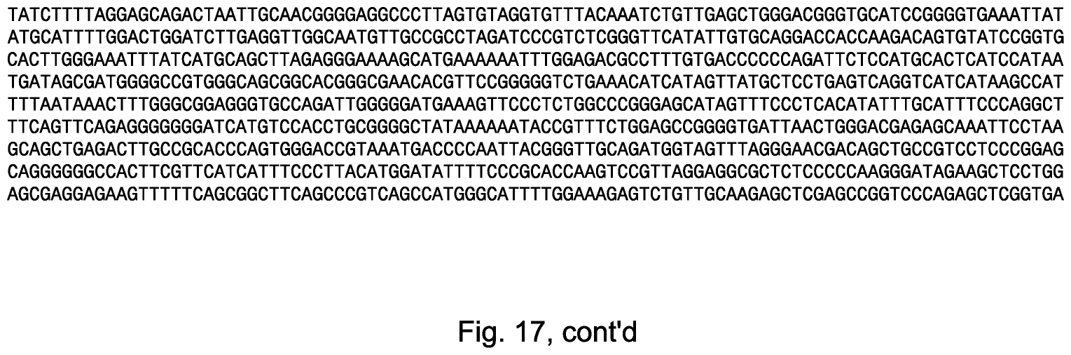

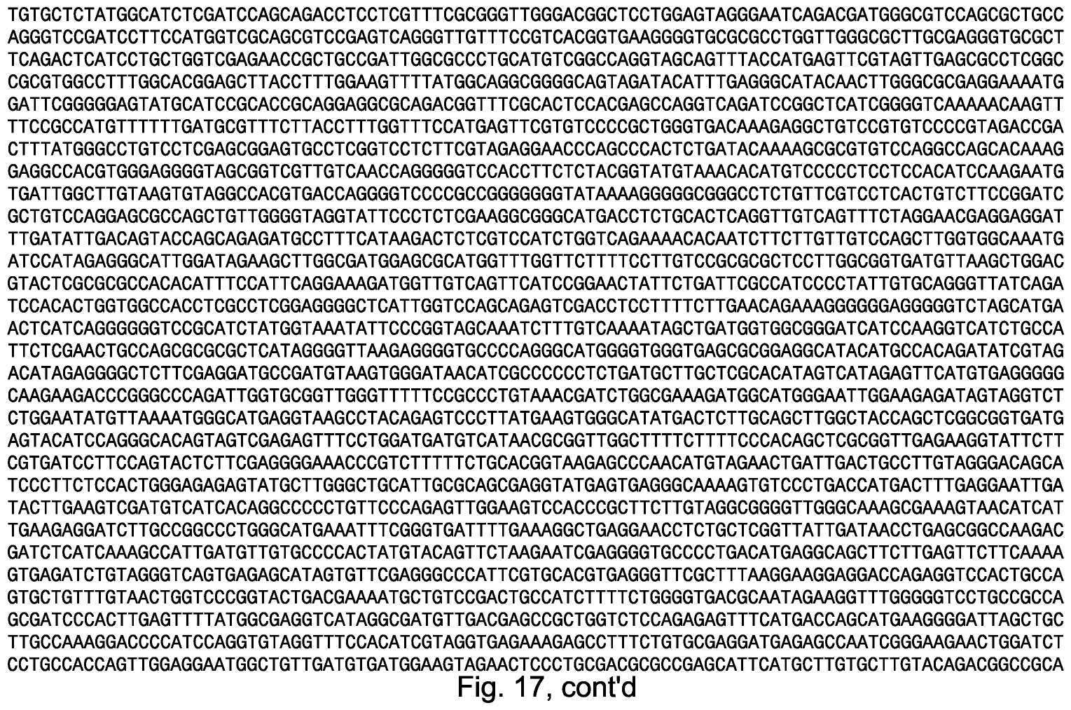

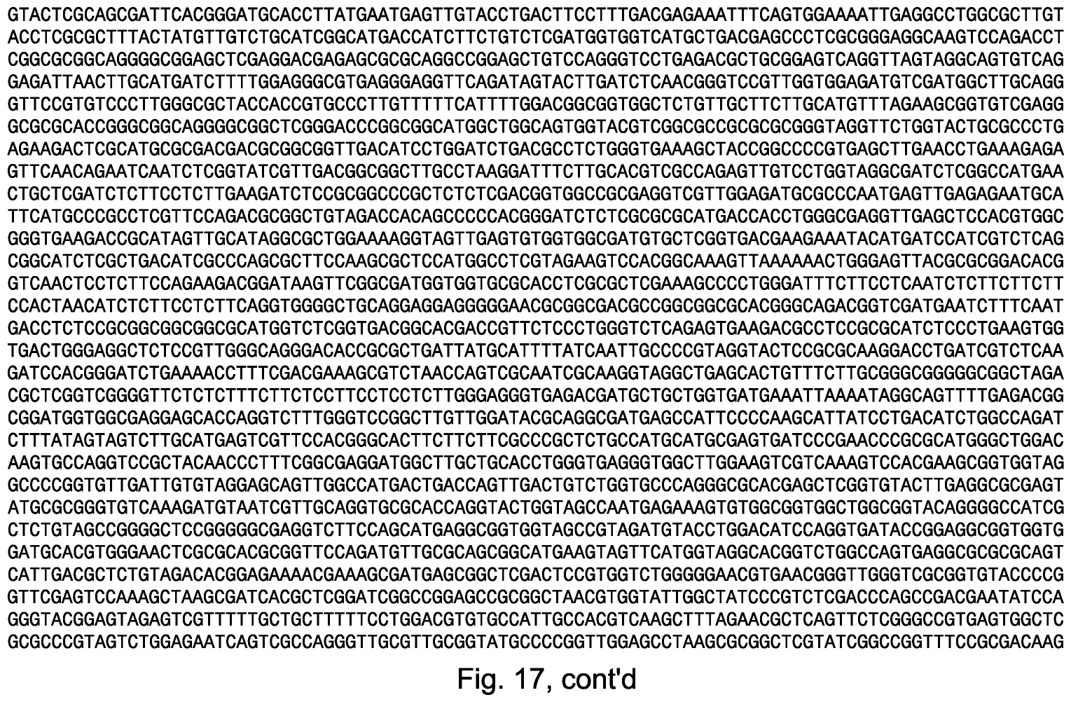

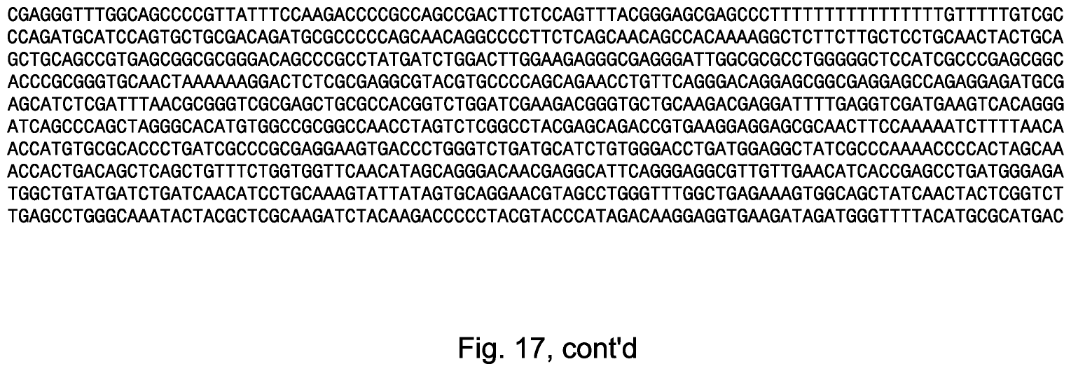

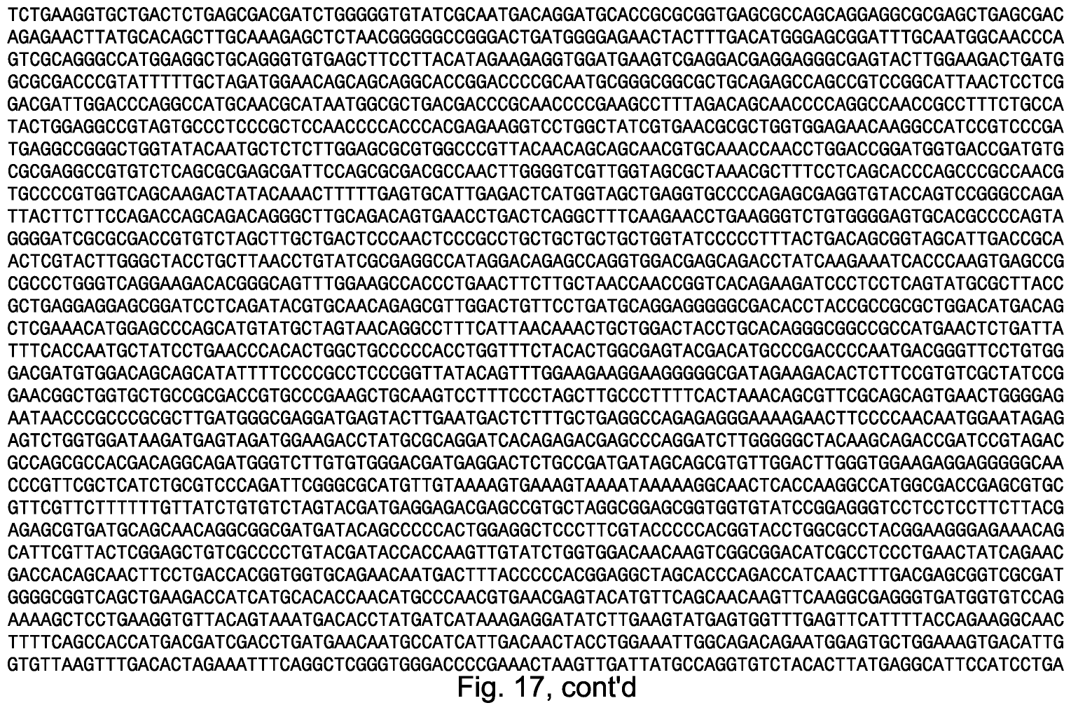

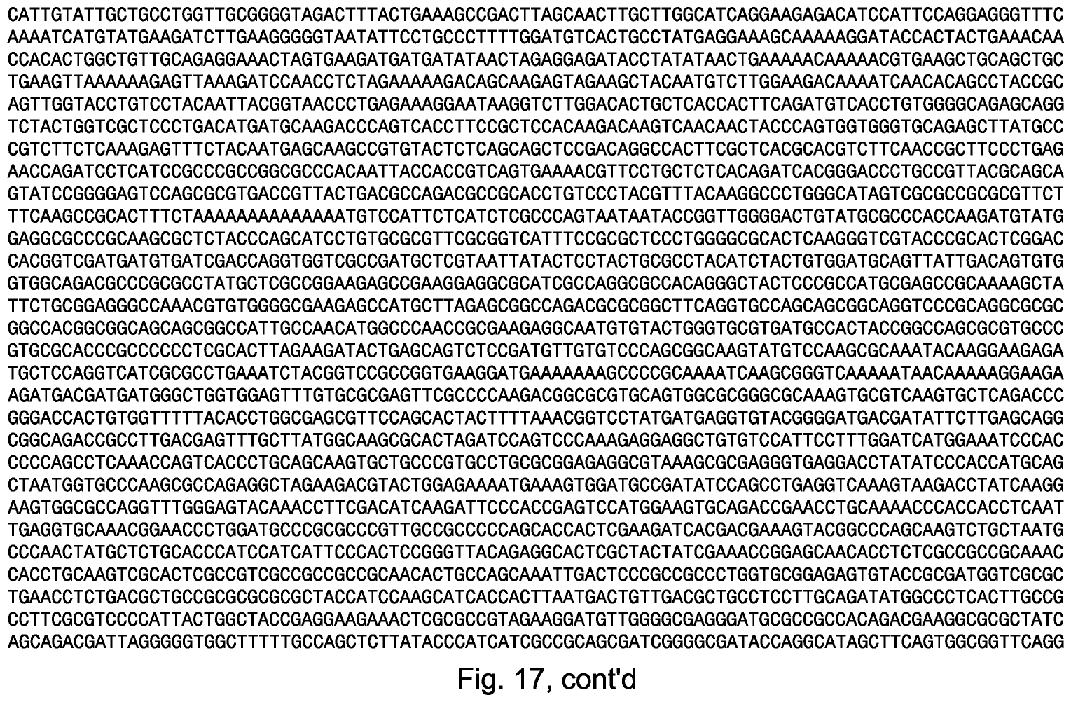

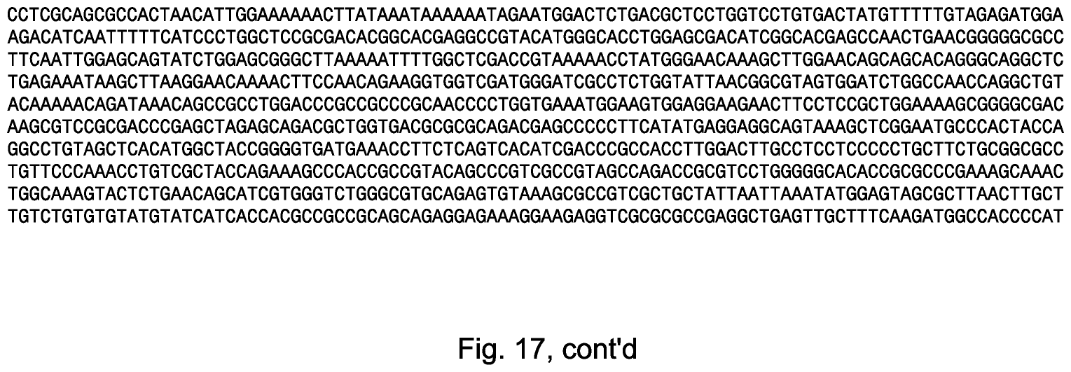

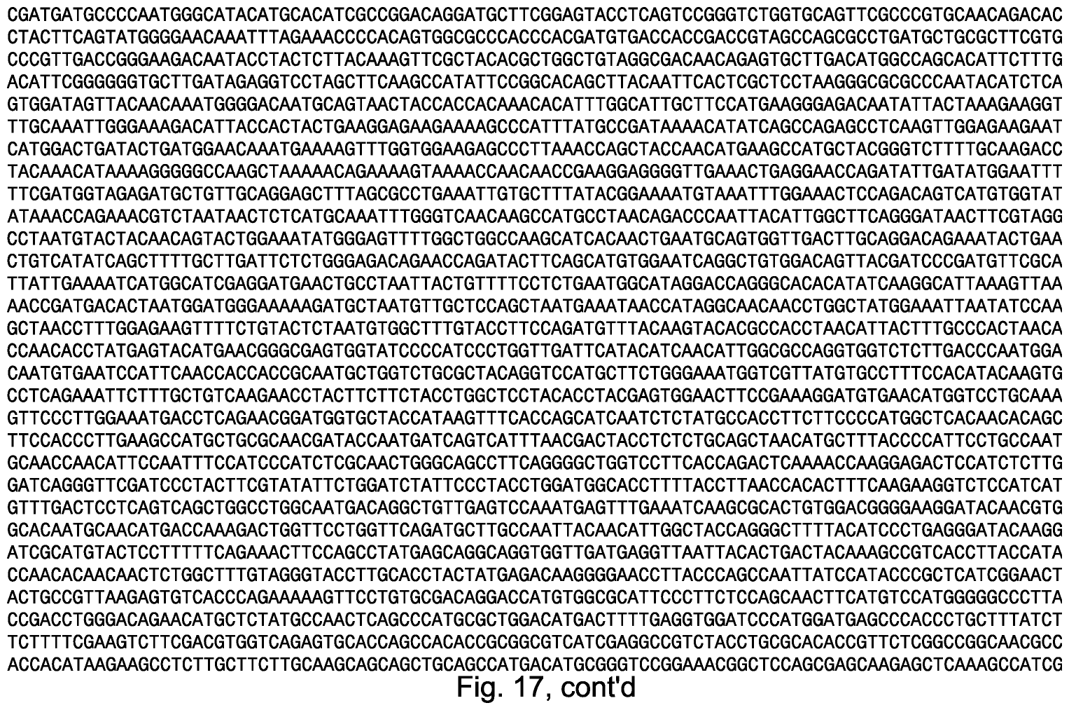

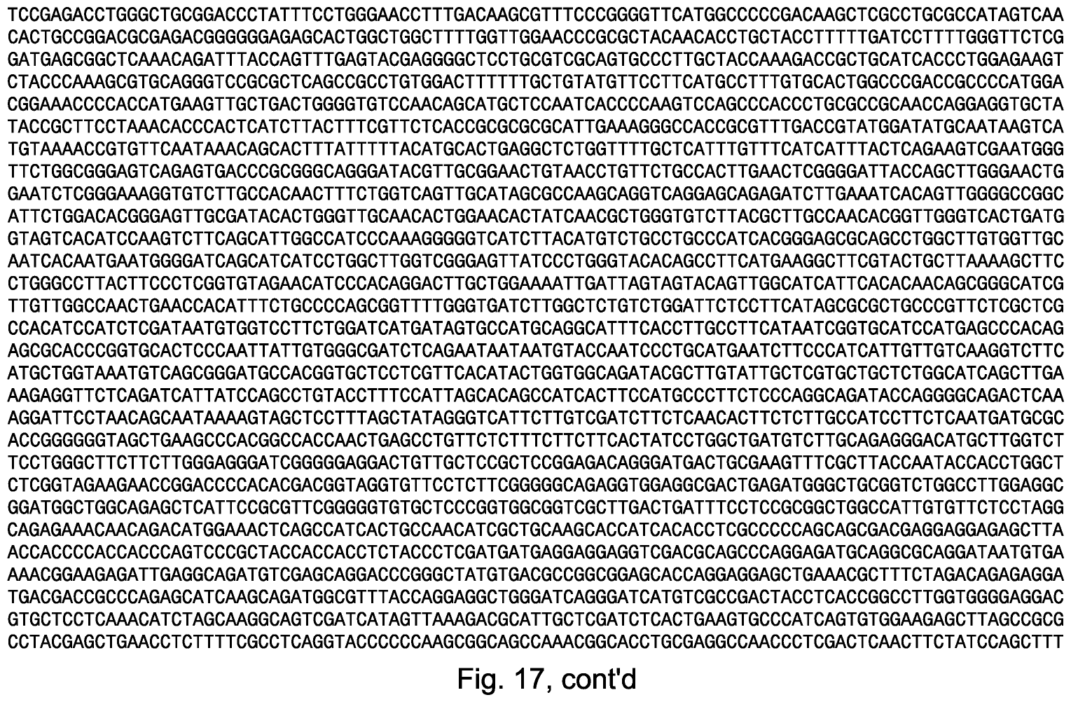

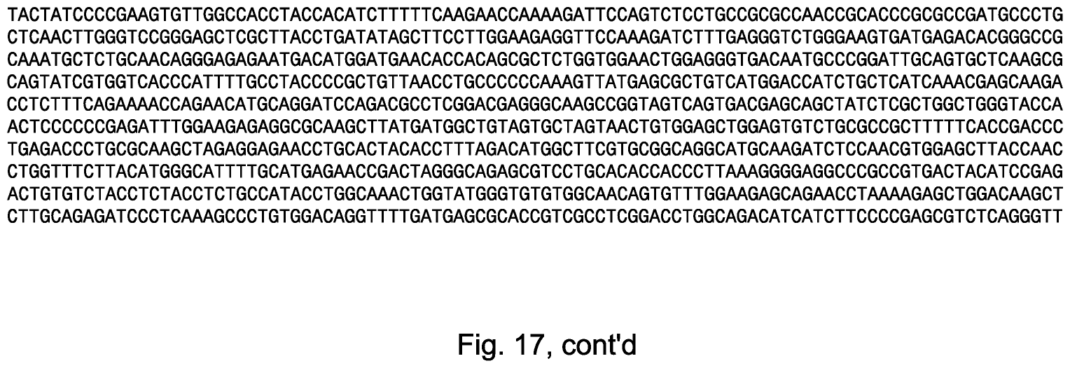

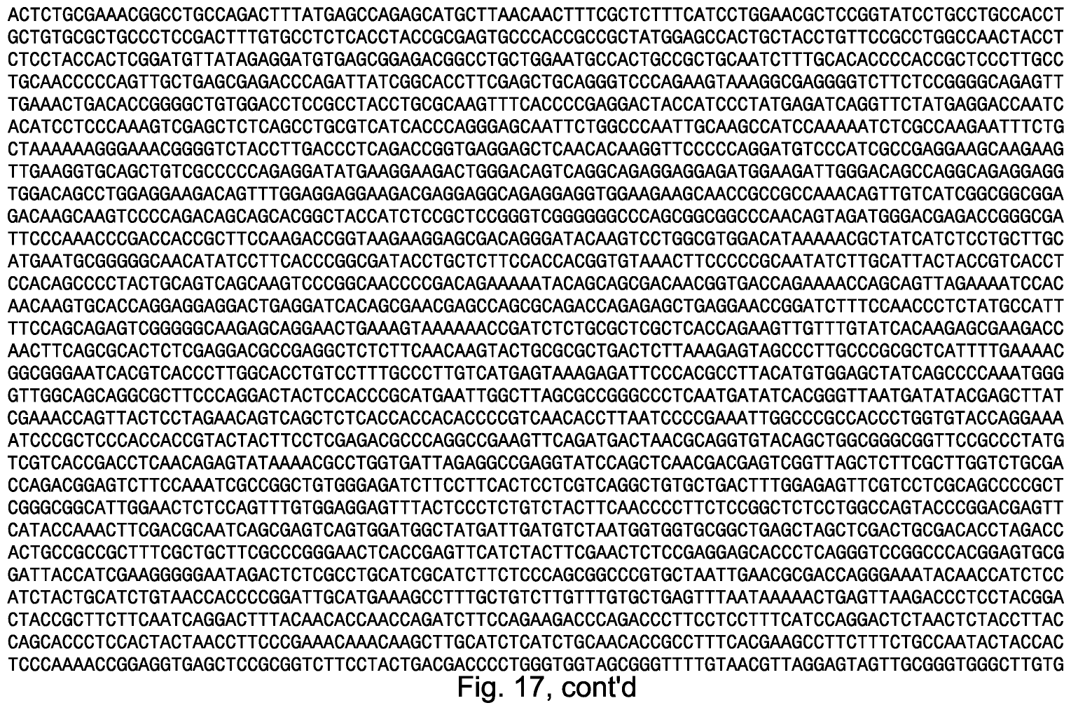

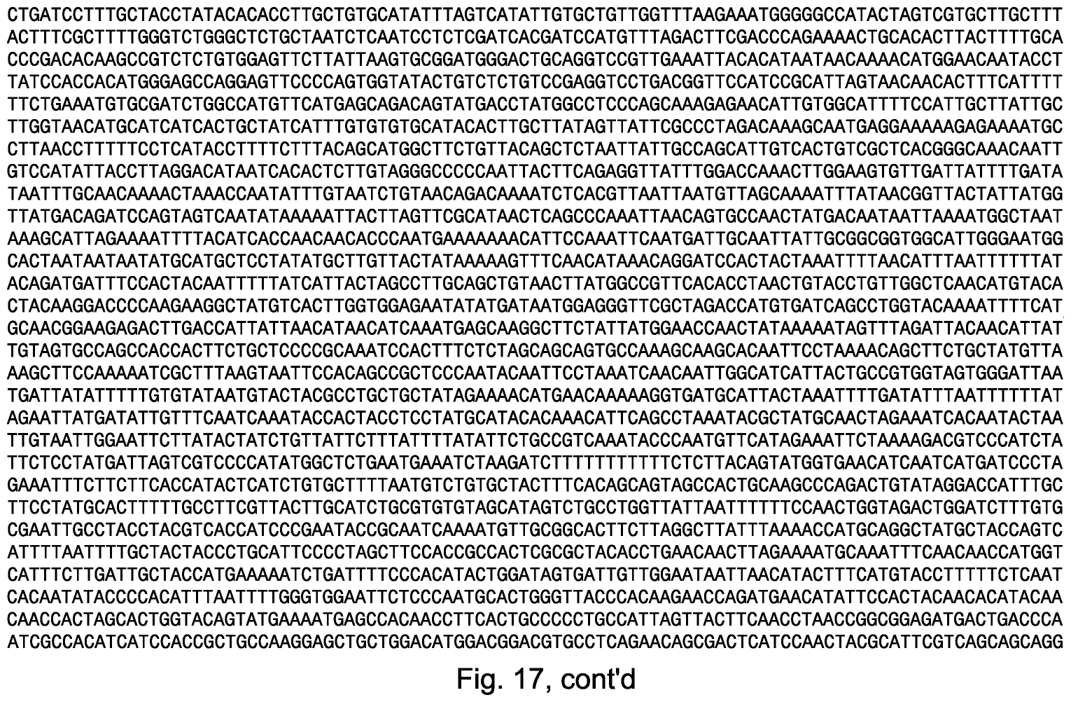

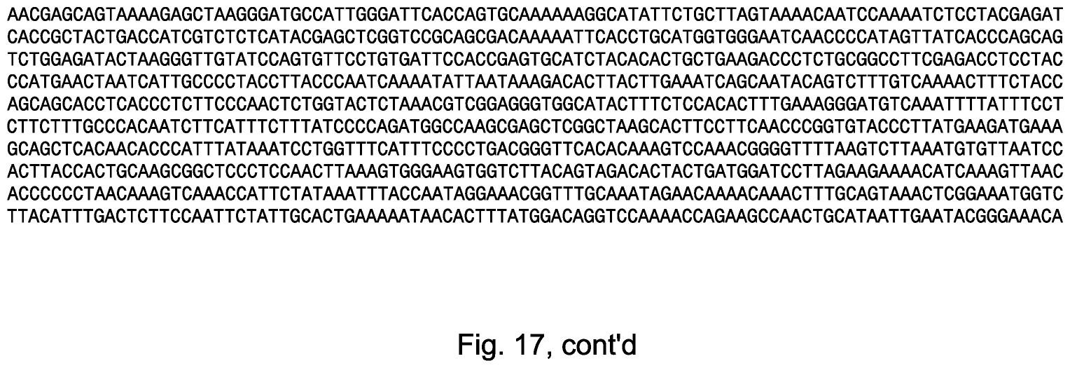

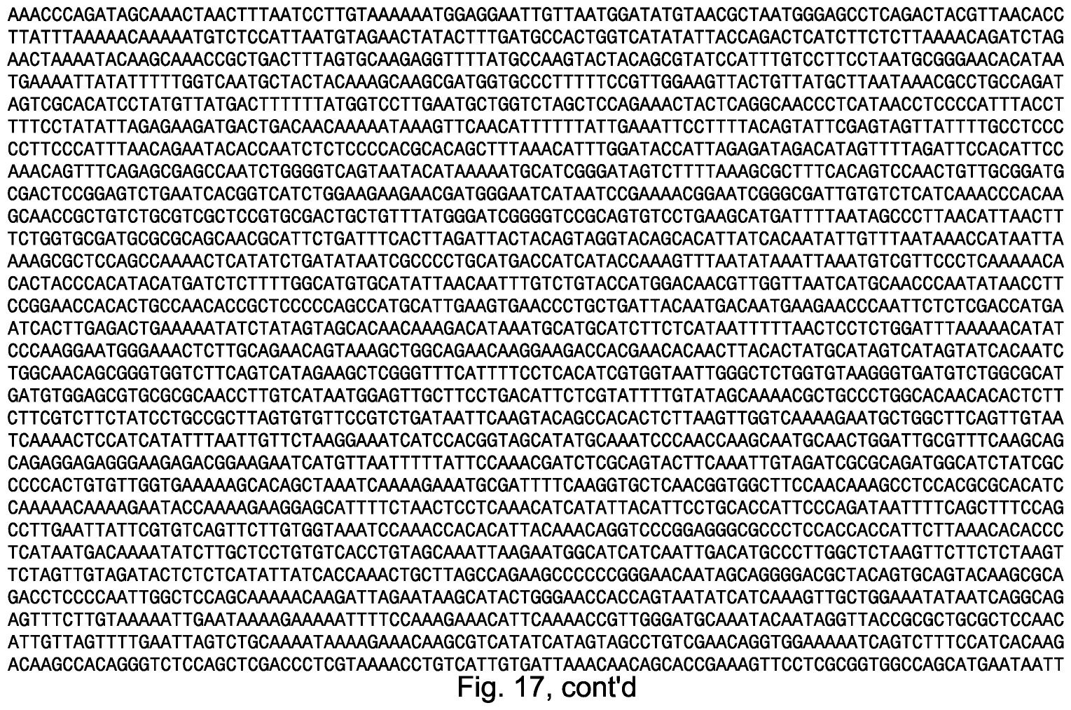

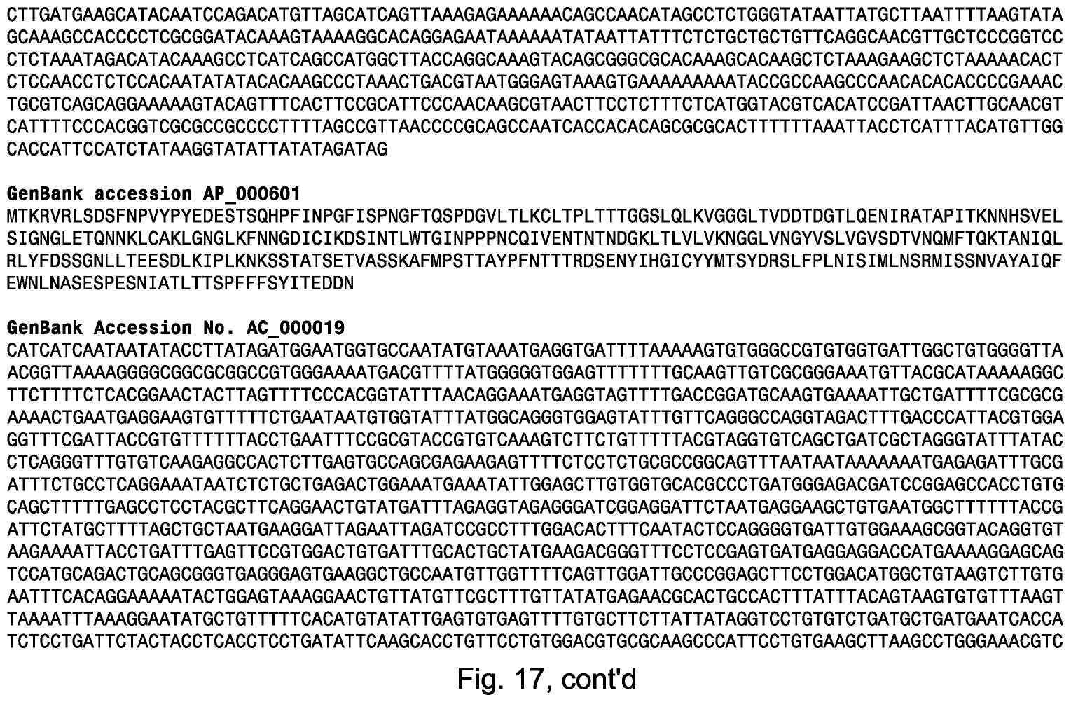

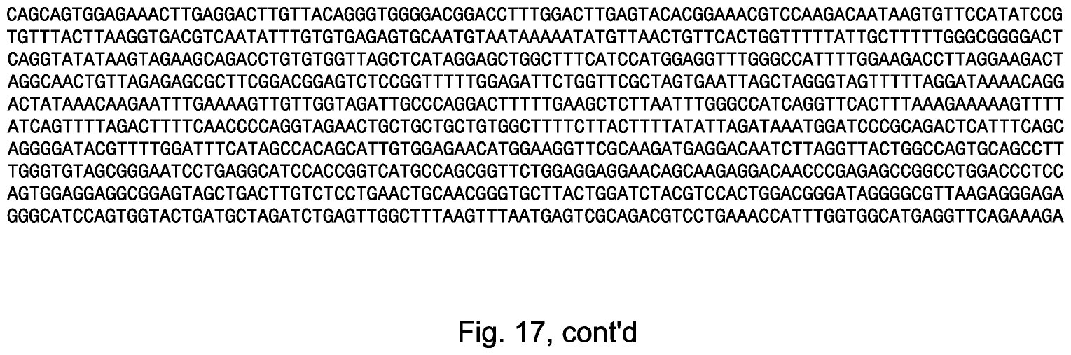

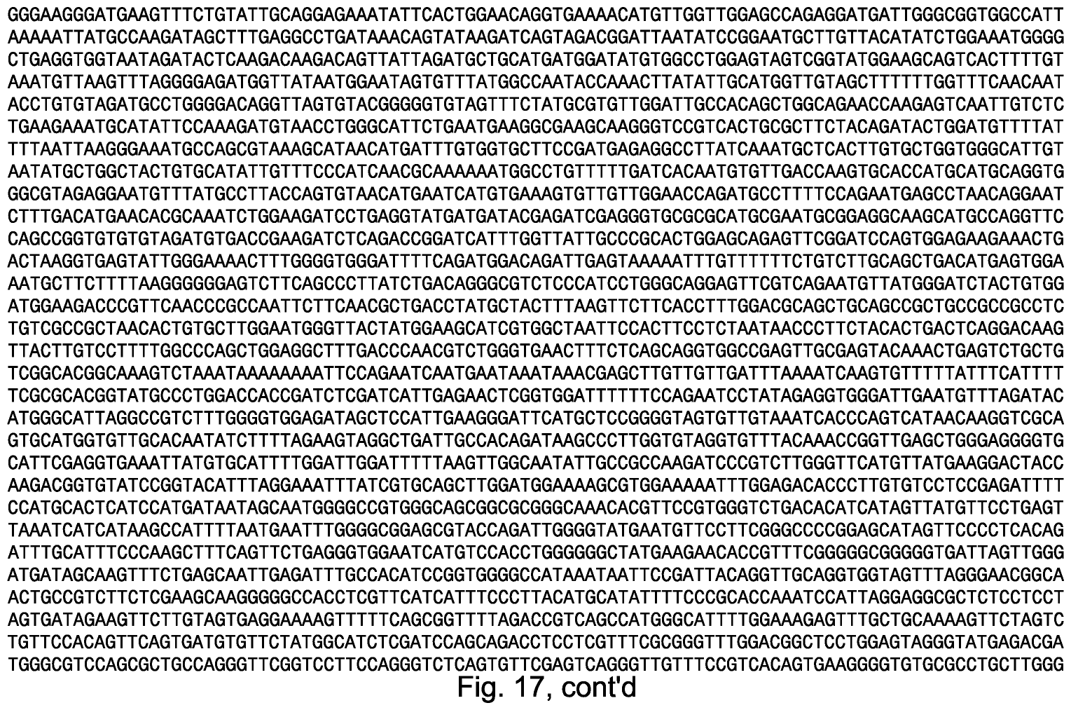

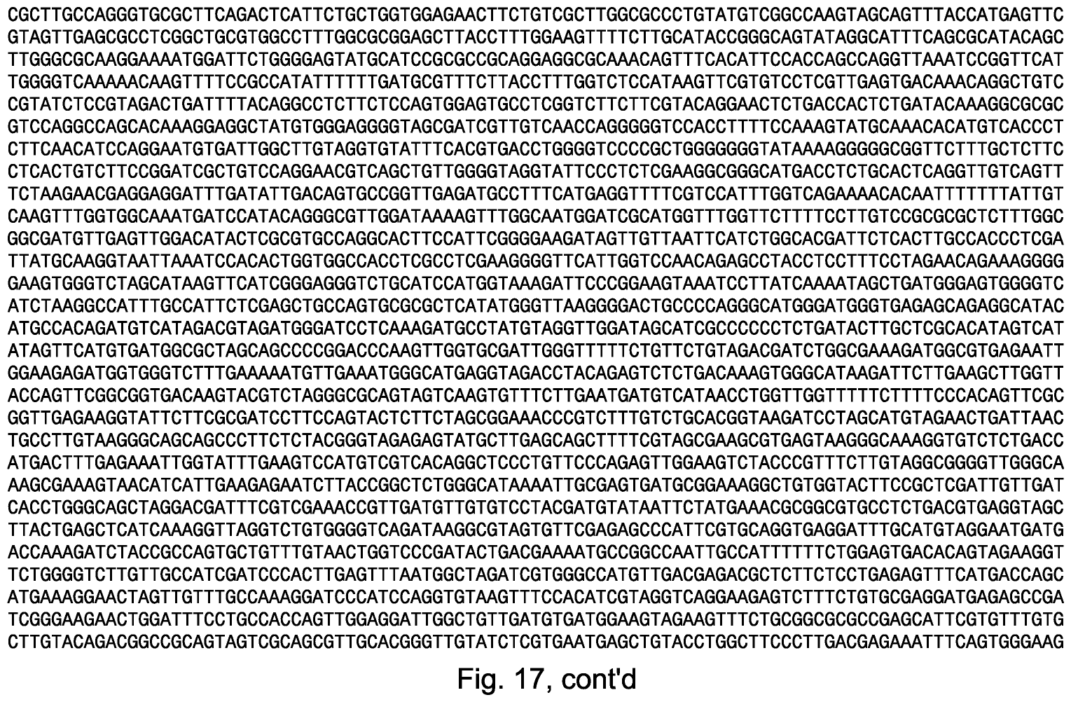

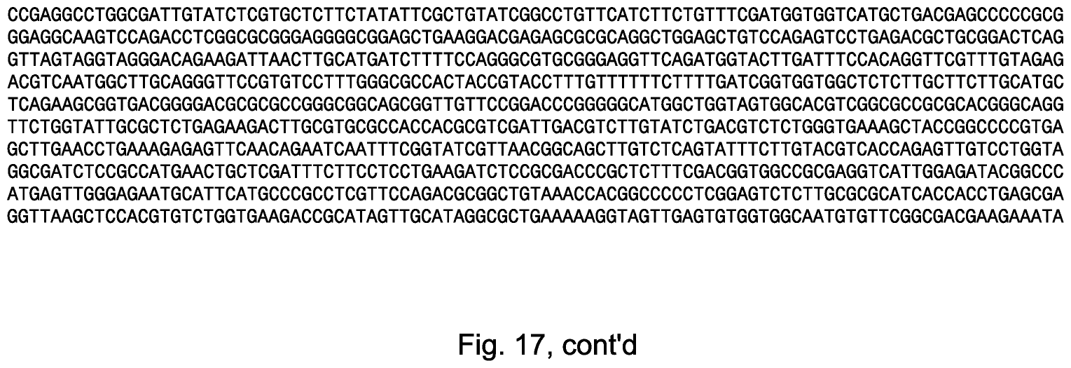

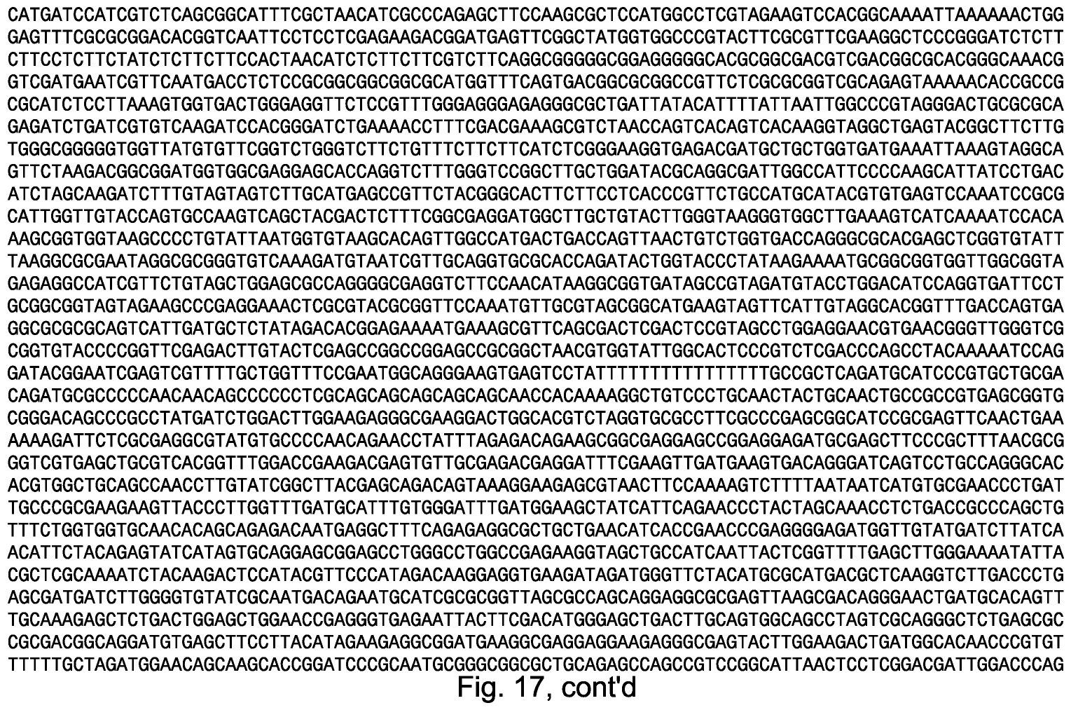

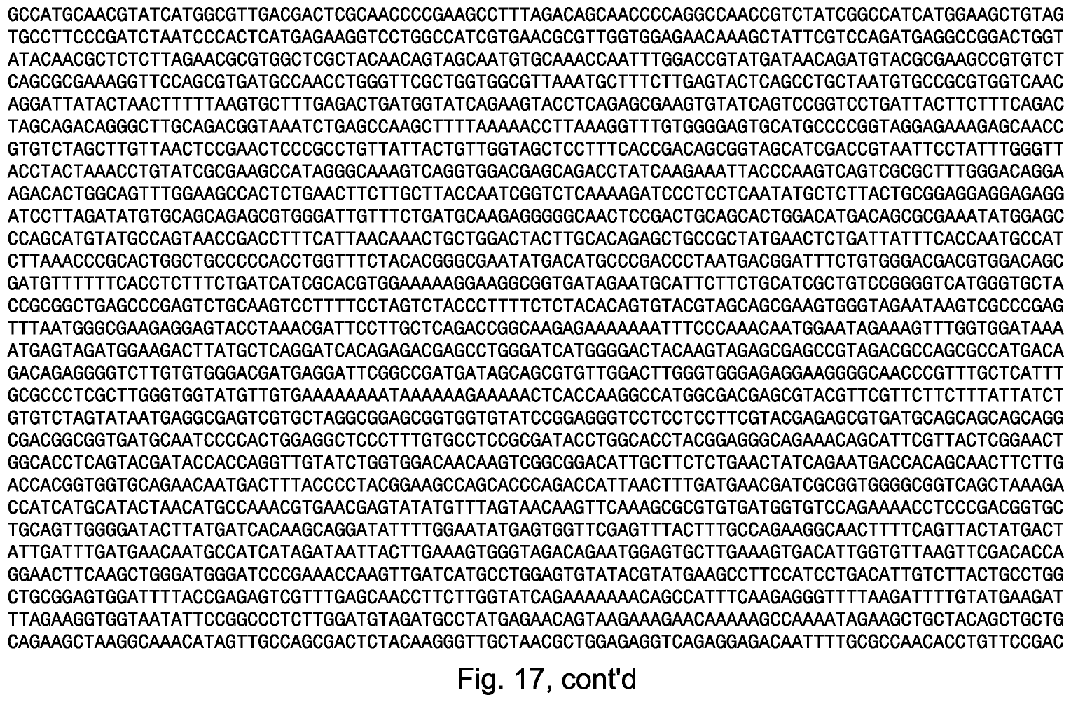

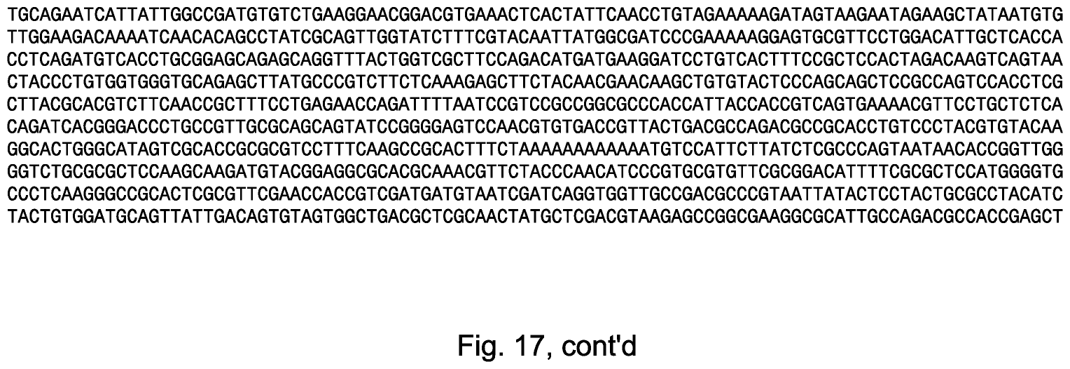

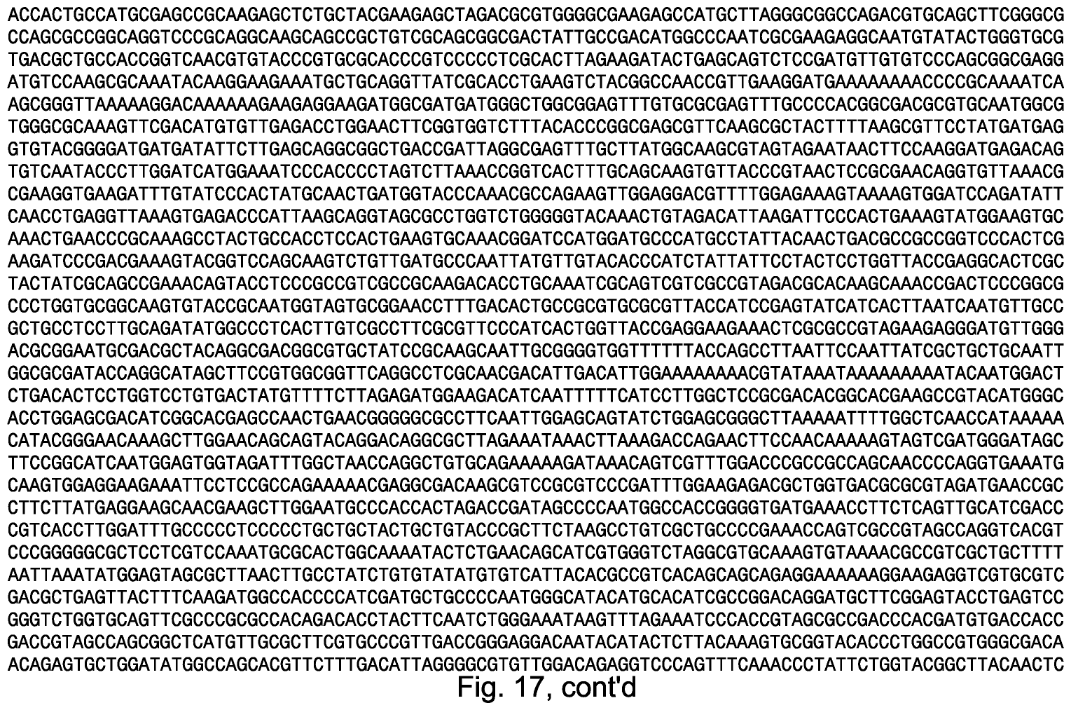

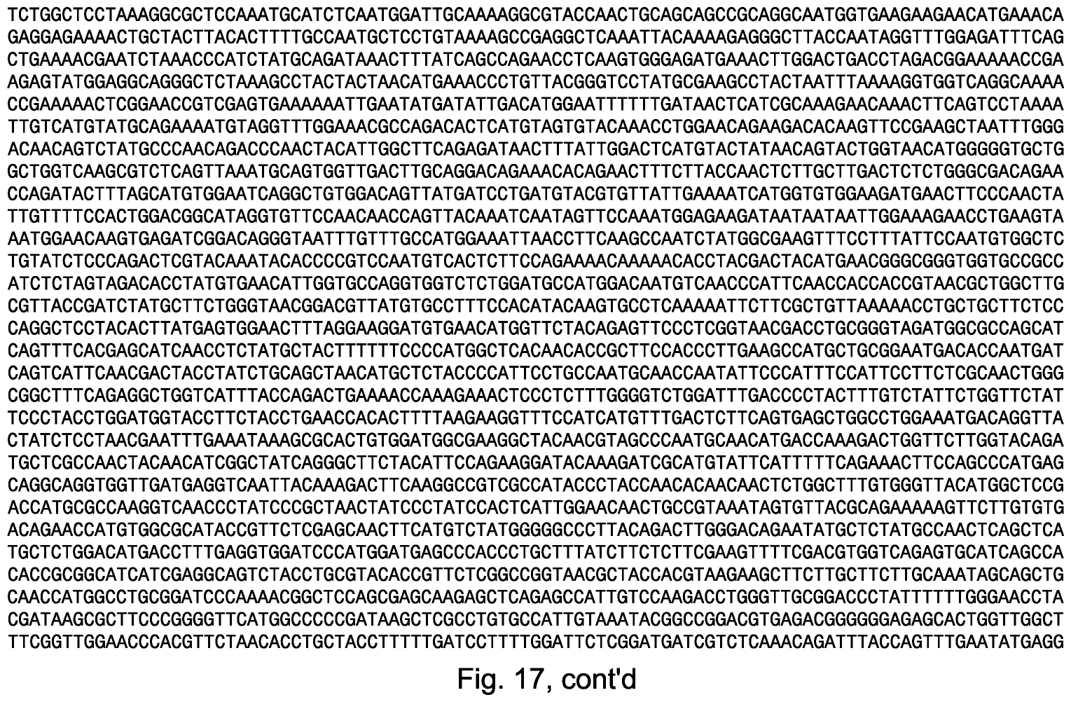

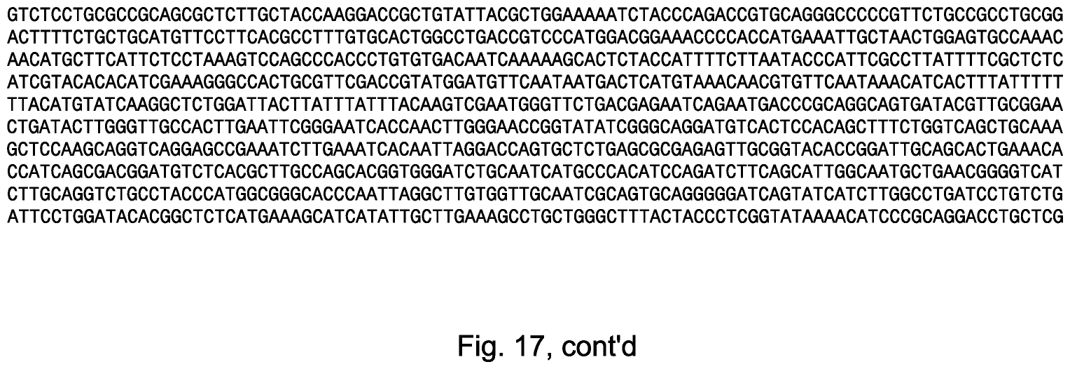

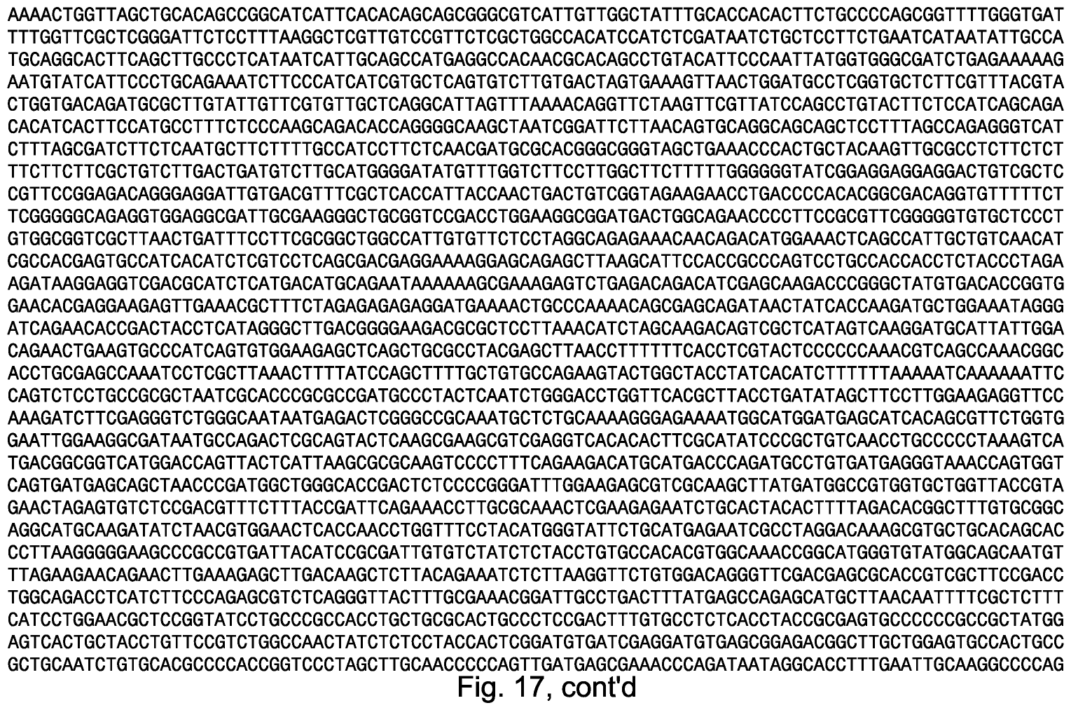

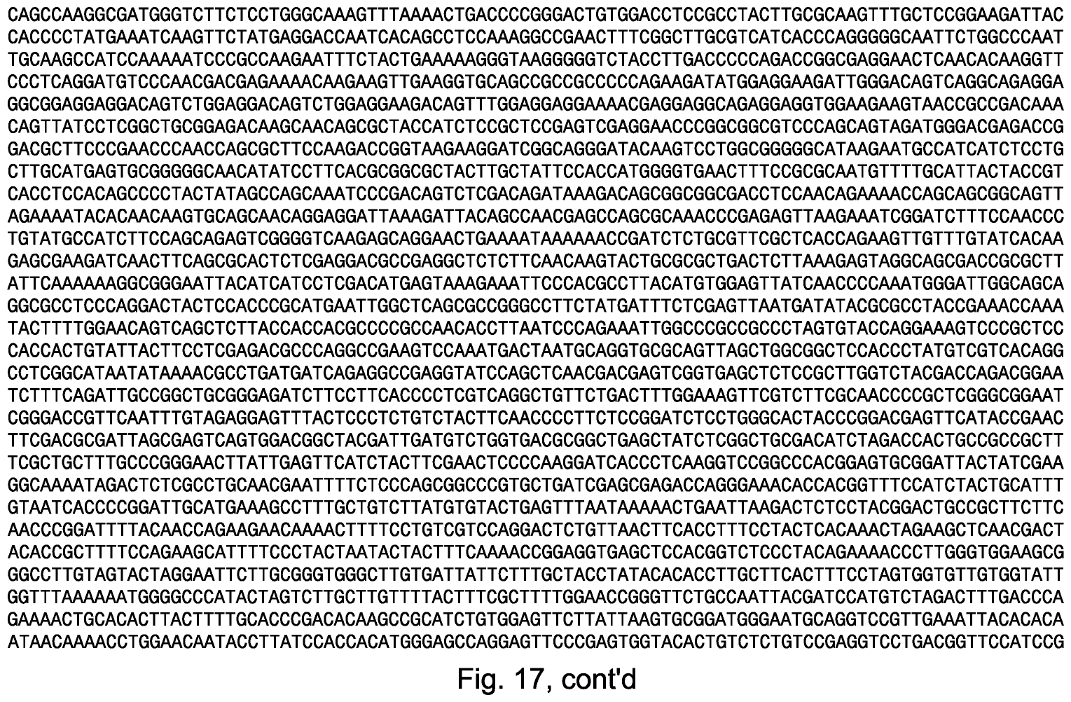

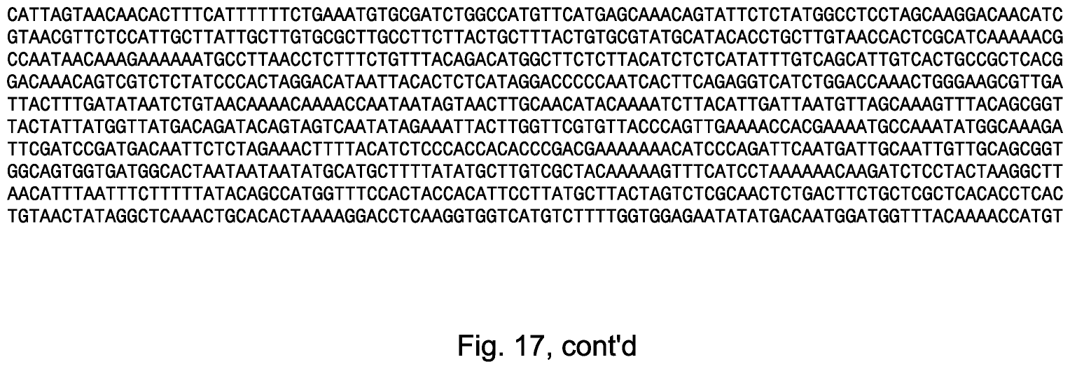

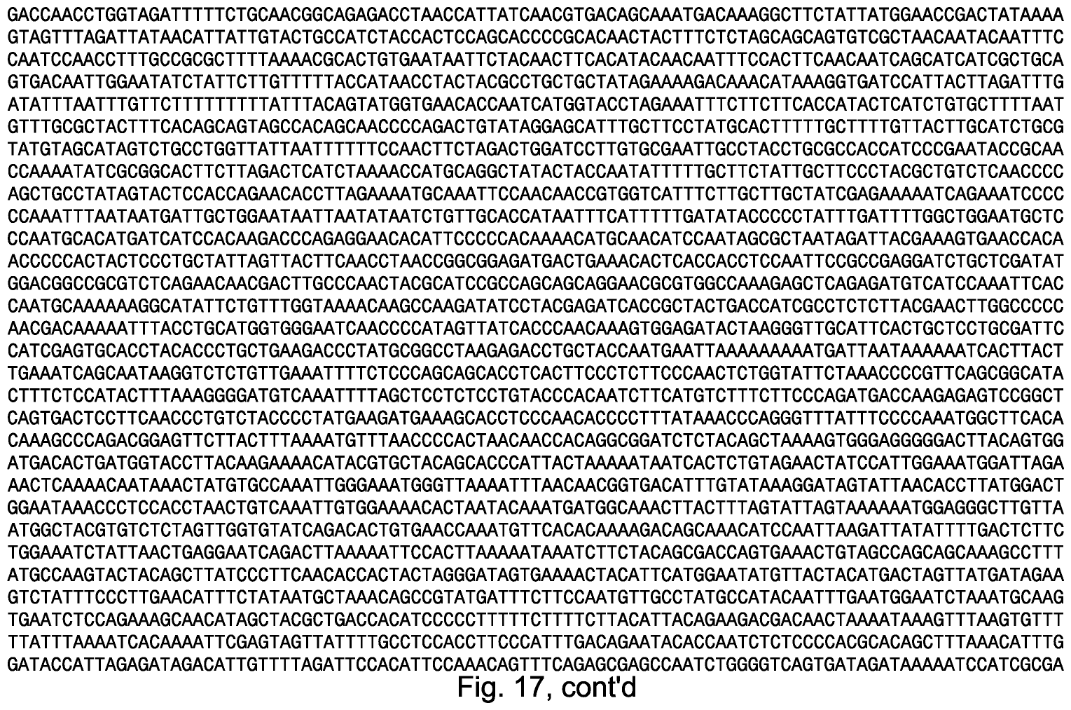

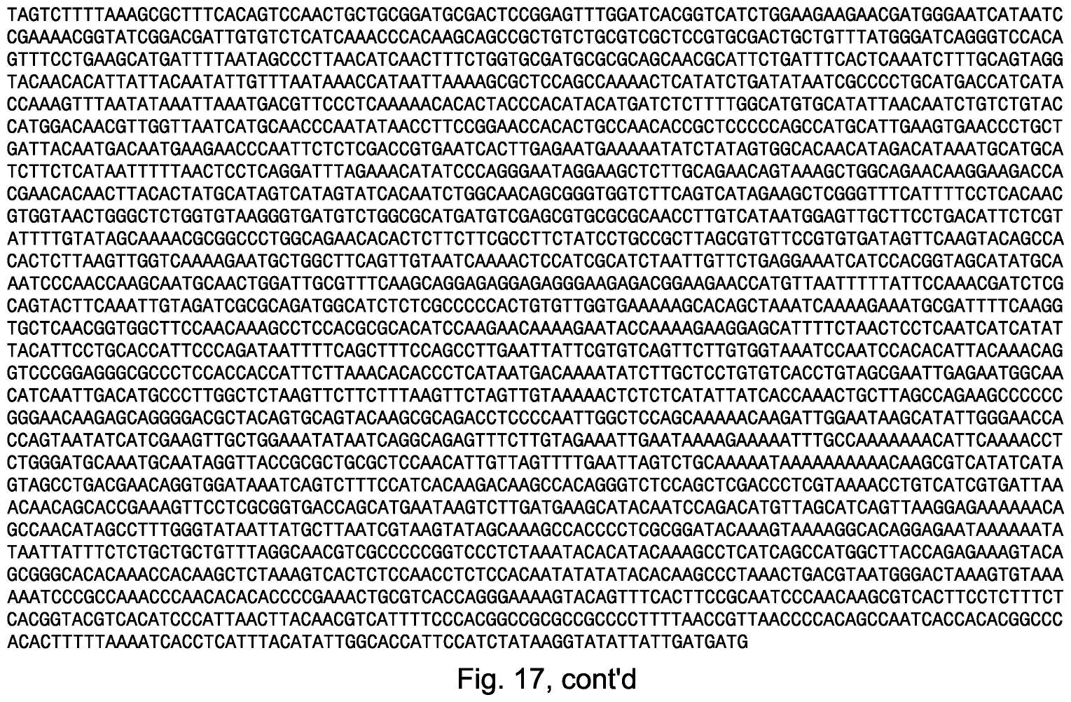

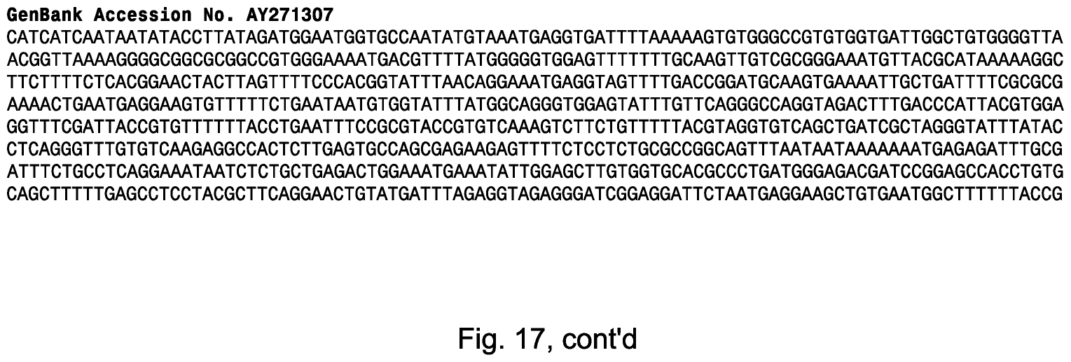

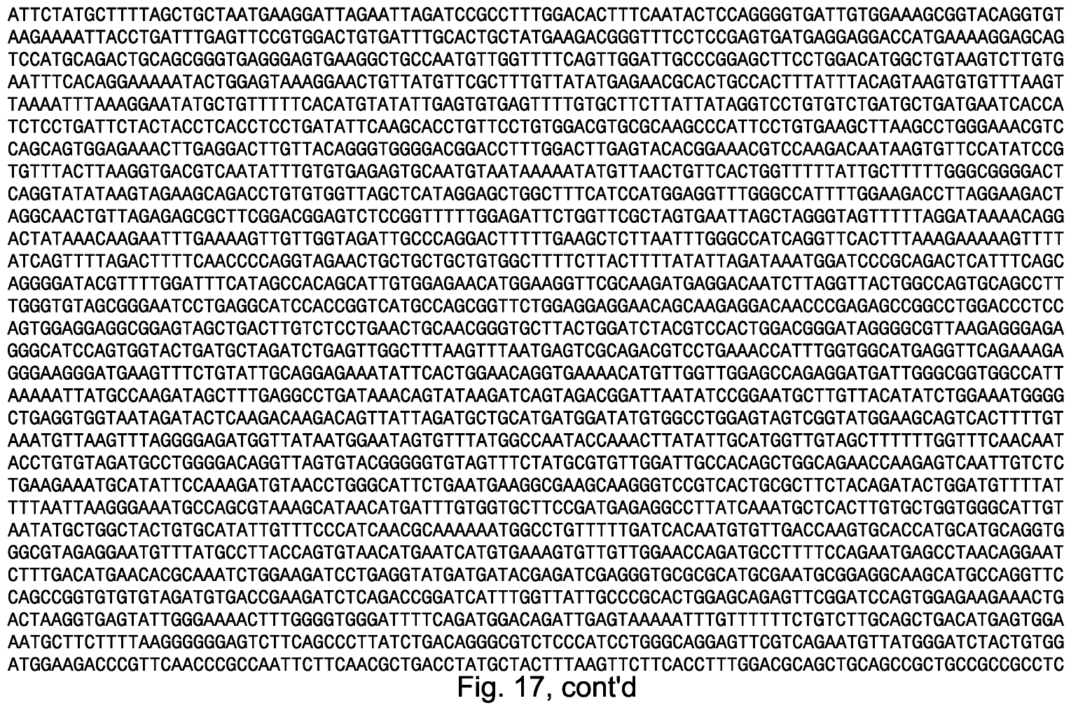

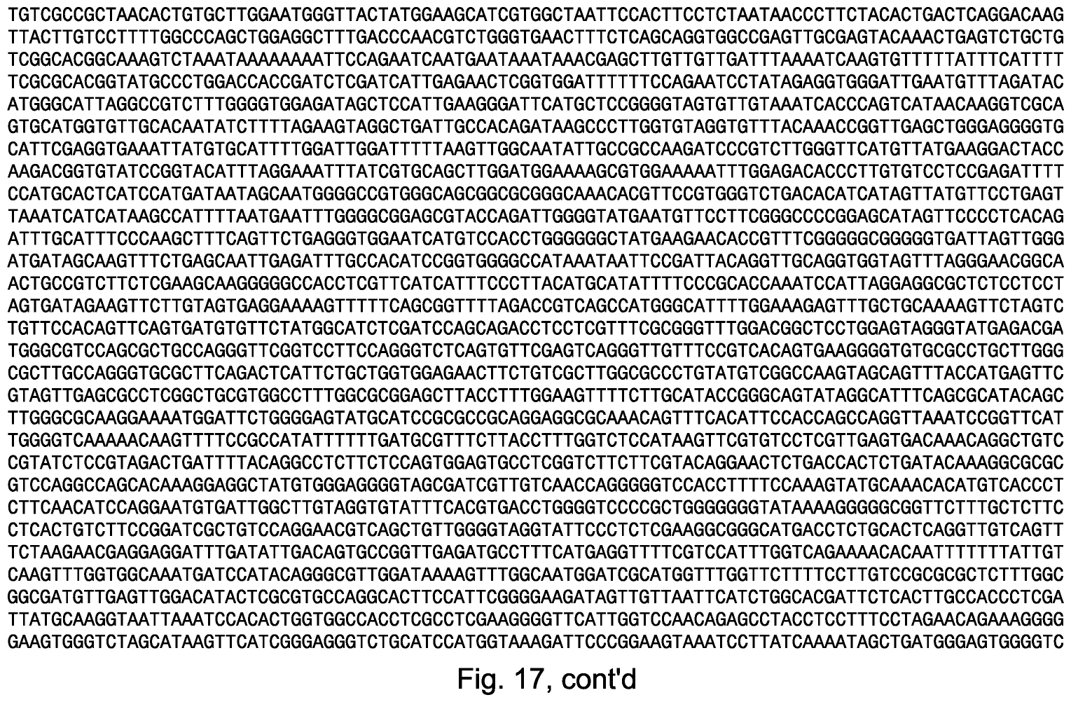

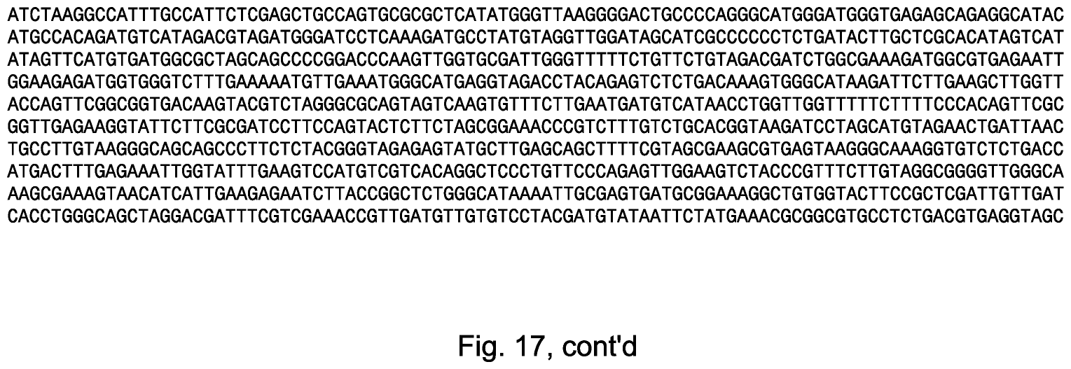

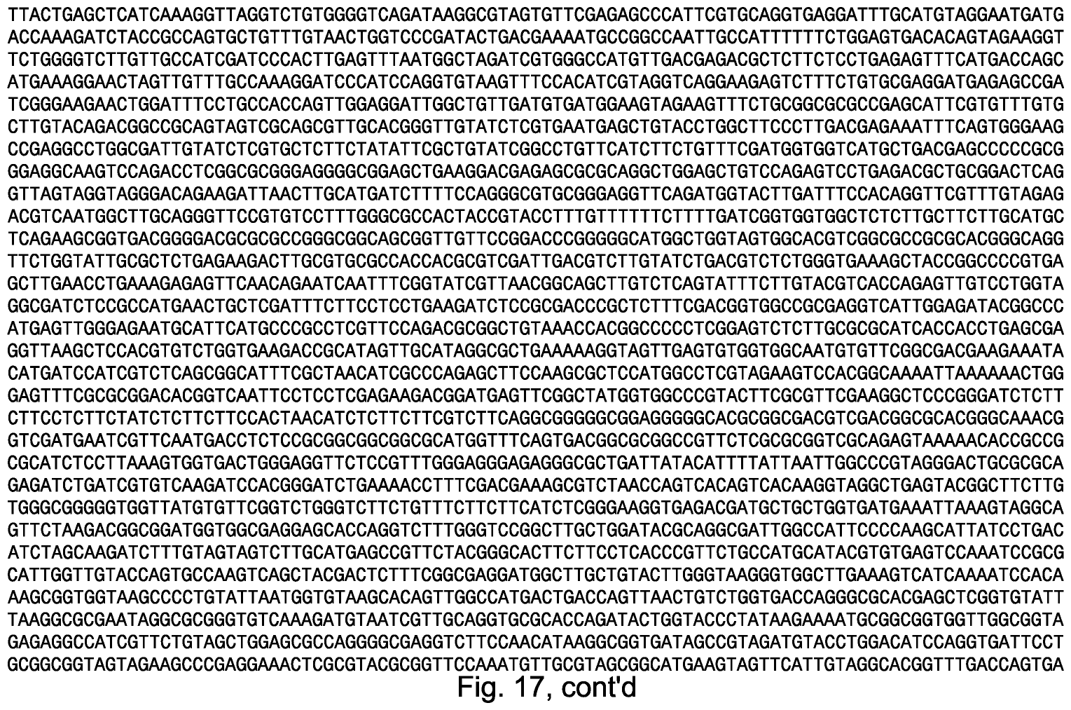

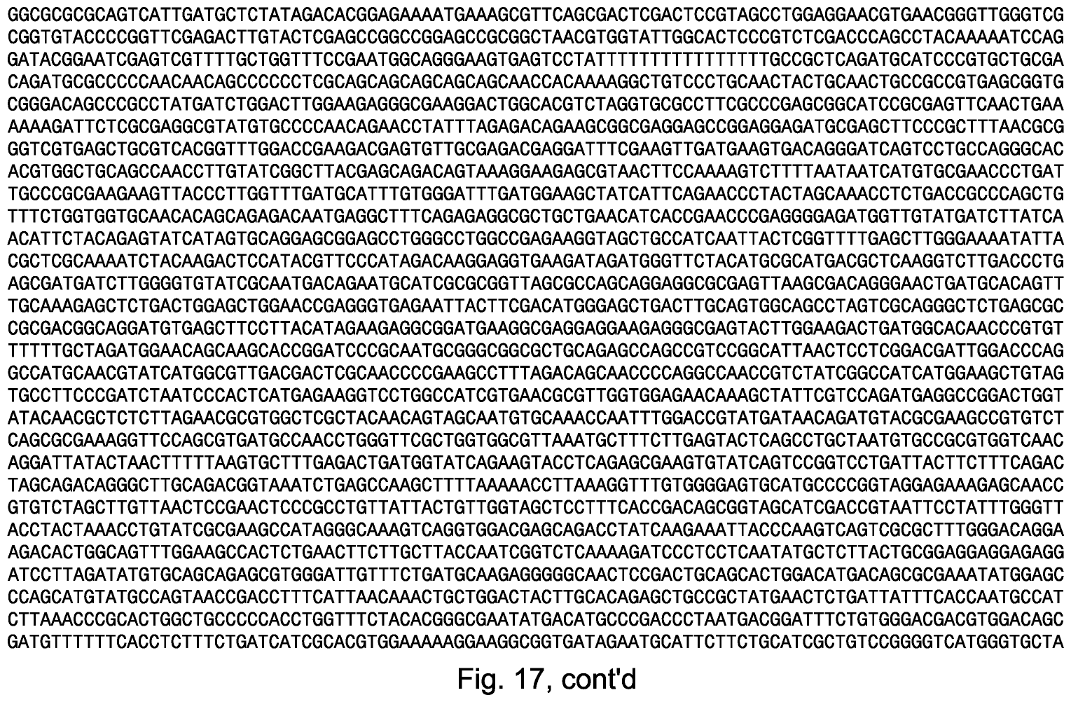

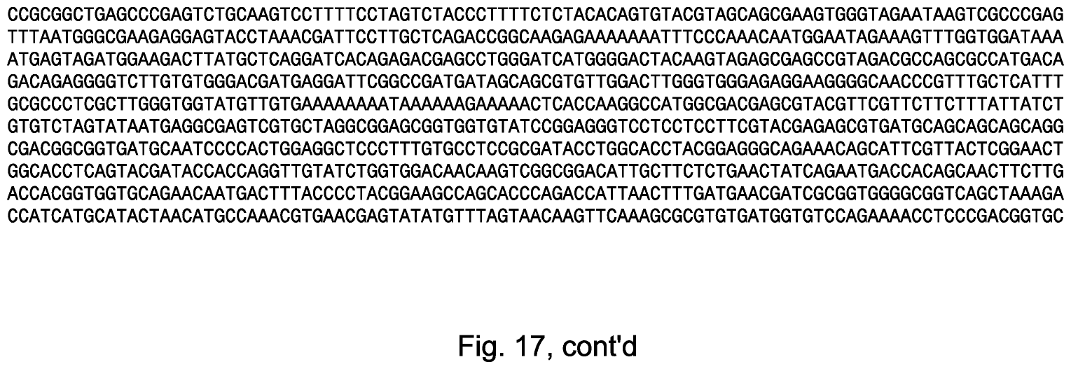

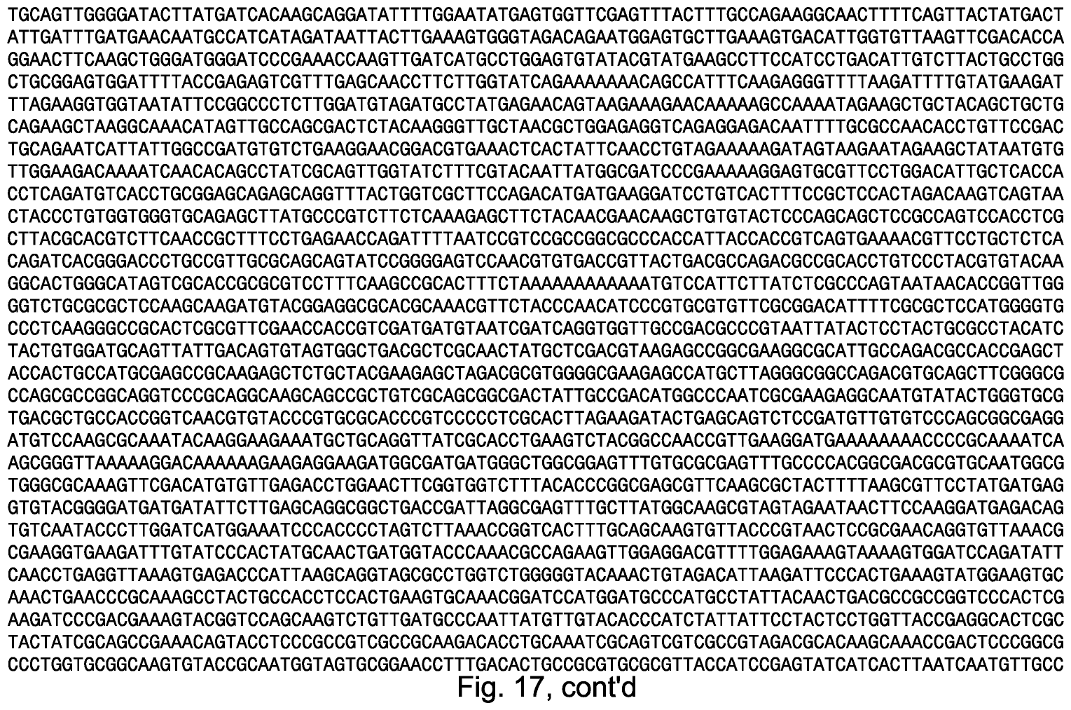

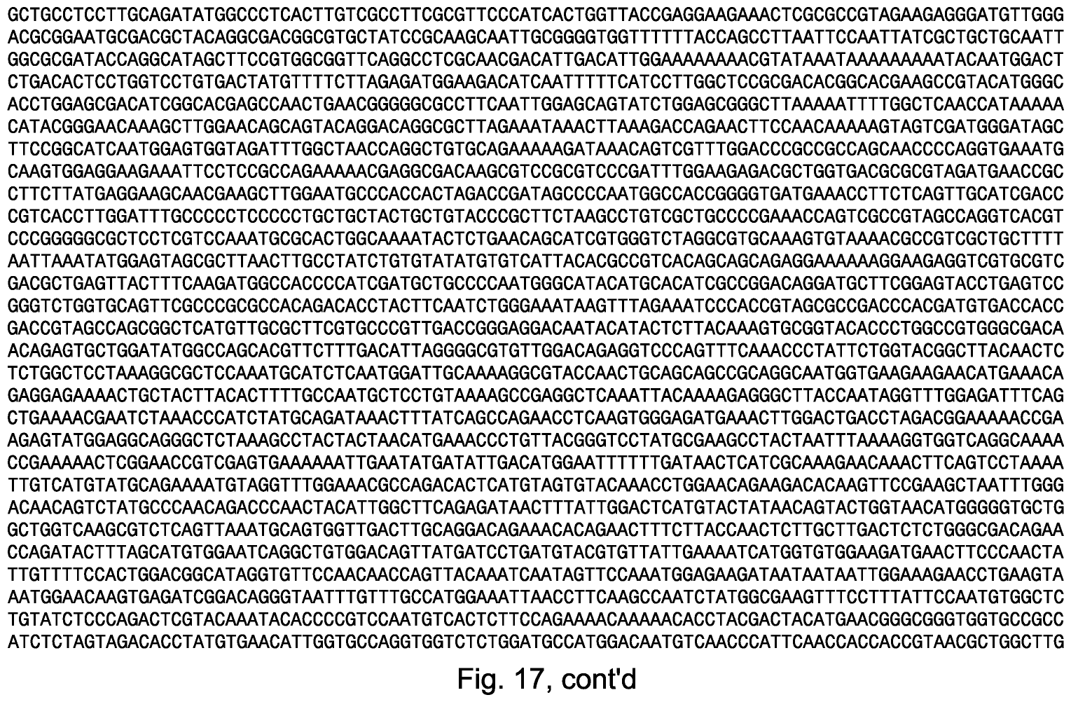

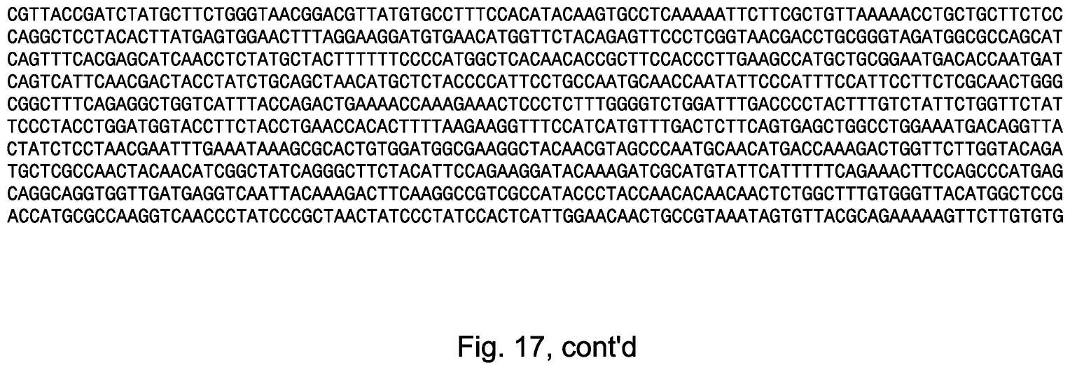

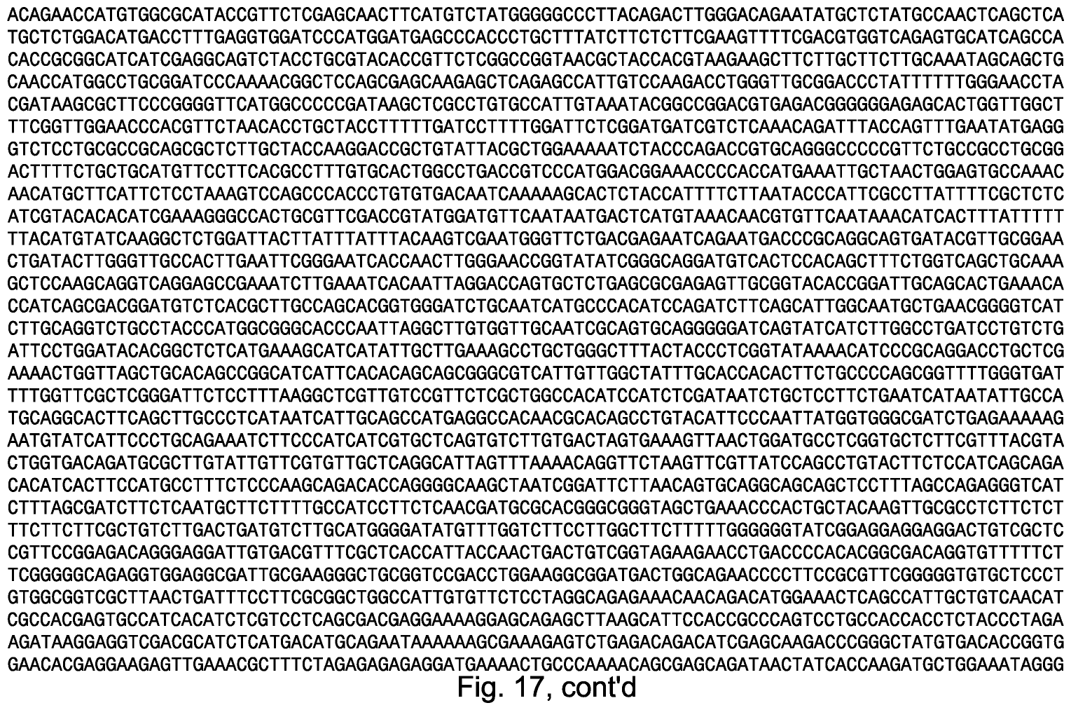

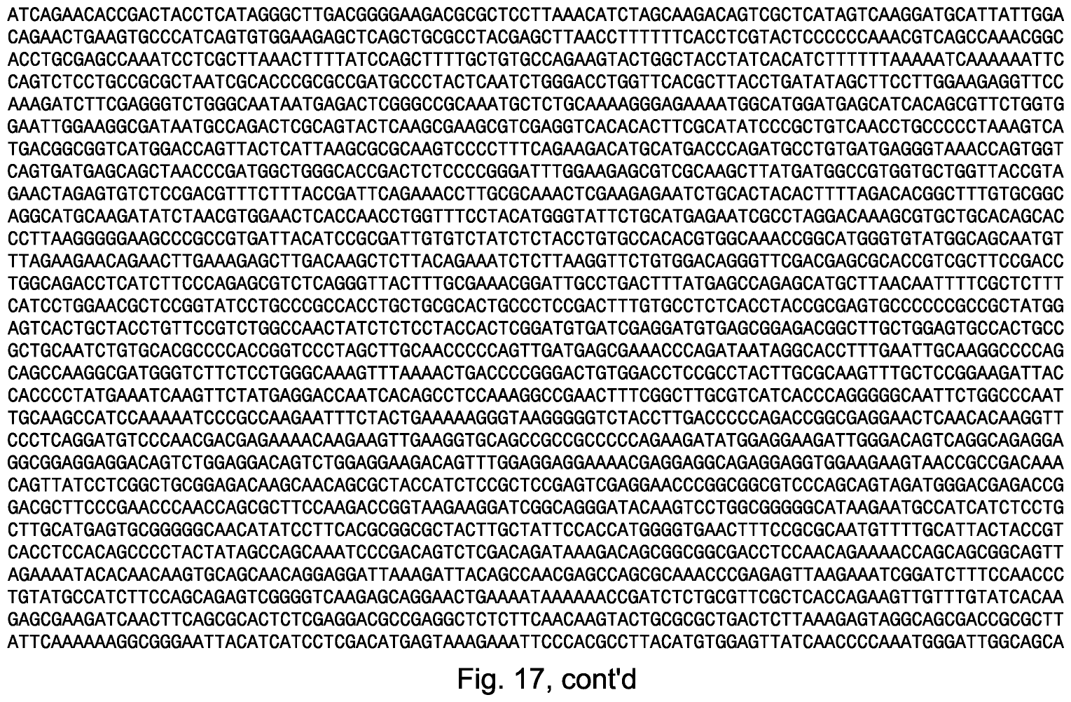

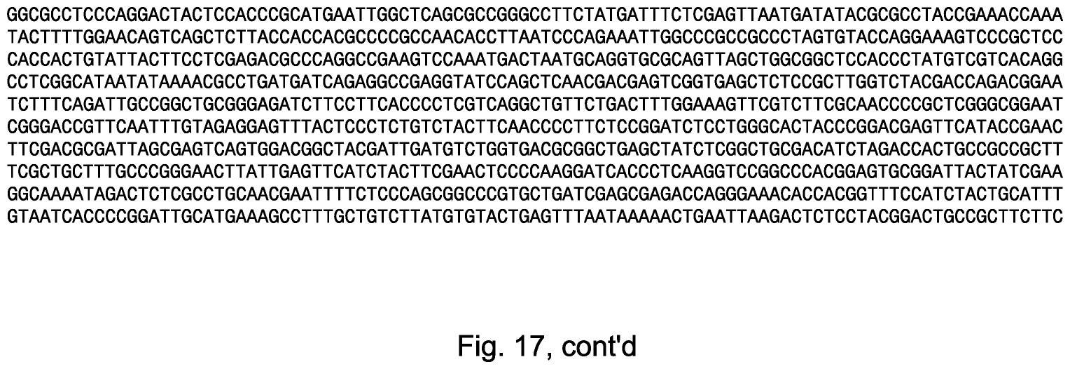

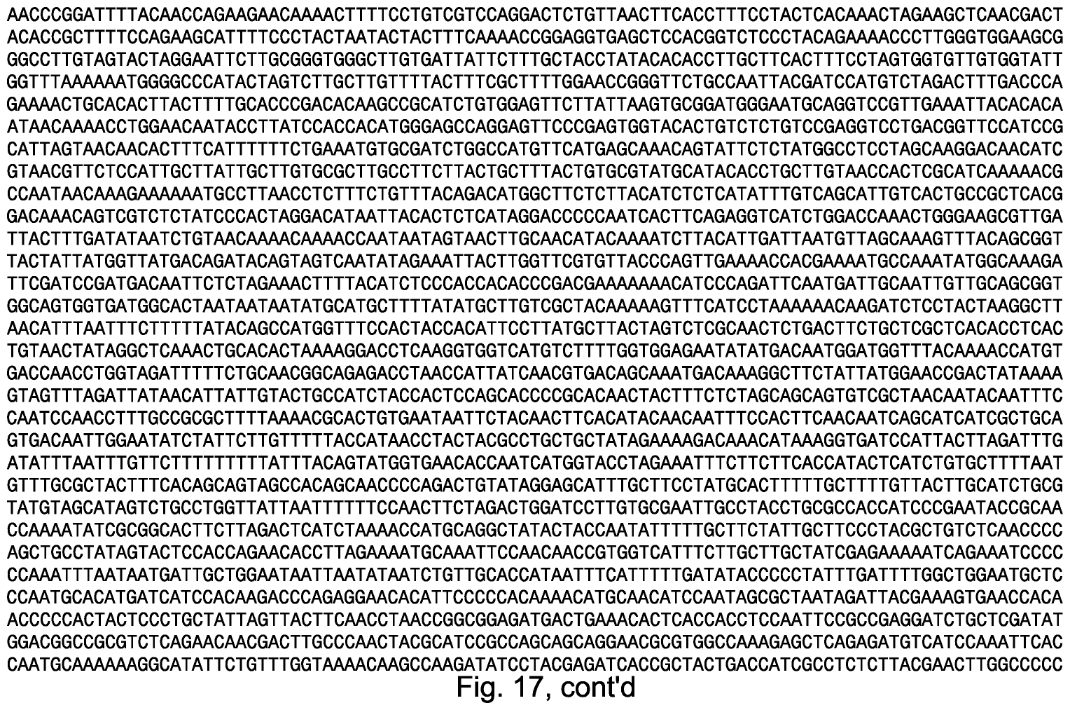

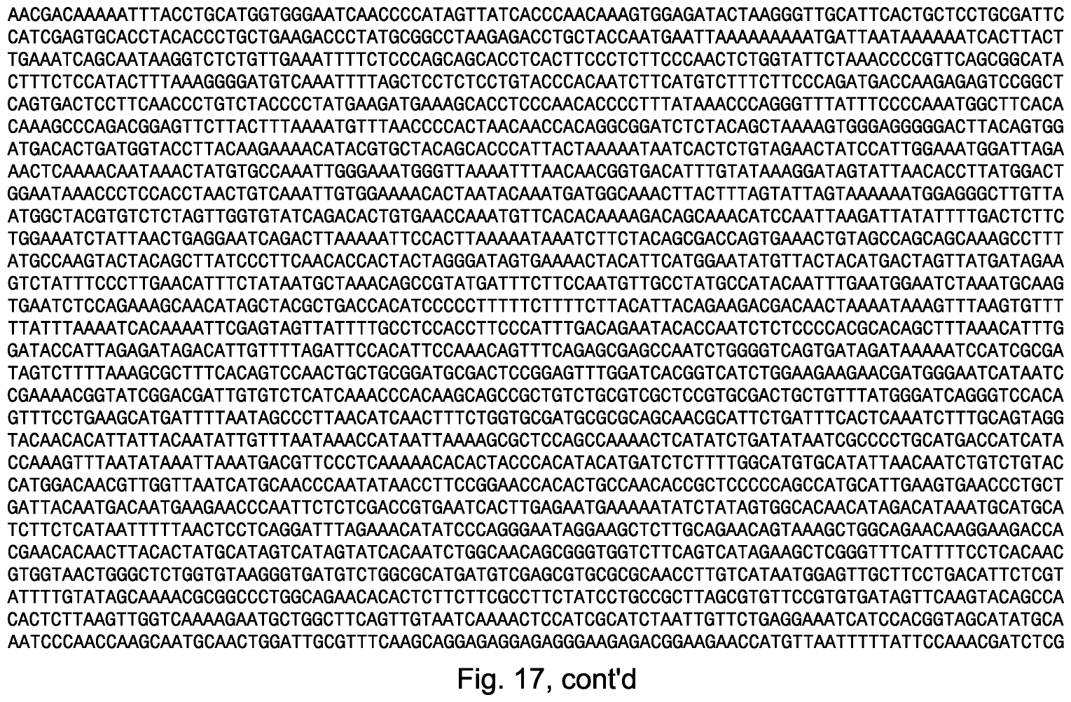

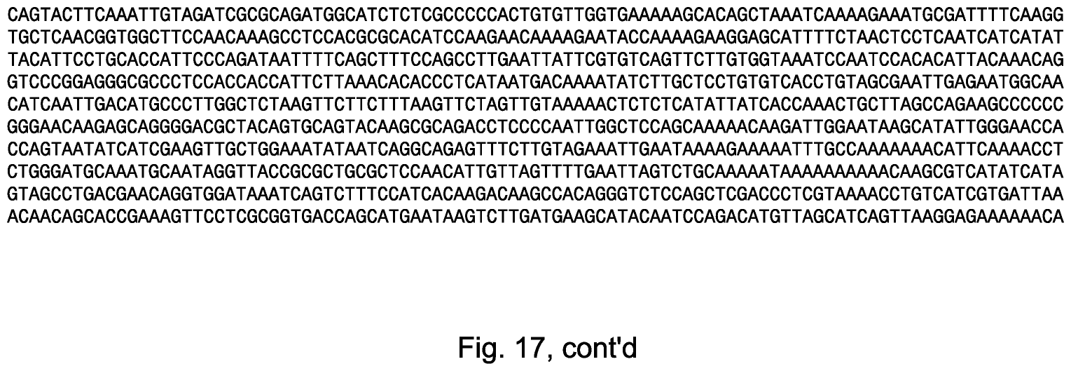

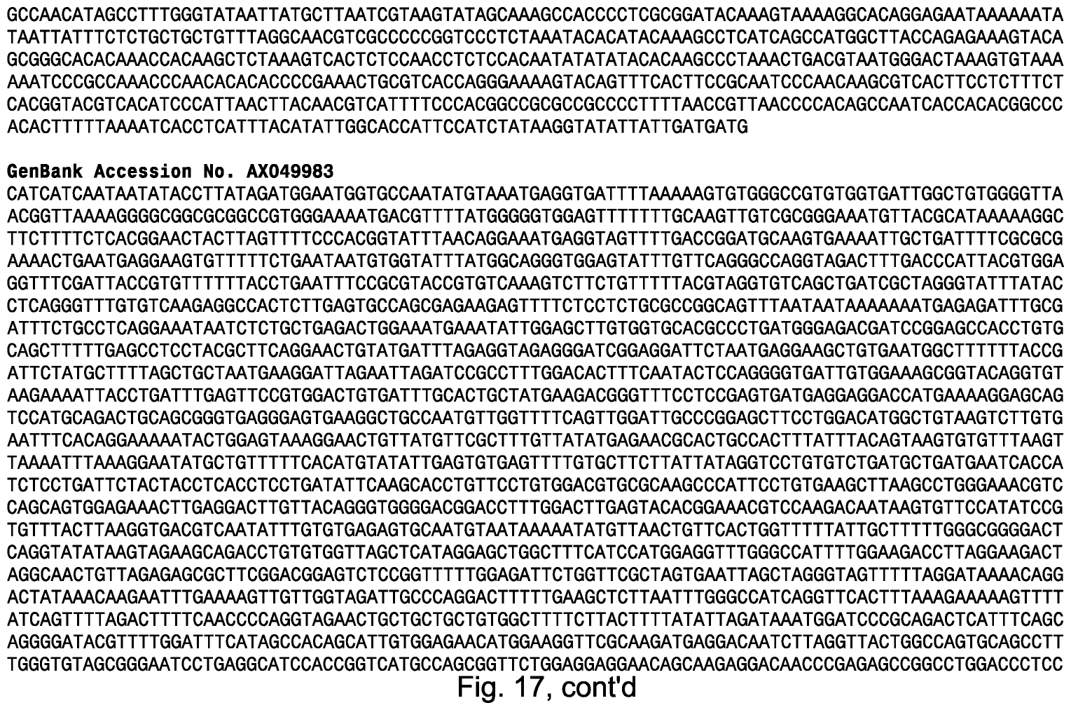

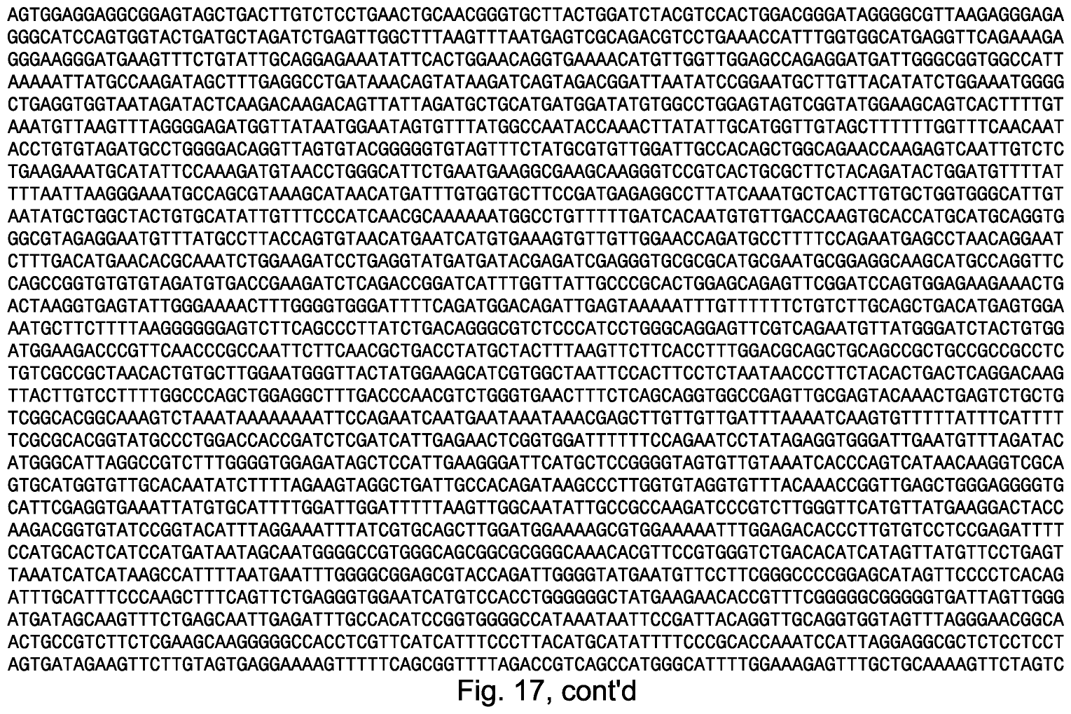

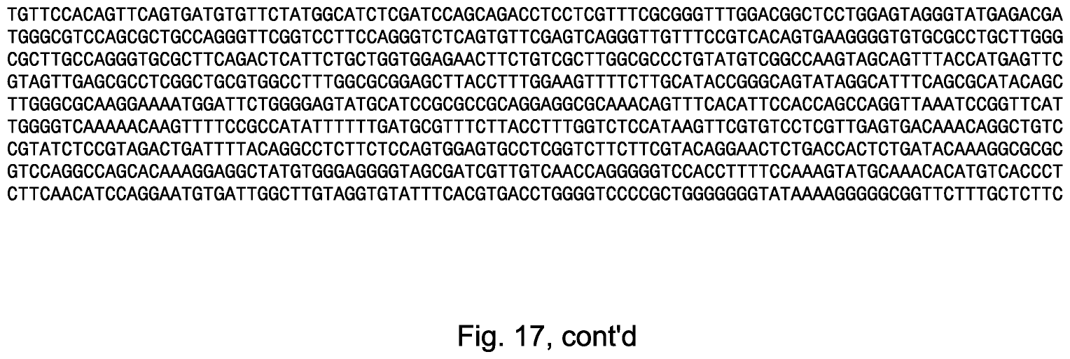

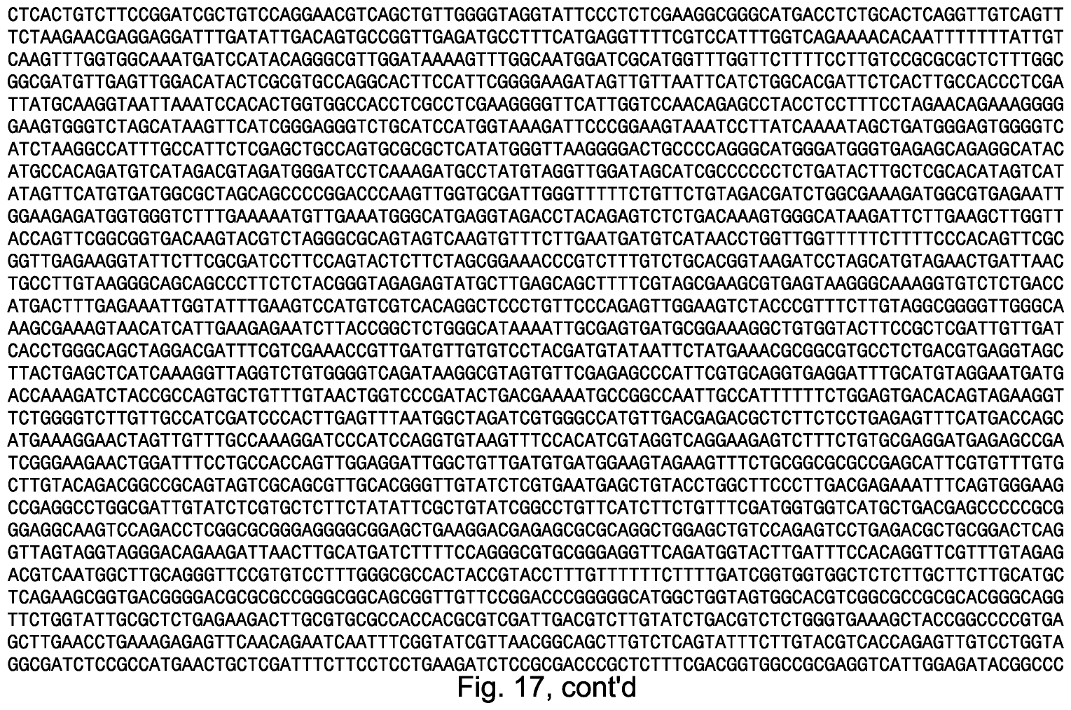

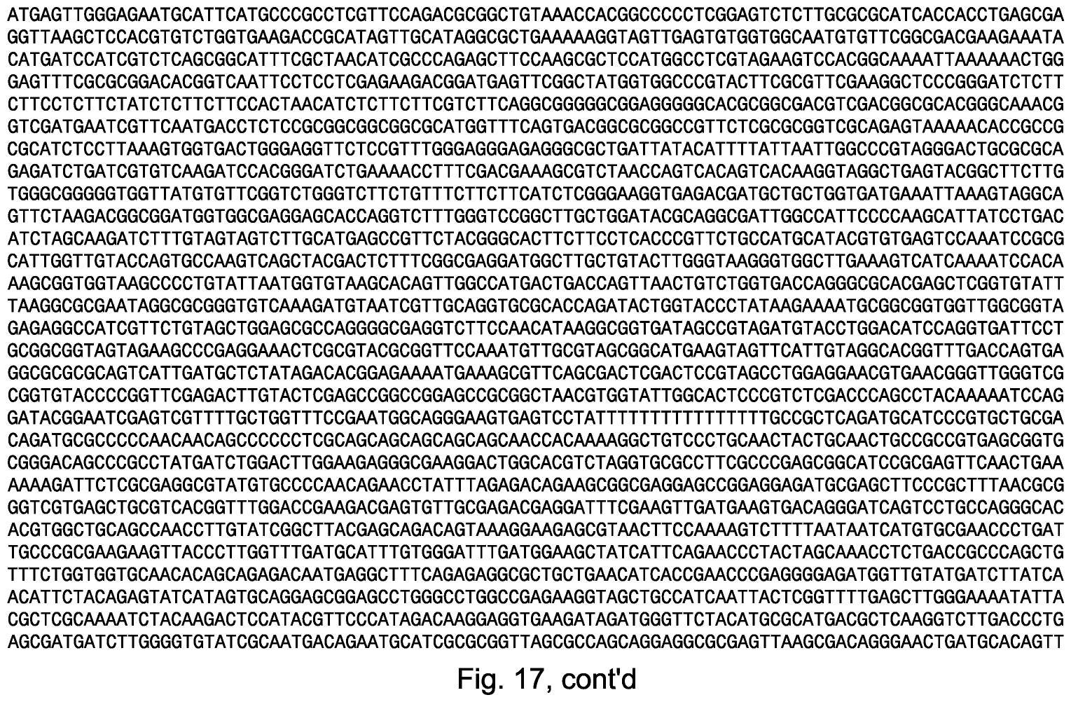

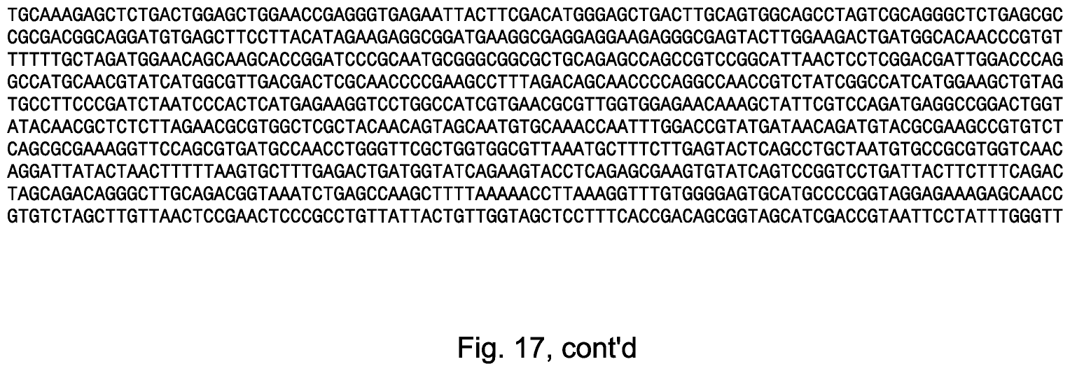

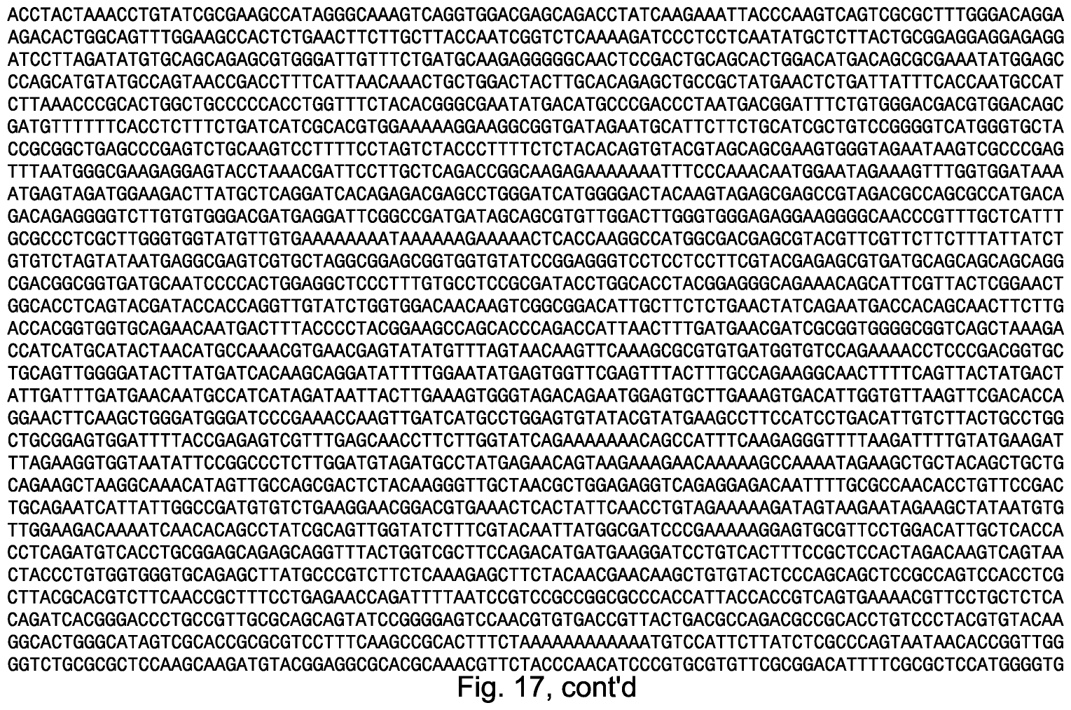

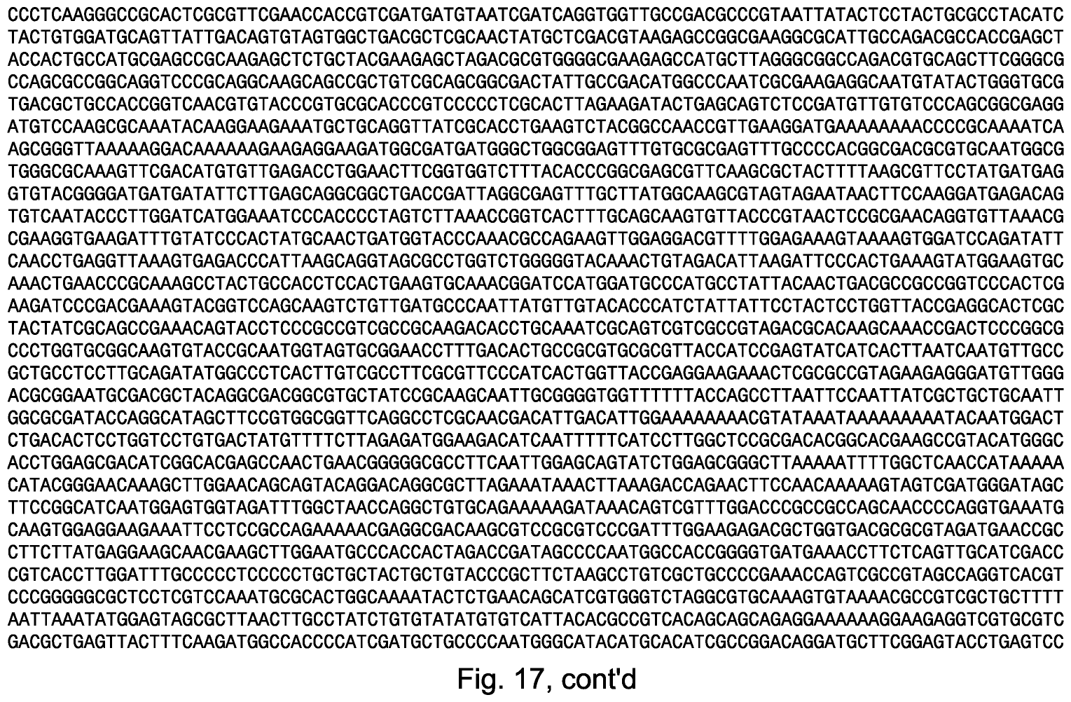

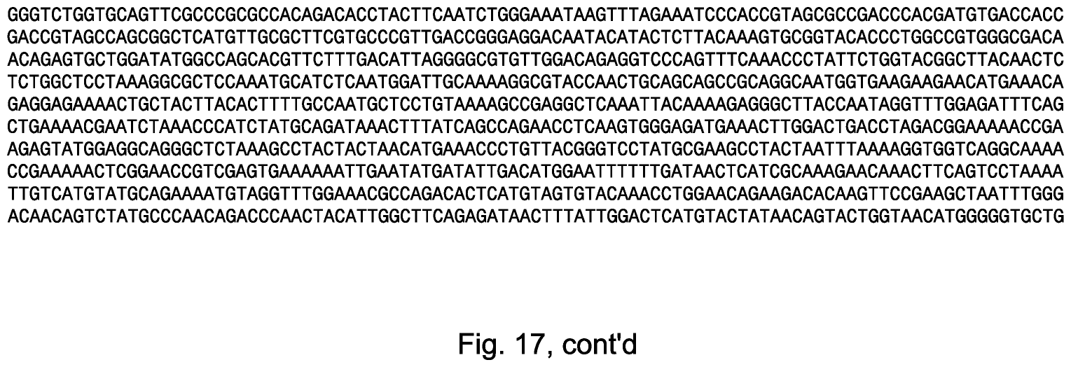

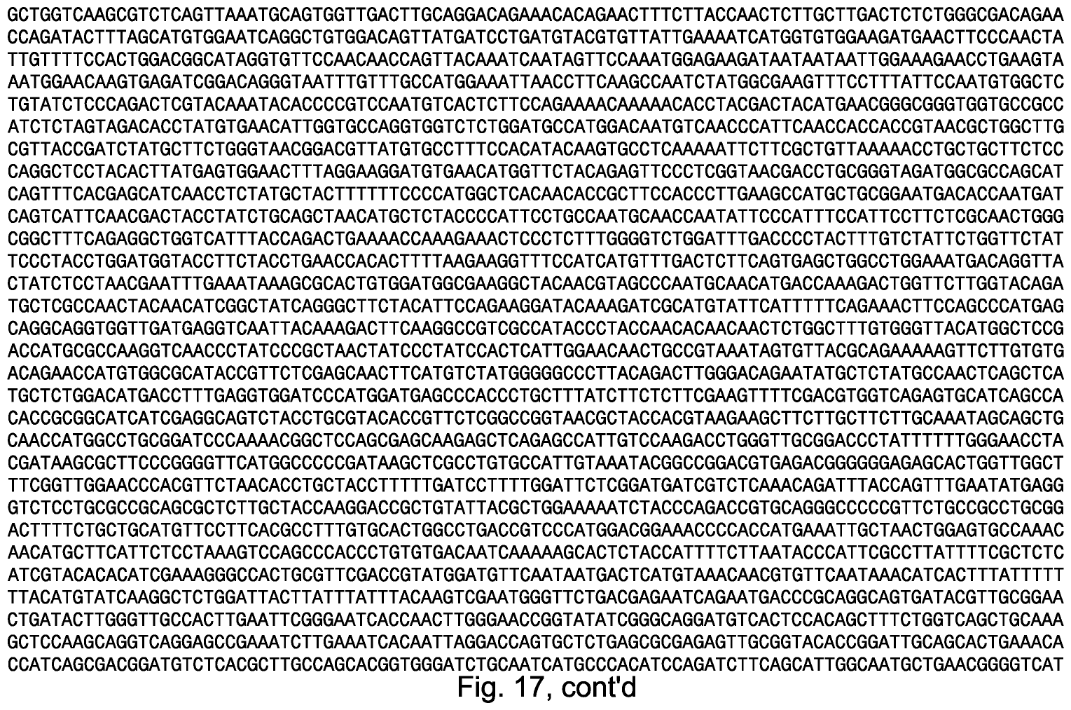

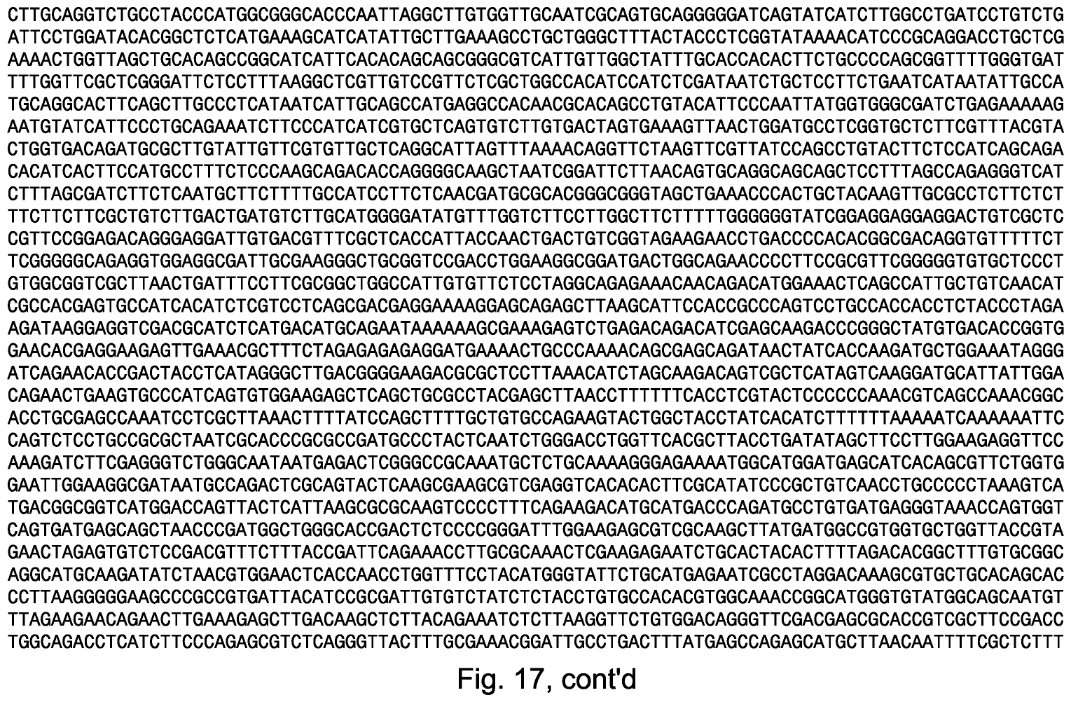

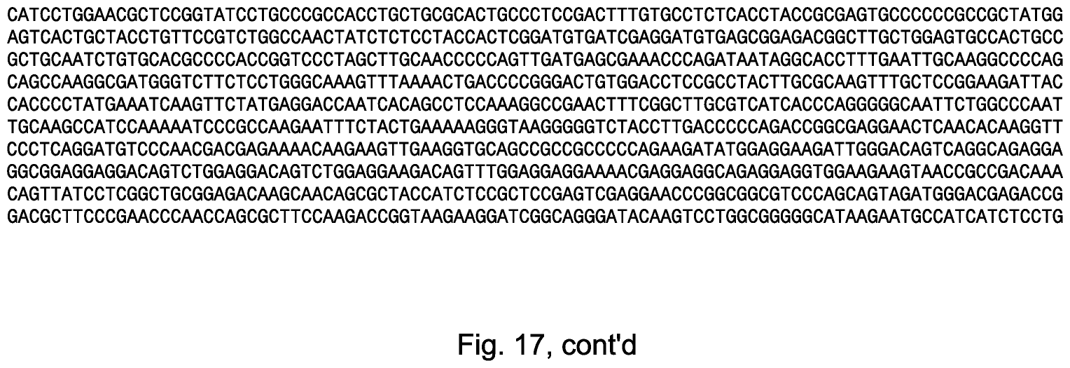

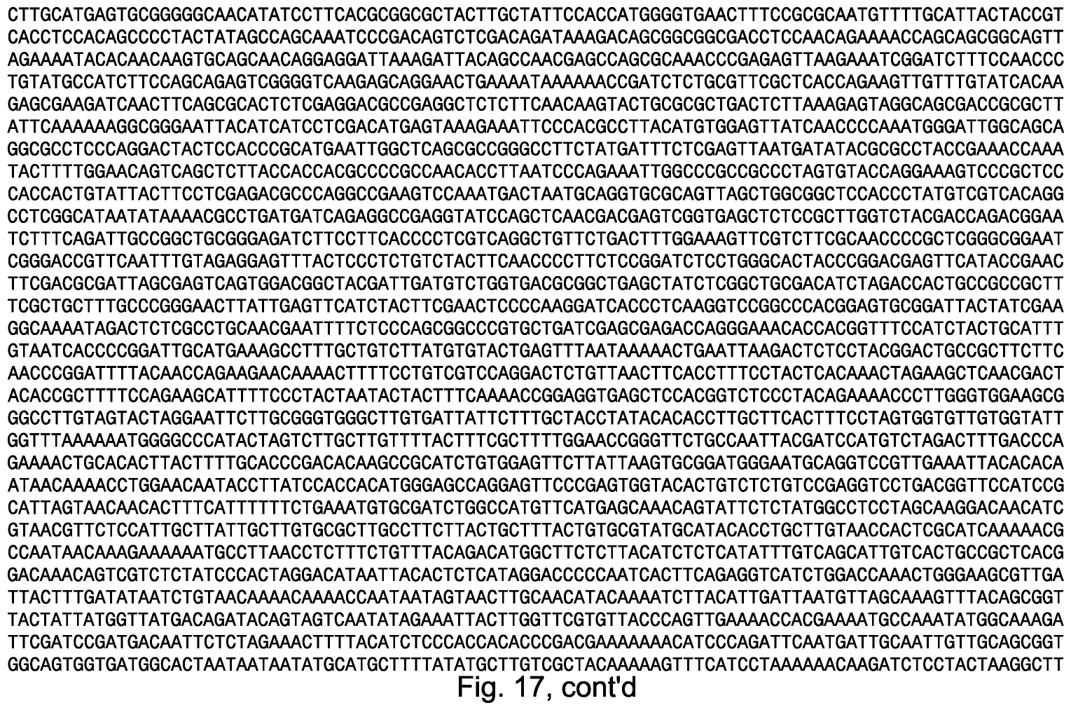

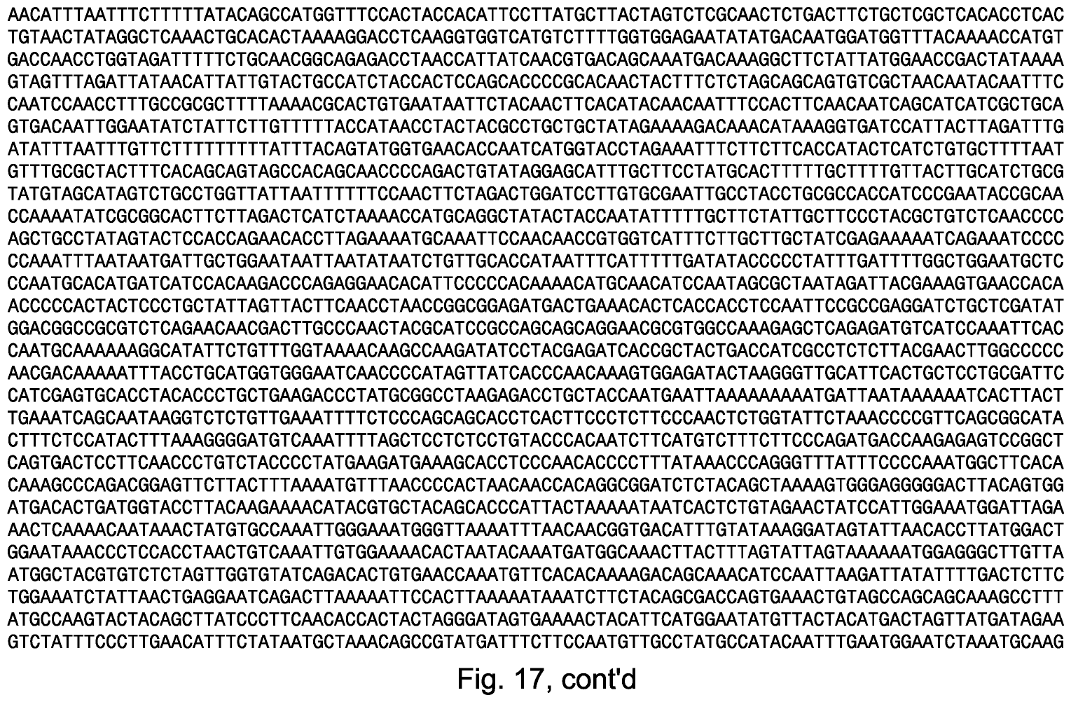

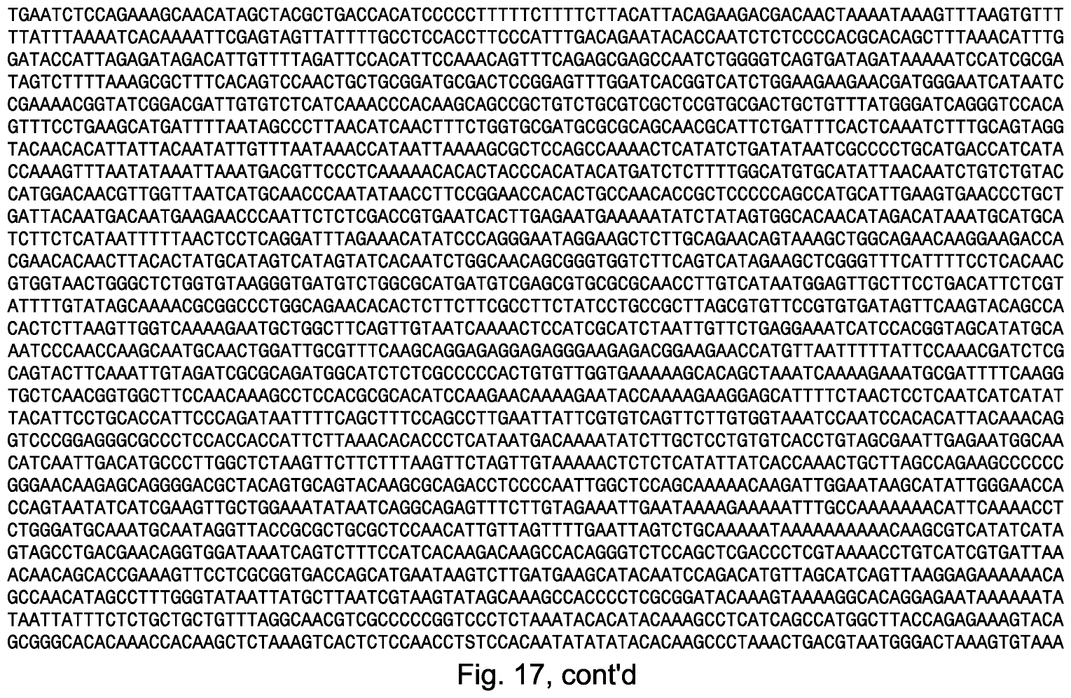

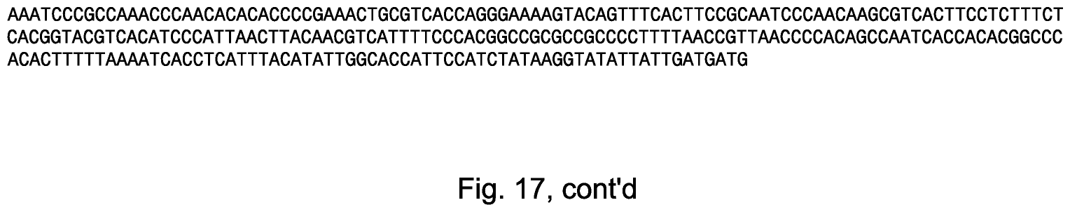

Various sequences corresponding to accession numbers disclosed herein are provided herein in FIG. 17. Those of skill in the art will appreciate that such sequences, including sequences disclosed in FIG. 17, can be referenced in whole (e.g., by an accession number), or in part (e.g., by reference to a nucleotide position and/or a set or range of nucleotide positions of a sequence and/or accession number).

Adenoviral genomes such as the Ad35 genome include DNA flanked on both ends by serotype-specific inverted terminal repeats (ITRs), which are understood to be cis elements that contribute to or are necessary for viral genome replication. Dependent on the serotype, ITRs can be, e.g., approximately 100-200 base pairs (e.g., about 160 base pairs) in length, with highest conservation at nucleotide positions (e.g., ˜50 base pairs) closest to the adenoviral genome termini. In various embodiments, Ad35 ITRs include 137 bp (e.g., a 5′ Ad35 that includes nucleotides 1-137 or 4-140 of GenBank Accession No. AY128640 and a 3′ ITR that includes nucleotides 34658-34794 of GenBank Accession No. AY128640). In various embodiments, an Ad35 5′ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% sequence identity) with a corresponding fragment of nucleotides 1-200 of GenBank Accession No. AY128640 and an Ad35 3′ ITR includes at least 80 nucleotides (e.g., at least 80, 90, 100, 110, 120, 130, 140, 150, 160, 170, 180, 190, or 200 nucleotides, e.g., a number of nucleotides having a lower bound of 80, 90, 100, 110, 120, or 130 nucleotides and an upper bound of 130, 140, 150, 160, 170, 180, 190, or 200 nucleotides, e.g., 137 nucleotides) having at least 80% sequence identity (e.g., at least 80%, 85%, 90%, 95%, 96%, 97%, 98%, or 99% sequence identity) with a corresponding fragment of nucleotides 34595-34794 of GenBank Accession No. AY128640. In various embodiments, an ITR is sufficient for one or both of Ad35 encapsidation and/or replication. In various embodiments, an Ad35 ITR sequence for Ad35 vectors differs in that the first 8 bp are CTATCTAT (SEQ ID NO: 12) rather than CATCATCA (SEQ ID NO: 13) (Wunderlich, J. Gen Viro. 95: 1574-1584, 2014).

Adenoviral genomes also include a cis-acting packaging sequence (e.g., a conditional or non-conditional packaging sequence, the packaging sequence sometimes represented by the symbol w), which can facilitate packaging of the viral genome into viral vectors. In various embodiments, a packaging sequence can be positioned in the 5′ portion of an Ad genome, with the 5′ ITR.

Natural adenoviral genomes encode several proteins including early transcriptional units, E1, E2, E3, and E4 and late transcriptional units which encode structural protein components of the adenoviral vector. Early (E) and late (L) transcription are divided by the onset of viral genome replication. Late transcription includes expression of proteins that make up the viral capsid. Adenoviral capsids include three types of proteins: fiber, penton, and hexon.

Ad35 fiber is a fiber protein trimer, each fiber protein includes an N-terminal tail domain that interacts with the pentameric penton base, a C-terminal globular knob domain (fiber knob) that functions as the attachment site for the host cell receptors, and a central shaft domain that connects the tail and the knob domains (shaft). In various embodiments an Ad35 fiber knob has at least 80% sequence identity (e.g., at least 80%, 85%, 90%, 95%, 96%, 97%, 98%, or 99% sequence identity) with reference fiber sequence GenBank accession AP_000601. In various embodiments, an Ad35 fiber knob includes amino acids 123 to 320 or 323 of a canonical wild-type Ad35 fiber protein. In various embodiments, an Ad35 fiber knob includes at least 60 amino acids (e.g., at least 60, 70, 80, 90, 100, 110, 120, 130, 140, 150, 160, 170, 180, 190, or 198 amino acids) having at least 80% sequence identity (e.g., at least 80%, 85%, 90%, 95%, 96%, 97%, 98%, or 99% sequence identity) with a corresponding fragment of amino acids 123 to 320 or 323 of a canonical wild-type Ad35 fiber protein.

An exemplary genome of a natural Ad35 adenovirus is known and publicly available (see, e.g., Gao et al., 2003 Gene Ther. 10(23): 1941-9; Reddy et al. 2003 Virology 311(2): 384-393; GenBank Accession No. AY128640). Table 1 provides an example summary of expression products encoded by an Ad35 genome (see Gao, Gene Ther. 10:1941-1949, 2003).

TABLE 1
Predicted translational features of the Ad35 genome.

| Features | From | To |
|---|---|---|
| 5′ (left) ITR | 1 | 137 |
| Packaging Sequence | 138 | 479 |

E1 and pIX regions (E1: 480-3400)

| | | | |
|---|---|---|---|
| E1A 261R | | 569 | 1148 |
| | Join* | 1233 | 1441 |
| E1A 230R | | 569 | 1055 |
| | Join | 1233 | 1441 |
| E1A 58R | | 569 | 640 |
| | Join | 1233 | 1337 |
| E1B 214R (small T | | 1611 | 2153 |
| antigen) | | | |
| E1B 494R (large T | | 1916 | 3400 |
| antigen) | | | |
| pIX | | 3484 | 3903 |
| ORF-1 | | 2366 | 2689 |

E2 and IVa2 regions (complementary strand) (E2: 3966-23415)

| | | | |
|---|---|---|---|
| IVa2 | | 5579 | 5590 |
| | Join | 3966 | 5300 |
| E2B DNA pol | | 5069 | 8437 |
| E2B pTP | | 8440 | 10 356 |
| E2A DBP | | 22 414 | 23 415 |
| ORF-2 | | 5988 | 6482 |
| ORF-3 | | 7847 | 8257 |
| ORF-4 | | 15 663 | 15 971 |
| ORF-5 | | 15 743 | 16 216 |
| ORF-6 | | 16 457 | 17 041 |
| ORF-7 | | 17 543 | 17 938 |
| ORF-8 | | 17 994 | 18 713 |
| ORF-9 | | 21 858 | 22 436 |
| ORF-10 | | 22 128 | 22 502 |
| ORF-11 | | 23 027 | 23 488 |

E3 region (E3: 27198-30622)

| | | |
|---|---|---|
| E3 12.2K protein | 27 198 | 27 515 |
| E3 15.0 K protein | 27 469 | 27 864 |
| E3 18.5K protein | 27 849 | 28 349 |
| E3 20.3K protein | 28 369 | 28 914 |
| E3 20.6K protein | 28 932 | 29 495 |
| E3 15.2K protein | 29 817 | 30 221 |
| E3 15.3K protein | 30 214 | 30 621 |
| ORF-12 | 25 693 | 26 019 |
| ORF-13 | 27 908 | 28 240 |
| 5′ (left) ITR | 1 | 137 |
| Packaging Sequence | 138 | 479 |

E4 region (complementary strand)

| | | |
|---|---|---|
| E4 299R | 32 075 | 32 974 |
| E4 145R | 33 604 | 34 041 |
| E4 125R | 34 038 | 34 415 |
| E4 117R | 33 254 | 33 607 |
| E4 122R | 32 877 | 33 245 |
| ORF-14 | 33 100 | 33 609 |
| VA RNA region | 10 433 | 10 594 |

L region

| | | | |
|---|---|---|---|
| L1 52, 55K | | 10 653 | 11 819 |
| LI IIIa | | 11 845 | 13 608 |
| L2 III (penton) | | 13 690 | 15 375 |
| L2 pVII | | 15 383 | 15 961 |
| L2 V | | 16 004 | 17 059 |
| L3 pVI | | 17 399 | 18 139 |
| L3 II (hexon) | | 18 255 | 21 113 |
| L3 23K (protease) | | 21 150 | 21 779 |
| L4 100K | | 23 446 | 25 884 |
| L4 22K | | 25 616 | 26 191 |
| L4 33K | | 25 616 | 25 934 |
| | Join | 26 104 | 26 465 |
| L4 pVIII | | 26 515 | 27 198 |
| L5 IV(fiber) | | 30 826 | 31 797 |
| Fiber Tail | | 30826 | 30951 |
| Fiber Knob | | 30952 | 31224 |
| Fiber Shaft | | 31225 | 31797 |
| 3′ (right) ITR | | 34658 | 34794 |

*“Join” indicates that corresponding mRNA sequences are joined by splicing

Other examples of Ad35 reference genomes can include GenBank Accession Nos. AC_000019, AY271307, and AX049983.

Ad35 genomes and vectors can be engineered for therapeutic use. One goal of certain such engineering can include rendering viral genomes and vectors deficient for propagation in a recipient cell or system, such as a human subject. Propagation deficiency increases the safety of administering the viral genome or vector to the recipient cell or system. Broadly, there are three recognized “generations” of adenoviral vectors and genomes engineered to reduce and/or eliminate replication of the virus in recipients. First-generation adenoviral vectors are engineered to remove genes E1 and E3. Without these genes, adenoviral vectors cannot replicate on their own but can be produced in E1-expressing mammalian cell lines (e.g., HEK293 cells). With only first-generation modifications, adenoviral vector cloning capacity is limited, and host immune response against the vector can be problematic for effective payload expression. Second-generation adenoviral vectors, in addition to E1/E3 removal, are engineered to remove non-structural genes E2 and E4, resulting in increased capacity and reduced immunogenicity. Third-generation adenoviral vectors (also referred to as gutless, high capacity adenoviral vectors, or helper-dependent adenoviral vectors (HDAd)) are engineered to remove all viral coding sequences, but retain the ITRs of the genome and a packaging sequence of the genome. HDAd genomes are helper-dependent because they do not encode proteins necessary for viral production: a helper-dependent genome can only be packaged into a vector if they are present in a cell that includes a nucleic acid sequence that provides viral proteins in trans. These helper-dependent vectors are also characterized by still greater capacity and further decreased immunogenicity. By deleting the viral coding sequences and leaving only the cis-acting elements necessary for genome replication (ITRs) and packaging, cellular immune response against the Ad vector is reduced. Helper-dependent adenoviral vectors (HDAd) engineered to lack all viral coding sequences can efficiently transduce a wide variety of cell types, and can mediate long-term transgene expression with negligible chronic toxicity. HDAd vectors have a large cloning capacity of up to, e.g., 37 kb, allowing for the delivery of large payloads. In various embodiments, retained portions of the reference genome can be identical in sequence to corresponding sequences of a reference genome or can have less than 100% identity with a reference genome, e.g., at least 99%, 98%, 97%, 96%, 95%, 90%, 85%, 80%, or 75% identity.

Because HDAd vectors do not encode the viral proteins required to produce viral particles, viral proteins must be provided in trans, e.g., expressed in and/or by cells in which the HDAd genome is present. In some HDAd vector systems, one viral genome (a helper genome) encodes some or all of the proteins (e.g., all of the structural viral proteins) required for vector production but has a conditional defect in its packaging sequence, making the helper genome less likely to be packaged into a vector under certain vector production conditions (e.g., under conditions that, and/or in the presence of an agent that, reduces function of the conditionally defective packaging sequence). Thus, in various embodiments, an HDAd donor viral genome includes (e.g., only includes) Ad ITRs, a payload (e.g., a therapeutic payload), and a functional packaging sequence (e.g., a wild-type packaging sequence or a functional fragment thereof), which allows the HDAd donor genome to be selectively packaged into HDAd vectors produced from structural components expressed from the helper genome that has a conditional packaging defect. In other words, Ad35 helper vectors and genomes can be used for production of Ad35 donor vectors.

In some HDAd vector systems, a helper genome utilizes a recombinase system (e.g., a Cre/loxP system) for conditional packaging. In certain such HDAd vector systems, a helper genome can include a packaging sequence (e.g., a complete packaging sequence or a functional fragment thereof (e.g., a fragment of the packaging sequence that is sufficient for packaging, required for packaging, or required for efficient packaging of the Ad genome into the capsid)) flanked by recombinase (e.g., loxP) sites so that contact with a corresponding recombinase (e.g., Cre recombinase) excises the packaging sequence from the helper genome by recombinase-mediated (e.g., Cre-mediated) site-specific recombination between the recombinase sites (e.g., loxP sites). The present disclosure includes, among other things, Ad35 helper vectors and genomes that include two recombinase sites that flank a packaging sequence, where the two recombinase sites are sites corresponding to (i.e., for, or acted upon by) the same recombinase. In various embodiments, a packaging sequence refers to the portion of a helper genome positioned between two recombinase sites as set forth herein, e.g., between the positions at which first and second recombinase sites are inserted or positioned in a helper genome.

Similar HDAd production systems have been developed using FLP (e.g., FLPe)/frt site-specific recombination, where FLP-mediated recombination between frt sites flanking the packaging sequence of the helper genome reduces or eliminates packaging of helper genomes in producer cells that express FLP.

Thus, examples of recombinase systems include the Flp/Frt system and Cre/loxP system, as well as others such as the Dre/rox system, the Vika/vox system, and the PhiC31 system. Cre is a site-specific DNA recombinase derived from bacteriophage P1 sequences. The Cre/loxP system is described in, for example, EP 02200009B1. Cre/loxP systems can include both canonical loxP sites and/or canonical Cre recombinase and/or variations of one or both. The recognition site of Cre protein is typically a nucleotide sequence of 34 base pairs (ATAACTTCGTATAATGTATGCTATACGAAGTTAT) (SEQ ID NO: 1), referred to as a loxP site. Variants of the lox recognition site that can be used include: lox2272 (ATAACTTCGTATAAaGTATcCTATACGAAGTTAT) (SEQ ID NO: 2); lox511 (ATAACTTCGTATAATGTATaCTATACGAAGTTAT) (SEQ ID NO: 3); lox66 (ATAACTTCGTATANNNTANNNTATACGAACGGTA) (SEQ ID NO: 4); lox71 (TACCGTTCGTATANNNTANNNTATACGAAGTTAT) (SEQ ID NO: 5); loxM2 (ATAACTTCGTATAAgaaAccaTATACGAAGTTAT) (SEQ ID NO: 6); loxM3 (ATAACTTCGTATAtaaTACCATATACGAAGTTAT) (SEQ ID NO: 7); loxM7 (ATAACTTCGTATAAgaTAGAATATACGAAGTTAT) (SEQ ID NO: 8); loxM11 (ATAACTTCGTATAcgaTAccaTATACGAAGTTAT) (SEQ ID NO: 9); and lox5171 (ATAACTTCGTATAATGTgTaCTATACGAAGTTAT) (SEQ ID NO: 10). Variants of Cre recombinase are also known and included herein as disclosed, for example, in Eroshenko and Church, Mutants of Cre recombinase with improved accuracy, Nature Communications volume 4, Article number: 2509 (2013), which is incorporated herein by reference in its entirety and in particular with respect to variants of Cre recombinase. The VCre/VloxP recombinase system was derived from Vibrio plasmid p0908. The sCre/SloxP system is described, e.g., in WO 2010/143606. The Flp/Frt DNA recombinase system was derived from Saccharomyces cerevisiae. The Flp/Frt system includes the recombinase Flp (flippase) that catalyzes DNA-recombination on its Frt recognition sites. In various embodiments, an Frt site includes the sequence GAAGTTCCTATTCtctagaaaGtATAGGAACTTC (SEQ ID NO: 11). Variant Frt sites are also included herein. For example, Senecoff et al. (1987) showed that most mutations within the FRT sequence cause minimal effects if present within only one of the two sites. Variants of the Flp protein include GenBank: ABD57356.1 and GenBank: ANW61888.1. The Dre/rox system is described, e.g., in U.S. Pat. Nos. 7,422,889 and 7,915,037B2. It generally includes a Dre recombinase derived from Enterobacteria phage D6 and a rox recognition site. The Vika/vox system is described, e.g., in U.S. Pat. No. 10,253,332. The PhiC31 recombinase recognizes the AttB/AttP binding sites. In various embodiments, a recombinase site of the present disclosure is a sequence that has at least 70% sequence identity (e.g., 70%, 75%, 80%, 95%, 90%, or 95% sequence identity with a sequence selected from SEQ ID NOs: 1-11).

An Ad35 packaging sequence can include up to five, six, or seven putative “A” repeats. For example, in various embodiments, an Ad35 packaging sequence can include one or more, or all, of AI, AII, AIII, AIV, AV, and/or AVI. In various embodiments, the present disclosure includes a recombinant Ad35 helper vector or genome that includes a packaging sequence flanked by recombinase sites. In various embodiments, an Ad35 packaging sequence refers to a nucleic acid sequence including nucleotides 138 to 504 of GenBank Accession No. AY128640 or a functional fragment thereof (e.g., a fragment of nucleotides 138 to 504 of GenBank Accession No. AY128640 that is sufficient for packaging, required for packaging, or required for efficient packaging of the Ad genome into the capsid) (e.g., such that flanking of the packaging sequence with recombinase sites and excision by recombination of the recombinase sites renders the vector or genome deficient for packaging, e.g., by at least 10% as compared to a reference including the packaging sequence, e.g., by at least 10%, 20%, 30%, 40%, 50%, 60%, 70%, 80%, 90%, 91%, 92%, 93%, 94%, 95%, 96%, 97%, 98%, 99%, or 100%, optionally wherein the reference includes the packaging sequence flanked by the recombines sites). In various embodiments, an Ad35 packaging sequence includes at least 80 nucleotides (e.g., at least 80, 90, 100, 110, 120, 130, 140, 150, 160, 170, 180, 190, 200, 225, 250, 275, or 300 nucleotides, e.g., a number of nucleotides having a lower bound of 80, 90, 100, 110, 120, 130, 140, or 150 nucleotides and an upper bound of 150, 160, 170, 180, 190, 200, 225, 250, 275, or 300 nucleotides) having at least 80% sequence identity (e.g., at least 80%, 85%, 90%, 95%, 96%, 97%, 98%, or 99% sequence identity) with a corresponding fragment of nucleotides 137-504 of GenBank Accession No. AY128640.

Those of skill in the art will appreciate that the term packaging sequence does not necessarily include all of the packaging elements present in a given vector or genome. For example, a helper genome can include recombinase direct repeats that flank a packaging sequence, where the flanked packaging sequence does not include all of the packaging elements present in the helper genome. Accordingly, in certain embodiments, one or two recombinase direct repeats of a helper genome are positioned within a larger packaging sequence, e.g., such that a larger packaging sequence is rendered noncontiguous by introduction of the one or two recombinase direct repeats. In various embodiments, recombinase direct repeats of a helper genome flank a fragment of the packaging sequence such that excision of the flanked packaging sequence by recombination of the recombinase direct repeats reduces or eliminates (more generally, disrupts) packaging of the helper genome and/or ability of the helper genome to be packaged.

In some embodiments, to prevent generation of replication competent Ad (RCA) as a consequence of homologous recombination between the helper and HDAd donor genomes present in producer cells, a “stuffer” sequence can be positioned or inserted into the E3 region to render any recombinants too large to be packaged and/or efficiently packaged.

In some embodiments, production of HDAd35 donor vectors can include transfection of a plasmid including the HDAd donor genome and transduction of a helper vector including a helper genome to the same cell, cells, or population of cells. The helper genome can rescue propagation of the Ad35 donor vector such that Ad35 donor vector can be produced and isolated. In various embodiments, an HDAd donor genome can be delivered to cells that express a recombinase for excision of the conditional packaging sequence of a helper vector (e.g., 293 cells (HEK293) that expresses Cre recombinase), optionally where the HDAd donor genome is delivered to the cells in a non-viral vector form, such as a bacterial plasmid form (e.g., where the HDAd donor genome is present in a bacterial plasmid (pHDAd) and/or is liberated by restriction enzyme digestion). The same cells can be transduced with the helper genome including a packaging sequence flanked by recombinase sites (e.g., loxP sites). Thus, producer cells can be transfected with the HDAd donor genome and transduced with a helper genome bearing a packaging sequence flanked by recombinase sites (e.g., loxP sites), where the cells express a recombinase (e.g., Cre) corresponding to the recombinase sites such that excision of the packaging sequence renders the helper virus genome deficient for packaging (e.g., unpackageable), but still able to provide all of the necessary trans-acting factors for production of HDAd donor vector including the HDAd donor genome. After excision of the packaging sequence, a helper genome is deficient for packaging (e.g., unpackageable) but still able to trans-complement the replication and packaging of the HDAd donor genome. HDAd vectors including the donor genome (e.g., a donor genome including a therapeutic payload) can be isolated from the producer cells. In general, some contamination of helper vectors and/or helper genomes in HDAd viral vectors and HDAd viral vector formulations can occur and can be tolerated. HDAd donor vectors can be further isolated and/or purified of any helper vectors by physical means. Various protocols are known in the art, e.g., at Palmer et al., 2009 Gene Therapy Protocols. Methods in Molecular Biology, Volume 433. Humana Press; Totowa, NJ: 2009. pp. 33-53.

Because the sequences of each viral genome are distinct at least for each serotype, the placement of recombinase sites to produce a helper viral genome cannot be predicted from available information relating to other serotypes. The present disclosure includes positions for insertion and/or placement of recombinase sites to flank a packaging sequence for use in an Ad35 helper genome.

In various embodiments, an Ad35 helper vector can include recombinase sites positioned or inserted to flank a packaging sequence, where a first recombinase site (e.g., a loxP site) is positioned or inserted between positions 136 and 249 (e.g., between 151 and 171, between 161 and 181, between 185 and 205, or between 214 and 234), and a second recombinase site (e.g., a loxP site) is positioned or inserted between positions 377 and 504 (e.g., between 392 and 412 or between 469 and 489) or between positions 3175 or 3200 and 3225 (e.g., between positions 3190 and 3210, e.g., in a genome including an E1 deletion such as a deletion of nucleotide positions 480-3199, 481-3199, or 482-3199).

In certain exemplary embodiments, a first recombinase site (e.g., a loxP site) is positioned or inserted between positions 151 and 171 and a second recombinase site (e.g., a loxP site) is positioned or inserted between positions 3190 or 3200 and 3210 (e.g., in a genome including an E1 deletion such as a deletion of nucleotide positions 480-3199, 481-3199, or 482-3199).

In certain exemplary embodiments, a first recombinase site (e.g., a loxP site) is positioned or inserted between positions 161 and 181 and a second recombinase site (e.g., a loxP site) is positioned or inserted between positions 392 and 412.

In certain exemplary embodiments, a first recombinase site (e.g., a loxP site) is positioned or inserted between positions 185 and 205 and a second recombinase site (e.g., a loxP site) is positioned or inserted between positions 469 and 489.

In certain exemplary embodiments, a first recombinase site (e.g., a loxP site) is positioned or inserted between 214 and 234 and a second recombinase site (e.g., a loxP site) is positioned or inserted between positions 392 and 412.

In various embodiments, an Ad35 helper vector can include recombinase sites positioned or inserted to flank a packaging sequence, where a first recombinase site (e.g., a loxP site) is positioned or inserted at (e.g., immediately adjacent to, e.g., before or after) a position selected from 161, 171, 195, and 224, and a second recombinase site (e.g., a loxP site) is at (e.g., immediately adjacent to, e.g., before or after) a position selected from 402, 479, and 3200 (e.g., in a genome including an E1 deletion such as a deletion of nucleotide positions 480-3199, 481-3199, or 482-3199).

In certain exemplary embodiments, a first recombinase site (e.g., a loxP site) is positioned or inserted at position 161 and a second recombinase site (e.g., a loxP site) is positioned or inserted at position 3200 (e.g., in a genome including an E1 deletion such as a deletion of nucleotide positions 480-3199, 481-3199, or 482-3199).

In certain exemplary embodiments, a first recombinase site (e.g., a loxP site) is positioned or inserted at position 171 and a second recombinase site (e.g., a loxP site) is positioned or inserted at position 402.

In certain exemplary embodiments, a first recombinase site (e.g., a loxP site) is positioned or inserted at position 195 and a second recombinase site (e.g., a loxP site) is positioned or inserted at position 479.

In certain exemplary embodiments, a first recombinase site (e.g., a loxP site) is positioned or inserted at position 224 and a second recombinase site (e.g., a loxP site) is positioned or inserted at position 402.

In at least one exemplary Ad35 helper vector, a recombinase site (e.g., a loxP element) is inserted after nucleotide 178 and a recombinase site (e.g., a loxP element) is inserted after nucleotide 437. Excision of the loxP-flanked sequence removes packaging sequence sequences AI to AVI. In certain such embodiments, deletion of nucleotides 345-3113 removes the E1 gene as well as packaging signal sequences which can include AVI and may include further packaging signal sequences. Accordingly, the flanked packaging sequence corresponds to positions 179-344. Vectors according to this description were shown to propagate.

In at least one exemplary Ad35 helper vector, a recombinase site (e.g., a loxP element) is inserted after nucleotide 178 and a recombinase site (e.g., a loxP element) is inserted after nucleotide 481, where nucleotides 179-365 are deleted (removing packaging sequence sequences AI to AV, such that remaining sequences which can include AVI and may include further packaging signal sequences are in the nucleic acid sequence flanked by the recombinase sites). In certain such embodiments, deletion of nucleotides 482-3113 removes the E1 gene. Accordingly, the flanked packaging sequence corresponds to positions 366-481. Vectors according to this description were shown to propagate.

In at least one exemplary Ad35 helper vector, a recombinase site (e.g., a loxP element) is inserted after nucleotide 154 and a recombinase site (e.g., a loxP element) is inserted after nucleotide 481. Accordingly, the flanked packaging sequence corresponds to positions 155-481. Vectors according to this description were shown to propagate. In certain such embodiments, deletion of nucleotides 482-3113 removes the E1 gene.

In at least one exemplary Ad35 helper vector, a recombinase site (e.g., a loxP element) is inserted after nucleotide 158 and a recombinase site (e.g., a loxP element) is inserted after nucleotide 480. Accordingly, the flanked packaging sequence corresponds to positions 159-480. Vectors according to this description were shown to propagate. In certain such embodiments, nucleotides 27388-30402 including the E3 region are deleted. In certain embodiments, the vector is an Ad35⁺⁺ vector.

In at least one exemplary Ad35 helper vector, a recombinase site (e.g., a loxP element) is inserted after nucleotide 158 and a recombinase site (e.g., a loxP element) is inserted after nucleotide 446. Accordingly, the flanked packaging sequence corresponds to positions 159-446. Vectors according to this description were shown to propagate. In certain such embodiments, nucleotides 27388-30402 including E3 region are deleted. In certain embodiments, the vector is an Ad35⁺⁺ vector.

In at least one exemplary Ad35 helper vector, a recombinase site (e.g., a loxP element) is inserted after nucleotide 179 and a recombinase site (e.g., a loxP element) is inserted after nucleotide 480. Accordingly, the flanked packaging sequence corresponds to positions 180-480. Vectors according to this description were shown to propagate. In certain such embodiments, nucleotides 27388-30402 including E3 region are deleted. In certain embodiments, the vector is an Ad35⁺⁺ vector.

In at least one exemplary Ad35 helper vector, a recombinase site (e.g., a loxP element) is inserted after nucleotide 206 and a recombinase site (e.g., a loxP element) is inserted after nucleotide 480. Accordingly, the flanked packaging sequence corresponds to positions 207-480. Vectors according to this description were shown to propagate. In certain such embodiments, nucleotides 27,388-30,402 including E3 region are deleted. In certain embodiments, nucleotides 27,607-30,409 or 27,609-30,402 are deleted. In certain embodiments, nucleotides 27,240-27,608 are not deleted.

In at least one exemplary Ad35 helper vector, a recombinase site (e.g., a loxP element) is inserted after nucleotide 139 and a recombinase site (e.g., a loxP element) is inserted after nucleotide 446. In certain such embodiments, nucleotides 27609-30402 are deleted. Accordingly, the flanked packaging sequence corresponds to positions 140-446.

In at least one exemplary Ad35 helper vector, a recombinase site (e.g., a loxP element) is inserted after nucleotide 158 and a recombinase site (e.g., a loxP element) is inserted after nucleotide 446. In certain such embodiments, nucleotides 27609-30402 are deleted. Accordingly, the flanked packaging sequence corresponds to positions 159-446.

In at least one exemplary Ad35 helper vector, a recombinase site (e.g., a loxP element) is inserted after nucleotide 179 and a recombinase site (e.g., a loxP element) is inserted after nucleotide 446. In certain such embodiments, nucleotides 27609-30402 are deleted. Accordingly, the flanked packaging sequence corresponds to positions 180-446.

In at least one exemplary Ad35 helper vector, a recombinase site (e.g., a loxP element) is inserted after nucleotide 201 and a recombinase site (e.g., a loxP element) is inserted after nucleotide 446. In certain such embodiments, nucleotides 27609-30402 are deleted. Accordingly, the flanked packaging sequence corresponds to positions 202-446.

In at least one exemplary Ad35 helper vector, a recombinase site (e.g., a loxP element) is inserted after nucleotide 158 and a recombinase site (e.g., a loxP element) is inserted after nucleotide 481. In certain such embodiments, nucleotides 27609-30402 are deleted. Accordingly, the flanked packaging sequence corresponds to positions 159-481.

In at least one exemplary Ad35 helper vector, a recombinase site (e.g., a loxP element) is inserted after nucleotide 179 and a recombinase site (e.g., a loxP element) is inserted after nucleotide 384. In certain such embodiments, nucleotides 27609-30402 are deleted. Accordingly, the flanked packaging sequence corresponds to positions 180-384.

In at least one exemplary Ad35 helper vector, a recombinase site (e.g., a loxP element) is inserted after nucleotide 179 and a recombinase site (e.g., a loxP element) is inserted after nucleotide 481. In certain such embodiments, nucleotides 27609-30402 are deleted. Accordingly, the flanked packaging sequence corresponds to positions 180-481.

In at least one exemplary Ad35 helper vector, a recombinase site (e.g., a loxP element) is inserted after nucleotide 206 and a recombinase site (e.g., a loxP element) is inserted after nucleotide 481. In certain such embodiments, nucleotides 27609-30402 are deleted. Accordingly, the flanked packaging sequence corresponds to positions 207-481.

In various embodiments, excision of a packaging sequence from an Ad35 helper genome reduces propagation of the vector by, e.g., at least 20%, 30%, 40%, 50%, 60%, 70%, 80%, 90%, 91%, 92%, 93%, 94%, 95%, 96%, 97%, 98%, 99%, 99.5%, 99.9%, or 100% (e.g., reduces propagation of the vector by a percentage having a lower bound of 20%, 30%, 40%, 50%, 60%, 70%, and an upper bound of 60%, 70%, 80%, 90%, 91%, 92%, 93%, 94%, 95%, 96%, 97%, 98%, 99%, 99.5%, 99.9%, or 100%), optionally where percent propagation is measured as the number of viral particles produced by propagation of excised vector (vector from which the recombinase site-flanked sequence has been excised) as compared to complete vector (vector from which the recombinase site-flanked sequence has not been excised) or as compared to wild-type Ad35 vector under comparable conditions.

As noted above, homologous recombination between a helper genome including a conditionally defective packaging sequence and a donor genome that includes a wild type or reference packaging sequence can eliminate one or more recombinase sites of a conditionally defective packaging sequence, which can result in contamination of produced donor vectors. This is at least in part because two recombinase sites flanking a packaging sequences are required for excision of the packaging sequence. Excision of one or more of the recombinase sites by recombinase site-excising homologous recombination produces a helper genome that, when contacted with a recombinase corresponding to the recombinase sites, is not rendered defective for packaging (referred to herein as a constitutively packageable helper genome). The present disclosure includes the recognition that packaging sequence inversion can reduce recombinase site-excising homologous recombination.

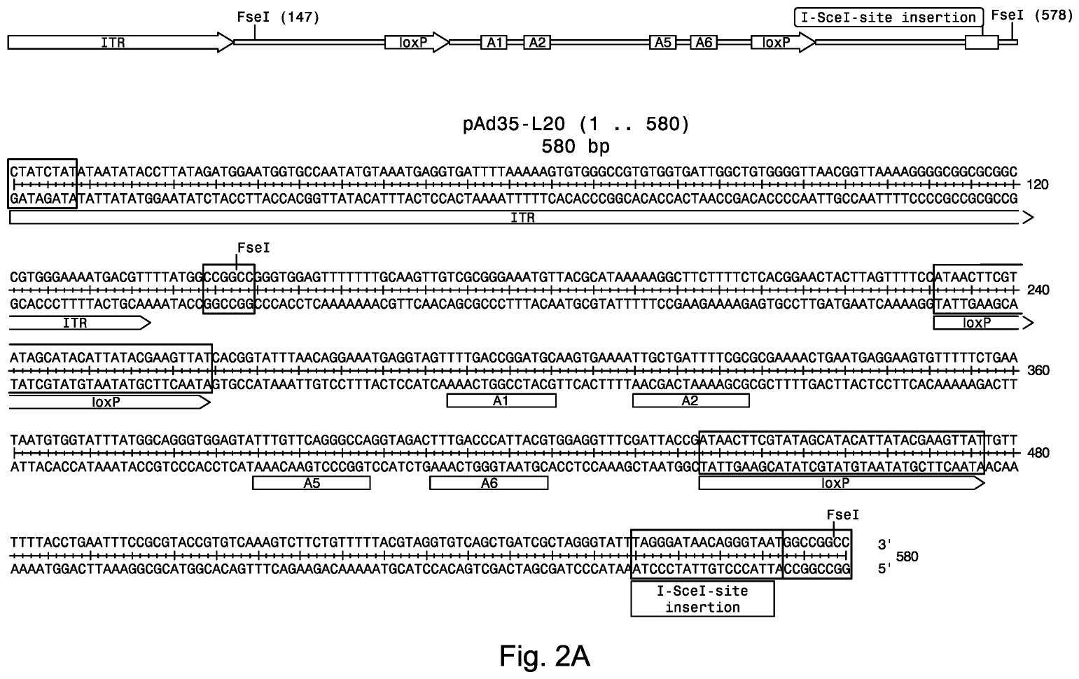

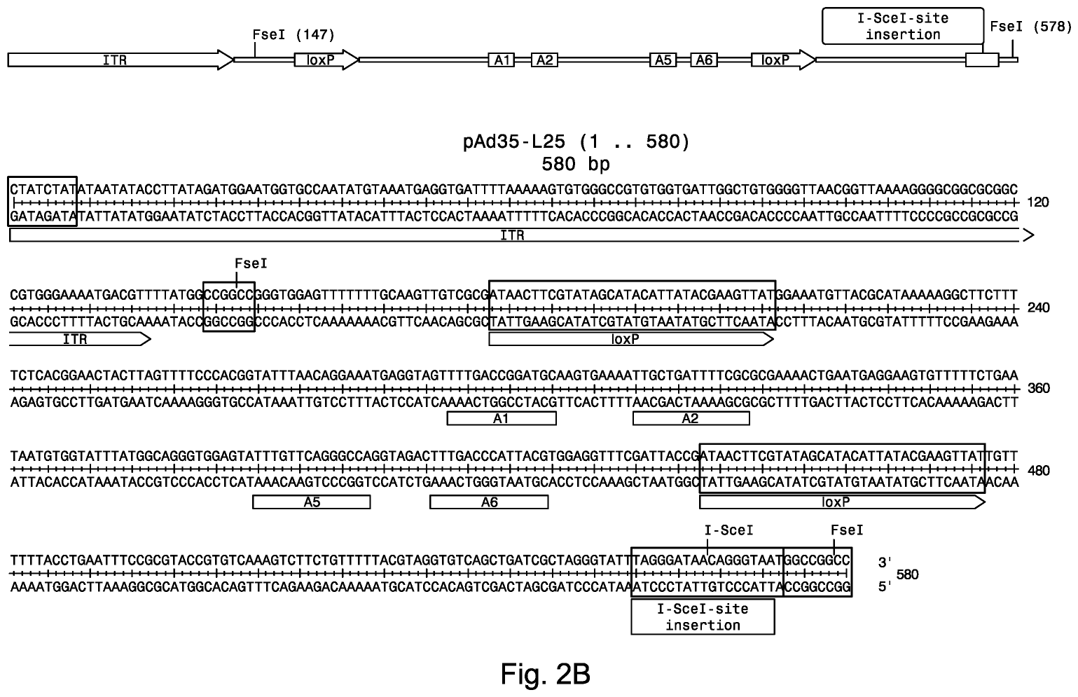

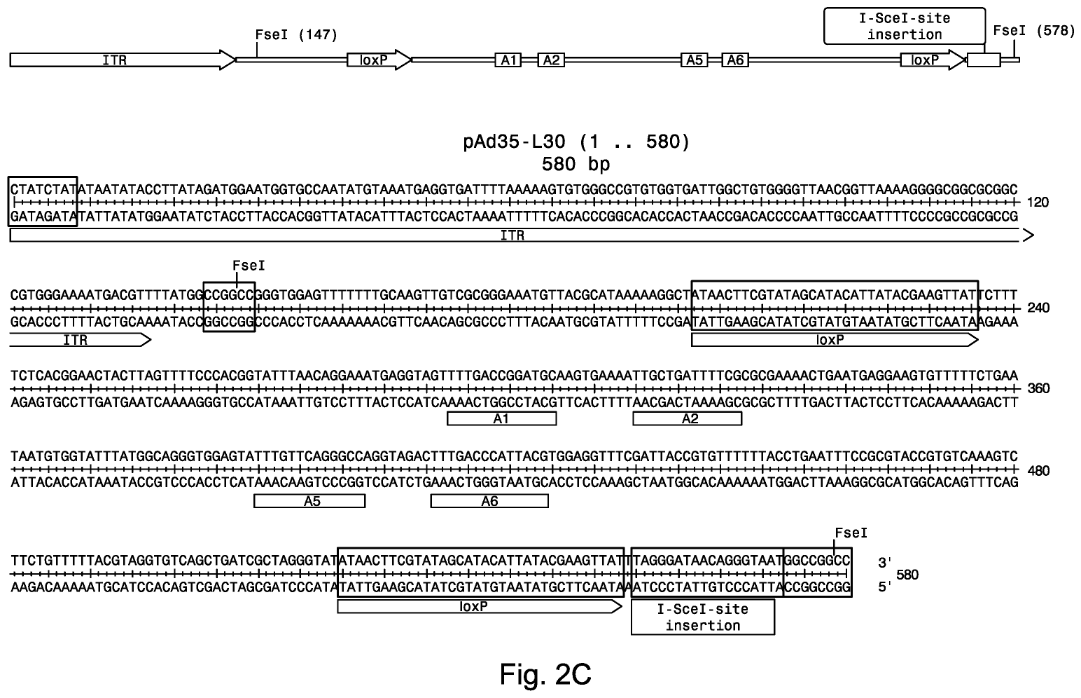

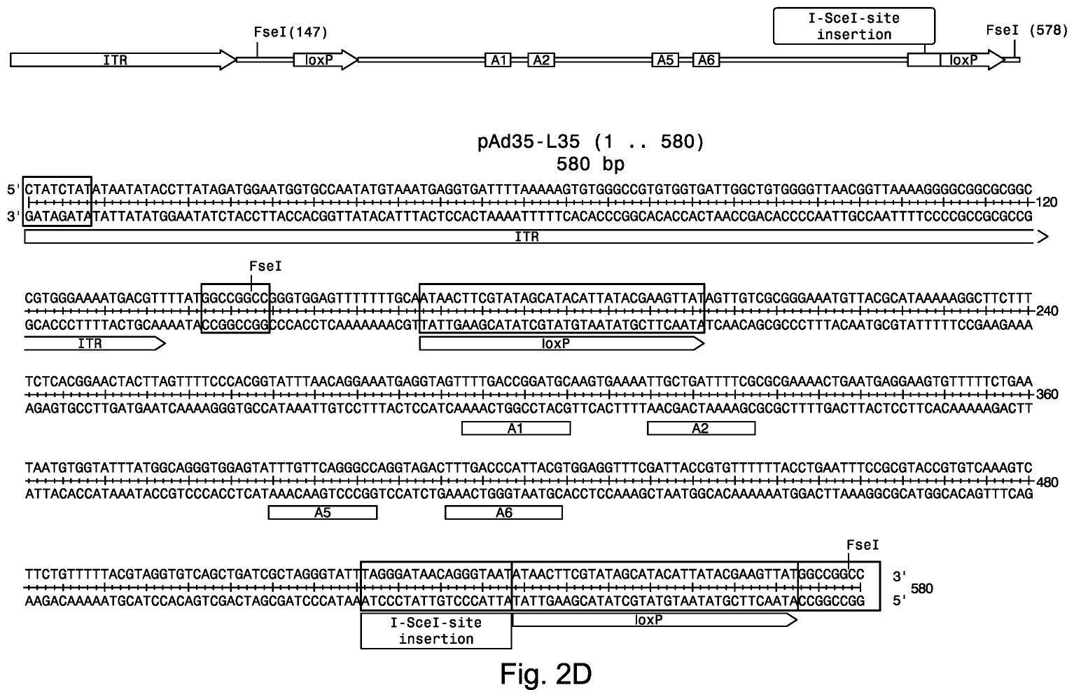

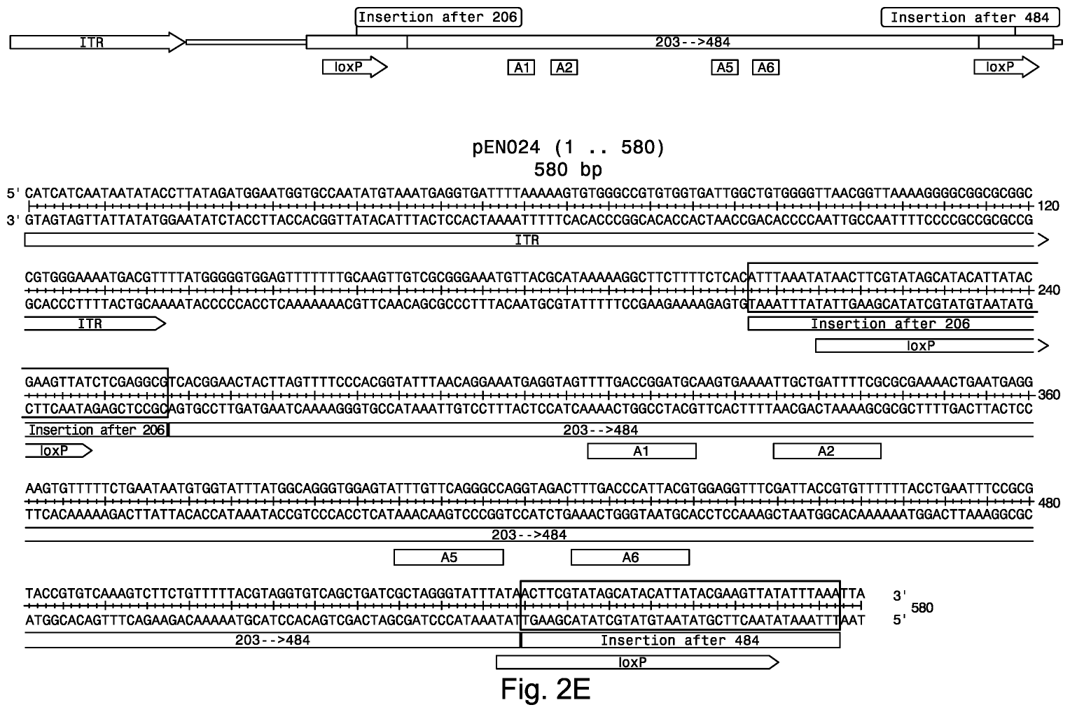

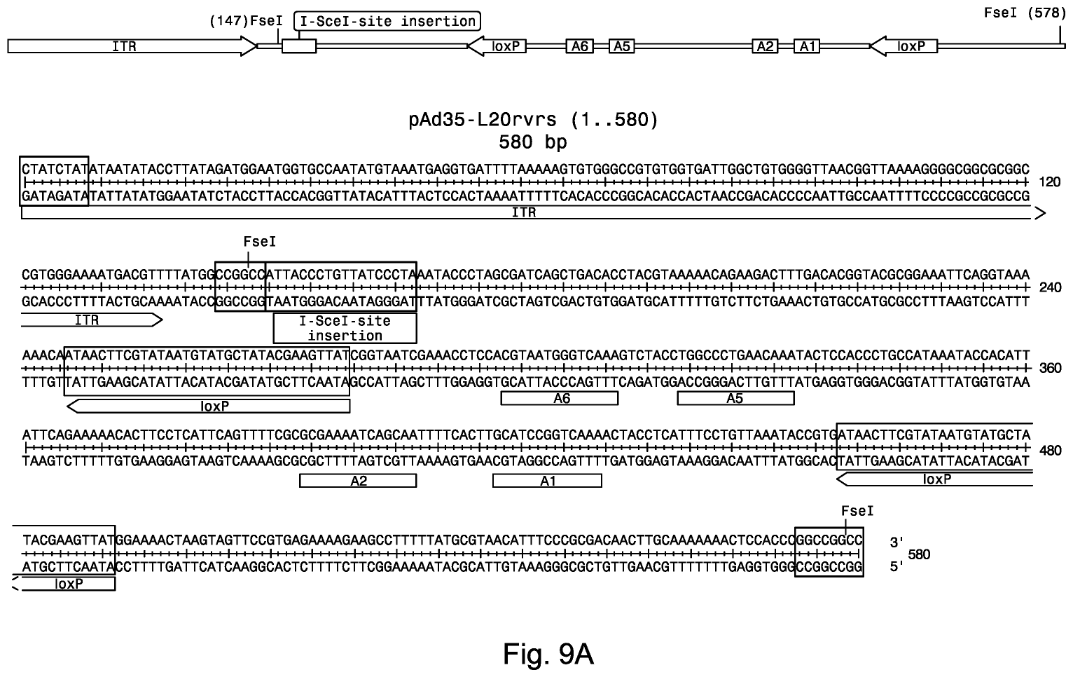

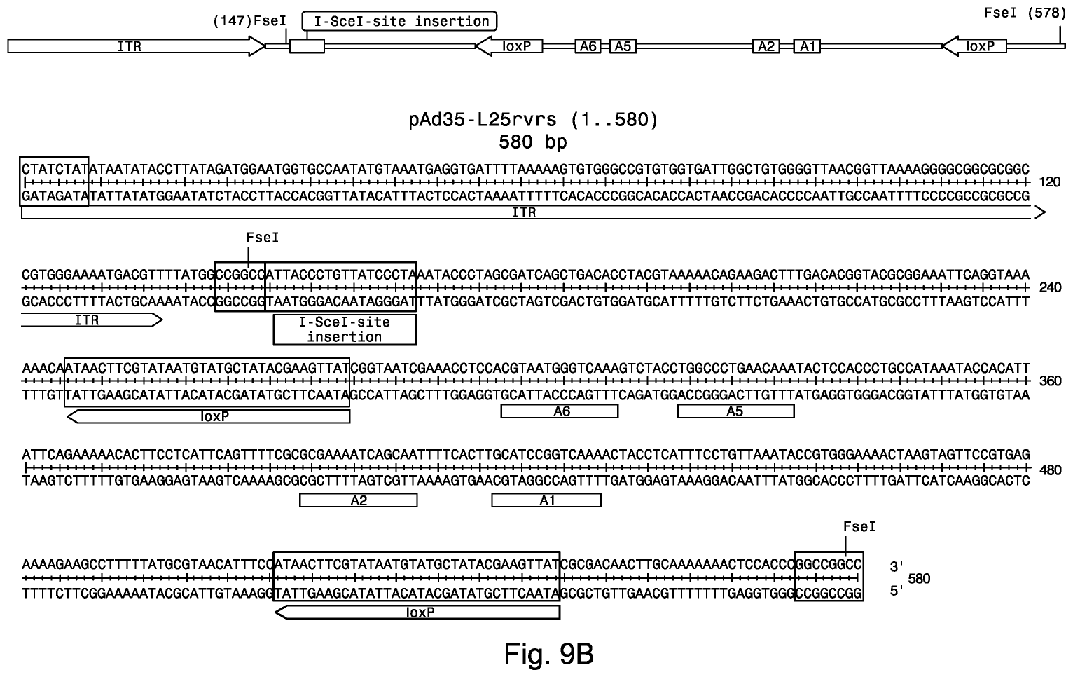

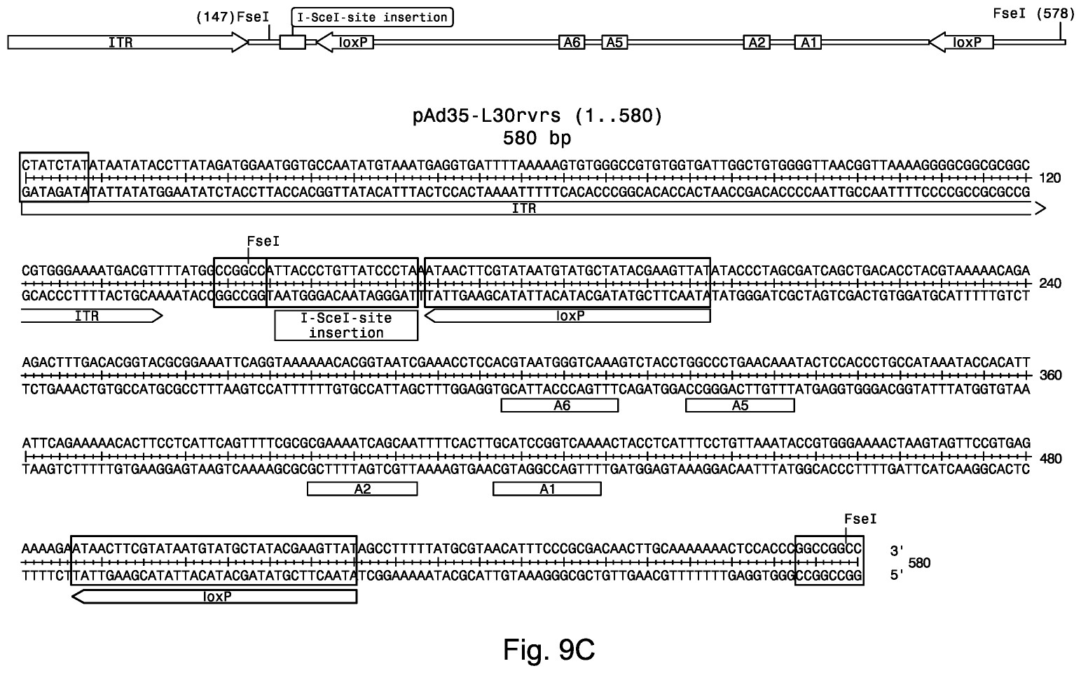

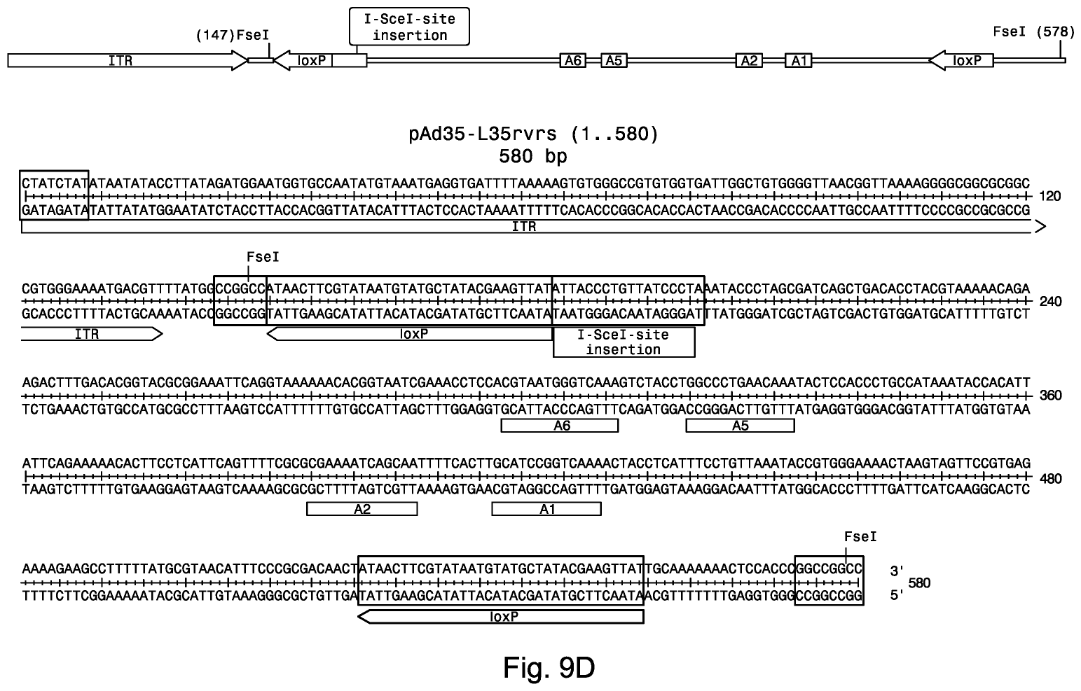

In some embodiments, a helper genome can include a packaging sequence inversion in that a sequence including the recombinase-site flanked packaging sequence of the helper genome is present in an orientation that differs from a wild-type or reference sequence, such as a reference adenoviral genome. As those of skill in the art will appreciate, nucleic acid sequences are ordered between 5′ and 3′ termini for a given strand, and can be present within a nucleic acid context, such as a strand of genomic DNA having a particular 5′ to 3′ sequence. The “orientation” of a nucleic acid sequence fragment present in a nucleic acid context can refer to whether the order of nucleotides in the fragment is the same as in a corresponding fragment of a wild type or reference nucleic acid context (e.g., a genomic sequence that includes a sequence corresponding to the nucleic acid sequence), or inverted in that a complementary sequence running in the opposite direction (a reverse complement of a corresponding wild-type or reference sequence) is present in the nucleic acid context instead. In various embodiments as used herein, the “orientation” of a flanked packaging sequence or other nucleic acid fragment of an adenoviral genome can refer to the order of nucleotides relative to an ITR, e.g., a 5′ITR or 3′ ITR. Inversion of a sequence comprising a recombinase-flanked packaging sequence can reduce and/or eliminate recombinase site-excising homologous recombination and thereby prevent production of constitutively packageable helper genomes. Sequence inversion is further illustrated by comparing FIGS. 2A-D to FIGS. 9A-D.

The present disclosure therefore includes, among other things, helper vectors and genomes that include two recombinase sites that flank a packaging sequence, where the two recombinase sites correspond to (i.e., are for, or acted upon by) the same recombinase, and where a sequence that includes the flanked packaging sequence is inverted. In various embodiments, an inverted sequence is or includes a packaging sequence, such as a recombinase site-flanked packaging sequence. In various embodiments, an inverted sequence includes a recombinase site-flanked packaging sequence and one or both of the recombinase sites that flank the packaging sequence.

In various embodiments, an inverted sequence includes additional nucleic acids that are not present in a flanked packaging sequence and/or are not present in the recombinase sites that flank the packaging sequence. In various embodiments one or both of the 5′ and 3′ ends of an inverted sequence include at least 1, 2, 3, 4, 5, 10, 20, 30, 40, 50, 75, 100, 150, 200, 250, 300, 350, 400, 450, 500, 1,000, or more nucleotides adjacent to a recombinase site. In various embodiments, an inverted sequence includes a number of nucleotides 5′ of a 5′ recombinase site of a flanked packaging sequence that has a lower bound of 1, 2, 3, 4, 5, 10, 20, 30, 40, 50, 75, 100, 150, 200, or 250 nucleotides and an upper bound of 10, 20, 30, 40, 50, 75, 100, 150, 200, 250, 300, 350, 400, 450, 500, or more nucleotides. In various embodiments, an inverted sequence includes a number of nucleotides 3′ of a 3′ recombinase site of a flanked packaging sequence that has a lower bound of 1, 2, 3, 4, 5, 10, 20, 30, 40, 50, 75, 100, 150, 200, or 250 nucleotides and an upper bound of 10, 20, 30, 40, 50, 75, 100, 150, 200, 250, 300, 350, 400, 450, 500, 1,000, or more nucleotides.

In various embodiments, an inverted sequence includes a gene. In various embodiments, an inverted sequence includes E1 or encompasses an E1 deletion. In various embodiments, an inverted sequence includes a gene encoding protein IX. In various embodiments an inverted sequence includes a gene encoding protein IVa2.

In various embodiments, an inverted sequence does not include an ITR (e.g., a 5′ ITR). In various embodiments, an inverted sequence does not include an exon and/or does not include a portion of an exon. In various embodiments, an inverted sequence is present in a viral genome at a position that does not correspond to its position in a reference or wild-type genome. In various embodiments, an inverted sequence does not include any portion of an E1 coding sequence, protein IX coding sequence, and/or protein IVa2 coding sequence.

In various embodiments, an inverted packaging sequence can include a recombinase-flanked packaging sequence according to any embodiment (e.g., including any recombinase site positions provided herein. In various embodiments, as provided herein, nucleic acid positions of an Ad35 vector of the present disclosure can be numbered according to a reference disclosed herein, e.g., GenBank accession number AY128640. In various embodiments, an inverted packaging sequence can include one or more, or all, of AI, AII, AIII, AIV, AV, and/or AVI, optionally wherein one or more, or all, of AI, AII, AIII, AIV, AV, and/or AVI are present within a sequence flanked by recombinase sites.

In some embodiments, the inverted sequence is or includes a sequence corresponding to a portion of a reference sequence that comprises a first end point between positions 119 and 169, and a second end point between positions 3175 and 3225 (e.g., in a genome that includes an E1 deletion such as a deletion of nucleotide positions 480-3199, 481-3199, or 482-3199). In some embodiments, the inverted sequence is or includes a sequence corresponding to a portion of a reference sequence that comprises a first end point between positions 119 and 169, and a second end point between positions 455 and 505.

In some embodiments, the inverted sequence is or includes a sequence corresponding to a portion of a reference sequence that comprises a first end point between positions 134 and 154, and a second end point between positions 3190 and 3210 (e.g., in a genome that includes an E1 deletion such as a deletion of nucleotide positions 480-3199, 481-3199, or 482-3199). In some embodiments, the inverted sequence is or includes a sequence corresponding to a portion of a reference sequence that comprises a first end point between positions 134 and 154, and a second end point between positions 470 and 490.

In some embodiments, the inverted sequence is or includes a sequence corresponding to a portion of a reference sequence that comprises a first end point at position 144, and a second end point at position 3200 (e.g., in a genome that includes an E1 deletion such as a deletion of nucleotide positions 480-3199, 481-3199, or 482-3199). In some embodiments, the inverted sequence is or includes a sequence corresponding to a portion of a reference sequence that comprises a first end point at position 144, and a second end point at position 480.

In certain exemplary embodiments,
(i) a first recombinase site (e.g., a loxP site) positioned between nucleotide positions corresponding to 136 and 249 of GenBank Accession No. AY128640;
(ii) a second recombinase site (e.g., a loxP site) positioned between nucleotide positions corresponding to 377 and 504 or 3175 and 3225 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 151 and 171, 161 and 181, 185 and 205, or 214 and 234 of GenBank Accession No. AY128640;
(ii) a second recombinase site (e.g., a loxP site) positioned between nucleotide positions corresponding to 392 and 412 or 469 and 489 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 151 and 171, 161 and 181, 185 and 205, or 214 and 234 of GenBank Accession No. AY128640;
(ii) a second recombinase site (e.g., a loxP site) positioned between nucleotide positions corresponding to 3190 and 3210 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 151 and 171 of GenBank Accession No. AY128640;
(ii) a second recombinase site (e.g., a loxP site) positioned between nucleotide positions corresponding to 3190 and 3210 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 161 and 181 of GenBank Accession No. AY128640;
(ii) a second recombinase site (e.g., a loxP site) positioned between nucleotide positions corresponding to 392 and 412 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 185 and 205 of GenBank Accession No. AY128640;
(ii) a second recombinase site (e.g., a loxP site) positioned between nucleotide positions corresponding to 469 and 489 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) positioned between nucleotide positions corresponding to 214 and 234 of GenBank Accession No. AY128640;
(ii) a second recombinase site (e.g., a loxP site) positioned between nucleotide positions corresponding to 392 and 412 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) at a nucleotide position corresponding to 161, 171, 195, or 224 of GenBank Accession No. AY128640;
(ii) a second recombinase site (e.g., a loxP site) at a nucleotide position corresponding to 402, 479, or 3200 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) at a nucleotide position corresponding to 161 of GenBank Accession No. AY128640;
(ii) a second recombinase site (e.g., a loxP site) at a nucleotide position corresponding to 3200 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) at a nucleotide position corresponding to 171 of GenBank Accession No. AY128640;
(ii) a second recombinase site (e.g., a loxP site) at a nucleotide position corresponding to 402 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) at a nucleotide position corresponding to 195 of GenBank Accession No. AY128640;
(ii) a second recombinase site (e.g., a loxP site) at a nucleotide position corresponding to 479 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

In certain exemplary embodiments,
(i) a first recombinase site (e.g., a loxP site) at a nucleotide position corresponding to 224 of GenBank Accession No. AY128640;
(ii) a second recombinase site (e.g., a loxP site) a second direct repeat at a nucleotide position corresponding to 402 of GenBank Accession No. AY128640; and
(iii) an inversion of a sequence that includes a first end point that is between positions 119 and 169 (e.g., between positions 134 and 154, e.g., at position 144), and a second end point that is between positions 3175 and 3225 (e.g., between positions 3190 and 3210, e.g., at position 3200) or that is between positions 455 and 505 (e.g., between positions 470 and 490, e.g., at position 480).

The present disclosure further includes that in certain embodiments a recombinase site is positioned at a nucleotide position as described herein. For example, in various embodiments that are not limiting the to the scope of the present disclosure, a first recombinase site is at one or more of positions 139, 140, 154, 155, 158, 159, 178, 179, 180, 201, 202, 206, 207, 343, 344, 364, 365, 366, 383, 384, 437, 445, 446, 479, 480, and/or 481. In various particular embodiments that are not limiting the to the scope of the present disclosure, a first recombinase site is at one or more of positions 139, 140, 154, 155, 158, 159, 178, 179, 180, 201, 202, 206, 207, 343, 344, 364, 365, and/or 366. In various particular embodiments that are not limiting the to the scope of the present disclosure, a second recombinase site is at one or more of positions 343, 344, 364, 365, 366, 383, 384, 437, 445, 446, 479, 480, and/or 481. In various particular embodiments, first and second recombinase sites are not at positions selected from the following first and second positions: 139 and 446, 154 and 481, 158 and 446, 158 and 480, 158 and 481, 178 and 344, 178 and 437, 178 and 481, 179 and 384, 179 and 446, 179 and 480, 179 and 481, 201 and 446, 206 and 480, 206 and 481, and/or 365 and 481. In various particular embodiments that are not limiting the to the scope of the present disclosure, a first recombinase site is at a position that is between nucleotide 130 and nucleotide 400 (e.g., between nucleotides 138 and 180, 138 and 200, 138 and 220, 138 and 240, 138 and 260, 138 and 280, 138 and 300, 138 and 320, 138 and 340, 138 and 360, 138 and 366, 138 and 380, or 138 and 400). In various particular embodiments that are not limiting the to the scope of the present disclosure, a second recombinase site is at one or more of positions between nucleotide 300 and nucleotide 550 (e.g., between nucleotides 344 and 360, 344 and 380, 344 and 400, 344 and 420, 344 and 440, 344 and 460, 344 and 480, 344 and 481, 344 and 500, 344 and 520, 344 and 540, or 344 and 550).

The present disclosure further includes that in certain embodiments a recombines site is not positioned at a nucleotide position as described herein. For example, in various embodiments that are not limiting the to the scope of the present disclosure, a first recombinase site is not at one or more of positions 139, 140, 154, 155, 158, 159, 178, 179, 180, 201, 202, 206, 207, 343, 344, 364, 365, 366, 383, 384, 437, 445, 446, 479, 480, and/or 481. In various particular embodiments that are not limiting the to the scope of the present disclosure, a first recombinase site is not at one or more of positions 139, 140, 154, 155, 158, 159, 178, 179, 180, 201, 202, 206, 207, 343, 344, 364, 365, and/or 366. In various particular embodiments that are not limiting the to the scope of the present disclosure, a second recombinase site is not at one or more of positions 343, 344, 364, 365, 366, 383, 384, 437, 445, 446, 479, 480, and/or 481. In various particular embodiments, first and second recombinase sites are not at positions selected from the following first and second positions: 139 and 446, 154 and 481, 158 and 446, 158 and 480, 158 and 481, 178 and 344, 178 and 437, 178 and 481, 179 and 384, 179 and 446, 179 and 480, 179 and 481, 201 and 446, 206 and 480, 206 and 481, and/or 365 and 481. In various particular embodiments that are not limiting the to the scope of the present disclosure, a first recombinase site is not at a position that is between nucleotide 130 and nucleotide 400 (e.g., between nucleotides 138 and 180, 138 and 200, 138 and 220, 138 and 240, 138 and 260, 138 and 280, 138 and 300, 138 and 320, 138 and 340, 138 and 360, 138 and 366, 138 and 380, or 138 and 400). In various particular embodiments that are not limiting the to the scope of the present disclosure, a second recombinase site is not at one or more of positions between nucleotide 300 and nucleotide 550 (e.g., between nucleotides 344 and 360, 344 and 380, 344 and 400, 344 and 420, 344 and 440, 344 and 460, 344 and 480, 344 and 481, 344 and 500, 344 and 520, 344 and 540, or 344 and 550).

Those of skill in the art will appreciate that there are a variety of means by which an inversion can be produced within a larger sequence such as an adenoviral genome. Such inversions can be produced using various available tools of molecular biology. For example, in certain embodiments, an inverted sequence can by synthesized or isolated and inserted into a target sequence by various means known in the art. In certain embodiments, a sequence for inversion can be positioned between two copies of a palindromic restriction site, such that contacting a sequence including the sequence for inversion flanked by the restriction sites can result in an inversion in accordance with various methods known in the art.

To provide one non-limiting example of a technique for producing an inversion of a sequence within an adenoviral genome, FseI sites can be inserted at positions that flank a packaging sequence or sequence including a packaging sequence, optionally wherein the packaging sequence is flanked by recombinase sites that are in turn between the FseI sites. Such a sequence can be digested with FseI to excise the FseI site-flanked sequence, which excised sequence can then be re-ligated into the digested sequence in the opposite orientation.

The present disclosure includes, among other things, Ad35 helper genomes as disclosed herein and Ad35 helper vectors that include the same. The present disclosure further includes use of Ad35 helper genomes and vectors in a method or composition for production of HDAd35 donor vectors. The present disclosure further includes cells that include Ad35 helper vectors and/or Ad35 helper genomes (and optionally further include an HDAd35 donor genome), e.g., for production of HDAd35 donor vectors. The present disclosure further includes use of such cells in a method or composition for production of HDAd35 donor vectors. In certain such cells, viral proteins encoded and expressed by the helper genome can be utilized in production of HDAd35 donor vectors in which the HDAd35 donor genome is packaged. Accordingly, the present disclosure includes methods of production of HDAd35 donor vectors by culturing cells that include an HDAd35 donor genome and an Ad35 helper genome. In some embodiments the cells encode and express a recombinase that corresponds to recombinase direct repeats that flank a packaging sequence of the Ad35 helper vector. In some embodiments, the flanked packaging sequence of the Ad35 helper genome has been excised.

In various embodiments, an Ad35 helper genome includes a conditional (e.g., frt-site or loxP-site flanked) packaging sequence and encodes all of the necessary proteins for production of Ad35 virions into which a donor genome can be packaged. In some embodiments the Ad35 helper genome encodes all Ad35 coding sequences.

An additional optional engineering consideration can be engineering of a helper genome having a size that permits separation of helper vector from HDAd35 donor vector by centrifugation, e.g., by CsCl ultracentrifugation. One means of achieving this result is to increase the size of the helper genome as compared to a typical Ad35 genome. In particular, adenoviral genomes can be increased by engineering to at least 104% of wild-type length. Certain helper vectors of the present disclosure can accommodate a payload and/or stuffer sequence.

The present disclosure includes that in various embodiments a vector or genome of the present disclosure such as an Ad35 helper genome can include a selection of components each selected from, or having at least 75% sequence identity (e.g., at least 75%, 80%, 85%, 90%, 95%, 96%, 97%, 98%, 99%, or 100% identity) to, a corresponding sequence of an Ad35 reference genome.

In various embodiments, an Ad35 vector such as a helper vector or donor vector includes, or an Ad35 helper genome encodes, fiber knob mutations as compared to a reference or canonical Ad35 fiber knob, where the mutations increase affinity of the vector, fiber, and/or fiber knob with CD46 (see, e.g. Table 2). In various embodiments, an Ad35 vector such as a helper vector or donor vector includes, or an Ad35 helper genome encodes, an Ad35++ fiber knob. An Ad35++ fiber knob is a fiber knob that includes mutations as compared to a reference or canonical Ad35 fiber knob, where the mutations increase affinity with CD46, e.g., optionally wherein the increase is an increase of up to or at least 1.1-fold, e.g., up to or at least 1, 2, 3, 4, 5, 10, 15, 20, or 25-fold. Increased affinity with CD46 can increase efficiency of target cell transduction and/or decrease the multiplicity of infection (MOI) required to achieve a target level of transduction (Li and Lieber, FEBS Letters, 593(24): 3623-3648, 2019). In various embodiments, an Ad35++ fiber knob includes at least one mutation selected from Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, an Ad35++ fiber knob includes each of the following mutations: Ile192Val, Asp207Gly (or Glu207Gly in certain Ad35 sequences), Asn217Asp, Thr226Ala, Thr245Ala, Thr254Pro, Ile256Leu, Ile256Val, Arg259Cys, and Arg279His. In various embodiments, amino acid numbering of an Ad35 fiber is according to GenBank accession AP_000601 or an amino acid sequence corresponding thereto, e.g., where position 207 is Glu or Asp. In various embodiments, an Ad35 fiber has an amino acid sequence according to GenBank accession AP_000601. Further description of Ad35++ fiber knob mutations is found in Wang 2008 J. Virol. 82(21): 10567-10579, which is incorporated herein by reference in its entirety and with respect to fiber knobs.

TABLE 2
Mutated Ad35 Knob increased binding to CD46

| | Kd (Oleks) |
|---|---|
| A1: Asn217Asp Thr245Pro Ile256Leu* | A1 4.82 nM |
| A2: Asp207Gly Thr245Ala* | A2 0.629 nM |
| A3: Asp207Gly Thr226Ala* | A3 1.407 nM |
| A8: Ile192Val Ile256Val | A8 13.6850 nM |
| B1: Asp207Gly* | B1 1.774 nM |
| B2: wtAd35(207Asp) | B2 14.98 nM |
| B3: Asn217Asp* | B3 16.85 nM |
| B4: Thr245Ala* | B4 7.64 nM |
| B5: Ile256Leu* | B5 10.96 nM |
| B6: Ad3 | B6 no binding |
| B7: Ad11 | B7 11.22 nM |
| M1: Arg279Cys** | M1 no binding |
| M3: Arg279His** | M3 no binding |
| wtAd35* | 13.7 nM |
| wtAd35* | 15.36 nM |
| AA: Asp207Gly Thr245Ala Ile256Leu* | 0.943 nM |

*Published in Wang et al. (J. Virol., 82(21): 10567-10579, 2008)
**Published in Wang et al. (J. Virol. 81(23): 12785-12792, 2007)

### EXAMPLES

The present disclosure includes the identification of positions within an Ad35 packaging sequence at which recombinase sites can be advantageously positioned to produce an Ad35 genome (e.g., a helper genome) conditionally deficient for packaging. Identification of these positions and genomes allows constructions of safer and/or more efficient helper-dependent adenoviral vectors and vector systems. The present Examples provide, among other things, the successful production and use of adenoviral genomes, including adenoviral helper genomes, provided herein, including adenoviral helper genomes that include a conditionally defective packaging sequence and/or an inverted packaging sequences.

### Example 1: Design of Ad35 Helper Genomes

The present Example includes the identification of positions within an Ad35 genome, and particularly within the Ad35 packaging sequence, at which recombinase sites can be positioned for efficient switching between packaging competence and packaging deficiency. Insertion or positioning of the recombinase sites into the packaging sequence will not abrogate Ad35 genome packaging. However, excision of the recombinase-flanked sequences will reduce and/or eliminates packaging of the genome.

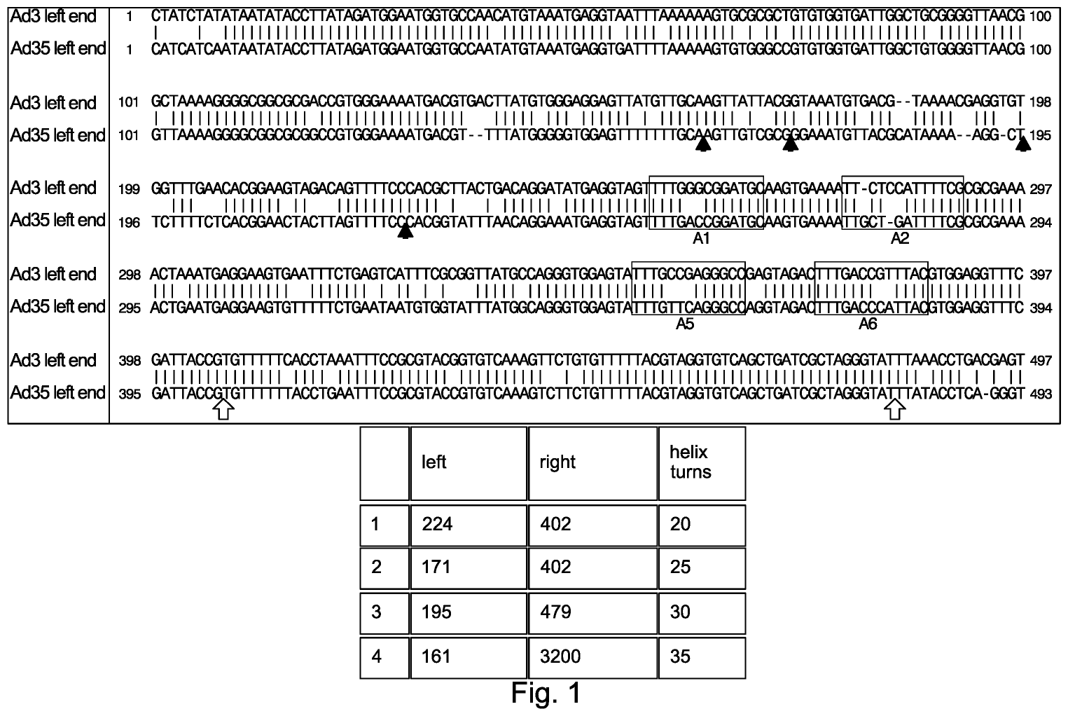

The present Example includes alignment of adenoviral packaging sequences to identify putative packaging signals in the Ad35 genome, as shown in FIG. 1. The present Example further includes selection of particular locations for placement of 5′ (left) and 3′ (right) recombinase sites. Selected left recombinase sites include Ad35 genome positions 224, 171, 195, and 161. Selected right recombinase sites include Ad35 genome positions 402, 479, and 3200. The present Example further includes particular pairings of left and right recombinase site positions: position 224 with position 402, position 171 with position 402, position 195 with position 479, and position 161 with position 3200 (e.g., in a genome including an E1 deletion such as a deletion of nucleotide positions 480-3199, 481-3199, or 482-3199). These positions and pairing are shown in FIG. 1, and are additionally described in the remainder of this Example.

### Exemplary Construct 1

Construct 1 includes recombinase sites positioned to generate a conditionally defective packaging sequence of an Ad35 helper genome. Inserted LoxP sites are shown in the context of nucleotides 1-402 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. In addition, the sequence CCGGCC (SEQ ID NO: 14) was inserted to create a recognition site for the restriction enzyme FseI (TTATGGCCGGCCGGGTGGAGTTTTTTTGCAAGTIGTCGCGGGAAATGTTACGCATAA AAAGGCTTCTTTTCTCACGGAACTACTTAGTTTTCC) (SEQ ID NO: 15). Further sequence information is provided below.

Sequence corresponding to AY128640 nucleotides 1-402 engineered
to include a conditionally defective packaging sequence
(LoxP sequences underlined)
(SEQ ID NO: 16)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCGGGTGGAGTTTTTTTGCAAGT
TGTCGCGGGAAATGTTACGCATAAAAAGGCTTCTTTTCTCACGGAACTACTTAGTTT
TCCATAACTTCGTATAGCATACATTATACGAAGTTATCACGGTATTTAACAGGAAAT
GAGGTAGTTTTGACCGGATGCAAGTGAAAATTGCTGATTTTCGCGCGAAAACTGAAT
GAGGAAGTGTTTTTCTGAATAATGTGGTATTTATGGCAGGGTGGAGTATTTGTTCAG
GGCCAGGTAGACTTTGACCCATTACGTGGAGGTTTCGATTACCGATAACTTCGTATA
GCATACATTATACGAAGTTAT

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
LoxP sequence
(SEQ ID NO: 18)
ATAACTTCGTATAGCATACATTATACGAAGTTAT
LoxP-flanked sequence (identified packaging signals A1, A2, A5 and A6 underlined)
(SEQ ID NO: 19)
CACGGTATTTAACAGGAAATGAGGTAGTTTTGACCGGATGCAAGTGAAAATTGCTG
ATTTTCGCGCGAAAACTGAATGAGGAAGTGTTTTTCTGAATAATGTGGTATTTATGG
CAGGGTGGAGTATTTGTTCAGGGCCAGGTAGACTTTGACCCATTACGTGGAGGTTTC
GATTACCG

FIG. 2A shows a region (the left end) of an Ad35 helper genome that includes a sequence according to Construct 1.

### Exemplary Construct 2

Construct 2 includes recombinase sites positioned to generate a conditionally defective packaging sequence of an Ad35 helper genome. Inserted LoxP sites are shown in the context of nucleotides 1-402 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. In addition, the sequence CCGGCC (SEQ ID NO: 14) was inserted to create a recognition site for the restriction enzyme FseI (TTATGGCCGGCCGGGTGGAGTTTTTTTGCAAGTTGTCGCG; SEQ ID NO: 20). Further sequence information is provided below.

Sequence corresponding to AY128640 nucleotides 1-402 engineered to include
a conditionally defective packaging sequence (LoxP sequences underlined)
(SEQ ID NO: 21)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCGGGTGGAGTTTTTTTGCAAGT
TGTCGCGATAACTTCGTATAGCATACATTATACGAAGTTATGGAAATGTTACGCATA
AAAAGGCTTCTTTTCTCACGGAACTACTTAGTTTTCCCACGGTATTTAACAGGAAAT
GAGGTAGTTTTGACCGGATGCAAGTGAAAATTGCTGATTTTCGCGCGAAAACTGAAT
GAGGAAGTGTTTTTCTGAATAATGTGGTATTTATGGCAGGGTGGAGTATTTGTTCAG
GGCCAGGTAGACTTTGACCCATTACGTGGAGGTTTCGATTACCGATAACTTCGTATA
GCATACATTATACGAAGTTAT

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
LoxP sequence
(SEQ ID NO: 18)
ATAACTTCGTATAGCATACATTATACGAAGTTAT
LoxP-flanked sequence (identified packaging signals A1, A2, A5 and A6 underlined)
(SEQ ID NO: 22)
GGAAATGTTACGCATAAAAAGGCTTCTTTTCTCACGGAACTACTTAGTTTTCCCACG
GTATTTAACAGGAAATGAGGTAGTTTTGACCGGATGCAAGTGAAAATTGCTGATTTT
CGCGCGAAAACTGAATGAGGAAGTGTTTTTCTGAATAATGTGGTATTTATGGCAGGG
TGGAGTATTTGTTCAGGGCCAGGTAGACTTTGACCCATTACGTGGAGGTTTCGATTA
CCG

FIG. 2B shows a region (the left end) of an Ad35 helper genome that includes a sequence according to Construct 2.

### Exemplary Construct 3

Construct 3 includes recombinase sites positioned to generate a conditionally defective packaging sequence of an Ad35 helper genome. Inserted LoxP sites are shown in the context of nucleotides 1-479 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. In addition, the sequence CCGGCC (SEQ ID NO: 14) was inserted to create a recognition site for the restriction enzyme FseI (TTATGGCCGGCCGGGTGGAGTTTTTTTGCAAGTTGTCGCGGGAAATGTTACGCATAA AAAGGCT; SEQ ID NO: 23). Further sequence information is provided below.

Sequence corresponding to AY128640 nucleotides 1-479 engineered to include a
conditionally defective packaging sequence (LoxP sequences underlined)
(SEQ ID NO: 24)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCGGGTGGAGTTTTTTTGCAAGT
TGTCGCGGGAAATGTTACGCATAAAAAGGCTATAACTTCGTATAGCATACATTATAC
GAAGTTATTCTTTTCTCACGGAACTACTTAGTTTTCCCACGGTATTTAACAGGAAATG
AGGTAGTTTTGACCGGATGCAAGTGAAAATTGCTGATTTTCGCGCGAAAACTGAATG
AGGAAGTGTTTTTCTGAATAATGTGGTATTTATGGCAGGGTGGAGTATTTGTTCAGG
GCCAGGTAGACTTTGACCCATTACGTGGAGGTTTCGATTACCGTGTTTTTTACCTGAA
TTTCCGCGTACCGTGTCAAAGTCTTCTGTTTTTACGTAGGTGTCAGCTGATCGCTAGG
GTATATAACTTCGTATAGCATACATTATACGAAGTTAT

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
LoxP sequence
(SEQ ID NO: 18)
ATAACTTCGTATAGCATACATTATACGAAGTTAT
LoxP-flanked sequence (identified packaging signals A1, A2, A5 and A6 underlined)
(SEQ ID NO: 25)
TCTTTTCTCACGGAACTACTTAGTTTTCCCACGGTATTTAACAGGAAATGAGGTAGTT
TTGACCGGATGCAAGTGAAAATTGCTGATTTTCGCGCGAAAACTGAATGAGGAAGT
GTTTTTCTGAATAATGTGGTATTTATGGCAGGGTGGAGTATTTGTTCAGGGCCAGGT
AGACTTTGACCCATTACGTGGAGGTTTCGATTACCGTGTTTTTTACCTGAATTTCCGC
GTACCGTGTCAAAGTCTTCTGTTTTTACGTAGGTGTCAGCTGATCGCTAGGGTAT

FIG. 2C shows a region (the left end) of an Ad35 helper genome that includes a sequence according to Construct 3.

### Exemplary Construct 4

Construct 4 includes recombinase sites positioned to generate a conditionally defective packaging sequence of an Ad35 helper genome. Inserted LoxP sites are shown in the context of nucleotides 1-480 of the reference Ad35 sequence in GenBank accession number AY128640. The LoxP sites flank a packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. In addition, the sequence CCGGCC (SEQ ID NO: 14) was inserted to create a recognition site for the restriction enzyme FseI (TTATGGCCGGCCGGGTGGAGTTTTTTTGCA; SEQ ID NO: 26). Further sequence information is provided below.

Sequence corresponding to AY128640 nucleotides 1-480 engineered to include a
conditionally defective packaging sequence (LoxP sequences underlined)
(SEQ ID NO: 27)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCGGGTGGAGTTTTTTTGCAATA
ACTTCGTATAGCATACATTATACGAAGTTATAGTTGTCGCGGGAAATGTTACGCATA
AAAAGGCTTCTTTTCTCACGGAACTACTTAGTTTTCCCACGGTATTTAACAGGAAAT
GAGGTAGTTTTGACCGGATGCAAGTGAAAATTGCTGATTTTCGCGCGAAAACTGAAT
GAGGAAGTGTTTTTCTGAATAATGTGGTATTTATGGCAGGGTGGAGTATTTGTTCAG
GGCCAGGTAGACTTTGACCCATTACGTGGAGGTTTCGATTACCGTGTTTTTTACCTGA
ATTTCCGCGTACCGTGTCAAAGTCTTCTGTTTTTACGTAGGTGTCAGCTGATCGCTAG
GGTATTTAGGGATAACAGGGTAATATAACTTCGTATAGCATACATTATACGAAGTTA
T

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
LoxP sequence
(SEQ ID NO: 18)
ATAACTTCGTATAGCATACATTATACGAAGTTAT
LoxP-flanked sequence (identified packaging signals A1, A2, A5 and A6 underlined;
sequence inserted in place of a deletion of the early E1 gene extending from
base pairs 481-3199 bold and italicized)
(SEQ ID NO: 28)
AGTTGTCGCGGGAAATGTTACGCATAAAAAGGCTTCTTTTCTCACGGAACTACTTAG
TTTTCCCACGGTATTTAACAGGAAATGAGGTAGTTTTGACCGGATGCAAGTGAAAAT
TGCTGATTTTCGCGCGAAAACTGAATGAGGAAGTGTTTTTCTGAATAATGTGGTATT
TATGGCAGGGTGGAGTATTTGTTCAGGGCCAGGTAGACTTTGACCCATTACGTGGAG
GTTTCGATTACCGTGTTTTTTACCTGAATTTCCGCGTACCGTGTCAAAGTCTTCTGTTT
TTACGTAGGTGTCAGCTGATCGCTAGGGTATTTAGGGATAACAGGGTAAT

FIG. 2D shows a region (the left end) of an Ad35 helper genome that includes a sequence according to Construct 4.

### Example 2: Analysis of Ad35 Helper Genome Propagation and Stability

The present Example demonstrates that Ad35 helper genomes including recombinase-flanked packaging sequences according to the present disclosure are stable and can be propagated without detectable genome rearrangement.

A helper genome can be present in a plasmid or in a viral vector. Plasmid forms can be used to transfect target cells for production of helper vectors (which helper vectors include the Ad35 helper genome) or for production of donor vectors (which donor vectors do not include the Ad35 helper genome). Four plasmids encoding E1-deleted Ad35 helper genomes (designated pEN025, pEN026, pEN027, and pEN028), were each transfected into HEK293 cells and propagated to determine whether viable helper viruses could be rescued. Each of pEN025, pEN026, pEN027, and pEN028 included a construct according to Constructs 1-4 in Example 1, respectively.

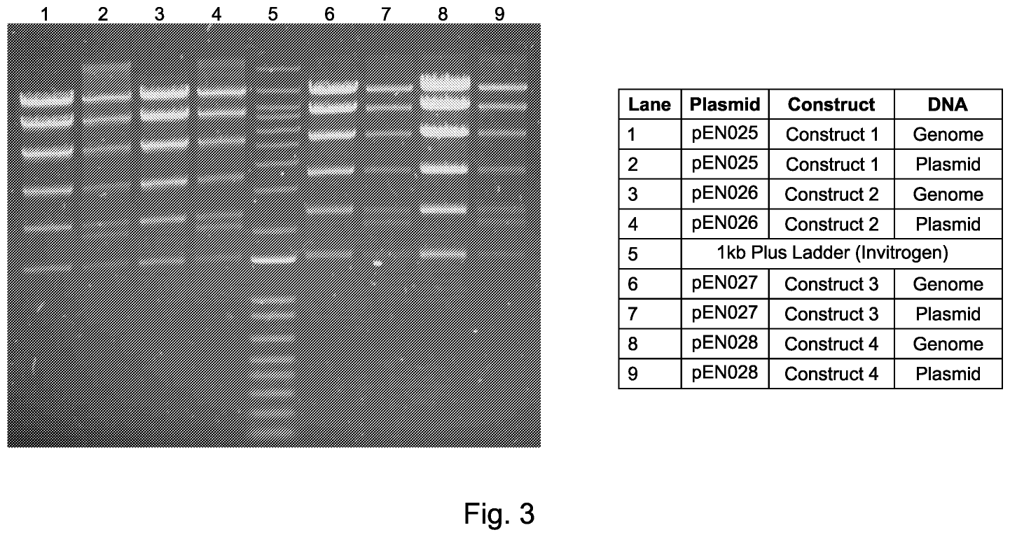

Rescued E1-deleted adenoviruses were purified using standard methods (see, e.g., Su et al. doi:10.1101/pdb.prot095547 Cold Spring Harb Protoc 2019) and viral genomes were isolated from purified helper vectors. Isolated Ad35 helper genomes were digested with BsrGI alone, and starting plasmids were digested with BsrGI and SwaI (which excises the plasmid backbone sequence) for comparison. Digestion products were analyzed by gel electrophoresis (FIG. 3).

To determine whether the Ad35 helper genomes were stable during propagation the restriction patterns obtained by digesting isolated adenoviral genomic DNA were compared to the restriction patterns obtained by digesting starting plasmids with the restriction enzymes BsrG1 and SwaI. Analysis of the restriction patterns on a gel showed the expected banding pattern and expected band sizes (FIG. 3), demonstrating that that Ad35 helper genomes including recombinase-flanked packaging sequences as disclosed herein are genetically stable and can be propagated without detectable genome rearrangement in large-scale preparations.

### Example 3: Analysis of Recombinase-Mediated Excision of Recombinase-Flanked Packaging Sequences in Ad35 Helper Genomes

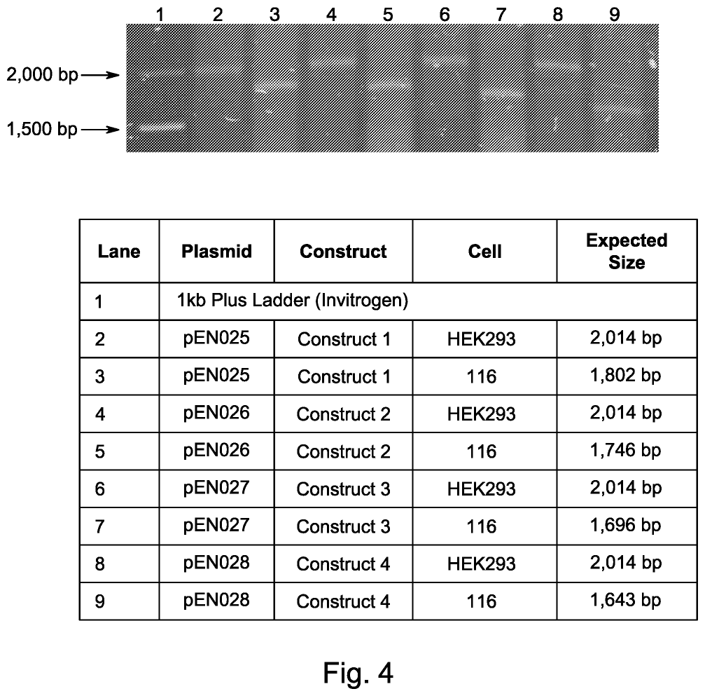

The present Example demonstrates the recombinase-mediated deletion of recombinase-flanked packaging sequences in Ad35 helper genomes. Plasmids including Ad35 helper genomes (pEN025, pEN026, pEN027, and pEN028) were linearized by digestion with SwaI (which excised the plasmid backbone sequence) and transfected into each of two cell types: HEK293 cells that do not express Cre recombinase, and 116 cells modified from HEK293 cells to express Cre recombinase. Thus, excision of loxP flanked sequences is expected in the 116 cells but not the HEK293 cells. DNA was isolated from transfected cells and digested with the restriction enzyme ApaI. Digestion of the Ad35 helper genome with restriction enzyme ApaI is expected to produce a 2014 bp fragment. A smaller DNA fragment is expected if the Ad35 helper genome has undergone recombination to mediate deletion of the recombinase-flanked packaging sequence. Restriction results were analyzed by gel electrophoresis (FIG. 4). The expected band sizes were observed for DNA isolated from HEK293 cells transfected with the Ad35 helper genomes (FIG. 4—lanes 2, 4, 6, and 8) and for DNA isolated from 116 cells transfected with the Ad35 helper genomes (FIG. 4—lanes 3, 5, 7, and 9). Data therefore show successful Cre-mediated excision of flanked packaging sequences from all helper genomes in the presence of recombinase.

### Example 4: Analysis of Helper-Dependent Adenovirus (HDAd) Production Using Ad35 Helper Vectors with Genomes Including Recombinase-Flanked Packaging Sequences

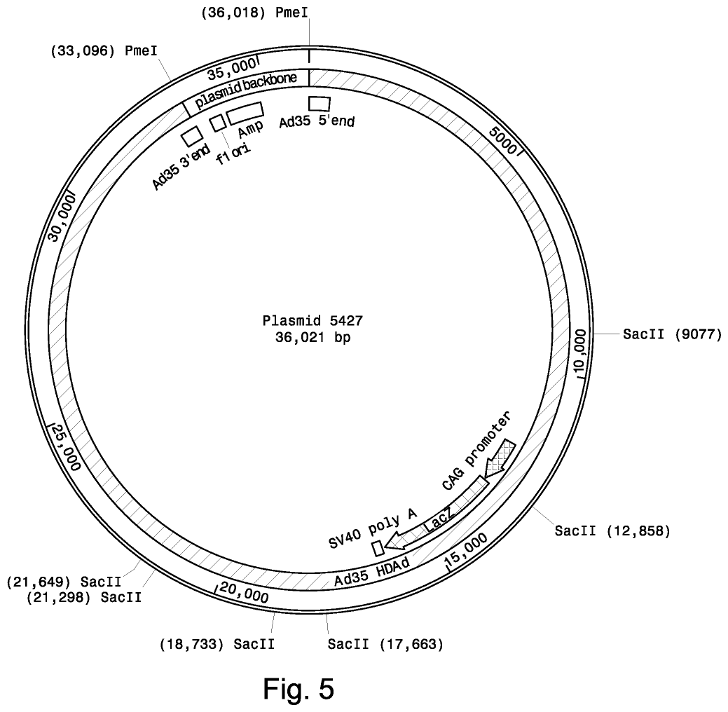

The present Example demonstrates the production of helper-dependent adenovirus (HDAd) using Ad35 helper vectors with genomes including recombinase-flanked packaging sequences. Ad35 helper vectors were purified from HEK293 cells transfected with plasmids including Ad35 helper genomes with recombinase-flanked packaging sequences (pEN025, pEN026, pEN027, and pEN028, and pEN024). Helper-dependent adenoviral vectors were then produced according to standard procedures (see Palmer and Ng, Methods Mol Biol. 2008; 433:33-53) in 116 cells using the purified Ad35 helper vectors and transfecting plasmid 5427, a plasmid that encodes a helper-dependent genome that includes terminal sequences derived from Ad35 and includes a cassette for expression of beta-galactosidase (FIG. 5). HDAd viral particles produced using Ad35 helper vectors from pEN026 and pEN028 were isolated and subsequently used to achieve production of secondary HDAd preparations by co-infection of 116 cells with the HDAd viral particles from plasmid 5427 and Ad35 helper viral particles from pEN026 or pEN028 (respectively).

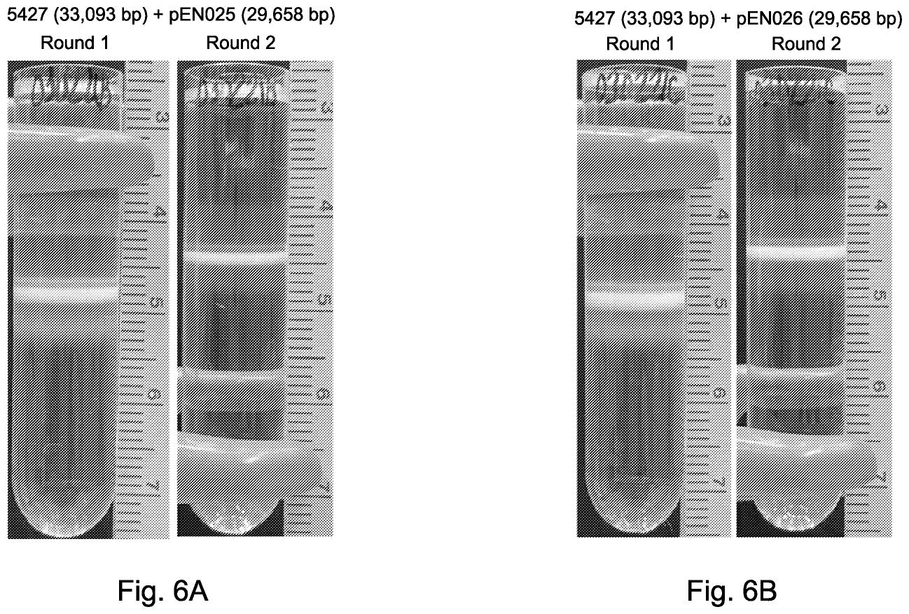

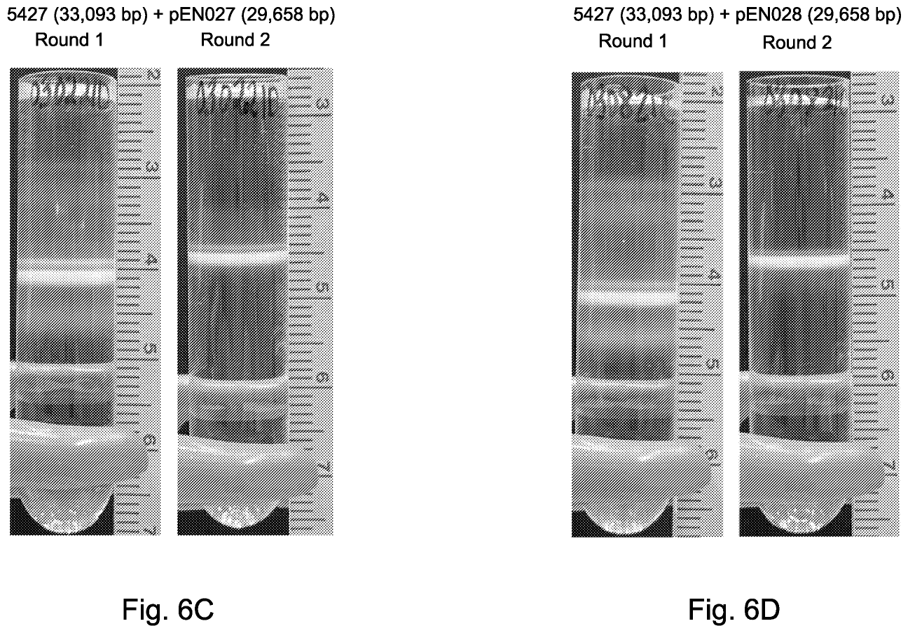

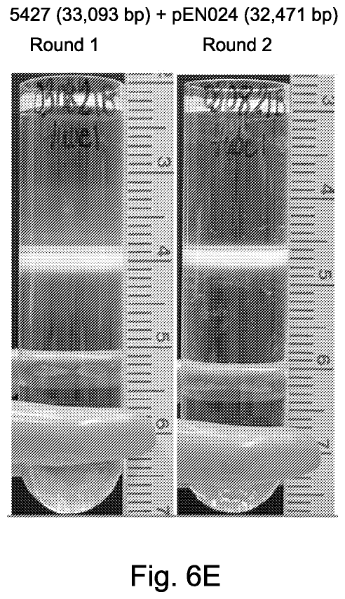

Helper-dependent adenovirus (HDAd) preparations were purified by using two consecutive cesium chloride continuous gradients (FIG. 6A-E). Purified HDAd preparations were characterized using several approaches. The physical titer or yield of the purified virus preparations was determined by spectrophotometry and can be expressed as the total number of purified viral particles (vp) or the number of viral particles per volume (vp/ml). The infectivity of the purified HDAd preparations was determined by using the purified helper-dependent viruses to infect cultured HEK293 cells and staining the cells to determine their expression of beta-galactosidase (as described in Parks et al., PNAS. 1996:93(24):13565-13570). Infected cells were expected to express beta-galactosidase. Infectivity was represented in terms of blue-forming units (BFU), which is the number of cells that showing blue staining indicating positive expression of beta-galactosidase encoded by the cassette in HDAd genome. Infectivity can be further represented as the BFU per volume of purified virus (BFU/ml) and/or the ratio between the total number of viral particles and the BFU (vp:BFU).

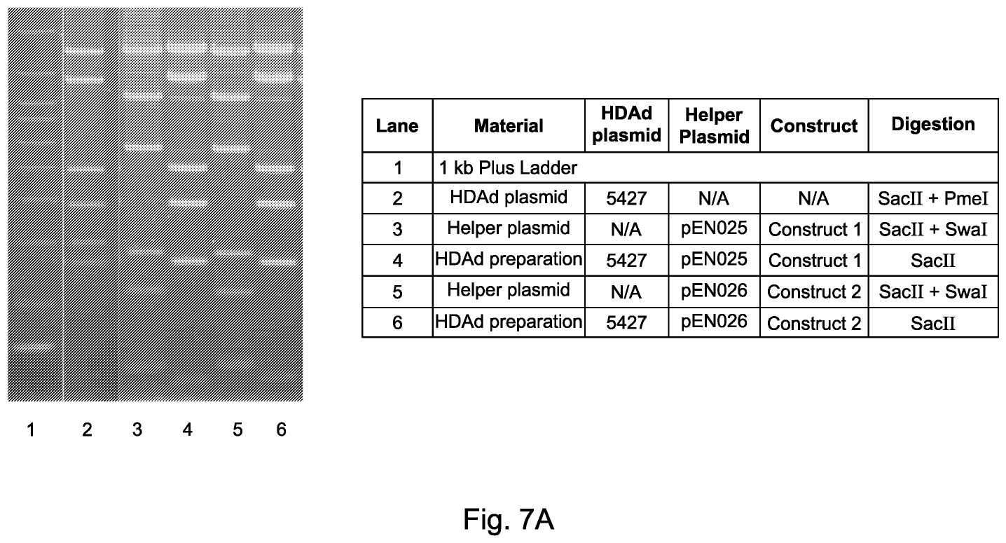

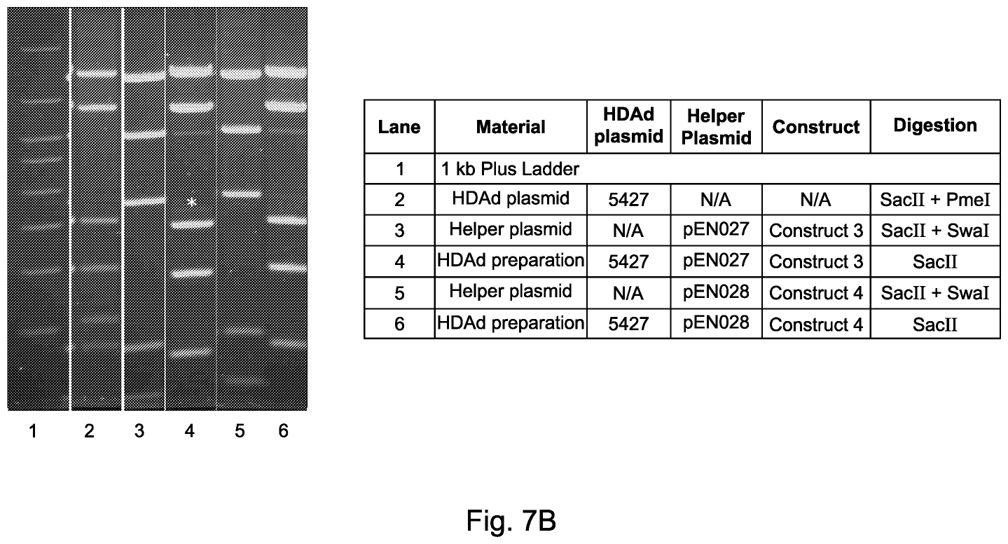

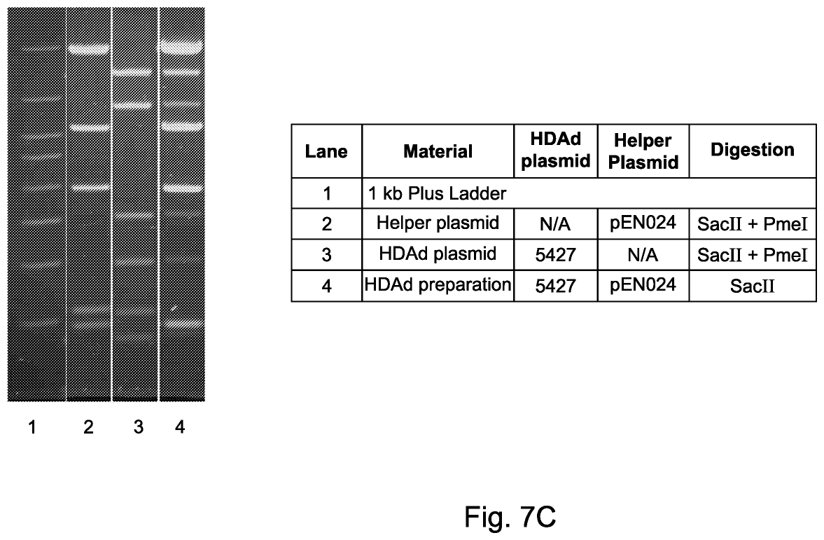

Further characterization of the purified HDAd preparations was performed using DNA isolated from the purified HDAd preparations. Isolated DNA was digested using restriction enzyme (SacII) and the restriction pattern was compared to the restriction pattern obtained by digestion using restriction enzymes (SacII and PmeI) of the starting HDAd plasmid and the restriction pattern obtained by digestion using restriction enzymes (SacII and SwaI for pEN025, pEN026, pEN027, and pEN028; or SacII and PmeI for pEN024) of the starting Ad35 helper plasmids. Analysis of the restriction patterns on a gel showed the expected banding pattern and expected band sizes (FIG. 7A-C), indicating successful HDAd production. While in FIGS. 7A and 7B the restriction patterns of the HDAd preparations demonstrate a low level of helper virus contamination, in FIG. 7C, the banding pattern in lane 4 demonstrates comparatively greater helper virus contamination. The HDAd preparation examined in FIG. 7C was prepared using Ad35 helper vectors produced using a plasmid (pEN024) encoding an Ad35 helper vector genome that includes the construct of FIG. 2E. Notwithstanding, vectors, genomes, and conditional packaging sequences analyzed in FIGS. 7A-C are advantageous and useful for various methods and compositions provided herein. Additionally, the Ad35 helper contamination fraction in the purified preparation was determined using quantitative PCR of DNA isolated from the purified HDAd preparation.

Table 3 shows the results from experiments to characterize the purified HDAd preparations. Table 4 shows results from secondary preparations, including estimated helper fraction (%).

TABLE 3
Characterization of Purified HDAd Preparations

| Helper | | Yield | Yield | Infectivity | Infectivity | Helper |
|---|---|---|---|---|---|---|
| plasmid | Construct | (vp) | (vp/ml) | (BFU/ml) | (vp:BFU) | fraction (%) |
| pEN025 | Construct 1 | 2.54e12 | 7.9e11 | 5.39e10 | 14.7:1 | 8.99 |
| pEN026 | Construct 2 | 3.59e12 | 1.25e12 | 1.05e11 | 11.9:1 | 8.10 |
| pEN027# | Construct 3 | 2.73e12 | 1.06e12 | 4.59e10 | 21.4:1 | 5.93 |
| pEN028 | Construct 4 | 2.43e12 | 9.51e11 | 7.22e11 | 13.2:1 | 7.05 |
| pEN024 | | 1.32e13 | 2.28e12 | 2.29e10 | 99.6:1 | 89.2 |

“#” indicates that evidence of genome rearrangement was observed in HDAd preparations generated using this helper plasmid (indicated by band below asterisk in FIG. 7B).

Notwithstanding, vectors, genomes, and conditional packaging sequences associated with such helper plasmids can be advantageous and useful for certain methods and compositions provided herein.

TABLE 4
Characterization of Secondary HDAd Preparations

| Helper | | Yield | Infectivity | Helper |
|---|---|---|---|---|
| plasmid | Construct | (vp) | (vp:BFU) | fraction (%) |
| pEN026 | Construct 2 | 6.66e12 | 8.35:1 | 2.9 |
| pEN028 | Construct 4 | 3.82e12 | 7.86:1 | 3.1 |

### Example 5: Design of Ad35 Helper Genomes Including Inverted Packaging Sequences

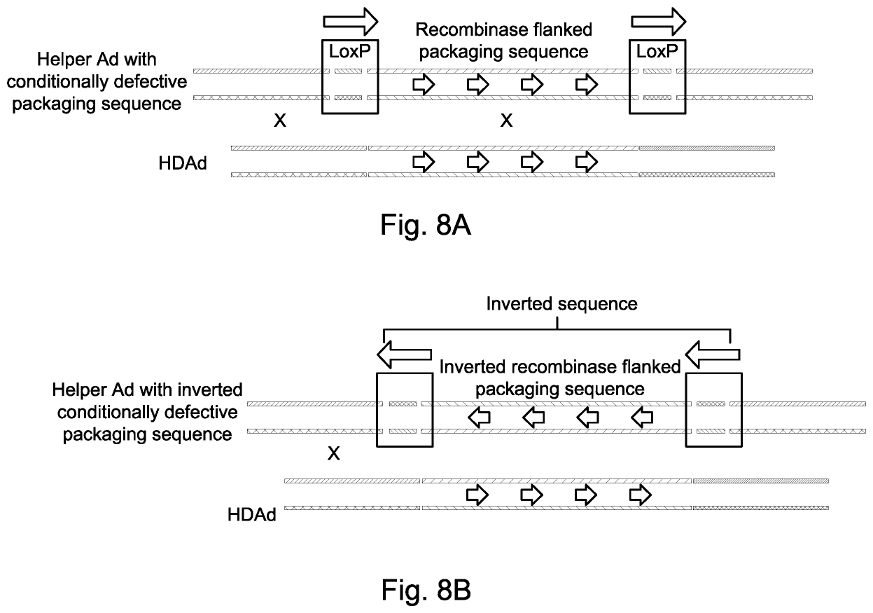

The present Example demonstrates the design of Ad35 helper genomes that include inverted packaging sequences. The present Example is based at least in part on the recognition that use of an inverted packaging sequence in an Ad35 helper vector can reduce and/or eliminate recombinase site-excising homologous recombination (compare FIG. 8A and FIG. 8B). Inversion of sequences comprising a conditionally defective packaging sequence—thereby generating an inverted recombinase-flanked packaging sequences—will reduce and/or eliminate recombinase site-excising homologous recombination, as shown in FIG. 8B. Sequence elements included within an inverted sequence are herein referred to as inverted sequence elements (e.g., a recombinase-flanked packaging sequence included within an inverted sequence is referred to as an inverted recombinase-flanked packaging sequence). A person of skill in the art would appreciate from the present disclosure that the orientation of the packaging sequence is not critical to its function (see, e.g., Palmer and Ng, Mol Ther. 2003; 8:8460852) and would further appreciate from the present disclosure that an inverted conditionally defective packaging sequence as disclosed herein is packaging competent. An inverted recombinase site flanked packaging sequence can further be excised by recombination upon contact with a corresponding recombinase and prevent packaging of a helper genome.

The present Example particularly includes Ad35 helper genomes including inverted packaging sequences as set forth below.

### Exemplary Construct 5

Construct 5 (FIG. 9A) corresponds to Construct 1 (FIG. 2A) but includes a packaging sequence inversion. The inverted recombinase-flanked packing sequence is shown in the context of nucleotides 1-480 of the reference Ad35 sequence in GenBank accession number AY128640. Positions of inserted sequence elements are identified based on their correspondence with positions of the reference Ad35 genome sequence, including if present in Construct 5 in an inverted orientation. Two inserted LoxP sites—one at position 224 and the other at position 402-flank a packaging sequence of the Ad35 genome, so that Cre recombinase-mediated deletion of the flanked packaging sequence will render the genome deficient for packaging. The sequence AGGGATAACAGGGTAAT (SEQ ID NO: 29) was inserted in place of a deletion of the early E1 gene extending from base pairs 481-3199 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 34) was inserted at position 3200 to create a second recognition site for the restriction enzyme FseI. An inverted recombinase-flanked packing sequence was generated by inversion of a sequence comprising the recombinase-flanked packaging sequence. The inverted sequence for Construct 5 includes the sequence flanked by the two FseI sites at positions 143 and 3200—which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. FIG. 9A shows a region of an Ad35 helper genome that includes Construct 5. Sequence information is provided below.

Sequence corresponding to AY128640 nucleotides 1-480 engineered to include
an inverted recombinase-flanked packing sequence (inverted sequence
underlined; FseI recognition sites bold and italicized)
(SEQ ID NO: 30)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCATTACCCTGTTATCCCTAAAT
ACCCTAGCGATCAGCTGACACCTACGTAAAAACAGAAGACTTTGACACGGTACGCG
GAAATTCAGGTAAAAAACAATAACTTCGTATAATGTATGCTATACGAAGTTATCGGT
AATCGAAACCTCCACGTAATGGGTCAAAGTCTACCTGGCCCTGAACAAATACTCCAC
CCTGCCATAAATACCACATTATTCAGAAAAACACTTCCTCATTCAGTTTTCGCGCGA
AAATCAGCAATTTTCACTTGCATCCGGTCAAAACTACCTCATTTCCTGTTAAATACCG
TGATAACTTCGTATAATGTATGCTATACGAAGTTATGGAAAACTAAGTAGTTCCGTG
AGAAAAGAAGCCTTTTTATGCGTAACATTTCCCGCGACAACTTGCAAAAAAACTCCA
CCCGGCCGGCC

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
Inverted LoxP sequence
(SEQ ID NO: 1)
ATAACTTCGTATAATGTATGCTATACGAAGTTAT
Inverted sequence (inverted LoxP sequences underlined; inverted recombinase-flanked
packaging sequence bold and italicized)
(SEQ ID NO: 32)
ATTACCCTGTTATCCCTAAATACCCTAGCGATCAGCTGACACCTACGTAAAAACAGA
AGACTTTGACACGGTACGCGGAAATTCAGGTAAAAAACAATAACTTCGTATAATGT
ATGCTATACGAAGTTATCGGTAATCGAAACCTCCACGTAATGGGTCAAAGTCTACCTG
GCCCTGAACAAATACTCCACCCTGCCATAAATACCACATTATTCAGAAAAACACTTCCT
CATTCAGTTTTCGCGCGAAAATCAGCAATTTTCACTTGCATCCGGTCAAAACTACCTCA
TTTCCTGTTAAATACCGTGATAACTTCGTATAATGTATGCTATACGAAGTTATGGAAA
ACTAAGTAGTTCCGTGAGAAAAGAAGCCTTTTTATGCGTAACATTTCCCGCGACAAC
TTGCAAAAAAACTCCACCC
Inverted recombinase-flanked packaging sequence (identified inverted packaging
signals A1, A2, A5 and A6 underlined)
(SEQ ID NO: 33)
CGGTAATCGAAACCTCCACGTAATGGGTCAAAGTCTACCTGGCCCTGAACAAATACT
CCACCCTGCCATAAATACCACATTATTCAGAAAAACACTTCCTCATTCAGTTTTCGC
GCGAAAATCAGCAATTTTCACTTGCATCCGGTCAAAACTACCTCATTTCCTGTTAAA
TACCGTG

### Exemplary Construct 6

Construct 6 (FIG. 9B) corresponds to Construct 1 (FIG. 2B) but includes a packaging sequence inversion. The inverted recombinase-flanked packing sequence is shown in the context of nucleotides 1-480 of the reference Ad35 sequence in GenBank accession number AY128640. Positions of inserted sequence elements are identified based on their correspondence with positions of the reference Ad35 genome sequence, including if present in Construct 6 in an inverted orientation. Two inserted LoxP sites-one at position 171 and the other at position 402-flank a packaging sequence of the Ad35 genome, so that Cre recombinase-mediated deletion of the flanked packaging sequence will render the genome deficient for packaging. The sequence AGGGATAACAGGGTAAT (SEQ ID NO: 29) was inserted in place of a deletion of the early E1 gene extending from base pairs 481-3199 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 34) was inserted at position 3200 to create a second recognition site for the restriction enzyme FseI. An inverted recombinase-flanked packing sequence was generated by inversion of a sequence comprising the recombinase-flanked packaging sequence. The inverted sequence for Construct 6 includes the sequence flanked by the two FseI sites at positions 143 and 3200—which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. FIG. 9B shows a region of an Ad35 helper genome that includes Construct 6. Sequence information is provided below.

Sequence corresponding to AY128640 nucleotides 1-480 engineered to include
an inverted recombinase-flanked packing sequence (inverted sequence
underlined; FseI recognition sites bold and italicized)
(SEQ ID NO: 35)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCATTACCCTGTTATCCCTAAAT
ACCCTAGCGATCAGCTGACACCTACGTAAAAACAGAAGACTTTGACACGGTACGCG
GAAATTCAGGTAAAAAACAATAACTTCGTATAATGTATGCTATACGAAGTTATCGGT
AATCGAAACCTCCACGTAATGGGTCAAAGTCTACCTGGCCCTGAACAAATACTCCAC
CCTGCCATAAATACCACATTATTCAGAAAAACACTTCCTCATTCAGTTTTCGCGCGA
AAATCAGCAATTTTCACTTGCATCCGGTCAAAACTACCTCATTTCCTGTTAAATACCG
TGGGAAAACTAAGTAGTTCCGTGAGAAAAGAAGCCTTTTTATGCGTAACATTTCCAT
AACTTCGTATAATGTATGCTATACGAAGTTATCGCGACAACTTGCAAAAAAACTCCA
CCCGGCCGGCC

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
Inverted LoxP sequence
(SEQ ID NO: 1)
ATAACTTCGTATAATGTATGCTATACGAAGTTAT
Inverted sequence (inverted LoxP sequences underlined; inverted recombinase-
flanked packaging sequence bold and italicized)
(SEQ ID NO: 31)
ATTACCCTGTTATCCCTAAATACCCTAGCGATCAGCTGACACCTACGTAAAAACAGA
AGACTTTGACACGGTACGCGGAAATTCAGGTAAAAAACAATAACTTCGTATAATGT
ATGCTATACGAAGTTATCGGTAATCGAAACCTCCACGTAATGGGTCAAAGTCTACCTG
GCCCTGAACAAATACTCCACCCTGCCATAAATACCACATTATTCAGAAAAACACTTCCT
CATTCAGTTTTCGCGCGAAAATCAGCAATTTTCACTTGCATCCGGTCAAAACTACCTCA
TTTCCTGTTAAATACCGTGGGAAAACTAAGTAGTTCCGTGAGAAAAGAAGCCTTTTTAT
GCGTAACATTTCCATAACTTCGTATAATGTATGCTATACGAAGTTATCGCGACAACTT
GCAAAAAAACTCCACCC
Inverted recombinase-flanked packaging sequence (identified inverted
packaging signals A1, A2, A5 and A6 underlined)
(SEQ ID NO: 36)
CGGTAATCGAAACCTCCACGTAATGGGTCAAAGTCTACCTGGCCCTGAACAAATACT
CCACCCTGCCATAAATACCACATTATTCAGAAAAACACTTCCTCATTCAGTTTTCGC
GCGAAAATCAGCAATTTTCACTTGCATCCGGTCAAAACTACCTCATTTCCTGTTAAA
TACCGTGGGAAAACTAAGTAGTTCCGTGAGAAAAGAAGCCTTTTTATGCGTAACATT
TCC

### Exemplary Construct 7

Construct 7 (FIG. 9C) corresponds to Construct 1 (FIG. 2C) but includes a packaging sequence inversion. The inverted recombinase-flanked packing sequence is shown in the context of nucleotides 1-480 of the reference Ad35 sequence in GenBank accession number AY128640. Positions of inserted sequence elements are identified based on their correspondence with positions of the reference Ad35 genome sequence, including if present in Construct 7 in an inverted orientation. Two inserted LoxP sites—one at position 195 and the other at position 479—flank a packaging sequence of the Ad35 genome, so that Cre recombinase-mediated deletion of the flanked packaging sequence will render the genome deficient for packaging. The sequence AGGGATAACAGGGTAAT (SEQ ID NO: 29) was inserted in place of a deletion of the early E1 gene extending from base pairs 481-3199 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 34) was inserted at position 3200 to create a second recognition site for the restriction enzyme FseI. An inverted recombinase-flanked packing sequence was generated by inversion of a sequence comprising the recombinase-flanked packaging sequence. The inverted sequence for Construct 7 includes the sequence flanked by the two FseI sites at positions 143 and 3200-which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. FIG. 9C shows a region of an Ad35 helper genome that includes Construct 7. Sequence information is provided below.

Sequence corresponding to AY128640 nucleotides 1-480 engineered to include
an inverted recombinase-flanked packing sequence (inverted sequence
underlined; FseI recognition sites bold and italicized)
(SEQ ID NO: 37)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCATTACCCTGTTATCCCTAAAT
AACTTCGTATAATGTATGCTATACGAAGTTATATACCCTAGCGATCAGCTGACACCT
ACGTAAAAACAGAAGACTTTGACACGGTACGCGGAAATTCAGGTAAAAAACACGGT
AATCGAAACCTCCACGTAATGGGTCAAAGTCTACCTGGCCCTGAACAAATACTCCAC
CCTGCCATAAATACCACATTATTCAGAAAAACACTTCCTCATTCAGTTTTCGCGCGA
AAATCAGCAATTTTCACTTGCATCCGGTCAAAACTACCTCATTTCCTGTTAAATACCG
TGGGAAAACTAAGTAGTTCCGTGAGAAAAGAATAACTTCGTATAATGTATGCTATAC
GAAGTTATAGCCTTTTTATGCGTAACATTTCCCGCGACAACTTGCAAAAAAACTCCA
CCCGGCCGGCC

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
Inverted LoxP sequence
(SEQ ID NO: 1)
ATAACTTCGTATAATGTATGCTATACGAAGTTAT
Inverted sequence finverted LoxP sequences underlined; inverted recombinase-
flanked packaging sequence bold and italicized)
(SEQ ID NO: 38)
ATTACCCTGTTATCCCTAAATAACTTCGTATAATGTATGCTATACGAAGTTATATACC
CTAGCGATCAGCTGACACCTACGTAAAAACAGAAGACTTTGACACGGTACGCGGAAAT
TCAGGTAAAAAACACGGTAATCGAAACCTCCACGTAATGGGTCAAAGTCTACCTGGCC
CTGAACAAATACTCCACCCTGCCATAAATACCACATTATTCAGAAAAACACTTCCTCAT
TCAGTTTTCGCGCGAAAATCAGCAATTTTCACTTGCATCCGGTCAAAACTACCTCATTT
CCTGTTAAATACCGTGGGAAAACTAAGTAGTTCCGTGAGAAAAGAATAACTTCGTATA
ATGTATGCTATACGAAGTTATAGCCTTTTTATGCGTAACATTTCCCGCGACAACTTGC
AAAAAAACTCCACCC
Inverted recombinase-flanked packaging sequence (identified inverted
packaging signals A1, A2, A5 and A6 underlined)
(SEQ ID NO: 39)
ATACCCTAGCGATCAGCTGACACCTACGTAAAAACAGAAGACTTTGACACGGTACG
CGGAAATTCAGGTAAAAAACACGGTAATCGAAACCTCCACGTAATGGGTCAAAGTC
TACCTGGCCCTGAACAAATACTCCACCCTGCCATAAATACCACATTATTCAGAAAAA
CACTTCCTCATTCAGTTTTCGCGCGAAAATCAGCAATTTTCACTTGCATCCGGTCAAA
ACTACCTCATTTCCTGTTAAATACCGTGGGAAAACTAAGTAGTTCCGTGAGAAAAGA

### Exemplary Construct 8

Construct 8 (FIG. 9D) corresponds to Construct 1 (FIG. 2D) but includes a packaging sequence inversion. The inverted recombinase-flanked packing sequence is shown in the context of nucleotides 1-480 of the reference Ad35 sequence in GenBank accession number AY128640. Positions of inserted sequence elements are identified based on their correspondence with positions of the reference Ad35 genome sequence, including if present in Construct 8 in an inverted orientation. Two inserted LoxP sites—one at position 161 and the other at position 3200-flank a packaging sequence of the Ad35 genome, so that Cre recombinase-mediated deletion of the flanked packaging sequence will render the genome deficient for packaging. The sequence AGGGATAACAGGGTAAT (SEQ ID NO: 29) was inserted in place of a deletion of the early E1 gene extending from base pairs 481-3199 to create a recognition site for the restriction enzyme I-SceI. In addition, the sequence CCGGCC (SEQ ID NO: 14) was inserted at position 143 to create a first recognition site for the restriction enzyme FseI, and the sequence GGCCGGCC (SEQ ID NO: 34) was inserted immediately downstream of the LoxP site inserted at position 3200 to create a second recognition site for the restriction enzyme FseI. An inverted recombinase-flanked packing sequence was generated by inversion of a sequence comprising the recombinase-flanked packaging sequence. The inverted sequence for Construct 8 includes the sequence flanked by the two FseI sites at positions 143 and 3200—which includes the two LoxP sites, the recombinase-flanked packaging sequence, and the I-SceI recognition site. The inverted LoxP sites flank the inverted recombinase-flanked packaging sequence of the Ad35 genome so that Cre recombinase-mediated deletion of the flanked sequences will render the genome deficient for packaging. In the ITR, CTATCTAT (SEQ ID NO: 12) was used in place of the canonical CATCATCA (SEQ ID NO: 13) in the reference sequence, based on the publication by Wunderlich et al., J Gen Virol. 2014; 95:1574-1584. FIG. 9D shows a region of an Ad35 helper genome that includes Construct 8. Sequence information is provided below.

Sequence corresponding to AY128640 nucleotides 1-480 engineered to include
an inverted recombinase-flanked packing sequence (inverted sequence
underlined; FseI recognition sites bold and italicized)
(SEQ ID NO: 40)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTTTTATGGCCGGCCATAACTTCGTATAATGTATG
CTATACGAAGTTATATTACCCTGTTATCCCTAAATACCCTAGCGATCAGCTGACACC
TACGTAAAAACAGAAGACTTTGACACGGTACGCGGAAATTCAGGTAAAAAACACGG
TAATCGAAACCTCCACGTAATGGGTCAAAGTCTACCTGGCCCTGAACAAATACTCCA
CCCTGCCATAAATACCACATTATTCAGAAAAACACTTCCTCATTCAGTTTTCGCGCG
AAAATCAGCAATTTTCACTTGCATCCGGTCAAAACTACCTCATTTCCTGTTAAATACC
GTGGGAAAACTAAGTAGTTCCGTGAGAAAAGAAGCCTTTTTATGCGTAACATTTCCC
GCGACAACTATAACTTCGTATAATGTATGCTATACGAAGTTATTGCAAAAAAACTCC
ACCCGGCCGGCC

Components of the above sequence are further described below.

ITR (CTATCTAT (SEQ ID NO: 12) sequence used in place of the canonical CATCATCA
(SEQ ID NO: 13) underlined)
(SEQ ID NO: 17)
CTATCTATATAATATACCTTATAGATGGAATGGTGCCAATATGTAAATGAGGTGATT
TTAAAAAGTGTGGGCCGTGTGGTGATTGGCTGTGGGGTTAACGGTTAAAAGGGGCG
GCGCGGCCGTGGGAAAATGACGTT
Inverted LoxP sequence
(SEQ ID NO: 1)
ATAACTTCGTATAATGTATGCTATACGAAGTTAT
Inverted sequence (inverted LoxP sequences underlined; inverted recombinase-
flanked packaging sequence bold and italicized)
(SEQ ID NO: 41)
ATAACTTCGTATAATGTATGCTATACGAAGTTATATTACCCTGTTATCCCTAAATACC
CTAGCGATCAGCTGACACCTACGTAAAAACAGAAGACTTTGACACGGTACGCGGAAAT
TCAGGTAAAAAACACGGTAATCGAAACCTCCACGTAATGGGTCAAAGTCTACCTGGCC
CTGAACAAATACTCCACCCTGCCATAAATACCACATTATTCAGAAAAACACTTCCTCAT
TCAGTTTTCGCGCGAAAATCAGCAATTTTCACTTGCATCCGGTCAAAACTACCTCATTT
CCTGTTAAATACCGTGGGAAAACTAAGTAGTTCCGTGAGAAAAGAAGCCTTTTTATGC
GTAACATTTCCCGCGACAACTATAACTTCGTATAATGTATGCTATACGAAGTTATTGC
AAAAAAACTCCACCC
Inverted recombinase-flanked packaging sequence (identified inverted packaging
signals A1, A2, A5 and A6 underlined)
(SEQ ID NO: 42)
ATTACCCTGTTATCCCTAAATACCCTAGCGATCAGCTGACACCTACGTAAAAACAGA
AGACTTTGACACGGTACGCGGAAATTCAGGTAAAAAACACGGTAATCGAAACCTCC
ACGTAATGGGTCAAAGTCTACCTGGCCCTGAACAAATACTCCACCCTGCCATAAATA
CCACATTATTCAGAAAAACACTTCCTCATTCAGTTTTCGCGCGAAAATCAGCAATTT
TCACTTGCATCCGGTCAAAACTACCTCATTTCCTGTTAAATACCGTGGGAAAACTAA
GTAGTTCCGTGAGAAAAGAAGCCTTTTTATGCGTAACATTTCCCGCGACAACT

### Example 6: Analysis of Ad35 Helper Genome Propagation and Stability

The present Example demonstrates successful use of Ad35 helper genomes as disclosed herein that include an inverted packaging sequence. The present Example further demonstrates that Ad35 helper genomes including inverted, recombinase-flanked packaging sequences according to the present disclosure are stable and can be propagated without detectable genome rearrangement.

Broadly, a helper genome can be present in a plasmid or in a viral vector. Plasmid forms can be used to transfect target cells for production of helper vectors (which helper vectors include the Ad35 helper genome) or for production of donor vectors (which donor vectors do not include the Ad35 helper genome). In the present Example, two plasmids encoding E1-deleted Ad35 helper genomes (designated pEN0056 and pEN0057), were each transfected into HEK293 cells and propagated to determine whether viable helper viruses could be rescued. Each of pEN0056 and pEN0057 included a construct according to Constructs 7 and 8 in Example 5, respectively.

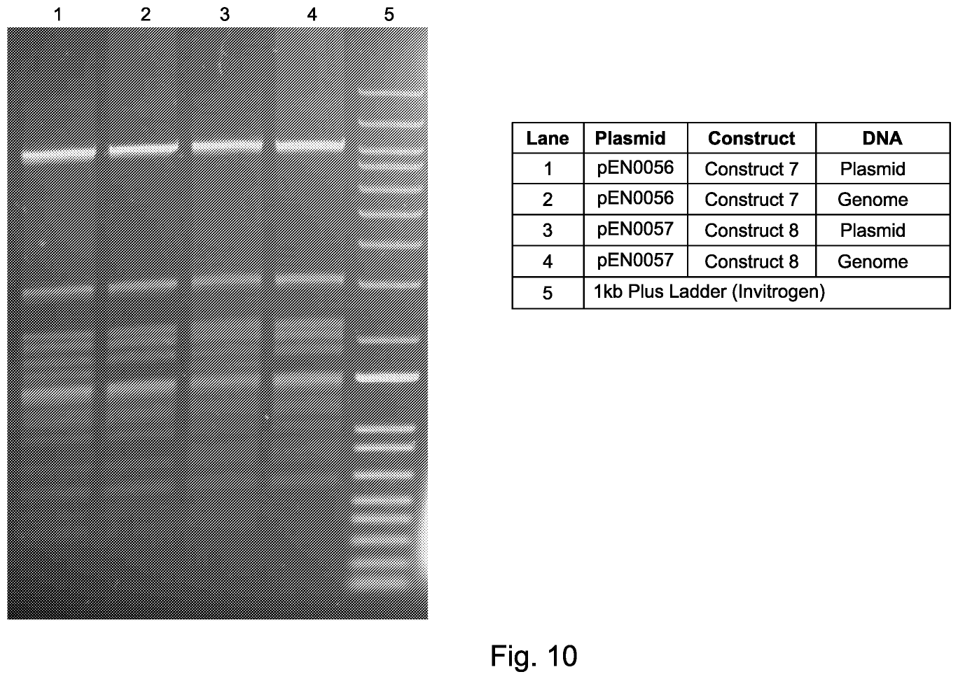

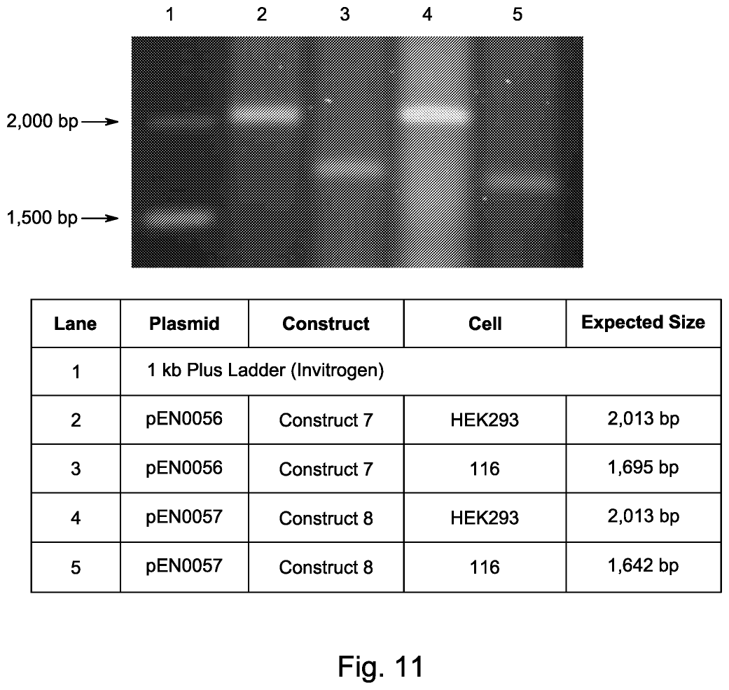

Rescued E1-deleted adenoviruses were purified using standard methods (see, e.g., Su et al. doi: 10.1101/pdb.prot095547 Cold Spring Harb Protoc 2019) and viral genomes were isolated from purified helper vectors. Isolated Ad35 helper genomes were digested with XmnI alone, and starting plasmids were digested with XmnI and SwaI (which excises the plasmid backbone sequence) for comparison. Digestion products were analyzed by gel electrophoresis (FIG. 10).

To determine whether the Ad35 helper genomes were stable during propagation the restriction patterns obtained by digesting isolated adenoviral genomic DNA were compared to the restriction patterns obtained by digesting starting plasmids with the restriction enzymes XmnI and SwaI. Analysis of the restriction patterns on a gel showed the expected banding pattern and expected band sizes (FIG. 10), demonstrating that that Ad35 helper genomes including inverted recombinase-flanked packaging sequences as disclosed herein are genetically stable and can be propagated without detectable genome rearrangement in large-scale preparations.

### Example 7: Analysis of Recombinase-Mediated Excision of Inverted Recombinase-Flanked Packaging Sequences in Ad35 Helper Genomes

The present Example demonstrates the recombinase-mediated deletion of inverted recombinase-flanked packaging sequences in Ad35 helper genomes. Plasmids including Ad35 helper genomes (pEN0056 and pEN0057) were linearized by digestion with SwaI (which excised the plasmid backbone sequence) and transfected into each of two cell types: HEK293 cells that do not express Cre recombinase, and 116 cells modified from HEK293 cells to express Cre recombinase. Thus, excision of loxP flanked sequences is expected in the 116 cells but not the HEK293 cells. DNA was isolated from transfected cells and digested with the restriction enzyme ApaI. Digestion of the Ad35 helper genome with restriction enzyme ApaI is expected to produce a 2013 bp fragment. A smaller DNA fragment is expected if the Ad35 helper genome has undergone recombination to mediate deletion of the inverted recombinase-flanked packaging sequence. Restriction results were analyzed by gel electrophoresis (FIG. 11). The expected band sizes were observed for DNA isolated from HEK293 cells transfected with the Ad35 helper genomes (FIG. 11—lanes 2 and 4) and for DNA isolated from 116 cells transfected with the Ad35 helper genomes (FIG. 11—lanes 3 and 5). Data therefore show successful Cre-mediated excision of flanked packaging sequences from all helper genomes in the presence of recombinase.

### Example 8: Analysis of Helper-Dependent Adenovirus (HDAd) Production Using Ad35 Helper Vectors with Genomes Including Inverted Recombinase-Flanked Packaging Sequences

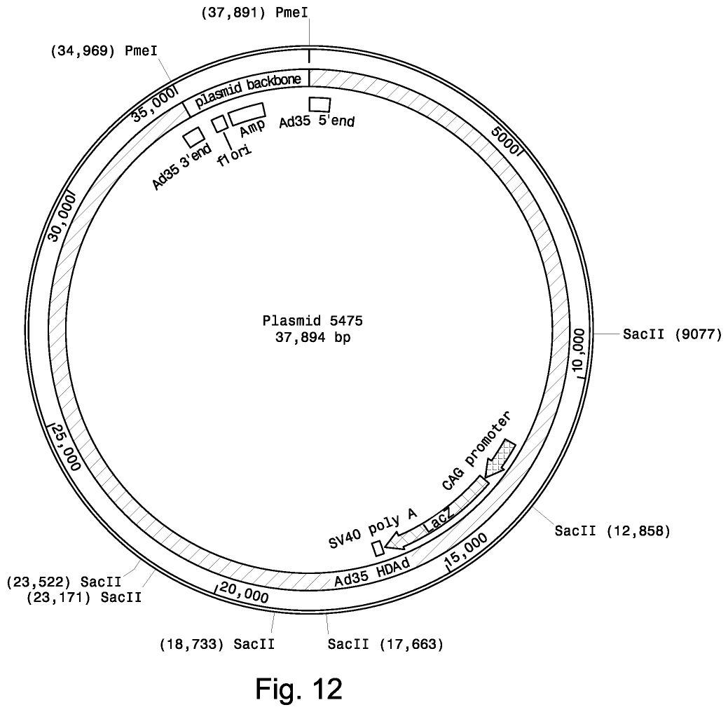

The present Example demonstrates the production of helper-dependent adenovirus (HDAd) using Ad35 helper vectors with genomes including inverted recombinase-flanked packaging sequences. Ad35 helper vectors were purified from HEK293 cells transfected with plasmids including Ad35 helper genomes with inverted recombinase-flanked packaging sequences (pEN0056 and pEN0057). Helper-dependent adenoviral vectors were produced according to standard procedures (see Palmer and Ng, Methods Mol Biol. 2008; 433:33-53) in 116 cells using the purified Ad35 helper vectors and transfecting plasmid 5475, a plasmid that encodes a helper-dependent genome that includes terminal sequences derived from Ad35 and includes a cassette for expression of beta-galactosidase (FIG. 12). HDAd viral particles produced using Ad35 helper vectors from pEN0056 and pEN0057 were isolated and subsequently used to achieve production of secondary HDAd preparations by co-infection of 116 cells with the HDAd viral particles from plasmid 5475 and Ad35 helper viral particles from pEN0056 and pEN0057 (respectively).

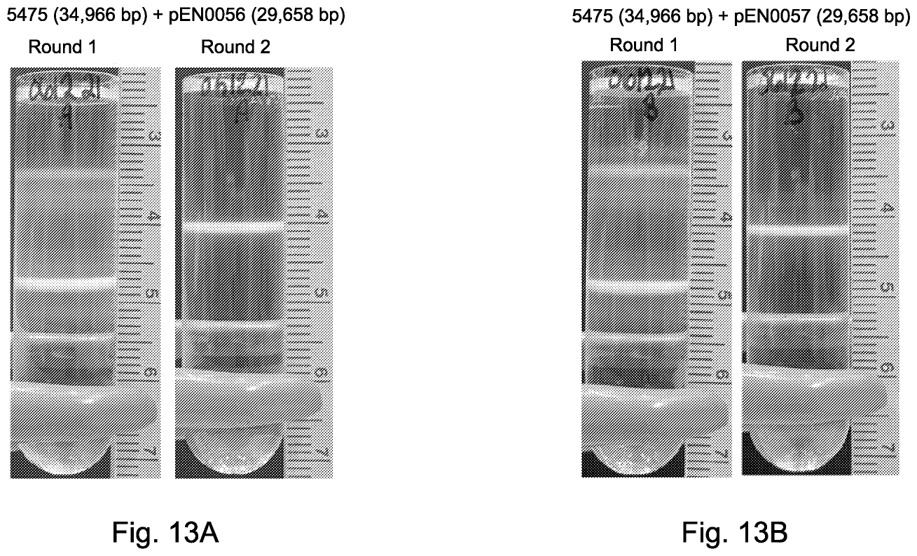

Helper-dependent adenovirus (HDAd) preparations were purified by using two consecutive cesium chloride continuous gradients (FIG. 13A-B). Purified HDAd preparations were characterized using several approaches. The physical titer or yield of the purified virus preparations was determined by spectrophotometry and can be expressed as the total number of purified viral particles (vp) or the number of viral particles per volume (vp/ml). The infectivity of the purified HDAd preparations was determined by using the purified helper-dependent viruses to infect cultured HEK293 cells and staining the cells to determine their expression of beta-galactosidase (as described in Parks et al., PNAS. 1996:93(24):13565-13570). Infected cells were expected to express beta-galactosidase. Infectivity was represented in terms of blue-forming units (BFU), which is the number of cells that showing blue staining indicating positive expression of beta-galactosidase encoded by the cassette in HDAd genome. Infectivity can be further represented as the BFU per volume of purified virus (BFU/ml) and/or the ratio between the total number of viral particles and the BFU (vp:BFU).

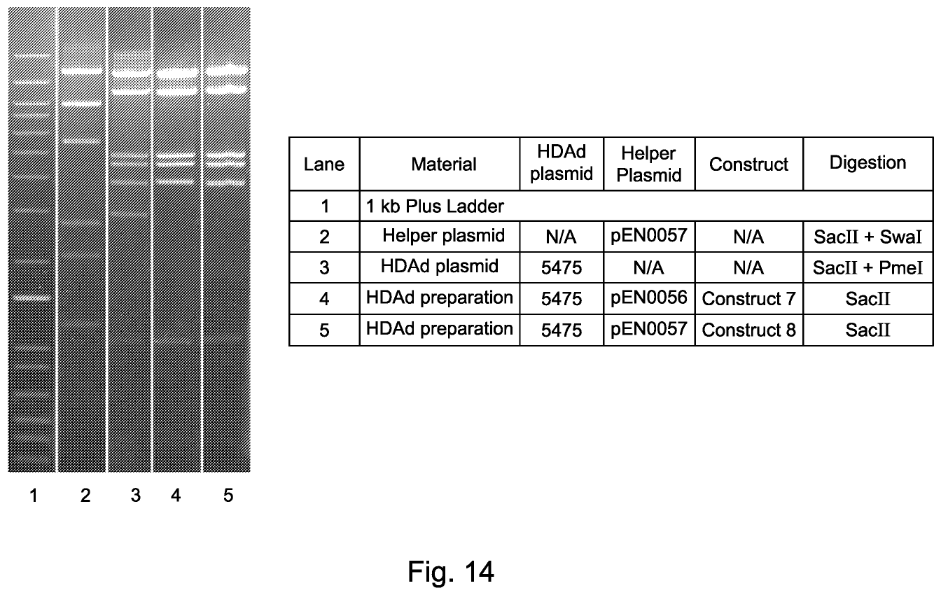

Further characterization of the purified HDAd preparations was performed using DNA isolated from the purified HDAd preparations. Isolated DNA was digested using restriction enzyme (SacII) and the restriction pattern was compared to the restriction pattern obtained by digestion using restriction enzymes (SacII and PmeI) of the starting HDAd plasmid and the restriction pattern obtained by digestion using restriction enzymes (SacII and SwaI) of the starting Ad35 helper plasmids. Analysis of the restriction patterns on a gel showed the expected banding pattern and expected band sizes (FIG. 14), indicating successful HDAd production. Vectors, genomes, and conditional packaging sequences analyzed in FIG. 14 are advantageous and useful for various methods and compositions provided herein. Additionally, the Ad35 helper contamination fraction in the purified preparation was determined using quantitative PCR of DNA isolated from the purified HDAd preparation.

Table 5 shows the results from experiments to characterize the purified HDAd preparations. Table 6 shows results from secondary preparations, including estimated helper fraction (%).

TABLE 5
Characterization of Purified HDAd Preparations

| Helper | | Yield | Yield | Infectivity | Infectivity | Helper |
|---|---|---|---|---|---|---|
| plasmid | Construct | (vp) | (vp/ml) | BFU/ml) | (vp:BFU) | fraction (%) |
| pEN0056 | Construct 7 | 3.23e12 | 1.126e12 | 5.69e10 | 19.8:1 | 1.44 |
| pEN0057 | Construct 8 | 2.45e12 | 7.806e11 | 2.44e10 | 32:1 | 1.67 |

TABLE 6
Characterization of Secondary HDAd Preparations

| Helper | | Yield | Infectivity | Helper |
|---|---|---|---|---|
| plasmid | Construct | (vp) | (vp:BFU) | fraction (%) |
| pEN0057 | Construct 8 | 5e12 | 17.6:1 | 0.95 |

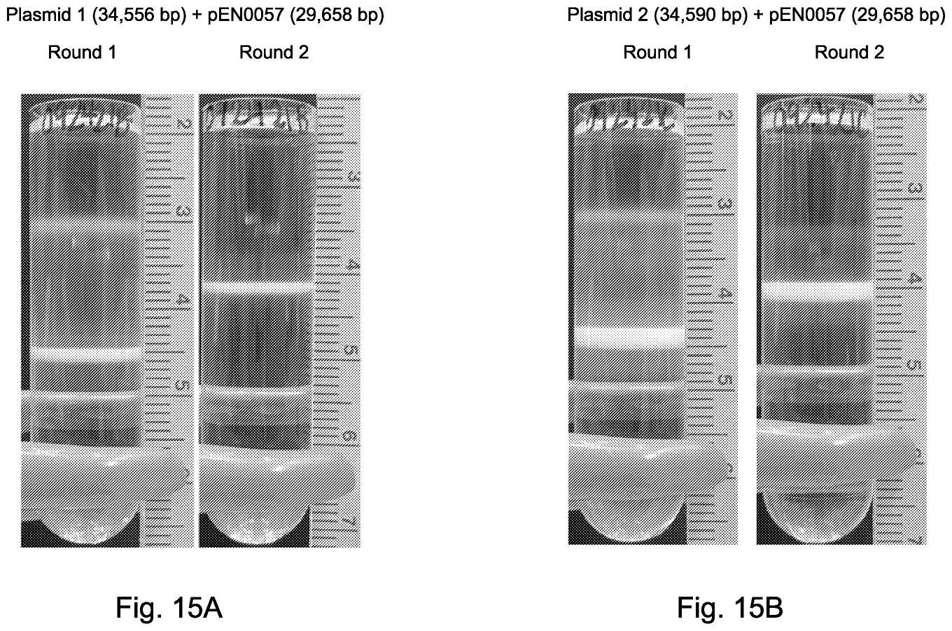

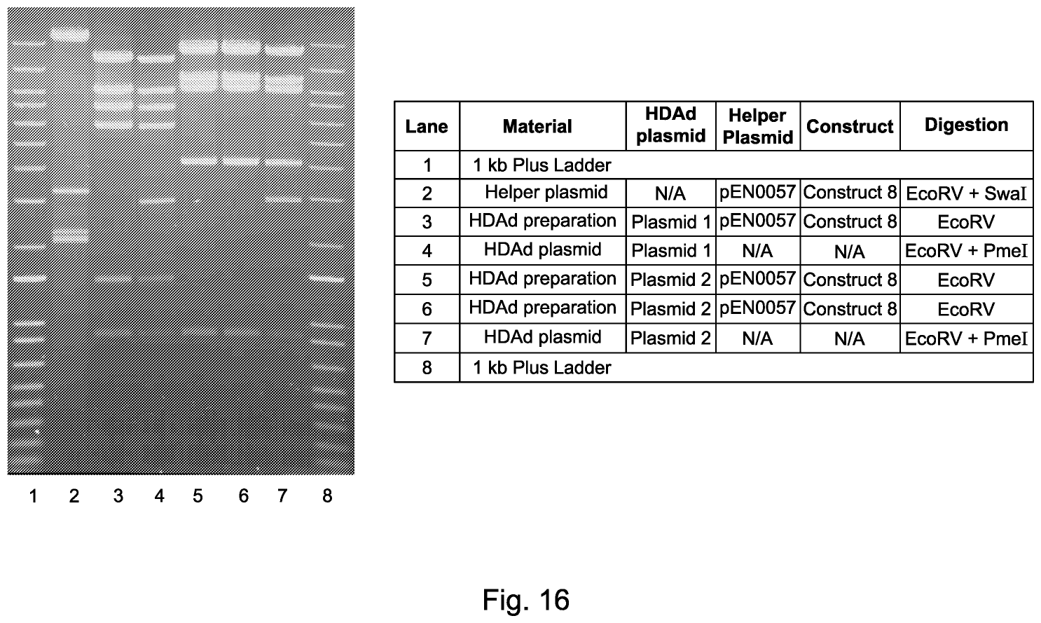

To further demonstrate production of helper-dependent adenovirus (HDAd) using Ad35 helper vectors with genomes including inverted, recombinase-flanked packaging sequences, HDAd vectors were produced in 116 cells using a purified Ad35 helper vector (from pEN0057) and transfecting one of two exemplary plasmids (plasmid 1 and plasmid 2). Plasmid 1 and plasmid 2 encode exemplary helper-dependent genomes that each include terminal sequences derived from Ad35 and includes an exemplary transgene payload, each of plasmids 1 and 2 including a different exemplary transgene payload. HDAd preparations were purified by using two consecutive cesium chloride continuous gradients (FIG. 15A-B). Purified HDAd preparations were characterized as described above. DNA isolated from the purified HDAd preparations was digested using restriction enzyme (EcoRV) and the restriction pattern was compared to the restriction pattern obtained by digestion using restriction enzymes (EcoRV and PmeI) of the starting HDAd plasmids and the restriction pattern obtained by digestion using restriction enzymes (EcoRV and SwaI) of the starting Ad35 helper plasmid. Analysis of the restriction patterns on a gel showed the expected banding pattern and expected band sizes (FIG. 16), indicating successful HDAd production.

Table 7 shows the results from experiments to characterize the purified HDAd preparations.

TABLE 7
Characterization of Purified HDAd Preparations

| Helper | | HDAd | Yield | Helper |
|---|---|---|---|---|
| plasmid | Construct | plasmid | (vp) | fraction (%) |
| pEN0057 | Construct 8 | Plasmid 1 | 2.08e12 | 0.75 |
| pEN0057 | Construct 8 | Plasmid 2 | 4.27e12 | 0.75 |
| pEN0057 | Construct 8 | Plasmid 2 | 3.74e12 | 0.81 |

### OTHER EMBODIMENTS

While we have described a number of embodiments, it is apparent that our disclosure and examples also provide other embodiments that utilize or are encompassed by the compositions and methods described herein. Therefore, it will be appreciated that the scope of disclosure is to be defined by that which may be understood from the disclosure rather than by the specific embodiments that have been represented by way of example. Limitations described with respect to one aspect of the disclosure, in certain embodiments, be practiced with respect to other aspects of the disclosure. For example, limitations of claims that depend directly or indirectly from a certain independent claim presented herein serve as support for those limitations being presented in additional dependent claims of one or more other independent claims.

All references cited herein are hereby incorporated by reference.

What is claimed is:

1. A recombinant adenoviral helper genome comprising:
a 5′ Ad35 inverted terminal repeat (ITR);
a 3′ Ad35 ITR; and
recombinase direct repeats flanking an Ad35 packaging sequence, wherein the recombinase direct repeats comprise a first recombinase direct repeat between nucleotide positions corresponding to 155 and 171, 161 and 181, 185 and 205, or 214 and 234 of GenBank Accession No. AY128640 and a second recombinase direct repeat between nucleotide positions corresponding to 377 and 504 or 3175 and 3225 of GenBank Accession No. AY128640.

2. The helper genome of claim 1, wherein the recombinase direct repeats comprise a first recombinase direct repeat between nucleotide positions corresponding to 155 and 171, 161 and 181, 185 and 205, or 214 and 234 of GenBank Accession No. AY128640 and a second recombinase direct repeat between nucleotide positions corresponding to 392 and 412 or 469 and 489 of GenBank Accession No. AY128640.

3. The helper genome of claim 1, wherein the recombinase direct repeats comprise a first recombinase direct repeat between nucleotide positions corresponding to 155 and 171, 161 and 181, 185 and 205, or 214 and 234 of GenBank Accession No. AY128640 and a second recombinase direct repeat between nucleotide positions corresponding to 3190 and 3210 of GenBank Accession No. AY128640.

4. The helper genome of claim 1, wherein the recombinase direct repeats comprise a first recombinase direct repeat between nucleotide positions corresponding to 155 and 171 of GenBank Accession No. AY128640 and a second recombinase direct repeat between nucleotide positions corresponding to 3190 and 3210 of GenBank Accession No. AY128640.

5. The helper genome of claim 1, wherein the recombinase direct repeats comprise a first recombinase direct repeat between nucleotide positions corresponding to 161 and 181 of GenBank Accession No. AY128640 and a second recombinase direct repeat between nucleotide positions corresponding to 392 and 412 of GenBank Accession No. AY128640.

6. The helper genome of claim 1, wherein the recombinase direct repeats comprise a first recombinase direct repeat between nucleotide positions corresponding to 185 and 205 of GenBank Accession No. AY128640 and a second recombinase direct repeat between nucleotide positions corresponding to 469 and 489 of GenBank Accession No. AY128640.

7. The helper genome of claim 1, wherein the recombinase direct repeats comprise a first recombinase direct repeat between nucleotide positions corresponding to 214 and 234 of GenBank Accession No. AY128640 and a second recombinase direct repeat between nucleotide positions corresponding to 392 and 412 of GenBank Accession No. AY128640.

8. The helper genome of claim 1, wherein the recombinase direct repeats comprise a first recombinase direct repeat at a nucleotide position corresponding to 161, 171, 195, or 224 of GenBank Accession No. AY128640 and a second recombinase direct repeat at a nucleotide position corresponding to 402, 479, or 3200 of GenBank Accession No. AY128640.

9. The helper genome of claim 1, wherein the recombinase direct repeats comprise a first recombinase direct repeat at a nucleotide position corresponding to 161 of GenBank Accession No. AY128640 and a second recombinase direct repeat at a nucleotide position corresponding to 3200 of GenBank Accession No. AY128640.

10. The helper genome of claim 1, wherein the recombinase direct repeats comprise a first recombinase direct repeat at a nucleotide position corresponding to 171 of GenBank Accession No. AY128640 and a second recombinase direct repeat at a nucleotide position corresponding to 402 of GenBank Accession No. AY128640.

11. The helper genome of claim 1, wherein the recombinase direct repeats comprise a first recombinase direct repeat at a nucleotide position corresponding to 195 of GenBank Accession No. AY128640 and a second recombinase direct repeat at a nucleotide position corresponding to 479 of GenBank Accession No. AY128640.

12. The helper genome of claim 1, wherein the recombinase direct repeats comprise a first recombinase direct repeat at a nucleotide position corresponding to 224 of GenBank Accession No. AY128640 and a second recombinase direct repeat at a nucleotide position corresponding to 402 of GenBank Accession No. AY128640.

13. The helper genome of any one of claims 1-12, wherein the recombinase direct repeats that flank the Ad35 packaging sequence are FRT, loxP, rox, vox, AttB, or AttP sites.

14. The helper genome of any one of claims 1-13, wherein the recombinase direct repeats that flank the Ad35 packaging sequence are loxP sites.

15. A recombinant adenoviral helper vector comprising the Ad35 helper genome of any one of claims 1-14.

16. A recombinant adenoviral vector production system comprising:
(i) the Ad35 helper genome of any one of claims 1-14 or the helper vector of claim 15, and
(ii) an HDAd35 donor genome, the HDAd35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

17. A method of producing a recombinant helper-dependent adenoviral donor vector, the method comprising isolating the recombinant helper-dependent Ad35 donor vector from a culture of cells, wherein the cells comprise:
a recombinant Ad35 helper genome of any one of claims 1-14 or a recombinant adenoviral helper vector of claim 15; and
a recombinant helper-dependent Ad35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

18. The helper genome, helper vector, system, or method of any one of claims 1-17, wherein the helper genome comprises a nucleic acid sequence that encodes an Ad35 fiber knob.

19. The genome, vector, system, or method of claim 18, wherein the Ad35 fiber knob comprises a mutation that increases affinity with CD46.

20. The helper genome, helper vector, system, or method of claim 18 or claim 19, wherein the Ad35 fiber knob comprises one or more mutations:
selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or
comprising each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

21. The helper genome, helper vector, system, or method of any one of claims 1-20, wherein the helper genome is present in a cell that comprises a nucleic acid encoding a recombinase for recombination of the direct repeats.

22. The helper genome, helper vector, system, or method of claim 21, wherein the recombinase is a Flp, Cre, Dre, Vika, or PhiC31 recombinase.

23. The helper genome, helper vector, system, or method of claim 21 or claim 22, wherein the cell is a HEK293 cell, optionally wherein the cell is a HEK293 cell that encodes or expresses Cre recombinase, optionally wherein the HEK293 cell that encodes or expresses Cre recombinase is a 116 cell.

24. The helper genome, helper vector, system, or method of any one of claims 1-23, wherein the Ad35 helper genome comprises an inverted packaging sequence.

25. A recombinant adenoviral helper genome comprising:
a 5′ Ad35 inverted terminal repeat (ITR);
a 3′ Ad35 ITR; and
recombinase direct repeats flanking an Ad35 packaging sequence, wherein the recombinase direct repeats comprise a first recombinase direct repeat between nucleotide positions corresponding to 136 and 249 of GenBank Accession No. AY128640 and a second recombinase direct repeat between nucleotide positions corresponding to 377 and 504 or 3175 and 3225 of GenBank Accession No. AY128640
wherein the Ad35 helper genome comprises an inverted packaging sequence.

26. The helper genome of claim 25, wherein the recombinase direct repeats comprise a first recombinase direct repeat between nucleotide positions corresponding to 151 and 171, 161 and 181, 185 and 205, or 214 and 234 of GenBank Accession No. AY128640 and a second recombinase direct repeat between nucleotide positions corresponding to 392 and 412 or 469 and 489 of GenBank Accession No. AY128640.

27. The helper genome of claim 25, wherein the recombinase direct repeats comprise a first recombinase direct repeat between nucleotide positions corresponding to 151 and 171, 161 and 181, 185 and 205, or 214 and 234 of GenBank Accession No. AY128640 and a second recombinase direct repeat between nucleotide positions corresponding to 3190 and 3210 of GenBank Accession No. AY128640.

28. The helper genome of claim 25, wherein the recombinase direct repeats comprise a first recombinase direct repeat between nucleotide positions corresponding to 151 and 171 of GenBank Accession No. AY128640 and a second recombinase direct repeat between nucleotide positions corresponding to 3190 and 3210 of GenBank Accession No. AY128640.

29. The helper genome of any one of claims 25-28, wherein the recombinase direct repeats that flank the Ad35 packaging sequence are FRT, loxP, rox, vox, AttB, or AttP sites.

30. The helper genome of any one of claims 25-29, wherein the recombinase direct repeats that flank the Ad35 packaging sequence are loxP sites.

31. A recombinant adenoviral helper vector comprising the Ad35 helper genome of any one of claims 25-30.

32. A recombinant adenoviral vector production system comprising:
(i) the Ad35 helper genome of any one of claims 25-30 or the helper vector of claim 31, and
(ii) an HDAd35 donor genome, the HDAd35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

33. A method of producing a recombinant helper-dependent adenoviral donor vector, the method comprising isolating the recombinant helper-dependent Ad35 donor vector from a culture of cells, wherein the cells comprise:
a recombinant Ad35 helper genome of any one of claims 25-30 or a recombinant adenoviral helper vector of claim 31; and
a recombinant helper-dependent Ad35 donor genome comprising:
a 5′ Ad35 inverted terminal repeat (ITR);
a 3′ Ad35 ITR;
an Ad35 packaging sequence; and
a nucleic acid sequence encoding at least one heterologous expression product.

34. The helper genome, helper vector, system, or method of any one of claims 25-33, wherein the helper genome comprises a nucleic acid sequence that encodes an Ad35 fiber knob.

35. The genome, vector, system, or method of claim 34, wherein the Ad35 fiber knob comprises a mutation that increases affinity with CD46.

36. The helper genome, helper vector, system, or method of claim 34 or claim 35, wherein the Ad35 fiber knob comprises one or more mutations:
selected from Ile192Val, Asp207Gly (or Glu207Gly), Asn217Asp, Thr226Ala, Thr245Ala, Thr254Pro, Ile256Leu, Ile256Val, Arg259Cys, and Arg279His; or
comprising each of mutations Ile192Val, Asp207Gly (or Glu207Gly), Asn217Asp, Thr226Ala, Thr245Ala, Thr254Pro, Ile256Leu, Ile256Val, Arg259Cys, and Arg279His.

37. The helper genome, helper vector, system, or method of any one of claims 25-36, wherein the helper genome is present in a cell that comprises a nucleic acid encoding a recombinase for recombination of the direct repeats.

38. The helper genome, helper vector, system, or method of claim 37, wherein the recombinase is a Flp, Cre, Dre, Vika, or PhiC31 recombinase.

39. The helper genome, helper vector, system, or method of claim 37 or claim 38, wherein the cell is a HEK293 cell, optionally wherein the cell is a HEK293 cell that encodes or expresses Cre recombinase, optionally wherein the HEK293 cell that encodes or expresses Cre recombinase is a 116 cell.

40. The helper genome, helper vector, system, or method of any one of claims 24-39, wherein the inverted packaging sequence comprises the Ad35 packaging sequence and one or both of the first recombinase direct repeat and the second recombinase direct repeat.

41. The helper genome, helper vector, system, or method of any one of claims 24-40, wherein the inverted packaging sequence comprises, or comprises a first end point at, a nucleotide position between nucleotide positions corresponding to 119 and 169 of AY128640 or 134 and 154 of AY128640.

42. The helper genome, helper vector, system, or method of any one of claims 24-41, wherein the inverted packaging sequence comprises, or comprises a first end point at, a nucleotide position corresponding to position 144 of AY128640.

43. The helper genome, helper vector, system, or method of any one of claims 24-42, wherein the inverted packaging sequence comprises, or comprises a second end point at, a nucleotide position between nucleotide positions corresponding to 3175 and 3225 of AY128640 or 3190 and 3210 of AY128640.

44. The helper genome, helper vector, system, or method of any one of claims 24-43, wherein the inverted packaging sequence comprises, or comprises a second end point at, a nucleotide position corresponding to position 3200 of AY128640.

45. The helper genome, helper vector, system, or method of any one of claims 24-44, wherein the inverted packaging sequence comprises, or comprises a second end point at, a nucleotide position between nucleotide positions corresponding to 455 and 505 of AY128640 or 470 and 490 of AY128640.

46. The helper genome, helper vector, system, or method of any one of claims 24-45, wherein the inverted packaging sequence comprises, or comprises a second end point at, a nucleotide position corresponding to position 480 of AY128640.

47. A recombinant recombinase site-flanked adenoviral serotype 35 (Ad35) packaging sequence, wherein recombinase direct repeats flank an Ad35 packaging sequence, wherein the Ad35 packaging sequence corresponds to a fragment of GenBank Accession No. AY128640 having a first end point between nucleotide positions corresponding to 136 and 249 of GenBank Accession No. AY128640 and a second end point between nucleotide positions corresponding to 377 and 504 or 3175 and 3225 of GenBank Accession No. AY128640.

48. The recombinant packaging sequence of claim 47, wherein the first end point is between nucleotide positions corresponding to 155 and 171, 161 and 181, 185 and 205, or 214 and 234 of GenBank Accession No. AY128640 and the second end point is between nucleotide positions corresponding to 392 and 412 or 469 and 489 of GenBank Accession No. AY128640.

49. The recombinant packaging sequence of claim 47, wherein the first end point is between nucleotide positions corresponding to 155 and 171, 161 and 181, 185 and 205, or 214 and 234 of GenBank Accession No. AY128640 and the second end point is between nucleotide positions corresponding to 3190 and 3210 of GenBank Accession No. AY128640.

50. The recombinant packaging sequence of claim 47, wherein the first end point is between nucleotide positions corresponding to 155 and 171 of GenBank Accession No. AY128640 and the second end point is between nucleotide positions corresponding to 3190 and 3210 of GenBank Accession No. AY128640.

51. The recombinant packaging sequence of claim 47, wherein the first end point is between nucleotide positions corresponding to 161 and 181 of GenBank Accession No. AY128640 and the second end point is between nucleotide positions corresponding to 392 and 412 of GenBank Accession No. AY128640.

52. The recombinant packaging sequence of claim 47, wherein the first end point is between nucleotide positions corresponding to 185 and 205 of GenBank Accession No. AY128640 and the second end point is between nucleotide positions corresponding to 469 and 489 of GenBank Accession No. AY128640.

53. The recombinant packaging sequence of claim 47, wherein the first end point is between nucleotide positions corresponding to 214 and 234 of GenBank Accession No. AY128640 and the second end point is between nucleotide positions corresponding to 392 and 412 of GenBank Accession No. AY128640.

54. The recombinant packaging sequence of claim 47, wherein the first end point is at a nucleotide position corresponding to 161, 162, 171, 172, 195, 196, 224, or 225 of GenBank Accession No. AY128640 and the second end point is at a nucleotide position corresponding to 402, 479, or 3200 of GenBank Accession No. AY128640.

55. The recombinant packaging sequence of claim 47, wherein the first end point is at a nucleotide position corresponding to 161 or 162 of GenBank Accession No. AY128640 and the second end point is at a nucleotide position corresponding to 3200 of GenBank Accession No. AY128640.

56. The recombinant packaging sequence of claim 47, wherein the first end point is at a nucleotide position corresponding to 171 or 172 of GenBank Accession No. AY128640 and the second end point is at a nucleotide position corresponding to 402 of GenBank Accession No. AY128640.

57. The recombinant packaging sequence of claim 47, wherein the first end point is at a nucleotide position corresponding to 195 or 196 of GenBank Accession No. AY128640 and the second end point is at a nucleotide position corresponding to 479 of GenBank Accession No. AY128640.

58. The recombinant packaging sequence of claim 47, wherein the first end point is at a nucleotide position corresponding to 224 or 225 of GenBank Accession No. AY128640 and the second end point is at a nucleotide position corresponding to 402 of GenBank Accession No. AY128640.

59. The recombinant packaging sequence of any one of claims 47-58, wherein the packaging sequence is present in an adenoviral genome and is inverted, optionally wherein the packaging sequence is inverted as compared to a 5′ ITR of the adenoviral genome.

60. A recombinant adenoviral helper genome comprising:
a 5′ Ad35 inverted terminal repeat (ITR);
a 3′ Ad35 ITR; and
an inverted sequence comprising an Ad35 packaging sequence,
wherein the inverted sequence comprises, or comprises a first end point at, a nucleotide position between nucleotide positions corresponding to 119 and 169 of AY128640 or 134 and 154 of AY128640, optionally wherein the inverted sequence comprises, or comprises a first end point at, a nucleotide position corresponding to position 144 of AY128640, and
wherein (i) the inverted sequence comprises, or comprises a second end point at, a nucleotide position between nucleotide positions corresponding to 3175 and 3225 of AY128640 or 3190 and 3210 of AY128640, optionally wherein the inverted sequence comprises, or comprises a second end point at, a nucleotide position corresponding to position 3200 of AY128640, or (ii) the inverted sequence comprises, or comprises a second end point at, a nucleotide position between nucleotide positions corresponding to 455 and 505 of AY128640 or 470 and 490 of AY128640, optionally wherein the inverted sequence comprises, or comprises a second end point at, a nucleotide position corresponding to position 480 of AY128640.

61. The recombinant adenoviral helper genome of claim 60, wherein recombinase direct repeats flank the Ad35 packaging sequence.


SEQUENCE LISTING (55 sequences)

SEQ ID NO: 1 - DNA, 34 bp, Artificial Sequence
  Description of Artificial Sequence Synthetic oligonucleotide
ataacttcgtataatgtatgctatacgaagttat

SEQ ID NO: 2 - DNA, 34 bp, Artificial Sequence
  Description of Artificial Sequence Synthetic oligonucleotide
ataacttcgtataaagtatcctatacgaagttat

SEQ ID NO: 3 - DNA, 34 bp, Artificial Sequence
  Description of Artificial Sequence Synthetic oligonucleotide
ataacttcgtataatgtatactatacgaagttat

SEQ ID NO: 4 - DNA, 34 bp, Artificial Sequence
  Description of Artificial Sequence Synthetic oligonucleotide
  modified_base (14)..(16): a, c, t, g, unknown or other
  modified_base (19)..(21): a, c, t, g, unknown or other
ataacttcgtatannntannntatacgaacggta

SEQ ID NO: 5 - DNA, 34 bp, Artificial Sequence
  Description of Artificial Sequence Synthetic oligonucleotide
  modified_base (14)..(16): a, c, t, g, unknown or other
  modified_base (19)..(21): a, c, t, g, unknown or other
taccgttcgtatannntannntatacgaagttat

SEQ ID NO: 6 - DNA, 34 bp, Artificial Sequence
  Description of Artificial Sequence Synthetic oligonucleotide
ataacttcgtataagaaaccatatacgaagttat

SEQ ID NO: 7 - DNA, 34 bp, Artificial Sequence
  Description of Artificial Sequence Synthetic oligonucleotide
ataacttcgtatataataccatatacgaagttat

SEQ ID NO: 8 - DNA, 34 bp, Artificial Sequence
  Description of Artificial Sequence Synthetic oligonucleotide
ataacttcgtataagatagaatatacgaagttat

SEQ ID NO: 9 - DNA, 34 bp, Artificial Sequence
  Description of Artificial Sequence Synthetic oligonucleotide
ataacttcgtatacgataccatatacgaagttat

SEQ ID NO: 10 - DNA, 34 bp, Artificial Sequence
  Description of Artificial Sequence Synthetic oligonucleotide
ataacttcgtataatgtgtactatacgaagttat

SEQ ID NO: 11 - DNA, 34 bp, Artificial Sequence
  Description of Artificial Sequence Synthetic oligonucleotide
gaagttcctattctctagaaagtataggaacttc

SEQ ID NO: 12 - DNA, 8 bp, Artificial Sequence
  Description of Artificial Sequence Synthetic oligonucleotide
ctatctat

SEQ ID NO: 13 - DNA, 8 bp, Artificial Sequence
  Description of Artificial Sequence Synthetic oligonucleotide
catcatca

SEQ ID NO: 14 - DNA, 6 bp, Artificial Sequence
  Description of Artificial Sequence Synthetic oligonucleotide
ccggcc

SEQ ID NO: 15 - DNA, 93 bp, Artificial Sequence
  Description of Artificial Sequence Synthetic oligonucleotide
ttatggccggccgggtggagtttttttgcaagttgtcgcgggaaatgttacgcataaaaaggcttcttttctcacggaactacttagttttcc

SEQ ID NO: 16 - DNA, 476 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgggaaatgttacgcataaaaaggcttcttttctcacggaactacttagttttccataacttcgtatagcatacattatacgaagttatcacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgataacttcgtatagcatacattatacgaagttat

SEQ ID NO: 17 - DNA, 137 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgtt

SEQ ID NO: 18 - DNA, 34 bp, Artificial Sequence
  Description of Artificial Sequence Synthetic oligonucleotide
ataacttcgtatagcatacattatacgaagttat

SEQ ID NO: 19 - DNA, 178 bp, Artificial Sequence
  Description of Artificial Sequence Synthetic polynucleotide
cacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccg

SEQ ID NO: 20 - DNA, 40 bp, Artificial Sequence
  Description of Artificial Sequence Synthetic oligonucleotide
ttatggccggccgggtggagtttttttgcaagttgtcgcg

SEQ ID NO: 21 - DNA, 476 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgataacttcgtatagcatacattatacgaagttatggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgataacttcgtatagcatacattatacgaagttat

SEQ ID NO: 22 - DNA, 231 bp, Artificial Sequence
  Description of Artificial Sequence Synthetic polynucleotide
ggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccg

SEQ ID NO: 23 - DNA, 64 bp, Artificial Sequence
  Description of Artificial Sequence Synthetic oligonucleotide
ttatggccggccgggtggagtttttttgcaagttgtcgcgggaaatgttacgcataaaaaggct

SEQ ID NO: 24 - DNA, 553 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgggaaatgttacgcataaaaaggctataacttcgtatagcatacattatacgaagttattcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatataacttcgtatagcatacattatacgaagttat

SEQ ID NO: 25 - DNA, 284 bp, Artificial Sequence
  Description of Artificial Sequence Synthetic polynucleotide
tcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtat

SEQ ID NO: 26 - DNA, 30 bp, Artificial Sequence
  Description of Artificial Sequence Synthetic oligonucleotide
ttatggccggccgggtggagtttttttgca

SEQ ID NO: 27 - DNA, 572 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaataacttcgtatagcatacattatacgaagttatagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttagggataacagggtaatataacttcgtatagcatacattatacgaagttat

SEQ ID NO: 28 - DNA, 337 bp, Artificial Sequence
  Description of Artificial Sequence Synthetic polynucleotide
agttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttagggataacagggtaat

SEQ ID NO: 29 - DNA, 17 bp, Artificial Sequence
  Description of Artificial Sequence Synthetic oligonucleotide
agggataacagggtaat

SEQ ID NO: 30 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccattaccctgttatccctaaataccctagcgatcagctgacacctacgtaaaaacagaagactttgacacggtacgcggaaattcaggtaaaaaacaataacttcgtataatgtatgctatacgaagttatcggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgataacttcgtataatgtatgctatacgaagttatggaaaactaagtagttccgtgagaaaagaagcctttttatgcgtaacatttcccgcgacaacttgcaaaaaaactccacccggccggcc

SEQ ID NO: 31 - DNA, 423 bp, Artificial Sequence
  Description of Artificial Sequence Synthetic polynucleotide
attaccctgttatccctaaataccctagcgatcagctgacacctacgtaaaaacagaagactttgacacggtacgcggaaattcaggtaaaaaacaataacttcgtataatgtatgctatacgaagttatcggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttccataacttcgtataatgtatgctatacgaagttatcgcgacaacttgcaaaaaaactccaccc

SEQ ID NO: 32 - DNA, 423 bp, Artificial Sequence
  Description of Artificial Sequence Synthetic polynucleotide
attaccctgttatccctaaataccctagcgatcagctgacacctacgtaaaaacagaagactttgacacggtacgcggaaattcaggtaaaaaacaataacttcgtataatgtatgctatacgaagttatcggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgataacttcgtataatgtatgctatacgaagttatggaaaactaagtagttccgtgagaaaagaagcctttttatgcgtaacatttcccgcgacaacttgcaaaaaaactccaccc

SEQ ID NO: 33 - DNA, 178 bp, Artificial Sequence
  Description of Artificial Sequence Synthetic polynucleotide
cggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtg

SEQ ID NO: 34 - DNA, 8 bp, Artificial Sequence
  Description of Artificial Sequence Synthetic oligonucleotide
ggccggcc

SEQ ID NO: 35 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccattaccctgttatccctaaataccctagcgatcagctgacacctacgtaaaaacagaagactttgacacggtacgcggaaattcaggtaaaaaacaataacttcgtataatgtatgctatacgaagttatcggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttccataacttcgtataatgtatgctatacgaagttatcgcgacaacttgcaaaaaaactccacccggccggcc

SEQ ID NO: 36 - DNA, 231 bp, Artificial Sequence
  Description of Artificial Sequence Synthetic polynucleotide
cggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttcc

SEQ ID NO: 37 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccattaccctgttatccctaaataacttcgtataatgtatgctatacgaagttatataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaataacttcgtataatgtatgctatacgaagttatagcctttttatgcgtaacatttcccgcgacaacttgcaaaaaaactccacccggccggcc

SEQ ID NO: 38 - DNA, 423 bp, Artificial Sequence
  Description of Artificial Sequence Synthetic polynucleotide
attaccctgttatccctaaataacttcgtataatgtatgctatacgaagttatataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaataacttcgtataatgtatgctatacgaagttatagcctttttatgcgtaacatttcccgcgacaacttgcaaaaaaactccaccc

SEQ ID NO: 39 - DNA, 284 bp, Artificial Sequence
  Description of Artificial Sequence Synthetic polynucleotide
ataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaaga

SEQ ID NO: 40 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccataacttcgtataatgtatgctatacgaagttatattaccctgttatccctaaataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttcccgcgacaactataacttcgtataatgtatgctatacgaagttattgcaaaaaaactccacccggccggcc

SEQ ID NO: 41 - DNA, 423 bp, Artificial Sequence
  Description of Artificial Sequence Synthetic polynucleotide
ataacttcgtataatgtatgctatacgaagttatattaccctgttatccctaaataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttcccgcgacaactataacttcgtataatgtatgctatacgaagttattgcaaaaaaactccaccc

SEQ ID NO: 42 - DNA, 337 bp, Artificial Sequence
  Description of Artificial Sequence Synthetic polynucleotide
attaccctgttatccctaaataccctagcgatcagctgacacctacgtaaaaacagaagactttgacacggtacgcggaaattcaggtaaaaaacacggtaatcgaaacctccacgtaatgggtcaaagtctacctggccctgaacaaatactccaccctgccataaataccacattattcagaaaaacacttcctcattcagttttcgcgcgaaaatcagcaattttcacttgcatccggtcaaaactacctcatttcctgttaaataccgtgggaaaactaagtagttccgtgagaaaagaagcctttttatgcgtaacatttcccgcgacaact

SEQ ID NO: 43 - DNA, 497 bp, Human mastadenovirus B
ctatctatataatataccttatagatggaatggtgccaacatgtaaatgaggtaatttaaaaaagtgcgcgctgtgtggtgattggctgcggggttaacggctaaaaggggcggcgcgaccgtgggaaaatgacgtgacttatgtgggaggagttatgttgcaagttattacggtaaatgtgacgtaaaacgaggtgtggtttgaacacggaagtagacagttttcccacgcttactgacaggatatgaggtagttttgggcggatgcaagtgaaaattctccattttcgcgcgaaaactaaatgaggaagtgaatttctgagtcatttcgcggttatgccagggtggagtatttgccgagggccgagtagactttgaccgtttacgtggaggtttcgattaccgtgtttttcacctaaatttccgcgtacggtgtcaaagttctgtgtttttacgtaggtgtcagctgatcgctagggtatttaaacctgacgagt

SEQ ID NO: 44 - DNA, 493 bp, Unknown
  Description of Unknown Human adenovirus 35 sequence
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggt

SEQ ID NO: 45 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgggaaatgttacgcataaaaaggcttcttttctcacggaactacttagttttccataacttcgtatagcatacattatacgaagttatcacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgataacttcgtatagcatacattatacgaagttattgttttttacctgaatttccgcgtaccgtgtcaaagtcttctgtttttacgtaggtgtcagctgatcgctagggtatttagggataacagggtaatggccggcc

SEQ ID NO: 46 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgataacttcgtatagcatacattatacgaagttatggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgataacttcgtatagcatacattatacgaagttattgttttttacctgaatttccgcgtaccgtgtcaaagtcttctgtttttacgtaggtgtcagctgatcgctagggtatttagggataacagggtaatggccggcc

SEQ ID NO: 47 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaagttgtcgcgggaaatgttacgcataaaaaggctataacttcgtatagcatacattatacgaagttattcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatataacttcgtatagcatacattatacgaagttatttagggataacagggtaatggccggcc

SEQ ID NO: 48 - DNA, 580 bp, Artificial Sequence
  Description of Artificial Sequence Synthetic polynucleotide
ctatctatataatataccttatagatggaatggtgccaatatgtaaatgaggtgattttaaaaagtgtgggccgtgtggtgattggctgtggggttaacggttaaaaggggcggcgcggccgtgggaaaatgacgttttatggccggccgggtggagtttttttgcaataacttcgtatagcatacattatacgaagttatagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttagggataacagggtaatataacttcgtatagcatacattatacgaagttatggccggcc

SEQ ID NO: 49 - DNA, 580 bp, Artificial Sequence
  Description of Artificial Sequence Synthetic polynucleotide
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacatttaaatataacttcgtatagcatacattatacgaagttatctcgaggcgtcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttataacttcgtatagcatacattatacgaagttatatttaaatta

SEQ ID NO: 50 - DNA, 34794 bp, Unknown
  Description of Unknown Human adenovirus 35 sequence
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcaatactccaggggtgattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgatattcaagcacctgttcctgtggacgtgcgcaagcccattcctgtgaagcttaagcctgggaaacgtccagcagtggagaaacttgaggacttgttacagggtggggacggacctttggacttgagtacacggaaacgtccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagagcgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatccagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagccagaggatgattgggaggtggccattaaaaattatgccaagatagctttgaggcctgataaacagtataagatcagtagacggattaatatccggaatgcttgttacatatctggaaatggggctgaggtggtaatagatactcaagacaagacagttattagatgctgcatgatggatatgtggcctggagtagtcggtatggaagcagtcacttttgtaaatgttaagtttaggggagatggttataatggaatagtgtttatggccaataccaaacttatattgcatggttgtagcttttttggtttcaacaatacctgtgtagatgcctggggacaggttagtgtacgggggtgtagtttctatgcgtgttggattgccacagctggcagaaccaagagtcaattgtctctgaagaaatgcatattccaaagatgtaacctgggcattctgaatgaaggcgaagcaagggtccgtcactgcgcttctacagatactggatgttttattttaattaagggaaatgccagcgtaaagcataacatgatttgtggtgcttccgatgagaggccttatcaaatgctcacttgtgctggtgggcattgtaatatgctggctactgtgcatattgtttcccatcaacgcaaaaaatggcctgtttttgatcacaatgtgttgaccaagtgcaccatgcatgcaggtgggcgtagaggaatgtttatgccttaccagtgtaacatgaatcatgtgaaagtgttgttggaaccagatgccttttccagaatgagcctaacaggaatctttgacatgaacacgcaaatctggaagatcctgaggtatgatgatacgagatcgagggtgcgcgcatgcgaatgcggaggcaagcatgccaggttccagccggtgtgtgtagatgtgaccgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgacatgagtggaaatgcttcttttaaggggggagtcttcagcccttatctgacagggcgtctcccatcctgggcaggagttcgtcagaatgttatgggatctactgtggatggaagacccgttcaacccgccaattcttcaacgctgacctatgctactttaagttcttcacctttggacgcagctgcagccgctgccgccgcctctgtcgccgctaacactgtgcttggaatgggttactatggaagcatcgtggctaattccacttcctctaataacccttctacactgactcaggacaagttacttgtccttttggcccagctggaggctttgacccaacgtctgggtgaactttctcagcaggtggccgagttgcgagtacaaactgagtctgctgtcggcacggcaaagtctaaataaaaaaaattccagaatcaatgaataaataaacgagcttgttgttgatttaaaatcaagtgtttttatttcatttttcgcgcacggtatgccctggaccaccgatctcgatcattgagaactcggtggattttttccagaatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcgaggtgaaattatgtgcattttggattggatttttaagttggcaatattgccgccaagatcccgtcttgggttcatgttatgaaggactaccaagacggtgtatccggtacatttaggaaatttatcgtgcagcttggatggaaaagcgtggaaaaatttggagacacccttgtgtcctccgagattttccatgcactcatccatgataatagcaatggggccgtgggcagcggcgcgggcaaacacgttccgtgggtctgacacatcatagttatgttcctgagttaaatcatcataagccattttaatgaatttggggcggagcgtaccagattggggtatgaatgttccttcgggccccggagcatagttcccctcacagatttgcatttcccaagctttcagttctgagggtggaatcatgtccacctggggggctatgaagaacaccgtttcgggggcgggggtgattagttgggatgatagcaagtttctgagcaattgagatttgccacatccggtggggccataaataattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggaaagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatccgcgccgcaggaggcgcaaacagtttcacattccaccagccaggttaaatccggttcattggggtcaaaaacaagttttccgccatattttttgatgcgtttcttacctttggtctccataagttcgtgtcctcgttgagtgacaaacaggctgtccgtatctccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccagggcatgggatgggtgagagcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtccatgtcgtcacaggctccctgttcccagagttggaagtctacccgtttcttgtaggcggggttgggcaaagcgaaagtaacatcattgaagagaatcttaccggctctgggcataaaattgcgagtgatgcggaaaggctgtggtacttccgctcgattgttgatcacctgggcagctaggacgatttcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgggggcatggctggtagtggcacgtcggcgccgcgcacgggcaggttctggtattgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggttaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagcccctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctatttttttttttttttgccgctcagaatgcatcccgtgctgcgacagatgcgcccccaacaacagcccccctcgcagcagcagcagcagcaaccacaaaaggctgtccctgcaactactgcaactgccgccgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgagacgaggatttcgaagttgatgaagtgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtagctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctaatcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgatctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctgttattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggtgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtgttggacttgggtgggagaggaaggggcaacccgtttgctcatttgcgccctcgcttgggtggtatgttgtgaaaaaaaataaaaaagaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcagaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttggaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtttactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaatgtccattcttatctcgcccagtaataacaccggttggggtctgcgcgctccaagcaagatgtacggaggcgcacgcaaacgttctacccaacatcccgtgcgtgttcgcggacattttcgcgctccatggggtgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcaatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaaacgtataaataaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcaatggattgcaaaaggcgtaccaactgcagcagccgcaggcaatggtgaagaagaacatgaaacagaggagaaaactgctacttacacttttgccaatgctcctgtaaaagccgaggctcaaattacaaaagagggcttaccaataggtttggagatttcagctgaaaacgaatctaaacccatctatgcagataaactttatcagccagaacctcaagtgggagatgaaacttggactgacctagacggaaaaaccgaagagtatggaggcagggctctaaagcctactactaacatgaaaccctgttacgggtcctatgcgaagcctactaatttaaaaggtggtcaggcaaaaccgaaaaactcggaaccgtcgagtgaaaaaattgaatatgatattgacatggaattttttgataactcatcgcaaagaacaaacttcagtcctaaaattgtcatgtatgcagaaaatgtaggtttggaaacgccagacactcatgtagtgtacaaacctggaacagaagacacaagttccgaagctaatttgggacaacagtctatgcccaacagacccaactacattggcttcagagataactttattggactcatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactattgttttccactggacggcataggtgttccaacaaccagttacaaatcaatagttccaaatggagaagataataataattggaaagaacctgaagtaaatggaacaagtgagatcggacagggtaatttgtttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatctatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggcccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctctcatcgtacacacatcgaaagggccactgcgttcgaccgtatggatgttcaataatgactcatgtaaacaacgtgttcaataaacatcactttatttttttacatgtatcaaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctctgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcgggataaaacatcccgcaggacctgctcgaaaactggttagcctgcacagccggcatcattcacacagcagcgggcgtcattgttggctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcttcgtttacgtactggtgacagatgcgcttgtattgttcgtgttgctcaggcattagtttaaaacaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttttcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacagcgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagctgcgcctacgagcttaaccttttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtctatctctacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgccactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcttgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccgacagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcagcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtagaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaacttattgagttcatctacttcgaactccccaaggatcaccctcaaggtccggcccacggagtgcggattactatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacaaaacttttcctgtcgtccaggactctgttaacttcacctttcctactcacaaactagaagctcaacgactacaccgcttttccagaagcattttccctactaatactactttcaaaaccggaggtgagctccacggtctccctacagaaaacccttgggtggaagcgggccttgtagtactaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagactttgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatcccactaggacataattacactctcataggacccccaatcacttcagaggtcatctggaccaaactgggaagcgttgattactttgatataatctgtaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccaccaccccgcacaactactttctctagcagcagtgtcgctaacaatacaatttccaatccaacctttgccgcgcttttaaaacgcactgtgaataattctacaacttcacatacaacaatttccacttcaacaatcagcatcatcgctgcagtgacaattggaatatctattcttgtttttaccataacctactacgcctgctgctatagaaaagacaaacataaaggtgatccattacttagatttgatatttaatttgttctttttttttatttacagtatggtgaacaccaatcatggtacctagaaatttcttcttcaccatactcatctgtgcttttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaatccccccaaatttaataatgattgctggaataattaatataatctgttgcaccataatttcatttttgatataccccctatttgattttggctggaatgctcccaatgcacatgatcatccacaagacccagaggaacacattcccccacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacaacgacttgcccaactacgcatccgccagcagcaggaacgcgtggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccaacaaagtggagatactaagggttgcattcactgttcctgcgattccatcgagtgcacctacaccctgctgaagaccctatgcggcctaagagacctgctaccaatgaattaaaaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacaccccttttataacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctgttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgactccggagtttggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtttcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccaccagtaatatcatcgaagttgctggaaatataatcaggcagagtttcttgtagaaattgaataaaagaaaaatttgccaaaaaaacattcaaaacctctgggatgcaaatgcaataggttaccgcgctgcgctccaacattgttagttttgaattagtctgcaaaaataaaaaaaaaacaagcgtcatatcatagtagcctgacgaacaggtggataaatcagtctttccatcacaagacaagccacagggtctccagctcgaccctcgtaaaacctgtcatcgtgattaaacaacagcaccgaaagttcctcgcggtgaccagcatgaataagtcttgatgaagcatacaatccagacatgttagcatcagttaaggagaaaaaacagccaacatagcctttgggtataattatgcttaatcgtaagtatagcaaagccacccctcgcggatacaaagtaaaaggcacaggagaataaaaaatataattatttctctgctgctgtttaggcaacgtcgcccccggtccctctaaatacacatacaaagcctcatcagccatggcttaccagagaaagtacagcgggcacacaaaccacaagctctaaagtcactctccaacctctccacaatatatatacacaagccctaaactgacgtaatgggactaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttacaacgtcattttcccacggccgcgccgccccttttaaccgttaaccccacagccaatcaccacacggcccacactttttaaaatcacctcatttacatattggcaccattccatctataaggtatattattgatgatg

SEQ ID NO: 51 - DNA, 35343 bp, Human mastadenovirus B
ctatctatataatataccttatagatggaatggtgccaacatgtaaatgaggtaatttaaaaaagtgcgcgctgtgtggtgattggctgcggggttaacggctaaaaggggcggcgcgaccgtgggaaaatgacgtgacttatgtgggaggagttatgttgcaagttattacggtaaatgtgacgtaaaacgaggtgtggtttgaacacggaagtagacagttttcccacgcttactgacaggatatgaggtagttttgggcggatgcaagtgaaaattctccattttcgcgcgaaaactaaatgaggaagtgaatttctgagtcatttcgcggttatgccagggtggagtatttgccgagggccgagtagactttgaccgtttacgtggaggtttcgattaccgtgtttttcacctaaatttccgcgtacggtgtcaaagttctgtgtttttacgtaggtgtcagctgatcgctagggtatttaaacctgacgagttccgtcaagaggccactcttgagtgccagcgagaagagttttctcctccgcgccgcaagtcagttctgcgctttgaaaatgagacacctgcgcttcctgccacaggaggttatctccagtgagaccgggatcgaaatactggagtttgtggtaaataccctaatgggagacgacccggaaccgccagtgcagcctttcgatccacctacgctgcacgatctgtatgatttagagatagacgggccggaggatcccaatgaggaagctgtgaatgggttttttactgattctatgctgctagctgctgatgaaggattggacataaaccctcctcctgagacacttgttaccccaggggtggttgtggaaagcggcataggtgggaaaaaattgcctgatctgggagcagctgaaatggacttgcgttgttatgaagagggttttcctcccagtgatgatgaagatggggaaactgagcagtccatccataccgcagtaaatgagggagtaaaagctgccagcgatgtttttaagttggactgtccggagctgcctggacatggctgtaagtcttgtgaatttcacaggaataacactggaatgaaagaactattgtgctcgctttgctatatgagaatgcactgccactttatttacagtaagtgtatttaagtgaaatttaaaggaatagtgtagctatttaataactgttgaatggtagatttatgtttttttcttgcgattttttgtaggtcctgtgtctgatgatgagtcaccttctcctgattcaactacctcacctcctgaaattcaggcgcccgcacctgcaaacgtatgcaagcccattcctgtgaagcctaagcctgggaaacgccctgctgtggataagcttgaggacttgttggagggtggggatggacctttggaccttagtacccggaaactgccaaggcaatgagtgccctgcagctgtgtttatttaatgtgacgtcatgtaataaaattatgtcagctgctgagtgttttattacttcttgggtggggacttggatatataagtaggagcagatctgtgtggttagctcacagcaacctgctgccatccatggaggtttgggctatcttggaagacctcagacagactaagctactgctagaaaacgcctcggacggagtctctggcctttggagattctggttcggtggtgatctagctaggctagtgtttaggataaaacaggactacagggaagaatttgaaaagttattggacgatagtccgggactttttgaagctcttaacttgggtcatcaggctcattttaaggagaaggttttatcagttttagatttttctactcctggtagaactgctgctgctgtagcttttcttacttttatattggataaatggatccgccaaactcacttcagcaagggatacgttttggatttcatagcagcagctttgtggagaacatggaaggctcgcaggatgaggacaatcttagattactggccagtgcagcctctgggagtagcagggatactgagacacccaccgaccatgccagcggttctgcaggaggagcagcaggaggacaatccgagagccggcctggaccctccggtggaggagtagctgacctgtttcctgaactgcgacgggtgcttactaggtctacgaccagtggacagaacaggggaattaagagggagaggaatcctagtgggaataattcaagaaccgagttggctttaagtttaatgagccgcaggcgtcctgaaactgtttggtggcatgaggttcagagcgaaggcagggatgaagtttcaatattgcaggagaaatattcactagaacaacttaagacctgttggttggaacctgaggatgattgggaggtggccattaggaattatgctaagatatctctgaggcctgataaacaatatagaattactaagaagattaatattagaaatgcatgctacatatcagggaatggggcagaggttataatagatacacaagataaagcagtttttagatgttgtatgatgggtatgtggccaggggttgtcggcatggaagcagtaacacttatgaatattaggtttaaaggggatgggtataatggcattgtatttatggctaacactaagctgattctacatggttgtagcttttttgggtttaataatacgtgtgtagaagcttgggggcaagttagtgtgaggggttgtagtttttatgcatgctggattgcaacatcaggtagggtcaagagtcagttgtctgtgaagaaatgcatgtttgagagatgtaatcttggcatactgaatgaaggtgaagcaagggtccgccactgcgcagctacagaaactggctgcttcattctaataaagggaaatgccagtgtgaagcataatatgatctgtggacattcggatgagaggccttatcagatgctgacctgcgctggtggacattgcaatattcttgctaccgtgcatatcgtttcacatgcacgcaaaaaatggcctgtatttgaacataatgtgattaccaagtgcaccatgcacataggtggtcgcaggggaatgtttatgccttaccagtgtaacatgaatcatgtgaaggtaatgttggaaccagatgccttttccagagtgagcttaacaggaatctttgatatgaatattcaactatggaagatcctgagatatgatgacactaaaccaagggtgcgcgcatgcgaatgcggaggcaagcatgctagattccagccggtgtgcgtggatgtgactgaagacttgagacccgatcatttggtgcttgcctgcactggagcggagttcggttctagtggtgaagaaactgactaaagtgagtagtggggcaagatgtggatggggactttcaggttggtaaggtgggcagattgggtaaattttgttaatttctgtcttgcagctgccatgagtggaagcgcttcttttgaggggggagtatttagcccttatctgacgggcaggctcccaccatgggcaggagttcgtcagaatgtcatgggatccactgtggatgggagacccgtccagcccgccaattcctcaacgctgacctatgccactttgagttcgtcaccattggatgcagctgcagccgccgccgctactgctgccgccaacaccatccttggaatgggctattatggaagcatcgttgccaattccagttcctctaataacccttcaaccctggctgaggacaagctacttgttctcttggcgcagctcgaggccttaacccaacgcttaggcgaactgtctaagcaggtggcccagttgcgtgagcaaactgagtctgctgttgccacagcaaagtctaaataaagatctcaaatcaataaataaagaaatacttgatataaaacaaatgaatgtttatttgatttttcgcgcgcggtatgccctggaccatcggtctcgatcattgagaacgcggtggatcttttccagtaccctgtaaaggtgggattgaatgtttagatacatgggcattagtccgtctcgggggtggagatagctccattgaagagcctcttgctccggggtagtgttataaatcacccagtcatagcaaggtcggagtgcatggtgttgcacaatatcttttaggagcagactaattgcaacggggaggcccttagtgtaggtgtttacaaatctgttgagctgggacgggtgcatccggggtgaaattatatgcattttggactggatcttgaggttggcaatgttgccgcctagatcccgtctcgggttcatattgtgcaggaccaccaagacagtgtatccggtgcacttgggaaatttatcatgcagcttagagggaaaagcatgaaaaaatttggagacgcctttgtgaccccccagattctccatgcactcatccataatgatagcgatggggccgtgggcagcggcacgggcgaacacgttccgggggtctgaaacatcatagttatgctcctgagtcaggtcatcataagccattttaataaactttgggcggagggtgccagattgggggatgaaagttccctctggcccgggagcatagtttccctcacatatttgcatttcccaggctttcagttcagagggggggatcatgtccacctgcggggctataaaaaataccgtttctggagccggggtgattaactgggacgagagcaaattcctaagcagctgagacttgccgcacccagtgggaccgtaaatgaccccaattacgggttgcagatggtagtttagggaacgacagctgccgtcctcccggagcaggggggccacttcgttcatcatttcccttacatggatattttcccgcaccaagtccgttaggaggcgctctcccccaagggatagaagctcctggagcgaggagaagtttttcagcggcttcagcccgtcagccatgggcattttggaaagagtctgttgcaagagctcgagccggtcccagagctcggtgatgtgctctatggcatctcgatccagcagacctcctcgtttcgcgggttgggacggctcctggagtagggaatcagacgatgggcgtccagcgctgccagggtccgatccttccatggtcgcagcgtccgagtcagggttgtttccgtcacggtgaaggggtgcgcgcctggttgggcgcttgcgagggtgcgcttcagactcatcctgctggtcgagaaccgctgccgattggcgccctgcatgtcggccaggtagcagtttaccatgagttcgtagttgagcgcctcggccgcgtggcctttggcacggagcttacctttggaagttttatggcaggcggggcagtagatacatttgagggcatacaacttgggcgcgaggaaaatggattcgggggagtatgcatccgcaccgcaggaggcgcagacggtttcgcactccacgagccaggtcagatccggctcatcggggtcaaaaacaagttttccgccatgttttttgatgcgtttcttacctttggtttccatgagttcgtgtccccgctgggtgacaaagaggctgtccgtgtccccgtagaccgactttatgggcctgtcctcgagcggagtgcctcggtcctcttcgtagaggaacccagcccactctgatacaaaagcgcgtgtccaggccagcacaaaggaggccacgtgggaggggtagcggtcgttgtcaaccagggggtccaccttctctacggtatgtaaacacatgtccccctcctccacatccaagaatgtgattggcttgtaagtgtaggccacgtgaccaggggtccccgccgggggggtataaaagggggcgggcctctgttcgtcctcactgtcttccggatcgctgtccaggagcgccagctgttggggtaggtattccctctcgaaggcgggcatgacctctgcactcaggttgtcagtttctaggaacgaggaggatttgatattgacagtaccagcagagatgcctttcataagactctcgtccatctggtcagaaaacacaatcttcttgttgtccagcttggtggcaaatgatccatagagggcattggatagaagcttggcgatggagcgcatggtttggttcttttccttgtccgcgcgctccttggcggtgatgttaagctggacgtactcgcgcgccacacatttccattcaggaaagatggttgtcagttcatccggaactattctgattcgccatcccctattgtgcagggttatcagatccacactggtggccacctcgcctcggaggggctcattggtccagcagagtcgacctccttttcttgaacagaaaggggggagggggtctagcatgaactcatcaggggggtccgcatctatggtaaatattcccggtagcaaatctttgtcaaaatagctgatggtggcgggatcatccaaggtcatctgccattctcgaactgccagcgcgcgctcataggggttaagaggggtgccccagggcatggggtgggtgagcgcggaggcatacatgccacagatatcgtagacatagaggggctcttcgaggatgccgatgtaagtgggataacatcgcccccctctgatgcttgctcgcacatagtcatagagttcatgtgagggggcaagaagacccgggcccagattggtgcggttgggtttttccgccctgtaaacgatctggcgaaagatggcatgggaattggaagagatagtaggtctctggaatatgttaaaatgggcatgaggtaagcctacagagtcccttatgaagtgggcatatgactcttgcagcttggctaccagctcggcggtgatgagtacatccagggcacagtagtcgagagtttcctggatgatgtcataacgcggttggcttttcttttcccacagctcgcggttgagaaggtattcttcgtgatccttccagtactcttcgaggggaaacccgtctttttctgcacggtaagagcccaacatgtagaactgattgactgccttgtagggacagcatcccttctccactgggagagagtatgcttgggctgcattgcgcagcgaggtatgagtgagggcaaaagtgtccctgaccatgactttgaggaattgatacttgaagtcgatgtcatcacaggccccctgttcccagagttggaagtccacccgcttcttgtaggcggggttgggcaaagcgaaagtaacatcattgaagaggatcttgccggccctgggcatgaaatttcgggtgattttgaaaggctgaggaacctctgctcggttattgataacctgagcggccaagacgatctcatcaaagccattgatgttgtgccccactatgtacagttctaagaatcgaggggtgcccctgacatgaggcagcttcttgagttcttcaaaagtgagatctgtagggtcagtgagagcatagtgttcgagggcccattcgtgcacgtgagggttcgctttaaggaaggaggaccagaggtccactgccagtgctgtttgtaactggtcccggtactgacgaaaatgctgtccgactgccatcttttctggggtgacgcaatagaaggtttgggggtcctgccgccagcgatcccacttgagttttatggcgaggtcataggcgatgttgacgagccgctggtctccagagagtttcatgaccagcatgaaggggattagctgcttgccaaaggaccccatccaggtgtaggtttccacatcgtaggtgagaaagagcctttctgtgcgaggatgagagccaatcgggaagaactggatctcctgccaccagttggaggaatggctgttgatgtgatggaagtagaactccctgcgacgcgccgagcattcatgcttgtgcttgtacagacggccgcagtactcgcagcgattcacgggatgcaccttatgaatgagttgtacctgacttcctttgacgagaaatttcagtggaaaattgaggcctggcgcttgtacctcgcgctttactatgttgtctgcatcggcatgaccatcttctgtctcgatggtggtcatgctgacgagccctcgcgggaggcaagtccagacctcggcgcggcaggggcggagctcgaggacgagagcgcgcaggccggagctgtccagggtcctgagacgctgcggagtcaggttagtaggcagtgtcaggagattaacttgcatgatcttttggagggcgtgagggaggttcagatagtacttgatctcaacgggtccgttggtggagatgtcgatggcttgcagggttccgtgtcccttgggcgctaccaccgtgcccttgtttttcattttggacggcggtggctctgttgcttcttgcatgtttagaagcggtgtcgagggcgcgcaccgggcggcaggggcggctcgggacccggcggcatggctggcagtggtacgtcggcgccgcgcgcgggtaggttctggtactgcgccctgagaagactcgcatgcgcgacgacgcggcggttgacatcctggatctgacgcctctgggtgaaagctaccggccccgtgagcttgaacctgaaagagagttcaacagaatcaatctcggtatcgttgacggcggcttgcctaaggatttcttgcacgtcgccagagttgtcctggtaggcgatctcggccatgaactgctcgatctcttcctcttgaagatctccgcggcccgctctctcgacggtggccgcgaggtcgttggagatgcgcccaatgagttgagagaatgcattcatgcccgcctcgttccagacgcggctgtagaccacagcccccacgggatctctcgcgcgcatgaccacctgggcgaggttgagctccacgtggcgggtgaagaccgcatagttgcataggcgctggaaaaggtagttgagtgtggtggcgatgtgctcggtgacgaagaaatacatgatccatcgtctcagcggcatctcgctgacatcgcccagcgcttccaagcgctccatggcctcgtagaagtccacggcaaagttaaaaaactgggagttacgcgcggacacggtcaactcctcttccagaagacggataagttcggcgatggtggtgcgcacctcgcgctcgaaagcccctgggatttcttcctcaatctcttcttcttccactaacatctcttcctcttcaggtggggctgcaggaggagggggaacgcggcgacgccggcggcgcacgggcagacggtcgatgaatctttcaatgacctctccgcggcggcggcgcatggtctcggtgacggcacgaccgttctccctgggtctcagagtgaagacgcctccgcgcatctccctgaagtggtgactgggaggctctccgttgggcagggacaccgcgctgattatgcattttatcaattgccccgtaggtactccgcgcaaggacctgatcgtctcaagatccacgggatctgaaaacctttcgacgaaagcgtctaaccagtcgcaatcgcaaggtaggctgagcactgtttcttgcgggcgggggcggctagacgctcggtcggggttctctctttcttctccttcctcctcttgggagggtgagacgatgctgctggtgatgaaattaaaataggcagttttgagacggcggatggtggcgaggagcaccaggtctttgggtccggcttgttggatacgcaggcgatgagccattccccaagcattatcctgacatctggccagatctttatagtagtcttgcatgagtcgttccacgggcacttcttcttcgcccgctctgccatgcatgcgagtgatcccgaacccgcgcatgggctggacaagtgccaggtccgctacaaccctttcggcgaggatggcttgctgcacctgggtgagggtggcttggaagtcgtcaaagtccacgaagcggtggtaggccccggtgttgattgtgtaggagcagttggccatgactgaccagttgactgtctggtgcccagggcgcacgagctcggtgtacttgaggcgcgagtatgcgcgggtgtcaaagatgtaatcgttgcaggtgcgcaccaggtactggtagccaatgagaaagtgtggcggtggctggcggtacaggggccatcgctctgtagccggggctccgggggcgaggtcttccagcatgaggcggtggtagccgtagatgtacctggacatccaggtgataccggaggcggtggtggatgcacgtgggaactcgcgcacgcggttccagatgttgcgcagcggcatgaagtagttcatggtaggcacggtctggccagtgaggcgcgcgcagtcattgacgctctgtagacacggagaaaacgaaagcgatgagcggctcgactccgtggtctgggggaacgtgaacgggttgggtcgcggtgtaccccggttcgagtccaaagctaagcgatcacgctcggatcggccggagccgcggctaacgtggtattggctatcccgtctcgacccagccgacgaatatccagggtacggagtagagtcgtttttgctgctttttcctggacgtgtgccattgccacgtcaagctttagaacgctcagttctcgggccgtgagtggctcgcgcccgtagtctggagaatcagtcgccagggttgcgttgcggtatgccccggttggagcctaagcgcggctcgtatcggccggtttccgcgacaagcgagggtttggcagccccgttatttccaagaccccgccagccgacttctccagtttacgggagcgagcccttttttttttttttttgtttttgtcgcccagatgcatccagtgctgcgacagatgcgcccccagcaacaggccccttctcagcaacagccacaaaaggctcttcttgctcctgcaactactgcagctgcagccgtgagcggcgcgggacagcccgcctatgatctggacttggaagagggcgagggattggcgcgcctgggggctccatcgcccgagcggcacccgcgggtgcaactaaaaaaggactctcgcgaggcgtacgtgccccagcagaacctgttcagggacaggagcggcgaggagccagaggagatgcgagcatctcgatttaacgcgggtcgcgagctgcgccacggtctggatcgaagacgggtgctgcaagacgaggattttgaggtcgatgaagtcacagggatcagcccagctagggcacatgtggccgcggccaacctagtctcggcctacgagcagaccgtgaaggaggagcgcaacttccaaaaatcttttaacaaccatgtgcgcaccctgatcgcccgcgaggaagtgaccctgggtctgatgcatctgtgggacctgatggaggctatcgcccaaaaccccactagcaaaccactgacagctcagctgtttctggtggttcaacatagcagggacaacgaggcattcagggaggcgttgttgaacatcaccgagcctgatgggagatggctgtatgatctgatcaacatcctgcaaagtattatagtgcaggaacgtagcctgggtttggctgagaaagtggcagctatcaactactcggtcttgagcctgggcaaatactacgctcgcaagatctacaagaccccctacgtacccatagacaaggaggtgaagatagatgggttttacatgcgcatgactctgaaggtgctgactctgagcgacgatctgggggtgtatcgcaatgacaggatgcaccgcgcggtgagcgccagcaggaggcgcgagctgagcgacagagaacttatgcacagcttgcaaagagctctaacgggggccgggactgatggggagaactactttgacatgggagcggatttgcaatggcaacccagtcgcagggccatggaggctgcagggtgtgagcttccttacatagaagaggtggatgaagtcgaggacgaggagggcgagtacttggaagactgatggcgcgacccgtatttttgctagatggaacagcagcaggcaccggaccccgcaatgcgggcggcgctgcagagccagccgtccggcattaactcctcggacgattggacccaggccatgcaacgcataatggcgctgacgacccgcaaccccgaagcctttagacagcaaccccaggccaaccgcctttctgccatactggaggccgtagtgccctcccgctccaaccccacccacgagaaggtcctggctatcgtgaacgcgctggtggagaacaaggccatccgtcccgatgaggccgggctggtatacaatgctctcttggagcgcgtggcccgttacaacagcagcaacgtgcaaaccaacctggaccggatggtgaccgatgtgcgcgaggccgtgtctcagcgcgagcgattccagcgcgacgccaacttggggtcgttggtagcgctaaacgctttcctcagcacccagcccgccaacgtgccccgtggtcagcaagactatacaaactttttgagtgcattgagactcatggtagctgaggtgccccagagcgaggtgtaccagtccgggccagattacttcttccagaccagcagacagggcttgcagacagtgaacctgactcaggctttcaagaacctgaagggtctgtggggagtgcacgccccagtaggggatcgcgcgaccgtgtctagcttgctgactcccaactcccgcctgctgctgctgctggtatccccctttactgacagcggtagcattgaccgcaactcgtacttgggctacctgcttaacctgtatcgcgaggccataggacagagccaggtggacgagcagacctatcaagaaatcacccaagtgagccgcgccctgggtcaggaagacacgggcagtttggaagccaccctgaacttcttgctaaccaaccggtcacagaagatccctcctcagtatgcgcttaccgctgaggaggagcggatcctcagatacgtgcaacagagcgttggactgttcctgatgcaggagggggcgacacctaccgccgcgctggacatgacagctcgaaacatggagcccagcatgtatgctagtaacaggcctttcattaacaaactgctggactacctgcacagggcggccgccatgaactctgattatttcaccaatgctatcctgaacccacactggctgcccccacctggtttctacactggcgagtacgacatgcccgaccccaatgacgggttcctgtgggacgatgtggacagcagcatattttccccgcctcccggttatacagtttggaagaaggaagggggcgatagaagacactcttccgtgtcgctatccggaacggctggtgctgccgcgaccgtgcccgaagctgcaagtcctttccctagcttgcccttttcactaaacagcgttcgcagcagtgaactggggagaataacccgcccgcgcttgatgggcgaggatgagtacttgaatgactctttgctgaggccagagagggaaaagaacttccccaacaatggaatagagagtctggtggataagatgagtagatggaagacctatgcgcaggatcacagagacgagcccaggatcttgggggctacaagcagaccgatccgtagacgccagcgccacgacaggcagatgggtcttgtgtgggacgatgaggactctgccgatgatagcagcgtgttggacttgggtggaagaggagggggcaacccgttcgctcatctgcgtcccagattcgggcgcatgttgtaaaagtgaaagtaaaataaaaaggcaactcaccaaggccatggcgaccgagcgtgcgttcgttcttttttgttatctgtgtctagtacgatgaggagacgagccgtgctaggcggagcggtggtgtatccggagggtcctcctccttcttacgagagcgtgatgcagcaacaggcggcgatgatacagcccccactggaggctcccttcgtacccccacggtacctggcgcctacggaagggagaaacagcattcgttactcggagctgtcgcccctgtacgataccaccaagttgtatctggtggacaacaagtcggcggacatcgcctccctgaactatcagaacgaccacagcaacttcctgaccacggtggtgcagaacaatgactttacccccacggaggctagcacccagaccatcaactttgacgagcggtcgcgatggggcggtcagctgaagaccatcatgcacaccaacatgcccaacgtgaacgagtacatgttcagcaacaagttcaaggcgagggtgatggtgtccagaaaagctcctgaaggtgttacagtaaatgacacctatgatcataaagaggatatcttgaagtatgagtggtttgagttcattttaccagaaggcaacttttcagccaccatgacgatcgacctgatgaacaatgccatcattgacaactacctggaaattggcagacagaatggagtgctggaaagtgacattggtgttaagtttgacactagaaatttcaggctcgggtgggaccccgaaactaagttgattatgccaggtgtctacacttatgaggcattccatcctgacattgtattgctgcctggttgcggggtagactttactgaaagccgacttagcaacttgcttggcatcaggaagagacatccattccaggagggtttcaaaatcatgtatgaagatcttgaagggggtaatattcctgcccttttggatgtcactgcctatgaggaaagcaaaaaggataccactactgaaacaaccacactggctgttgcagaggaaactagtgaagatgatgatataactagaggagatacctatataactgaaaaacaaaaacgtgaagctgcagctgctgaagttaaaaaagagttaaagatccaacctctagaaaaagacagcaagagtagaagctacaatgtcttggaagacaaaatcaacacagcctaccgcagttggtacctgtcctacaattacggtaaccctgagaaaggaataaggtcttggacactgctcaccacttcagatgtcacctgtggggcagagcaggtctactggtcgctccctgacatgatgcaagacccagtcaccttccgctccacaagacaagtcaacaactacccagtggtgggtgcagagcttatgcccgtcttctcaaagagtttctacaatgagcaagccgtgtactctcagcagctccgacaggccacttcgctcacgcacgtcttcaaccgcttccctgagaaccagatcctcatccgcccgccggcgcccacaattaccaccgtcagtgaaaacgttcctgctctcacagatcacgggaccctgccgttacgcagcagtatccggggagtccagcgcgtgaccgttactgacgccagacgccgcacctgtccctacgtttacaaggccctgggcatagtcgcgccgcgcgttctttcaagccgcactttctaaaaaaaaaaaaaatgtccattctcatctcgcccagtaataataccggttggggactgtatgcgcccaccaagatgtatggaggcgcccgcaagcgctctacccagcatcctgtgcgcgttcgcggtcatttccgcgctccctggggcgcactcaagggtcgtacccgcactcggaccacggtcgatgatgtgatcgaccaggtggtcgccgatgctcgtaattatactcctactgcgcctacatctactgtggatgcagttattgacagtgtggtggcagacgcccgcgcctatgctcgccggaagagccgaaggaggcgcatcgccaggcgccacagggctactcccgccatgcgagccgcaaaagctattctgcggagggccaaacgtgtggggcgaagagccatgcttagagcggccagacgcgcggcttcaggtgccagcagcggcaggtcccgcaggcgcgcggccacggcggcagcagcggccattgccaacatggcccaaccgcgaagaggcaatgtgtactgggtgcgtgatgccactaccggccagcgcgtgcccgtgcgcacccgcccccctcgcacttagaagatactgagcagtctccgatgttgtgtcccagcggcaagtatgtccaagcgcaaatacaaggaagagatgctccaggtcatcgcgcctgaaatctacggtccgccggtgaaggatgaaaaaaagccccgcaaaatcaagcgggtcaaaaataacaaaaaggaagaagatgacgatgatgggctggtggagtttgtgcgcgagttcgccccaagacggcgcgtgcagtggcgcgggcgcaaagtgcgtcaagtgctcagacccgggaccactgtggtttttacacctggcgagcgttccagcactacttttaaacggtcctatgatgaggtgtacggggatgacgatattcttgagcaggcggcagaccgccttgacgagtttgcttatggcaagcgcactagatccagtcccaaagaggaggctgtgtccattcctttggatcatggaaatcccacccccagcctcaaaccagtcaccctgcagcaagtgctgcccgtgcctgcgcggagaggcgtaaagcgcgagggtgaggacctatatcccaccatgcagctaatggtgcccaagcgccagaggctagaagacgtactggagaaaatgaaagtggatgccgatatccagcctgaggtcaaagtaagacctatcaaggaagtggcgccaggtttgggagtacaaaccttcgacatcaagattcccaccgagtccatggaagtgcagaccgaacctgcaaaacccaccacctcaattgaggtgcaaacggaaccctggatgcccgcgcccgttgccgcccccagcaccactcgaagatcacgacgaaagtacggcccagcaagtctgctaatgcccaactatgctctgcacccatccatcattcccactccgggttacagaggcactcgctactatcgaaaccggagcaacacctctcgccgccgcaaaccacctgcaagtcgcactcgccgtcgccgccgccgcaacactgccagcaaattgactcccgccgccctggtgcggagagtgtaccgcgatggtcgcgctgaacctctgacgctgccgcgcgcgcgctaccatccaagcatcaccacttaatgactgttgacgctgcctccttgcagatatggccctcacttgccgccttcgcgtccccattactggctaccgaggaagaaactcgcgccgtagaaggatgttggggcgagggatgcgccgccacagacgaaggcgcgctatcagcagacgattagggggtggctttttgccagctcttatacccatcatcgccgcagcgatcggggcgataccaggcatagcttcagtggcggttcaggcctcgcagcgccactaacattggaaaaaacttataaataaaaaatagaatggactctgacgctcctggtcctgtgactatgtttttgtagagatggaagacatcaatttttcatccctggctccgcgacacggcacgaggccgtacatgggcacctggagcgacatcggcacgagccaactgaacgggggcgccttcaattggagcagtatctggagcgggcttaaaaattttggctcgaccgtaaaaacctatgggaacaaagcttggaacagcagcacagggcaggctctgagaaataagcttaaggaacaaaacttccaacagaaggtggtcgatgggatcgcctctggtattaacggcgtagtggatctggccaaccaggctgtacaaaaacagataaacagccgcctggacccgccgcccgcaacccctggtgaaatggaagtggaggaagaacttcctccgctggaaaagcggggcgacaagcgtccgcgacccgagctagagcagacgctggtgacgcgcgcagacgagcccccttcatatgaggaggcagtaaagctcggaatgcccactaccaggcctgtagctcacatggctaccggggtgatgaaaccttctcagtcacatcgacccgccaccttggacttgcctcctccccctgcttctgcggcgcctgttcccaaacctgtcgctaccagaaagcccaccgccgtacagcccgtcgccgtagccagaccgcgtcctgggggcacaccgcgcccgaaagcaaactggcaaagtactctgaacagcatcgtgggtctgggcgtgcagagtgtaaagcgccgtcgctgctattaattaaatatggagtagcgcttaacttgcttgtctgtgtgtatgtatcatcaccacgccgccgcagcagaggagaaaggaagaggtcgcgcgccgaggctgagttgctttcaagatggccaccccatcgatgatgccccaatgggcatacatgcacatcgccggacaggatgcttcggagtacctcagtccgggtctggtgcagttcgcccgtgcaacagacacctacttcagtatggggaacaaatttagaaaccccacagtggcgcccacccacgatgtgaccaccgaccgtagccagcgcctgatgctgcgcttcgtgcccgttgaccgggaagacaatacctactcttacaaagttcgctacacgctggctgtaggcgacaacagagtgcttgacatggccagcacattctttgacattcggggggtgcttgatagaggtcctagcttcaagccatattccggcacagcttacaattcactcgctcctaagggcgcgcccaatacatctcagtggatagttacaacaaatggggacaatgcagtaactaccaccacaaacacatttggcattgcttccatgaagggagacaatattactaaagaaggtttgcaaattgggaaagacattaccactactgaaggagaagaaaagcccatttatgccgataaaacatatcagccagagcctcaagttggagaagaatcatggactgatactgatggaacaaatgaaaagtttggtggaagagcccttaaaccagctaccaacatgaagccatgctacgggtcttttgcaagacctacaaacataaaagggggccaagctaaaaacagaaaagtaaaaccaacaaccgaaggaggggttgaaactgaggaaccagatattgatatggaatttttcgatggtagagatgctgttgcaggagctttagcgcctgaaattgtgctttatacggaaaatgtaaatttggaaactccagacagtcatgtggtatataaaccagaaacgtctaataactctcatgcaaatttgggtcaacaagccatgcctaacagacccaattacattggcttcagggataacttcgtaggcctaatgtactacaacagtactggaaatatgggagttttggctggccaagcatcacaactgaatgcagtggttgacttgcaggacagaaatactgaactgtcatatcagcttttgcttgattctctgggagacagaaccagatacttcagcatgtggaatcaggctgtggacagttacgatcccgatgttcgcattattgaaaatcatggcatcgaggatgaactgcctaattactgttttcctctgaatggcataggaccagggcacacatatcaaggcattaaagttaaaaccgatgacactaatggatgggaaaaagatgctaatgttgctccagctaatgaaataaccataggcaacaacctggctatggaaattaatatccaagctaacctttggagaagttttctgtactctaatgtggctttgtaccttccagatgtttacaagtacacgccacctaacattactttgcccactaacaccaacacctatgagtacatgaacgggcgagtggtatccccatccctggttgattcatacatcaacattggcgccaggtggtctcttgacccaatggacaatgtgaatccattcaaccaccaccgcaatgctggtctgcgctacaggtccatgcttctgggaaatggtcgttatgtgcctttccacatacaagtgcctcagaaattctttgctgtcaagaacctacttcttctacctggctcctacacctacgagtggaacttccgaaaggatgtgaacatggtcctgcaaagttcccttggaaatgacctcagaacggatggtgctaccataagtttcaccagcatcaatctctatgccaccttcttccccatggctcacaacacagcttccacccttgaagccatgctgcgcaacgataccaatgatcagtcatttaacgactacctctctgcagctaacatgctttaccccattcctgccaatgcaaccaacattccaatttccatcccatctcgcaactgggcagccttcaggggctggtccttcaccagactcaaaaccaaggagactccatctcttggatcagggttcgatccctacttcgtatattctggatctattccctacctggatggcaccttttaccttaaccacactttcaagaaggtctccatcatgtttgactcctcagtcagctggcctggcaatgacaggctgttgagtccaaatgagtttgaaatcaagcgcactgtggacggggaaggatacaacgtggcacaatgcaacatgaccaaagactggttcctggttcagatgcttgccaattacaacattggctaccagggcttttacatccctgagggatacaaggatcgcatgtactcctttttcagaaacttccagcctatgagcaggcaggtggttgatgaggttaattacactgactacaaagccgtcaccttaccataccaacacaacaactctggctttgtagggtaccttgcacctactatgagacaaggggaaccttacccagccaattatccatacccgctcatcggaactactgccgttaagagtgtcacccagaaaaagttcctgtgcgacaggaccatgtggcgcattcccttctccagcaacttcatgtccatgggggcccttaccgacctgggacagaacatgctctatgccaactcagcccatgcgctggacatgacttttgaggtggatcccatggatgagcccaccctgctttatcttcttttcgaagtcttcgacgtggtcagagtgcaccagccacaccgcggcgtcatcgaggccgtctacctgcgcacaccgttctcggccggcaacgccaccacataagaagcctcttgcttcttgcaagcagcagctgcagccatgacatgcgggtccggaaacggctccagcgagcaagagctcaaagccatcgtccgagacctgggctgcggaccctatttcctgggaacctttgacaagcgtttcccggggttcatggcccccgacaagctcgcctgcgccatagtcaacactgccggacgcgagacggggggagagcactggctggcttttggttggaacccgcgctacaacacctgctacctttttgatccttttgggttctcggatgagcggctcaaacagatttaccagtttgagtacgaggggctcctgcgtcgcagtgcccttgctaccaaagaccgctgcatcaccctggagaagtctacccaaagcgtgcagggtccgcgctcagccgcctgtggacttttttgctgtatgttccttcatgcctttgtgcactggcccgaccgccccatggacggaaaccccaccatgaagttgctgactggggtgtccaacagcatgctccaatcaccccaagtccagcccaccctgcgccgcaaccaggaggtgctataccgcttcctaaacacccactcatcttactttcgttctcaccgcgcgcgcattgaaagggccaccgcgtttgaccgtatggatatgcaataagtcatgtaaaaccgtgttcaataaacagcactttatttttacatgcactgaggctctggttttgctcatttgtttcatcatttactcagaagtcgaatgggttctggcgggagtcagagtgacccgcgggcagggatacgttgcggaactgtaacctgttctgccacttgaactcggggattaccagcttgggaactggaatctcgggaaaggtgtcttgccacaactttctggtcagttgcatagcgccaagcaggtcaggagcagagatcttgaaatcacagttggggccggcattctggacacgggagttgcgatacactgggttgcaacactggaacactatcaacgctgggtgtcttacgcttgccaacacggttgggtcactgatggtagtcacatccaagtcttcagcattggccatcccaaagggggtcatcttacatgtctgcctgcccatcacgggagcgcagcctggcttgtggttgcaatcacaatgaatggggatcagcatcatcctggcttggtcgggagttatccctgggtacacagccttcatgaaggcttcgtactgcttaaaagcttcctgggccttacttccctcggtgtagaacatcccacaggacttgctggaaaattgattagtagtacagttggcatcattcacacaacagcgggcatcgttgttggccaactgaaccacatttctgccccagcggttttgggtgatcttggctctgtctggattctccttcatagcgcgctgcccgttctcgctcgccacatccatctcgataatgtggtccttctggatcatgatagtgccatgcaggcatttcaccttgccttcataatcggtgcatccatgagcccacagagcgcacccggtgcactcccaattattgtgggcgatctcagaataataatgtaccaatccctgcatgaatcttcccatcattgttgtcaaggtcttcatgctggtaaatgtcagcgggatgccacggtgctcctcgttcacatactggtggcagatacgcttgtattgctcgtgctgctctggcatcagcttgaaagaggttctcagatcattatccagcctgtacctttccattagcacagccatcacttccatgcccttctcccaggcagataccaggggcagactcaaaggattcctaacagcaataaaagtagctcctttagctatagggtcattcttgtcgatcttctcaacacttctcttgccatccttctcaatgatgcgcaccggggggtagctgaagcccacggccaccaactgagcctgttctctttcttcttcactatcctggctgatgtcttgcagagggacatgcttggtcttcctgggcttcttcttgggagggatcgggggaggactgttgctccgctccggagacagggatgactgcgaagtttcgcttaccaataccacctggctctcggtagaagaaccggaccccacacgacggtaggtgttcctcttcgggggcagaggtggaggcgactgagatgggctgcggtctggccttggaggcggatggctggcagagctcattccgcgttcgggggtgtgctcccggtggcggtcgcttgactgatttcctccgcggctggccattgtgttctcctaggcagagaaacaacagacatggaaactcagccatcactgccaacatcgctgcaagcaccatcacacctcgcccccagcagcgacgaggaggagagcttaaccaccccaccacccagtcccgctaccaccacctctaccctcgatgatgaggaggaggtcgacgcagcccaggagatgcaggcgcaggataatgtgaaaacggaagagattgaggcagatgtcgagcaggacccgggctatgtgacgccggcggagcaccaggaggagctgaaacgctttctagacagagaggatgacgaccgcccagagcatcaagcagatggcgtttaccaggaggctgggatcagggatcatgtcgccgactacctcaccggccttggtggggaggacgtgctcctcaaacatctagcaaggcagtcgatcatagttaaagacgcattgctcgatctcactgaagtgcccatcagtgtggaagagcttagccgcgcctacgagctgaacctcttttcgcctcaggtaccccccaagcggcagccaaacggcacctgcgaggccaaccctcgactcaacttctatccagcttttactatccccgaagtgttggccacctaccacatctttttcaagaaccaaaagattccagtctcctgccgcgccaaccgcacccgcgccgatgccctgctcaacttgggtccgggagctcgcttacctgatatagcttccttggaagaggttccaaagatctttgagggtctgggaagtgatgagacacgggccgcaaatgctctgcaacagggagagaatgacatggatgaacaccacagcgctctggtggaactggagggtgacaatgcccggattgcagtgctcaagcgcagtatcgtggtcacccattttgcctaccccgctgttaacctgccccccaaagttatgagcgctgtcatggaccatctgctcatcaaacgagcaagacctctttcagaaaaccagaacatgcaggatccagacgcctcggacgagggcaagccggtagtcagtgacgagcagctatctcgctggctgggtaccaactccccccgagatttggaagagaggcgcaagcttatgatggctgtagtgctagtaactgtggagctggagtgtctgcgccgctttttcaccgaccctgagaccctgcgcaagctagaggagaacctgcactacacctttagacatggcttcgtgcggcaggcatgcaagatctccaacgtggagcttaccaacctggtttcttacatgggcattttgcatgagaaccgactagggcagagcgtcctgcacaccacccttaaaggggaggcccgccgtgactacatccgagactgtgtctacctctacctctgccatacctggcaaactggtatgggtgtgtggcaacagtgtttggaagagcagaacctaaaagagctggacaagctcttgcagagatccctcaaagccctgtggacaggttttgatgagcgcaccgtcgcctcggacctggcagacatcatcttccccgagcgtctcagggttactctgcgaaacggcctgccagactttatgagccagagcatgcttaacaactttcgctctttcatcctggaacgctccggtatcctgcctgccacctgctgtgcgctgccctccgactttgtgcctctcacctaccgcgagtgcccaccgccgctatggagccactgctacctgttccgcctggccaactacctctcctaccactcggatgttatagaggatgtgagcggagacggcctgctggaatgccactgccgctgcaatctttgcacaccccaccgctcccttgcctgcaacccccagttgctgagcgagacccagattatcggcaccttcgagctgcagggtcccagaagtaaaggcgaggggtcttctccggggcagagtttgaaactgacaccggggctgtggacctccgcctacctgcgcaagtttcaccccgaggactaccatccctatgagatcaggttctatgaggaccaatcacatcctcccaaagtcgagctctcagcctgcgtcatcacccagggagcaattctggcccaattgcaagccatccaaaaatctcgccaagaatttctgctaaaaaagggaaacggggtctaccttgaccctcagaccggtgaggagctcaacacaaggttcccccaggatgtcccatcgccgaggaagcaagaagttgaaggtgcagctgtcgcccccagaggatatgaaggaagactgggacagtcaggcagaggaggagatggaagattgggacagccaggcagaggaggtggacagcctggaggaagacagtttggaggaggaagacgaggaggcagaggaggtggaagaagcaaccgccgccaaacagttgtcatcggcggcggagacaagcaagtccccagacagcagcacggctaccatctccgctccgggtcggggggcccagcggcggcccaacagtagatgggacgagaccgggcgattcccaaacccgaccaccgcttccaagaccggtaagaaggagcgacagggatacaagtcctggcgtggacataaaaacgctatcatctcctgcttgcatgaatgcgggggcaacatatccttcacccggcgatacctgctcttccaccacggtgtaaacttcccccgcaatatcttgcattactaccgtcacctccacagcccctactgcagtcagcaagtcccggcaaccccgacagaaaaatacagcagcgacaacggtgaccagaaaaccagcagttagaaaatccacaacaagtgcaccaggaggaggactgaggatcacagcgaacgagccagcgcagaccagagagctgaggaaccggatctttccaaccctctatgccattttccagcagagtcgggggcaagagcaggaactgaaagtaaaaaaccgatctctgcgctcgctcaccagaagttgtttgtatcacaagagcgaagaccaacttcagcgcactctcgaggacgccgaggctctcttcaacaagtactgcgcgctgactcttaaagagtagcccttgcccgcgctcattttgaaaacggcgggaatcacgtcacccttggcacctgtcctttgcccttgtcatgagtaaagagattcccacgccttacatgtggagctatcagccccaaatggggttggcagcaggcgcttcccaggactactccacccgcatgaattggcttagcgccgggccctcaatgatatcacgggttaatgatatacgagcttatcgaaaccagttactcctagaacagtcagctctcaccaccacaccccgtcaacaccttaatccccgaaattggcccgccaccctggtgtaccaggaaaatcccgctcccaccaccgtactacttcctcgagacgcccaggccgaagttcagatgactaacgcaggtgtacagctggcgggcggttccgccctatgtcgtcaccgacctcaacagagtataaaacgcctggtgattagaggccgaggtatccagctcaacgacgagtcggttagctcttcgcttggtctgcgaccagacggagtcttccaaatcgccggctgtgggagatcttccttcactcctcgtcaggctgtgctgactttggagagttcgtcctcgcagccccgctcgggcggcattggaactctccagtttgtggaggagtttactccctctgtctacttcaaccccttctccggctctcctggccagtacccggacgagttcataccaaacttcgacgcaatcagcgagtcagtggatggctatgattgatgtctaatggtggtgcggctgagctagctcgactgcgacacctagaccactgccgccgctttcgctgcttcgcccgggaactcaccgagttcatctacttcgaactctccgaggagcaccctcagggtccggcccacggagtgcggattaccatcgaagggggaatagactctcgcctgcatcgcatcttctcccagcggcccgtgctaattgaacgcgaccagggaaatacaaccatctccatctactgcatctgtaaccaccccggattgcatgaaagcctttgctgtcttgtttgtgctgagtttaataaaaactgagttaagaccctcctacggactaccgcttcttcaatcaggactttacaacaccaaccagatcttccagaagacccagacccttcctcctttcatccaggactctaactctaccttaccagcaccctccactactaaccttcccgaaacaaacaagcttgcatctcatctgcaacaccgcctttcacgaagccttctttctgccaatactaccactcccaaaaccggaggtgagctccgcggtcttcctactgacgacccctgggtggtagcgggttttgtaacgttaggagtagttgcgggtgggcttgtgctgatcctttgctacctatacacaccttgctgtgcatatttagtcatattgtgctgttggtttaagaaatgggggccatactagtcgtgcttgctttactttcgcttttgggtctgggctctgctaatctcaatcctctcgatcacgatccatgtttagacttcgacccagaaaactgcacacttacttttgcacccgacacaagccgtctctgtggagttcttattaagtgcggatgggactgcaggtccgttgaaattacacataataacaaaacatggaacaataccttatccaccacatgggagccaggagttccccagtggtatactgtctctgtccgaggtcctgacggttccatccgcattagtaacaacactttcattttttctgaaatgtgcgatctggccatgttcatgagcagacagtatgacctatggcctcccagcaaagagaacattgtggcattttccattgcttattgcttggtaacatgcatcatcactgctatcatttgtgtgtgcatacacttgcttatagttattcgccctagacaaagcaatgaggaaaaagagaaaatgccttaacctttttcctcataccttttctttacagcatggcttctgttacagctctaattattgccagcattgtcactgtcgctcacgggcaaacaattgtccatattaccttaggacataatcacactcttgtagggcccccaattacttcagaggttatttggaccaaacttggaagtgttgattattttgatataatttgcaacaaaactaaaccaatatttgtaatctgtaacagacaaaatctcacgttaattaatgttagcaaaatttataacggttactattatggttatgacagatccagtagtcaatataaaaattacttagttcgcataactcagcccaaattaacagtgccaactatgacaataattaaaatggctaataaagcattagaaaattttacatcaccaacaacacccaatgaaaaaaacattccaaattcaatgattgcaattattgcggcggtggcattgggaatggcactaataataatatgcatgctcctatatgcttgttactataaaaagtttcaacataaacaggatccactactaaattttaacatttaattttttatacagatgatttccactacaatttttatcattactagccttgcagctgtaacttatggccgttcacacctaactgtacctgttggctcaacatgtacactacaaggaccccaagaaggctatgtcacttggtggagaatatatgataatggagggttcgctagaccatgtgatcagcctggtacaaaattttcatgcaacggaagagacttgaccattattaacataacatcaaatgagcaaggcttctattatggaaccaactataaaaatagtttagattacaacattattgtagtgccagccaccacttctgctccccgcaaatccactttctctagcagcagtgccaaagcaagcacaattcctaaaacagcttctgctatgttaaagcttccaaaaatcgctttaagtaattccacagccgctcccaatacaattcctaaatcaacaattggcatcattactgccgtggtagtgggattaatgattatatttttgtgtataatgtactacgcctgctgctatagaaaacatgaacaaaaaggtgatgcattactaaattttgatatttaattttttatagaattatgatattgtttcaatcaaataccactacctcctatgcatacacaaacattcagcctaaatacgctatgcaactagaaatcacaatactaattgtaattggaattcttatactatctgttattctttattttatattctgccgtcaaatacccaatgttcatagaaattctaaaagacgtcccatctattctcctatgattagtcgtccccatatggctctgaatgaaatctaagatctttttttttttctcttacagtatggtgaacatcaatcatgatccctagaaatttcttcttcaccatactcatctgtgcttttaatgtctgtgctactttcacagcagtagccactgcaagcccagactgtataggaccatttgcttcctatgcactttttgccttcgttacttgcatctgcgtgtgtagcatagtctgcctggttattaattttttccaactggtagactggatctttgtgcgaattgcctacctacgtcaccatcccgaataccgcaatcaaaatgttgcggcacttcttaggcttatttaaaaccatgcaggctatgctaccagtcattttaattttgctactaccctgcattcccctagcttccaccgccactcgcgctacacctgaacaacttagaaaatgcaaatttcaacaaccatggtcatttcttgattgctaccatgaaaaatctgattttcccacatactggatagtgattgttggaataattaacatactttcatgtacctttttctcaatcacaatataccccacatttaattttgggtggaattctcccaatgcactgggttacccacaagaaccagatgaacatattccactacaacacatacaacaaccactagcactggtacagtatgaaaatgagccacaaccttcactgccccctgccattagttacttcaacctaaccggcggagatgactgacccaatcgccacatcatccaccgctgccaaggagctgctggacatggacggacgtgcctcagaacagcgactcatccaactacgcattcgtcagcagcaggaacgagcagtaaaagagctaagggatgccattgggattcaccagtgcaaaaaaggcatattctgcttagtaaaacaatccaaaatctcctacgagatcaccgctactgaccatcgtctctcatacgagctcggtccgcagcgacaaaaattcacctgcatggtgggaatcaaccccatagttatcacccagcagtctggagatactaagggttgtatccagtgttcctgtgattccaccgagtgcatctacacactgctgaagaccctctgcggccttcgagacctcctacccatgaactaatcattgcccctaccttacccaatcaaaatattaataaagacacttacttgaaatcagcaatacagtctttgtcaaaactttctaccagcagcacctcaccctcttcccaactctggtactctaaacgtcggagggtggcatactttctccacactttgaaagggatgtcaaattttatttcctcttctttgcccacaatcttcatttctttatccccagatggccaagcgagctcggctaagcacttccttcaacccggtgtacccttatgaagatgaaagcagctcacaacacccatttataaatcctggtttcatttcccctgacgggttcacacaaagtccaaacggggttttaagtcttaaatgtgttaatccacttaccactgcaagcggctccctccaacttaaagtgggaagtggtcttacagtagacactactgatggatccttagaagaaaacatcaaagttaacacccccctaacaaagtcaaaccattctataaatttaccaataggaaacggtttgcaaatagaacaaaacaaactttgcagtaaactcggaaatggtcttacatttgactcttccaattctattgcactgaaaaataacactttatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactgacaacaaaaataaagttcaacattttttattgaaattccttttacagtattcgagtagttattttgcctcccccttcccatttaacagaatacaccaatctctccccacgcacagctttaaacatttggataccattagagatagacatagttttagattccacattccaaacagtttcagagcgagccaatctggggtcagtaatacataaaaatgcatcgggatagtcttttaaagcgctttcacagtccaactgttgcggatgcgactccggagtctgaatcacggtcatctggaagaagaacgatgggaatcataatccgaaaacggaatcgggcgattgtgtctcatcaaacccacaagcaaccgctgtctgcgtcgctccgtgcgactgctgtttatgggatcggggtccgcagtgtcctgaagcatgattttaatagcccttaacattaactttctggtgcgatgcgcgcagcaacgcattctgatttcacttagattactacagtaggtacagcacattatcacaatattgtttaataaaccataattaaaagcgctccagccaaaactcatatctgatataatcgcccctgcatgaccatcataccaaagtttaatataaattaaatgtcgttccctcaaaaacacactacccacatacatgatctcttttggcatgtgcatattaacaatttgtctgtaccatggacaacgttggttaatcatgcaacccaatataaccttccggaaccacactgccaacaccgctcccccagccatgcattgaagtgaaccctgctgattacaatgacaatgaagaacccaattctctcgaccatgaatcacttgagactgaaaaatatctatagtagcacaacaaagacataaatgcatgcatcttctcataatttttaactcctctggatttaaaaacatatcccaaggaatgggaaactcttgcagaacagtaaagctggcagaacaaggaagaccacgaacacaacttacactatgcatagtcatagtatcacaatctggcaacagcgggtggtcttcagtcatagaagctcgggtttcattttcctcacatcgtggtaattgggctctggtgtaagggtgatgtctggcgcatgatgtggagcgtgcgcgcaaccttgtcataatggagttgcttcctgacattctcgtattttgtatagcaaaacgctgccctggcacaacacactcttcttcgtcttctatcctgccgcttagtgtgttccgtctgataattcaagtacagccacactcttaagttggtcaaaagaatgctggcttcagttgtaatcaaaactccatcatatttaattgttctaaggaaatcatccacggtagcatatgcaaatcccaaccaagcaatgcaactggattgcgtttcaagcagcagaggagagggaagagacggaagaatcatgttaatttttattccaaacgatctcgcagtacttcaaattgtagatcgcgcagatggcatctatcgcccccactgtgttggtgaaaaagcacagctaaatcaaaagaaatgcgattttcaaggtgctcaacggtggcttccaacaaagcctccacgcgcacatccaaaaacaaaagaataccaaaagaaggagcattttctaactcctcaaacatcatattacattcctgcaccattcccagataattttcagctttccagccttgaattattcgtgtcagttcttgtggtaaatccaaaccacacattacaaacaggtcccggagggcgccctccaccaccattcttaaacacaccctcataatgacaaaatatcttgctcctgtgtcacctgtagcaaattaagaatggcatcatcaattgacatgcccttggctctaagttcttctctaagttctagttgtagatactctctcatattatcaccaaactgcttagccagaagccccccgggaacaatagcaggggacgctacagtgcagtacaagcgcagacctccccaattggctccagcaaaaacaagattagaataagcatactgggaaccaccagtaatatcatcaaagttgctggaaatataatcaggcagagtttcttgtaaaaattgaataaaagaaaaattttccaaagaaacattcaaaaccgttgggatgcaaatacaataggttaccgcgctgcgctccaacattgttagttttgaattagtctgcaaaataaaagaaacaagcgtcatatcatagtagcctgtcgaacaggtggaaaaatcagtctttccatcacaagacaagccacagggtctccagctcgaccctcgtaaaacctgtcattgtgattaaacaacagcaccgaaagttcctcgcggtggccagcatgaataattcttgatgaagcatacaatccagacatgttagcatcagttaaagagaaaaaacagccaacatagcctctgggtataattatgcttaattttaagtatagcaaagccacccctcgcggatacaaagtaaaaggcacaggagaataaaaaatataattatttctctgctgctgttcaggcaacgttgctcccggtccctctaaatagacatacaaagcctcatcagccatggcttaccaggcaaagtacagcgggcgcacaaagcacaagctctaaagaagctctaaaaacactctccaacctctccacaatatatacacaagccctaaactgacgtaatgggagtaaagtgaaaaaaaaataccgccaagcccaacacacaccccgaaactgcgtcagcaggaaaaagtacagtttcacttccgcattcccaacaagcgtaacttcctctttctcatggtacgtcacatccgattaacttgcaacgtcattttcccacggtcgcgccgccccttttagccgttaaccccgcagccaatcaccacacagcgcgcacttttttaaattacctcatttacatgttggcaccattccatctataaggtatattatatagatag

SEQ ID NO: 52 - protein, 323 aa, Unknown
  Description of Unknown Human adenovirus 35 sequence
MTKRVRLSDSFNPVYPYEDESTSQHPFINPGFISPNGFTQSPDGVLTLKCLTPLTTTGGSLQLKVGGGLTVDDTDGTLQENIRATAPITKNNHSVELSIGNGLETQNNKLCAKLGNGLKFNNGDICIKDSINTLWTGINPPPNCQIVENTNTNDGKLTLVLVKNGGLVNGYVSLVGVSDTVNQMFTQKTANIQLRLYFDSSGNLLTEESDLKIPLKNKSSTATSETVASSKAFMPSTTAYPFNTTTRDSENYIHGICYYMTSYDRSLFPLNISIMLNSRMISSNVAYAIQFEWNLNASESPESNIATLTTSPFFFSYITEDDN

SEQ ID NO: 53 - DNA, 34794 bp, Unknown
  Description of Unknown Human adenovirus 35 sequence
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcaatactccaggggtgattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgatattcaagcacctgttcctgtggacgtgcgcaagcccattcctgtgaagcttaagcctgggaaacgtccagcagtggagaaacttgaggacttgttacagggtggggacggacctttggacttgagtacacggaaacgtccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagagcgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatccagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagccagaggatgattgggcggtggccattaaaaattatgccaagatagctttgaggcctgataaacagtataagatcagtagacggattaatatccggaatgcttgttacatatctggaaatggggctgaggtggtaatagatactcaagacaagacagttattagatgctgcatgatggatatgtggcctggagtagtcggtatggaagcagtcacttttgtaaatgttaagtttaggggagatggttataatggaatagtgtttatggccaataccaaacttatattgcatggttgtagcttttttggtttcaacaatacctgtgtagatgcctggggacaggttagtgtacgggggtgtagtttctatgcgtgttggattgccacagctggcagaaccaagagtcaattgtctctgaagaaatgcatattccaaagatgtaacctgggcattctgaatgaaggcgaagcaagggtccgtcactgcgcttctacagatactggatgttttattttaattaagggaaatgccagcgtaaagcataacatgatttgtggtgcttccgatgagaggccttatcaaatgctcacttgtgctggtgggcattgtaatatgctggctactgtgcatattgtttcccatcaacgcaaaaaatggcctgtttttgatcacaatgtgttgaccaagtgcaccatgcatgcaggtgggcgtagaggaatgtttatgccttaccagtgtaacatgaatcatgtgaaagtgttgttggaaccagatgccttttccagaatgagcctaacaggaatctttgacatgaacacgcaaatctggaagatcctgaggtatgatgatacgagatcgagggtgcgcgcatgcgaatgcggaggcaagcatgccaggttccagccggtgtgtgtagatgtgaccgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgacatgagtggaaatgcttcttttaaggggggagtcttcagcccttatctgacagggcgtctcccatcctgggcaggagttcgtcagaatgttatgggatctactgtggatggaagacccgttcaacccgccaattcttcaacgctgacctatgctactttaagttcttcacctttggacgcagctgcagccgctgccgccgcctctgtcgccgctaacactgtgcttggaatgggttactatggaagcatcgtggctaattccacttcctctaataacccttctacactgactcaggacaagttacttgtccttttggcccagctggaggctttgacccaacgtctgggtgaactttctcagcaggtggccgagttgcgagtacaaactgagtctgctgtcggcacggcaaagtctaaataaaaaaaattccagaatcaatgaataaataaacgagcttgttgttgatttaaaatcaagtgtttttatttcatttttcgcgcacggtatgccctggaccaccgatctcgatcattgagaactcggtggattttttccagaatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcgaggtgaaattatgtgcattttggattggatttttaagttggcaatattgccgccaagatcccgtcttgggttcatgttatgaaggactaccaagacggtgtatccggtacatttaggaaatttatcgtgcagcttggatggaaaagcgtggaaaaatttggagacacccttgtgtcctccgagattttccatgcactcatccatgataatagcaatggggccgtgggcagcggcgcgggcaaacacgttccgtgggtctgacacatcatagttatgttcctgagttaaatcatcataagccattttaatgaatttggggcggagcgtaccagattggggtatgaatgttccttcgggccccggagcatagttcccctcacagatttgcatttcccaagctttcagttctgagggtggaatcatgtccacctggggggctatgaagaacaccgtttcgggggcgggggtgattagttgggatgatagcaagtttctgagcaattgagatttgccacatccggtggggccataaataattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggaaagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatccgcgccgcaggaggcgcaaacagtttcacattccaccagccaggttaaatccggttcattggggtcaaaaacaagttttccgccatattttttgatgcgtttcttacctttggtctccataagttcgtgtcctcgttgagtgacaaacaggctgtccgtatctccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccagggcatgggatgggtgagagcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtccatgtcgtcacaggctccctgttcccagagttggaagtctacccgtttcttgtaggcggggttgggcaaagcgaaagtaacatcattgaagagaatcttaccggctctgggcataaaattgcgagtgatgcggaaaggctgtggtacttccgctcgattgttgatcacctgggcagctaggacgatttcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgggggcatggctggtagtggcacgtcggcgccgcgcacgggcaggttctggtattgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagcccctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctattttttttttttttttgccgctcagatgcatcccgtgctgcgacagatgcgcccccaacaacagcccccctcgcagcagcagcagcagcaaccacaaaaggctgtccctgcaactactgcaactgccgccgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgagacgaggatttcgaagttgatgaagtgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtagctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgatctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctgttattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggtgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtgttggacttgggtgggagaggaaggggcaacccgtttgctcatttgcgccctcgcttgggtggtatgttgtgaaaaaaaataaaaaagaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcagaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttggaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtttactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaatgtccattcttatctcgcccagtaataacaccggttggggtctgcgcgctccaagcaagatgtacggaggcgcacgcaaacgttctacccaacatcccgtgcgtgttcgcggacattttcgcgctccatggggtgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcaatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaaacgtataaataaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcaatggattgcaaaaggcgtaccaactgcagcagccgcaggcaatggtgaagaagaacatgaaacagaggagaaaactgctacttacacttttgccaatgctcctgtaaaagccgaggctcaaattacaaaagagggcttaccaataggtttggagatttcagctgaaaacgaatctaaacccatctatgcagataaactttatcagccagaacctcaagtgggagatgaaacttggactgacctagacggaaaaaccgaagagtatggaggcagggctctaaagcctactactaacatgaaaccctgttacgggtcctatgcgaagcctactaatttaaaaggtggtcaggcaaaaccgaaaaactcggaaccgtcgagtgaaaaaattgaatatgatattgacatggaattttttgataactcatcgcaaagaacaaacttcagtcctaaaattgtcatgtatgcagaaaatgtaggtttggaaacgccagacactcatgtagtgtacaaacctggaacagaagacacaagttccgaagctaatttgggacaacagtctatgcccaacagacccaactacattggcttcagagataactttattggactcatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactattgttttccactggacggcataggtgttccaacaaccagttacaaatcaatagttccaaatggagaagataataataattggaaagaacctgaagtaaatggaacaagtgagatcggacagggtaatttgtttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatctatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggcccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctctcatcgtacacacatcgaaagggccactgcgttcgaccgtatggatgttcaataatgactcatgtaaacaacgtgttcaataaacatcactttatttttttacatgtatcaaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctctgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttggctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcttcgtttacgtactggtgacagatgcgcttgtattgttcgtgttgctcaggcattagtttaaaacaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttttcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacagcgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagctgcgcctacgagcttaaccttttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtctatctctacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgccactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcttgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccgacagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcagcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtagaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaacttattgagttcatctacttcgaactccccaaggatcaccctcaaggtccggcccacggagtgcggattactatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacaaaacttttcctgtcgtccaggactctgttaacttcacctttcctactcacaaactagaagctcaacgactacaccgcttttccagaagcattttccctactaatactactttcaaaaccggaggtgagctccacggtctccctacagaaaacccttgggtggaagcgggccttgtagtactaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagactttgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatcccactaggacataattacactctcataggacccccaatcacttcagaggtcatctggaccaaactgggaagcgttgattactttgatataatctgtaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcatcatcgctgcagtgacaattggaatatctattcttgtttttaccataacctactacgcctgctgctatagaaaagacaaacataaaggtgatccattacttagatttgatatttaatttgttctttttttttatttacagtatggtgaacaccaatcatggtacctagaaatttcttcttcaccatactcatctgtgcttttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaatccccccaaatttaataatgattgctggaataattaatataatctgttgcaccataatttcatttttgatataccccctatttgattttggctggaatgctcccaatgcacatgatcatccacaagacccagaggaacacattcccccacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacaacgacttgcccaactacgcatccgccagcagcaggaacgcgtggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccaacaaagtggagatactaagggttgcattcactgctcctgcgattccatcgagtgcacctacaccctgctgaagaccctatgcggcctaagagacctgctaccaatgaattaaaaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgactccggagtttggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtttcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccaccagtaatatcatcgaagttgctggaaatataatcaggcagagtttcttgtagaaattgaataaaagaaaaatttgccaaaaaaacattcaaaacctctgggatgcaaatgcaataggttaccgcgctgcgctccaacattgttagttttgaattagtctgcaaaaataaaaaaaaaacaagcgtcatatcatagtagcctgacgaacaggtggataaatcagtctttccatcacaagacaagccacagggtctccagctcgaccctcgtaaaacctgtcatcgtgattaaacaacagcaccgaaagttcctcgcggtgaccagcatgaataagtcttgatgaagcatacaatccagacatgttagcatcagttaaggagaaaaaacagccaacatagcctttgggtataattatgcttaatcgtaagtatagcaaagccacccctcgcggatacaaagtaaaaggcacaggagaataaaaaatataattatttctctgctgctgtttaggcaacgtcgcccccggtccctctaaatacacatacaaagcctcatcagccatggcttaccagagaaagtacagcgggcacacaaaccacaagctctaaagtcactctccaacctctccacaatatatatacacaagccctaaactgacgtaatgggactaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttacaacgtcattttcccacggccgcgccgccccttttaaccgttaaccccacagccaatcaccacacggcccacactttttaaaatcacctcatttacatattggcaccattccatctataaggtatattattgatgatg

SEQ ID NO: 54 - DNA, 34794 bp, Unknown; identical to SEQ ID NO: 53
  Description of Unknown Human adenovirus 35 sequence

SEQ ID NO: 55 - DNA, 34794 bp, Unknown
  Description of Unknown Adenoviridae family sequence
catcatcaataatataccttatagatggaatggtgccaatatgtaaatgaggtgattttaaaaagtgtgggccgtgtggtgattggctgtggggttaacggttaaaaggggcggcgcggccgtgggaaaatgacgttttatgggggtggagtttttttgcaagttgtcgcgggaaatgttacgcataaaaaggcttcttttctcacggaactacttagttttcccacggtatttaacaggaaatgaggtagttttgaccggatgcaagtgaaaattgctgattttcgcgcgaaaactgaatgaggaagtgtttttctgaataatgtggtatttatggcagggtggagtatttgttcagggccaggtagactttgacccattacgtggaggtttcgattaccgtgttttttacctgaatttccgcgtaccgtgtcaaagtcttctgtttttacgtaggtgtcagctgatcgctagggtatttatacctcagggtttgtgtcaagaggccactcttgagtgccagcgagaagagttttctcctctgcgccggcagtttaataataaaaaaatgagagatttgcgatttctgcctcaggaaataatctctgctgagactggaaatgaaatattggagcttgtggtgcacgccctgatgggagacgatccggagccacctgtgcagctttttgagcctcctacgcttcaggaactgtatgatttagaggtagagggatcggaggattctaatgaggaagctgtgaatggcttttttaccgattctatgcttttagctgctaatgaaggattagaattagatccgcctttggacactttcaatactccaggggtgattgtggaaagcggtacaggtgtaagaaaattacctgatttgagttccgtggactgtgatttgcactgctatgaagacgggtttcctccgagtgatgaggaggaccatgaaaaggagcagtccatgcagactgcagcgggtgagggagtgaaggctgccaatgttggttttcagttggattgcccggagcttcctggacatggctgtaagtcttgtgaatttcacaggaaaaatactggagtaaaggaactgttatgttcgctttgttatatgagaacgcactgccactttatttacagtaagtgtgtttaagttaaaatttaaaggaatatgctgtttttcacatgtatattgagtgtgagttttgtgcttcttattataggtcctgtgtctgatgctgatgaatcaccatctcctgattctactacctcacctcctgatattcaagcacctgttcctgtggacgtgcgcaagcccattcctgtgaagcttaagcctgggaaacgtccagcagtggagaaacttgaggacttgttacagggtggggacggacctttggacttgagtacacggaaacgtccaagacaataagtgttccatatccgtgtttacttaaggtgacgtcaatatttgtgtgagagtgcaatgtaataaaaatatgttaactgttcactggtttttattgctttttgggcggggactcaggtatataagtagaagcagacctgtgtggttagctcataggagctggctttcatccatggaggtttgggccattttggaagaccttaggaagactaggcaactgttagagagcgcttcggacggagtctccggtttttggagattctggttcgctagtgaattagctagggtagtttttaggataaaacaggactataaacaagaatttgaaaagttgttggtagattgcccaggactttttgaagctcttaatttgggccatcaggttcactttaaagaaaaagttttatcagttttagacttttcaaccccaggtagaactgctgctgctgtggcttttcttacttttatattagataaatggatcccgcagactcatttcagcaggggatacgttttggatttcatagccacagcattgtggagaacatggaaggttcgcaagatgaggacaatcttaggttactggccagtgcagcctttgggtgtagcgggaatcctgaggcatccaccggtcatgccagcggttctggaggaggaacagcaagaggacaacccgagagccggcctggaccctccagtggaggaggcggagtagctgacttgtctcctgaactgcaacgggtgcttactggatctacgtccactggacgggataggggcgttaagagggagagggcatccagtggtactgatgctagatctgagttggctttaagtttaatgagtcgcagacgtcctgaaaccatttggtggcatgaggttcagaaagagggaagggatgaagtttctgtattgcaggagaaatattcactggaacaggtgaaaacatgttggttggagccagaggatgattgggcggtggccattaaaaattatgccaagatagctttgaggcctgataaacagtataagatcagtagacggattaatatccggaatgcttgttacatatctggaaatggggctgaggtggtaatagatactcaagacaagacagttattagatgctgcatgatggatatgtggcctggagtagtcggtatggaagcagtcacttttgtaaatgttaagtttaggggagatggttataatggaatagtgtttatggccaataccaaacttatattgcatggttgtagcttttttggtttcaacaatacctgtgtagatgcctggggacaggttagtgtacgggggtgtagtttctatgcgtgttggattgccacagctggcagaaccaagagtcaattgtctctgaagaaatgcatattccaaagatgtaacctgggcattctgaatgaaggcgaagcaagggtccgtcactgcgcttctacagatactggatgttttattttaattaagggaaatgccagcgtaaagcataacatgatttgtggtgcttccgatgagaggccttatcaaatgctcacttgtgctggtgggcattgtaatatgctggctactgtgcatattgtttcccatcaacgcaaaaaatggcctgtttttgatcacaatgtgttgaccaagtgcaccatgcatgcaggtgggcgtagaggaatgtttatgccttaccagtgtaacatgaatcatgtgaaagtgttgttggaaccagatgccttttccagaatgagcctaacaggaatctttgacatgaacacgcaaatctggaagatcctgaggtatgatgatacgagatcgagggtgcgcgcatgcgaatgcggaggcaagcatgccaggttccagccggtgtgtgtagatgtgaccgaagatctcagaccggatcatttggttattgcccgcactggagcagagttcggatccagtggagaagaaactgactaaggtgagtattgggaaaactttggggtgggattttcagatggacagattgagtaaaaatttgttttttctgtcttgcagctgacatgagtggaaatgcttcttttaaggggggagtcttcagcccttatctgacagggcgtctcccatcctgggcaggagttcgtcagaatgttatgggatctactgtggatggaagacccgttcaacccgccaattcttcaacgctgacctatgctactttaagttcttcacctttggacgcagctgcagccgctgccgccgcctctgtcgccgctaacactgtgcttggaatgggttactatggaagcatcgtggctaattccacttcctctaataacccttctacactgactcaggacaagttacttgtccttttggcccagctggaggctttgacccaacgtctgggtgaactttctcagcaggtggccgagttgcgagtacaaactgagtctgctgtcggcacggcaaagtctaaataaaaaaaattccagaatcaatgaataaataaacgagcttgttgttgatttaaaatcaagtgtttttatttcatttttcgcgcacggtatgccctggaccaccgatctcgatcattgagaactcggtggattttttccagaatcctatagaggtgggattgaatgtttagatacatgggcattaggccgtctttggggtggagatagctccattgaagggattcatgctccggggtagtgttgtaaatcacccagtcataacaaggtcgcagtgcatggtgttgcacaatatcttttagaagtaggctgattgccacagataagcccttggtgtaggtgtttacaaaccggttgagctgggaggggtgcattcgaggtgaaattatgtgcattttggattggatttttaagttggcaatattgccgccaagatcccgtcttgggttcatgttatgaaggactaccaagacggtgtatccggtacatttaggaaatttatcgtgcagcttggatggaaaagcgtggaaaaatttggagacacccttgtgtcctccgagattttccatgcactcatccatgataatagcaatggggccgtgggcagcggcgcgggcaaacacgttccgtgggtctgacacatcatagttatgttcctgagttaaatcatcataagccattttaatgaatttggggcggagcgtaccagattggggtatgaatgttccttcgggccccggagcatagttcccctcacagatttgcatttcccaagctttcagttctgagggtggaatcatgtccacctggggggctatgaagaacaccgtttcgggggcgggggtgattagttgggatgatagcaagtttctgagcaattgagatttgccacatccggtggggccataaataattccgattacaggttgcaggtggtagtttagggaacggcaactgccgtcttctcgaagcaagggggccacctcgttcatcatttcccttacatgcatattttcccgcaccaaatccattaggaggcgctctcctcctagtgatagaagttcttgtagtgaggaaaagtttttcagcggttttagaccgtcagccatgggcattttggaaagagtttgctgcaaaagttctagtctgttccacagttcagtgatgtgttctatggcatctcgatccagcagacctcctcgtttcgcgggtttggacggctcctggagtagggtatgagacgatgggcgtccagcgctgccagggttcggtccttccagggtctcagtgttcgagtcagggttgtttccgtcacagtgaaggggtgtgcgcctgcttgggcgcttgccagggtgcgcttcagactcattctgctggtggagaacttctgtcgcttggcgccctgtatgtcggccaagtagcagtttaccatgagttcgtagttgagcgcctcggctgcgtggcctttggcgcggagcttacctttggaagttttcttgcataccgggcagtataggcatttcagcgcatacagcttgggcgcaaggaaaatggattctggggagtatgcatccgcgccgcaggaggcgcaaacagtttcacattccaccagccaggttaaatccggttcattggggtcaaaaacaagttttccgccatattttttgatgcgtttcttacctttggtctccataagttcgtgtcctcgttgagtgacaaacaggctgtccgtatctccgtagactgattttacaggcctcttctccagtggagtgcctcggtcttcttcgtacaggaactctgaccactctgatacaaaggcgcgcgtccaggccagcacaaaggaggctatgtgggaggggtagcgatcgttgtcaaccagggggtccaccttttccaaagtatgcaaacacatgtcaccctcttcaacatccaggaatgtgattggcttgtaggtgtatttcacgtgacctggggtccccgctgggggggtataaaagggggcggttctttgctcttcctcactgtcttccggatcgctgtccaggaacgtcagctgttggggtaggtattccctctcgaaggcgggcatgacctctgcactcaggttgtcagtttctaagaacgaggaggatttgatattgacagtgccggttgagatgcctttcatgaggttttcgtccatttggtcagaaaacacaatttttttattgtcaagtttggtggcaaatgatccatacagggcgttggataaaagtttggcaatggatcgcatggtttggttcttttccttgtccgcgcgctctttggcggcgatgttgagttggacatactcgcgtgccaggcacttccattcggggaagatagttgttaattcatctggcacgattctcacttgccaccctcgattatgcaaggtaattaaatccacactggtggccacctcgcctcgaaggggttcattggtccaacagagcctacctcctttcctagaacagaaagggggaagtgggtctagcataagttcatcgggagggtctgcatccatggtaaagattcccggaagtaaatccttatcaaaatagctgatgggagtggggtcatctaaggccatttgccattctcgagctgccagtgcgcgctcatatgggttaaggggactgccccagggcatgggatgggtgagagcagaggcatacatgccacagatgtcatagacgtagatgggatcctcaaagatgcctatgtaggttggatagcatcgcccccctctgatacttgctcgcacatagtcatatagttcatgtgatggcgctagcagccccggacccaagttggtgcgattgggtttttctgttctgtagacgatctggcgaaagatggcgtgagaattggaagagatggtgggtctttgaaaaatgttgaaatgggcatgaggtagacctacagagtctctgacaaagtgggcataagattcttgaagcttggttaccagttcggcggtgacaagtacgtctagggcgcagtagtcaagtgtttcttgaatgatgtcataacctggttggtttttcttttcccacagttcgcggttgagaaggtattcttcgcgatccttccagtactcttctagcggaaacccgtctttgtctgcacggtaagatcctagcatgtagaactgattaactgccttgtaagggcagcagcccttctctacgggtagagagtatgcttgagcagcttttcgtagcgaagcgtgagtaagggcaaaggtgtctctgaccatgactttgagaaattggtatttgaagtccatgtcgtcacaggctccctgttcccagagttggaagtctacccgtttcttgtaggcggggttgggcaaagcgaaagtaacatcattgaagagaatcttaccggctctgggcataaaattgcgagtgatgcggaaaggctgtggtacttccgctcgattgttgatcacctgggcagctaggacgatttcgtcgaaaccgttgatgttgtgtcctacgatgtataattctatgaaacgcggcgtgcctctgacgtgaggtagcttactgagctcatcaaaggttaggtctgtggggtcagataaggcgtagtgttcgagagcccattcgtgcaggtgaggatttgcatgtaggaatgatgaccaaagatctaccgccagtgctgtttgtaactggtcccgatactgacgaaaatgccggccaattgccattttttctggagtgacacagtagaaggttctggggtcttgttgccatcgatcccacttgagtttaatggctagatcgtgggccatgttgacgagacgctcttctcctgagagtttcatgaccagcatgaaaggaactagttgtttgccaaaggatcccatccaggtgtaagtttccacatcgtaggtcaggaagagtctttctgtgcgaggatgagagccgatcgggaagaactggatttcctgccaccagttggaggattggctgttgatgtgatggaagtagaagtttctgcggcgcgccgagcattcgtgtttgtgcttgtacagacggccgcagtagtcgcagcgttgcacgggttgtatctcgtgaatgagctgtacctggcttcccttgacgagaaatttcagtgggaagccgaggcctggcgattgtatctcgtgctcttctatattcgctgtatcggcctgttcatcttctgtttcgatggtggtcatgctgacgagcccccgcgggaggcaagtccagacctcggcgcgggaggggcggagctgaaggacgagagcgcgcaggctggagctgtccagagtcctgagacgctgcggactcaggttagtaggtagggacagaagattaacttgcatgatcttttccagggcgtgcgggaggttcagatggtacttgatttccacaggttcgtttgtagagacgtcaatggcttgcagggttccgtgtcctttgggcgccactaccgtacctttgttttttcttttgatcggtggtggctctcttgcttcttgcatgctcagaagcggtgacggggacgcgcgccgggcggcagcggttgttccggacccgggggcatggctggtagtggcacgtcggcgccgcgcacgggcaggttctggtattgcgctctgagaagacttgcgtgcgccaccacgcgtcgattgacgtcttgtatctgacgtctctgggtgaaagctaccggccccgtgagcttgaacctgaaagagagttcaacagaatcaatttcggtatcgttaacggcagcttgtctcagtatttcttgtacgtcaccagagttgtcctggtaggcgatctccgccatgaactgctcgatttcttcctcctgaagatctccgcgacccgctctttcgacggtggccgcgaggtcattggagatacggcccatgagttgggagaatgcattcatgcccgcctcgttccagacgcggctgtaaaccacggccccctcggagtctcttgcgcgcatcaccacctgagcgaggttaagctccacgtgtctggtgaagaccgcatagttgcataggcgctgaaaaaggtagttgagtgtggtggcaatgtgttcggcgacgaagaaatacatgatccatcgtctcagcggcatttcgctaacatcgcccagagcttccaagcgctccatggcctcgtagaagtccacggcaaaattaaaaaactgggagtttcgcgcggacacggtcaattcctcctcgagaagacggatgagttcggctatggtggcccgtacttcgcgttcgaaggctcccgggatctcttcttcctcttctatctcttcttccactaacatctcttcttcgtcttcaggcgggggcggagggggcacgcggcgacgtcgacggcgcacgggcaaacggtcgatgaatcgttcaatgacctctccgcggcggcggcgcatggtttcagtgacggcgcggccgttctcgcgcggtcgcagagtaaaaacaccgccgcgcatctccttaaagtggtgactgggaggttctccgtttgggagggagagggcgctgattatacattttattaattggcccgtagggactgcgcgcagagatctgatcgtgtcaagatccacgggatctgaaaacctttcgacgaaagcgtctaaccagtcacagtcacaaggtaggctgagtacggcttcttgtgggcgggggtggttatgtgttcggtctgggtcttctgtttcttcttcatctcgggaaggtgagacgatgctgctggtgatgaaattaaagtaggcagttctaagacggcggatggtggcgaggagcaccaggtctttgggtccggcttgctggatacgcaggcgattggccattccccaagcattatcctgacatctagcaagatctttgtagtagtcttgcatgagccgttctacgggcacttcttcctcacccgttctgccatgcatacgtgtgagtccaaatccgcgcattggttgtaccagtgccaagtcagctacgactctttcggcgaggatggcttgctgtacttgggtaagggtggcttgaaagtcatcaaaatccacaaagcggtggtaagcccctgtattaatggtgtaagcacagttggccatgactgaccagttaactgtctggtgaccagggcgcacgagctcggtgtatttaaggcgcgaataggcgcgggtgtcaaagatgtaatcgttgcaggtgcgcaccagatactggtaccctataagaaaatgcggcggtggttggcggtagagaggccatcgttctgtagctggagcgccaggggcgaggtcttccaacataaggcggtgatagccgtagatgtacctggacatccaggtgattcctgcggcggtagtagaagcccgaggaaactcgcgtacgcggttccaaatgttgcgtagcggcatgaagtagttcattgtaggcacggtttgaccagtgaggcgcgcgcagtcattgatgctctatagacacggagaaaatgaaagcgttcagcgactcgactccgtagcctggaggaacgtgaacgggttgggtcgcggtgtaccccggttcgagacttgtactcgagccggccggagccgcggctaacgtggtattggcactcccgtctcgacccagcctacaaaaatccaggatacggaatcgagtcgttttgctggtttccgaatggcagggaagtgagtcctattttttttttttttttgccgctcagatgcatcccgtgctgcgacagatgcgcccccaacaacagcccccctcgcagcagcagcagcagcaaccacaaaaggctgtccctgcaactactgcaactgccgccgtgagcggtgcgggacagcccgcctatgatctggacttggaagagggcgaaggactggcacgtctaggtgcgccttcgcccgagcggcatccgcgagttcaactgaaaaaagattctcgcgaggcgtatgtgccccaacagaacctatttagagacagaagcggcgaggagccggaggagatgcgagcttcccgctttaacgcgggtcgtgagctgcgtcacggtttggaccgaagacgagtgttgcgagacgaggatttcgaagttgatgaagtgacagggatcagtcctgccagggcacacgtggctgcagccaaccttgtatcggcttacgagcagacagtaaaggaagagcgtaacttccaaaagtcttttaataatcatgtgcgaaccctgattgcccgcgaagaagttacccttggtttgatgcatttgtgggatttgatggaagctatcattcagaaccctactagcaaacctctgaccgcccagctgtttctggtggtgcaacacagcagagacaatgaggctttcagagaggcgctgctgaacatcaccgaacccgaggggagatggttgtatgatcttatcaacattctacagagtatcatagtgcaggagcggagcctgggcctggccgagaaggtagctgccatcaattactcggttttgagcttgggaaaatattacgctcgcaaaatctacaagactccatacgttcccatagacaaggaggtgaagatagatgggttctacatgcgcatgacgctcaaggtcttgaccctgagcgatgatcttggggtgtatcgcaatgacagaatgcatcgcgcggttagcgccagcaggaggcgcgagttaagcgacagggaactgatgcacagtttgcaaagagctctgactggagctggaaccgagggtgagaattacttcgacatgggagctgacttgcagtggcagcctagtcgcagggctctgagcgccgcgacggcaggatgtgagcttccttacatagaagaggcggatgaaggcgaggaggaagagggcgagtacttggaagactgatggcacaacccgtgttttttgctagatggaacagcaagcaccggatcccgcaatgcgggcggcgctgcagagccagccgtccggcattaactcctcggacgattggacccaggccatgcaacgtatcatggcgttgacgactcgcaaccccgaagcctttagacagcaaccccaggccaaccgtctatcggccatcatggaagctgtagtgccttcccgatctaatcccactcatgagaaggtcctggccatcgtgaacgcgttggtggagaacaaagctattcgtccagatgaggccggactggtatacaacgctctcttagaacgcgtggctcgctacaacagtagcaatgtgcaaaccaatttggaccgtatgataacagatgtacgcgaagccgtgtctcagcgcgaaaggttccagcgtgatgccaacctgggttcgctggtggcgttaaatgctttcttgagtactcagcctgctaatgtgccgcgtggtcaacaggattatactaactttttaagtgctttgagactgatggtatcagaagtacctcagagcgaagtgtatcagtccggtcctgattacttctttcagactagcagacagggcttgcagacggtaaatctgagccaagcttttaaaaaccttaaaggtttgtggggagtgcatgccccggtaggagaaagagcaaccgtgtctagcttgttaactccgaactcccgcctgttattactgttggtagctcctttcaccgacagcggtagcatcgaccgtaattcctatttgggttacctactaaacctgtatcgcgaagccatagggcaaagtcaggtggacgagcagacctatcaagaaattacccaagtcagtcgcgctttgggacaggaagacactggcagtttggaagccactctgaacttcttgcttaccaatcggtctcaaaagatccctcctcaatatgctcttactgcggaggaggagaggatccttagatatgtgcagcagagcgtgggattgtttctgatgcaagagggggcaactccgactgcagcactggacatgacagcgcgaaatatggagcccagcatgtatgccagtaaccgacctttcattaacaaactgctggactacttgcacagagctgccgctatgaactctgattatttcaccaatgccatcttaaacccgcactggctgcccccacctggtttctacacgggcgaatatgacatgcccgaccctaatgacggatttctgtgggacgacgtggacagcgatgttttttcacctctttctgatcatcgcacgtggaaaaaggaaggcggtgatagaatgcattcttctgcatcgctgtccggggtcatgggtgctaccgcggctgagcccgagtctgcaagtccttttcctagtctacccttttctctacacagtgtacgtagcagcgaagtgggtagaataagtcgcccgagtttaatgggcgaagaggagtacctaaacgattccttgctcagaccggcaagagaaaaaaatttcccaaacaatggaatagaaagtttggtggataaaatgagtagatggaagacttatgctcaggatcacagagacgagcctgggatcatggggactacaagtagagcgagccgtagacgccagcgccatgacagacagaggggtcttgtgtgggacgatgaggattcggccgatgatagcagcgtgttggacttgggtgggagaggaaggggcaacccgtttgctcatttgcgccctcgcttgggtggtatgttgtgaaaaaaaataaaaaagaaaaactcaccaaggccatggcgacgagcgtacgttcgttcttctttattatctgtgtctagtataatgaggcgagtcgtgctaggcggagcggtggtgtatccggagggtcctcctccttcgtacgagagcgtgatgcagcagcagcaggcgacggcggtgatgcaatccccactggaggctccctttgtgcctccgcgatacctggcacctacggagggcagaaacagcattcgttactcggaactggcacctcagtacgataccaccaggttgtatctggtggacaacaagtcggcggacattgcttctctgaactatcagaatgaccacagcaacttcttgaccacggtggtgcagaacaatgactttacccctacggaagccagcacccagaccattaactttgatgaacgatcgcggtggggcggtcagctaaagaccatcatgcatactaacatgccaaacgtgaacgagtatatgtttagtaacaagttcaaagcgcgtgtgatggtgtccagaaaacctcccgacggtgctgcagttggggatacttatgatcacaagcaggatattttggaatatgagtggttcgagtttactttgccagaaggcaacttttcagttactatgactattgatttgatgaacaatgccatcatagataattacttgaaagtgggtagacagaatggagtgcttgaaagtgacattggtgttaagttcgacaccaggaacttcaagctgggatgggatcccgaaaccaagttgatcatgcctggagtgtatacgtatgaagccttccatcctgacattgtcttactgcctggctgcggagtggattttaccgagagtcgtttgagcaaccttcttggtatcagaaaaaaacagccatttcaagagggttttaagattttgtatgaagatttagaaggtggtaatattccggccctcttggatgtagatgcctatgagaacagtaagaaagaacaaaaagccaaaatagaagctgctacagctgctgcagaagctaaggcaaacatagttgccagcgactctacaagggttgctaacgctggagaggtcagaggagacaattttgcgccaacacctgttccgactgcagaatcattattggccgatgtgtctgaaggaacggacgtgaaactcactattcaacctgtagaaaaagatagtaagaatagaagctataatgtgttggaagacaaaatcaacacagcctatcgcagttggtatctttcgtacaattatggcgatcccgaaaaaggagtgcgttcctggacattgctcaccacctcagatgtcacctgcggagcagagcaggtttactggtcgcttccagacatgatgaaggatcctgtcactttccgctccactagacaagtcagtaactaccctgtggtgggtgcagagcttatgcccgtcttctcaaagagcttctacaacgaacaagctgtgtactcccagcagctccgccagtccacctcgcttacgcacgtcttcaaccgctttcctgagaaccagattttaatccgtccgccggcgcccaccattaccaccgtcagtgaaaacgttcctgctctcacagatcacgggaccctgccgttgcgcagcagtatccggggagtccaacgtgtgaccgttactgacgccagacgccgcacctgtccctacgtgtacaaggcactgggcatagtcgcaccgcgcgtcctttcaagccgcactttctaaaaaaaaaaaatgtccattcttatctcgcccagtaataacaccggttggggtctgcgcgctccaagcaagatgtacggaggcgcacgcaaacgttctacccaacatcccgtgcgtgttcgcggacattttcgcgctccatggggtgccctcaagggccgcactcgcgttcgaaccaccgtcgatgatgtaatcgatcaggtggttgccgacgcccgtaattatactcctactgcgcctacatctactgtggatgcagttattgacagtgtagtggctgacgctcgcaactatgctcgacgtaagagccggcgaaggcgcattgccagacgccaccgagctaccactgccatgcgagccgcaagagctctgctacgaagagctagacgcgtggggcgaagagccatgcttagggcggccagacgtgcagcttcgggcgccagcgccggcaggtcccgcaggcaagcagccgctgtcgcagcggcgactattgccgacatggcccaatcgcgaagaggcaatgtatactgggtgcgtgacgctgccaccggtcaacgtgtacccgtgcgcacccgtccccctcgcacttagaagatactgagcagtctccgatgttgtgtcccagcggcgaggatgtccaagcgcaaatacaaggaagaaatgctgcaggttatcgcacctgaagtctacggccaaccgttgaaggatgaaaaaaaaccccgcaaaatcaagcgggttaaaaaggacaaaaaagaagaggaagatggcgatgatgggctggcggagtttgtgcgcgagtttgccccacggcgacgcgtgcaatggcgtgggcgcaaagttcgacatgtgttgagacctggaacttcggtggtctttacacccggcgagcgttcaagcgctacttttaagcgttcctatgatgaggtgtacggggatgatgatattcttgagcaggcggctgaccgattaggcgagtttgcttatggcaagcgtagtagaataacttccaaggatgagacagtgtcaatacccttggatcatggaaatcccacccctagtcttaaaccggtcactttgcagcaagtgttacccgtaactccgcgaacaggtgttaaacgcgaaggtgaagatttgtatcccactatgcaactgatggtacccaaacgccagaagttggaggacgttttggagaaagtaaaagtggatccagatattcaacctgaggttaaagtgagacccattaagcaggtagcgcctggtctgggggtacaaactgtagacattaagattcccactgaaagtatggaagtgcaaactgaacccgcaaagcctactgccacctccactgaagtgcaaacggatccatggatgcccatgcctattacaactgacgccgccggtcccactcgaagatcccgacgaaagtacggtccagcaagtctgttgatgcccaattatgttgtacacccatctattattcctactcctggttaccgaggcactcgctactatcgcagccgaaacagtacctcccgccgtcgccgcaagacacctgcaaatcgcagtcgtcgccgtagacgcacaagcaaaccgactcccggcgccctggtgcggcaagtgtaccgcaatggtagtgcggaacctttgacactgccgcgtgcgcgttaccatccgagtatcatcacttaatcaatgttgccgctgcctccttgcagatatggccctcacttgtcgccttcgcgttcccatcactggttaccgaggaagaaactcgcgccgtagaagagggatgttgggacgcggaatgcgacgctacaggcgacggcgtgctatccgcaagcaattgcggggtggttttttaccagccttaattccaattatcgctgctgcaattggcgcgataccaggcatagcttccgtggcggttcaggcctcgcaacgacattgacattggaaaaaaaacgtataaataaaaaaaaatacaatggactctgacactcctggtcctgtgactatgttttcttagagatggaagacatcaatttttcatccttggctccgcgacacggcacgaagccgtacatgggcacctggagcgacatcggcacgagccaactgaacgggggcgccttcaattggagcagtatctggagcgggcttaaaaattttggctcaaccataaaaacatacgggaacaaagcttggaacagcagtacaggacaggcgcttagaaataaacttaaagaccagaacttccaacaaaaagtagtcgatgggatagcttccggcatcaatggagtggtagatttggctaaccaggctgtgcagaaaaagataaacagtcgtttggacccgccgccagcaaccccaggtgaaatgcaagtggaggaagaaattcctccgccagaaaaacgaggcgacaagcgtccgcgtcccgatttggaagagacgctggtgacgcgcgtagatgaaccgccttcttatgaggaagcaacgaagcttggaatgcccaccactagaccgatagccccaatggccaccggggtgatgaaaccttctcagttgcatcgacccgtcaccttggatttgccccctccccctgctgctactgctgtacccgcttctaagcctgtcgctgccccgaaaccagtcgccgtagccaggtcacgtcccgggggcgctcctcgtccaaatgcgcactggcaaaatactctgaacagcatcgtgggtctaggcgtgcaaagtgtaaaacgccgtcgctgcttttaattaaatatggagtagcgcttaacttgcctatctgtgtatatgtgtcattacacgccgtcacagcagcagaggaaaaaaggaagaggtcgtgcgtcgacgctgagttactttcaagatggccaccccatcgatgctgccccaatgggcatacatgcacatcgccggacaggatgcttcggagtacctgagtccgggtctggtgcagttcgcccgcgccacagacacctacttcaatctgggaaataagtttagaaatcccaccgtagcgccgacccacgatgtgaccaccgaccgtagccagcggctcatgttgcgcttcgtgcccgttgaccgggaggacaatacatactcttacaaagtgcggtacaccctggccgtgggcgacaacagagtgctggatatggccagcacgttctttgacattaggggcgtgttggacagaggtcccagtttcaaaccctattctggtacggcttacaactctctggctcctaaaggcgctccaaatgcatctcaatggattgcaaaaggcgtaccaactgcagcagccgcaggcaatggtgaagaagaacatgaaacagaggagaaaactgctacttacacttttgccaatgctcctgtaaaagccgaggctcaaattacaaaagagggcttaccaataggtttggagatttcagctgaaaacgaatctaaacccatctatgcagataaactttatcagccagaacctcaagtgggagatgaaacttggactgacctagacggaaaaaccgaagagtatggaggcagggctctaaagcctactactaacatgaaaccctgttacgggtcctatgcgaagcctactaatttaaaaggtggtcaggcaaaaccgaaaaactcggaaccgtcgagtgaaaaaattgaatatgatattgacatggaattttttgataactcatcgcaaagaacaaacttcagtcctaaaattgtcatgtatgcagaaaatgtaggtttggaaacgccagacactcatgtagtgtacaaacctggaacagaagacacaagttccgaagctaatttgggacaacagtctatgcccaacagacccaactacattggcttcagagataactttattggactcatgtactataacagtactggtaacatgggggtgctggctggtcaagcgtctcagttaaatgcagtggttgacttgcaggacagaaacacagaactttcttaccaactcttgcttgactctctgggcgacagaaccagatactttagcatgtggaatcaggctgtggacagttatgatcctgatgtacgtgttattgaaaatcatggtgtggaagatgaacttcccaactattgttttccactggacggcataggtgttccaacaaccagttacaaatcaatagttccaaatggagaagataataataattggaaagaacctgaagtaaatggaacaagtgagatcggacagggtaatttgtttgccatggaaattaaccttcaagccaatctatggcgaagtttcctttattccaatgtggctctgtatctcccagactcgtacaaatacaccccgtccaatgtcactcttccagaaaacaaaaacacctacgactacatgaacgggcgggtggtgccgccatctctagtagacacctatgtgaacattggtgccaggtggtctctggatgccatggacaatgtcaacccattcaaccaccaccgtaacgctggcttgcgttaccgatctatgcttctgggtaacggacgttatgtgcctttccacatacaagtgcctcaaaaattcttcgctgttaaaaacctgctgcttctcccaggctcctacacttatgagtggaactttaggaaggatgtgaacatggttctacagagttccctcggtaacgacctgcgggtagatggcgccagcatcagtttcacgagcatcaacctctatgctacttttttccccatggctcacaacaccgcttccacccttgaagccatgctgcggaatgacaccaatgatcagtcattcaacgactacctatctgcagctaacatgctctaccccattcctgccaatgcaaccaatattcccatttccattccttctcgcaactgggcggctttcagaggctggtcatttaccagactgaaaaccaaagaaactccctctttggggtctggatttgacccctactttgtctattctggttctattccctacctggatggtaccttctacctgaaccacacttttaagaaggtttccatcatgtttgactcttcagtgagctggcctggaaatgacaggttactatctcctaacgaatttgaaataaagcgcactgtggatggcgaaggctacaacgtagcccaatgcaacatgaccaaagactggttcttggtacagatgctcgccaactacaacatcggctatcagggcttctacattccagaaggatacaaagatcgcatgtattcatttttcagaaacttccagcccatgagcaggcaggtggttgatgaggtcaattacaaagacttcaaggccgtcgccataccctaccaacacaacaactctggctttgtgggttacatggctccgaccatgcgccaaggtcaaccctatcccgctaactatccctatccactcattggaacaactgccgtaaatagtgttacgcagaaaaagttcttgtgtgacagaaccatgtggcgcataccgttctcgagcaacttcatgtctatgggggcccttacagacttgggacagaatatgctctatgccaactcagctcatgctctggacatgacctttgaggtggatcccatggatgagcccaccctgctttatcttctcttcgaagttttcgacgtggtcagagtgcatcagccacaccgcggcatcatcgaggcagtctacctgcgtacaccgttctcggccggtaacgctaccacgtaagaagcttcttgcttcttgcaaatagcagctgcaaccatggcctgcggatcccaaaacggctccagcgagcaagagctcagagccattgtccaagacctgggttgcggaccctattttttgggaacctacgataagcgcttcccggggttcatggcccccgataagctcgcctgtgccattgtaaatacggccggacgtgagacggggggagagcactggttggctttcggttggaacccacgttctaacacctgctacctttttgatccttttggattctcggatgatcgtctcaaacagatttaccagtttgaatatgagggtctcctgcgccgcagcgctcttgctaccaaggaccgctgtattacgctggaaaaatctacccagaccgtgcagggcccccgttctgccgcctgcggacttttctgctgcatgttccttcacgcctttgtgcactggcctgaccgtcccatggacggaaaccccaccatgaaattgctaactggagtgccaaacaacatgcttcattctcctaaagtccagcccaccctgtgtgacaatcaaaaagcactctaccattttcttaatacccattcgccttattttcgctctcatcgtacacacatcgaaagggccactgcgttcgaccgtatggatgttcaataatgactcatgtaaacaacgtgttcaataaacatcactttatttttttacatgtatcaaggctctggattacttatttatttacaagtcgaatgggttctgacgagaatcagaatgacccgcaggcagtgatacgttgcggaactgatacttgggttgccacttgaattcgggaatcaccaacttgggaaccggtatatcgggcaggatgtcactccacagctttctggtcagctgcaaagctccaagcaggtcaggagccgaaatcttgaaatcacaattaggaccagtgctctgagcgcgagagttgcggtacaccggattgcagcactgaaacaccatcagcgacggatgtctcacgcttgccagcacggtgggatctgcaatcatgcccacatccagatcttcagcattggcaatgctgaacggggtcatcttgcaggtctgcctacccatggcgggcacccaattaggcttgtggttgcaatcgcagtgcagggggatcagtatcatcttggcctgatcctgtctgattcctggatacacggctctcatgaaagcatcatattgcttgaaagcctgctgggctttactaccctcggtataaaacatcccgcaggacctgctcgaaaactggttagctgcacagccggcatcattcacacagcagcgggcgtcattgttggctatttgcaccacacttctgccccagcggttttgggtgattttggttcgctcgggattctcctttaaggctcgttgtccgttctcgctggccacatccatctcgataatctgctccttctgaatcataatattgccatgcaggcacttcagcttgccctcataatcattgcagccatgaggccacaacgcacagcctgtacattcccaattatggtgggcgatctgagaaaaagaatgtatcattccctgcagaaatcttcccatcatcgtgctcagtgtcttgtgactagtgaaagttaactggatgcctcggtgctcttcgtttacgtactggtgacagatgcgcttgtattgttcgtgttgctcaggcattagtttaaaacaggttctaagttcgttatccagcctgtacttctccatcagcagacacatcacttccatgcctttctcccaagcagacaccaggggcaagctaatcggattcttaacagtgcaggcagcagctcctttagccagagggtcatctttagcgatcttctcaatgcttcttttgccatccttctcaacgatgcgcacgggcgggtagctgaaacccactgctacaagttgcgcctcttctctttcttcttcgctgtcttgactgatgtcttgcatggggatatgtttggtcttccttggcttctttttggggggtatcggaggaggaggactgtcgctccgttccggagacagggaggattgtgacgtttcgctcaccattaccaactgactgtcggtagaagaacctgaccccacacggcgacaggtgtttttcttcgggggcagaggtggaggcgattgcgaagggctgcggtccgacctggaaggcggatgactggcagaaccccttccgcgttcgggggtgtgctccctgtggcggtcgcttaactgatttccttcgcggctggccattgtgttctcctaggcagagaaacaacagacatggaaactcagccattgctgtcaacatcgccacgagtgccatcacatctcgtcctcagcgacgaggaaaaggagcagagcttaagcattccaccgcccagtcctgccaccacctctaccctagaagataaggaggtcgacgcatctcatgacatgcagaataaaaaagcgaaagagtctgagacagacatcgagcaagacccgggctatgtgacaccggtggaacacgaggaagagttgaaacgctttctagagagagaggatgaaaactgcccaaaacagcgagcagataactatcaccaagatgctggaaatagggatcagaacaccgactacctcatagggcttgacggggaagacgcgctccttaaacatctagcaagacagtcgctcatagtcaaggatgcattattggacagaactgaagtgcccatcagtgtggaagagctcagctgcgcctacgagcttaaccttttttcacctcgtactccccccaaacgtcagccaaacggcacctgcgagccaaatcctcgcttaaacttttatccagcttttgctgtgccagaagtactggctacctatcacatcttttttaaaaatcaaaaaattccagtctcctgccgcgctaatcgcacccgcgccgatgccctactcaatctgggacctggttcacgcttacctgatatagcttccttggaagaggttccaaagatcttcgagggtctgggcaataatgagactcgggccgcaaatgctctgcaaaagggagaaaatggcatggatgagcatcacagcgttctggtggaattggaaggcgataatgccagactcgcagtactcaagcgaagcgtcgaggtcacacacttcgcatatcccgctgtcaacctgccccctaaagtcatgacggcggtcatggaccagttactcattaagcgcgcaagtcccctttcagaagacatgcatgacccagatgcctgtgatgagggtaaaccagtggtcagtgatgagcagctaacccgatggctgggcaccgactctccccgggatttggaagagcgtcgcaagcttatgatggccgtggtgctggttaccgtagaactagagtgtctccgacgtttctttaccgattcagaaaccttgcgcaaactcgaagagaatctgcactacacttttagacacggctttgtgcggcaggcatgcaagatatctaacgtggaactcaccaacctggtttcctacatgggtattctgcatgagaatcgcctaggacaaagcgtgctgcacagcacccttaagggggaagcccgccgtgattacatccgcgattgtgtctatctctacctgtgccacacgtggcaaaccggcatgggtgtatggcagcaatgtttagaagaacagaacttgaaagagcttgacaagctcttacagaaatctcttaaggttctgtggacagggttcgacgagcgcaccgtcgcttccgacctggcagacctcatcttcccagagcgtctcagggttactttgcgaaacggattgcctgactttatgagccagagcatgcttaacaattttcgctctttcatcctggaacgctccggtatcctgcccgccacctgctgcgcactgccctccgactttgtgcctctcacctaccgcgagtgccccccgccgctatggagtcactgctacctgttccgtctggccaactatctctcctaccactcggatgtgatcgaggatgtgagcggagacggcttgctggagtgccactgccgctgcaatctgtgcacgccccaccggtccctagcttgcaacccccagttgatgagcgaaacccagataataggcacctttgaattgcaaggccccagcagccaaggcgatgggtcttctcctgggcaaagtttaaaactgaccccgggactgtggacctccgcctacttgcgcaagtttgctccggaagattaccacccctatgaaatcaagttctatgaggaccaatcacagcctccaaaggccgaactttcggcttgcgtcatcacccagggggcaattctggcccaattgcaagccatccaaaaatcccgccaagaatttctactgaaaaagggtaagggggtctaccttgacccccagaccggcgaggaactcaacacaaggttccctcaggatgtcccaacgacgagaaaacaagaagttgaaggtgcagccgccgcccccagaagatatggaggaagattgggacagtcaggcagaggaggcggaggaggacagtctggaggacagtctggaggaagacagtttggaggaggaaaacgaggaggcagaggaggtggaagaagtaaccgccgacaaacagttatcctcggctgcggagacaagcaacagcgctaccatctccgctccgagtcgaggaacccggcggcgtcccagcagtagatgggacgagaccggacgcttcccgaacccaaccagcgcttccaagaccggtaagaaggatcggcagggatacaagtcctggcgggggcataagaatgccatcatctcctgcttgcatgagtgcgggggcaacatatccttcacgcggcgctacttgctattccaccatggggtgaactttccgcgcaatgttttgcattactaccgtcacctccacagcccctactatagccagcaaatcccgacagtctcgacagataaagacagcggcggcgacctccaacagaaaaccagcagcggcagttagaaaatacacaacaagtgcagcaacaggaggattaaagattacagccaacgagccagcgcaaacccgagagttaagaaatcggatctttccaaccctgtatgccatcttccagcagagtcggggtcaagagcaggaactgaaaataaaaaaccgatctctgcgttcgctcaccagaagttgtttgtatcacaagagcgaagatcaacttcagcgcactctcgaggacgccgaggctctcttcaacaagtactgcgcgctgactcttaaagagtaggcagcgaccgcgcttattcaaaaaaggcgggaattacatcatcctcgacatgagtaaagaaattcccacgccttacatgtggagttatcaaccccaaatgggattggcagcaggcgcctcccaggactactccacccgcatgaattggctcagcgccgggccttctatgatttctcgagttaatgatatacgcgcctaccgaaaccaaatacttttggaacagtcagctcttaccaccacgccccgccaacaccttaatcccagaaattggcccgccgccctagtgtaccaggaaagtcccgctcccaccactgtattacttcctcgagacgcccaggccgaagtccaaatgactaatgcaggtgcgcagttagctggcggctccaccctatgtcgtcacaggcctcggcataatataaaacgcctgatgatcagaggccgaggtatccagctcaacgacgagtcggtgagctctccgcttggtctacgaccagacggaatctttcagattgccggctgcgggagatcttccttcacccctcgtcaggctgttctgactttggaaagttcgtcttcgcaaccccgctcgggcggaatcgggaccgttcaatttgtagaggagtttactccctctgtctacttcaaccccttctccggatctcctgggcactacccggacgagttcataccgaacttcgacgcgattagcgagtcagtggacggctacgattgatgtctggtgacgcggctgagctatctcggctgcgacatctagaccactgccgccgctttcgctgctttgcccgggaacttattgagttcatctacttcgaactccccaaggatcaccctcaaggtccggcccacggagtgcggattactatcgaaggcaaaatagactctcgcctgcaacgaattttctcccagcggcccgtgctgatcgagcgagaccagggaaacaccacggtttccatctactgcatttgtaatcaccccggattgcatgaaagcctttgctgtcttatgtgtactgagtttaataaaaactgaattaagactctcctacggactgccgcttcttcaacccggattttacaaccagaagaacaaaacttttcctgtcgtccaggactctgttaacttcacctttcctactcacaaactagaagctcaacgactacaccgcttttccagaagcattttccctactaatactactttcaaaaccggaggtgagctccacggtctccctacagaaaacccttgggtggaagcgggccttgtagtactaggaattcttgcgggtgggcttgtgattattctttgctacctatacacaccttgcttcactttcctagtggtgttgtggtattggtttaaaaaatggggcccatactagtcttgcttgttttactttcgcttttggaaccgggttctgccaattacgatccatgtctagactttgacccagaaaactgcacacttacttttgcacccgacacaagccgcatctgtggagttcttattaagtgcggatgggaatgcaggtccgttgaaattacacacaataacaaaacctggaacaataccttatccaccacatgggagccaggagttcccgagtggtacactgtctctgtccgaggtcctgacggttccatccgcattagtaacaacactttcattttttctgaaatgtgcgatctggccatgttcatgagcaaacagtattctctatggcctcctagcaaggacaacatcgtaacgttctccattgcttattgcttgtgcgcttgccttcttactgctttactgtgcgtatgcatacacctgcttgtaaccactcgcatcaaaaacgccaataacaaagaaaaaatgccttaacctctttctgtttacagacatggcttctcttacatctctcatatttgtcagcattgtcactgccgctcacggacaaacagtcgtctctatcccactaggacataattacactctcataggacccccaatcacttcagaggtcatctggaccaaactgggaagcgttgattactttgatataatctgtaacaaaacaaaaccaataatagtaacttgcaacatacaaaatcttacattgattaatgttagcaaagtttacagcggttactattatggttatgacagatacagtagtcaatatagaaattacttggttcgtgttacccagttgaaaaccacgaaaatgccaaatatggcaaagattcgatccgatgacaattctctagaaacttttacatctcccaccacacccgacgaaaaaaacatcccagattcaatgattgcaattgttgcagcggtggcagtggtgatggcactaataataatatgcatgcttttatatgcttgtcgctacaaaaagtttcatcctaaaaaacaagatctcctactaaggcttaacatttaatttctttttatacagccatggtttccactaccacattccttatgcttactagtctcgcaactctgacttctgctcgctcacacctcactgtaactataggctcaaactgcacactaaaaggacctcaaggtggtcatgtcttttggtggagaatatatgacaatggatggtttacaaaaccatgtgaccaacctggtagatttttctgcaacggcagagacctaaccattatcaacgtgacagcaaatgacaaaggcttctattatggaaccgactataaaagtagtttagattataacattattgtactgccatctaccactccagcaccccgcacaactactttctctagcagcagtgtcgctaacaatacaatttccaatccaacctttgccgcgcttttaaaacgcactgtgaataattctacaacttcacatacaacaatttccacttcaacaatcagcatcatcgctgcagtgacaattggaatatctattcttgtttttaccataacctactacgcctgctgctatagaaaagacaaacataaaggtgatccattacttagatttgatatttaatttgttctttttttttatttacagtatggtgaacaccaatcatggtacctagaaatttcttcttcaccatactcatctgtgcttttaatgtttgcgctactttcacagcagtagccacagcaaccccagactgtataggagcatttgcttcctatgcactttttgcttttgttacttgcatctgcgtatgtagcatagtctgcctggttattaattttttccaacttctagactggatccttgtgcgaattgcctacctgcgccaccatcccgaataccgcaaccaaaatatcgcggcacttcttagactcatctaaaaccatgcaggctatactaccaatatttttgcttctattgcttccctacgctgtctcaaccccagctgcctatagtactccaccagaacaccttagaaaatgcaaattccaacaaccgtggtcatttcttgcttgctatcgagaaaaatcagaaatccccccaaatttaataatgattgctggaataattaatataatctgttgcaccataatttcatttttgatataccccctatttgattttggctggaatgctcccaatgcacatgatcatccacaagacccagaggaacacattcccccacaaaacatgcaacatccaatagcgctaatagattacgaaagtgaaccacaacccccactactccctgctattagttacttcaacctaaccggcggagatgactgaaacactcaccacctccaattccgccgaggatctgctcgatatggacggccgcgtctcagaacaacgacttgcccaactacgcatccgccagcagcaggaacgcgtggccaaagagctcagagatgtcatccaaattcaccaatgcaaaaaaggcatattctgtttggtaaaacaagccaagatatcctacgagatcaccgctactgaccatcgcctctcttacgaacttggcccccaacgacaaaaatttacctgcatggtgggaatcaaccccatagttatcacccaacaaagtggagatactaagggttgcattcactgctcctgcgattccatcgagtgcacctacaccctgctgaagaccctatgcggcctaagagacctgctaccaatgaattaaaaaaaaatgattaataaaaaatcacttacttgaaatcagcaataaggtctctgttgaaattttctcccagcagcacctcacttccctcttcccaactctggtattctaaaccccgttcagcggcatactttctccatactttaaaggggatgtcaaattttagctcctctcctgtacccacaatcttcatgtctttcttcccagatgaccaagagagtccggctcagtgactccttcaaccctgtctacccctatgaagatgaaagcacctcccaacacccctttataaacccagggtttatttccccaaatggcttcacacaaagcccagacggagttcttactttaaaatgtttaaccccactaacaaccacaggcggatctctacagctaaaagtgggagggggacttacagtggatgacactgatggtaccttacaagaaaacatacgtgctacagcacccattactaaaaataatcactctgtagaactatccattggaaatggattagaaactcaaaacaataaactatgtgccaaattgggaaatgggttaaaatttaacaacggtgacatttgtataaaggatagtattaacaccttatggactggaataaaccctccacctaactgtcaaattgtggaaaacactaatacaaatgatggcaaacttactttagtattagtaaaaaatggagggcttgttaatggctacgtgtctctagttggtgtatcagacactgtgaaccaaatgttcacacaaaagacagcaaacatccaattaagattatattttgactcttctggaaatctattaactgaggaatcagacttaaaaattccacttaaaaataaatcttctacagcgaccagtgaaactgtagccagcagcaaagcctttatgccaagtactacagcttatcccttcaacaccactactagggatagtgaaaactacattcatggaatatgttactacatgactagttatgatagaagtctatttcccttgaacatttctataatgctaaacagccgtatgatttcttccaatgttgcctatgccatacaatttgaatggaatctaaatgcaagtgaatctccagaaagcaacatagctacgctgaccacatccccctttttcttttcttacattacagaagacgacaactaaaataaagtttaagtgtttttatttaaaatcacaaaattcgagtagttattttgcctccaccttcccatttgacagaatacaccaatctctccccacgcacagctttaaacatttggataccattagagatagacattgttttagattccacattccaaacagtttcagagcgagccaatctggggtcagtgatagataaaaatccatcgcgatagtcttttaaagcgctttcacagtccaactgctgcggatgcgactccggagtttggatcacggtcatctggaagaagaacgatgggaatcataatccgaaaacggtatcggacgattgtgtctcatcaaacccacaagcagccgctgtctgcgtcgctccgtgcgactgctgtttatgggatcagggtccacagtttcctgaagcatgattttaatagcccttaacatcaactttctggtgcgatgcgcgcagcaacgcattctgatttcactcaaatctttgcagtaggtacaacacattattacaatattgtttaataaaccataattaaaagcgctccagccaaaactcatatctgatataatcgcccctgcatgaccatcataccaaagtttaatataaattaaatgacgttccctcaaaaacacactacccacatacatgatctcttttggcatgtgcatattaacaatctgtctgtaccatggacaacgttggttaatcatgcaacccaatataaccttccggaaccacactgccaacaccgctcccccagccatgcattgaagtgaaccctgctgattacaatgacaatgaagaacccaattctctcgaccgtgaatcacttgagaatgaaaaatatctatagtggcacaacatagacataaatgcatgcatcttctcataatttttaactcctcaggatttagaaacatatcccagggaataggaagctcttgcagaacagtaaagctggcagaacaaggaagaccacgaacacaacttacactatgcatagtcatagtatcacaatctggcaacagcgggtggtcttcagtcatagaagctcgggtttcattttcctcacaacgtggtaactgggctctggtgtaagggtgatgtctggcgcatgatgtcgagcgtgcgcgcaaccttgtcataatggagttgcttcctgacattctcgtattttgtatagcaaaacgcggccctggcagaacacactcttcttcgccttctatcctgccgcttagcgtgttccgtgtgatagttcaagtacagccacactcttaagttggtcaaaagaatgctggcttcagttgtaatcaaaactccatcgcatctaattgttctgaggaaatcatccacggtagcatatgcaaatcccaaccaagcaatgcaactggattgcgtttcaagcaggagaggagagggaagagacggaagaaccatgttaatttttattccaaacgatctcgcagtacttcaaattgtagatcgcgcagatggcatctctcgcccccactgtgttggtgaaaaagcacagctaaatcaaaagaaatgcgattttcaaggtgctcaacggtggcttccaacaaagcctccacgcgcacatccaagaacaaaagaataccaaaagaaggagcattttctaactcctcaatcatcatattacattcctgcaccattcccagataattttcagctttccagccttgaattattcgtgtcagttcttgtggtaaatccaatccacacattacaaacaggtcccggagggcgccctccaccaccattcttaaacacaccctcataatgacaaaatatcttgctcctgtgtcacctgtagcgaattgagaatggcaacatcaattgacatgcccttggctctaagttcttctttaagttctagttgtaaaaactctctcatattatcaccaaactgcttagccagaagccccccgggaacaagagcaggggacgctacagtgcagtacaagcgcagacctccccaattggctccagcaaaaacaagattggaataagcatattgggaaccaccagtaatatcatcgaagttgctggaaatataatcaggcagagtttcttgtagaaattgaataaaagaaaaatttgccaaaaaaacattcaaaacctctgggatgcaaatgcaataggttaccgcgctgcgctccaacattgttagttttgaattagtctgcaaaaataaaaaaaaaacaagcgtcatatcatagtagcctgacgaacaggtggataaatcagtctttccatcacaagacaagccacagggtctccagctcgaccctcgtaaaacctgtcatcgtgattaaacaacagcaccgaaagttcctcgcggtgaccagcatgaataagtcttgatgaagcatacaatccagacatgttagcatcagttaaggagaaaaaacagccaacatagcctttgggtataattatgcttaatcgtaagtatagcaaagccacccctcgcggatacaaagtaaaaggcacaggagaataaaaaatataattatttctctgctgctgtttaggcaacgtcgcccccggtccctctaaatacacatacaaagcctcatcagccatggcttaccagagaaagtacagcgggcacacaaaccacaagctctaaagtcactctccaacctstccacaatatatatacacaagccctaaactgacgtaatgggactaaagtgtaaaaaatcccgccaaacccaacacacaccccgaaactgcgtcaccagggaaaagtacagtttcacttccgcaatcccaacaagcgtcacttcctctttctcacggtacgtcacatcccattaacttacaacgtcattttcccacggccgcgccgccccttttaaccgttaaccccacagccaatcaccacacggcccacactttttaaaatcacctcatttacatattggcaccattccatctataaggtatattattgatgatg